SEQUENCE LISTING (14 sequences)

SEQ ID NO: 1 - DNA, 17 bp, Artificial Sequence
  Description of Artificial Sequence Synthetic primer
gactgcgtaccaattca

SEQ ID NO: 2 - DNA, 17 bp, Artificial Sequence
  Description of Artificial Sequence Synthetic primer
gatgagtcctgaccgaa

SEQ ID NO: 3 - DNA, 17 bp, Artificial Sequence
  Description of Artificial Sequence Synthetic primer
gatgagtcctgaccgac

SEQ ID NO: 4 - DNA, 24 bp, Artificial Sequence
  Description of Artificial Sequence Synthetic primer
tttggagcagtgacaggatcagac

SEQ ID NO: 5 - DNA, 24 bp, Artificial Sequence
  Description of Artificial Sequence Synthetic primer
agagagcctgcgtccttatctcac

SEQ ID NO: 6 - DNA, 25 bp, Artificial Sequence
  Description of Artificial Sequence Synthetic primer
aaactgtccttcaaggtagtcaaca

SEQ ID NO: 7 - DNA, 25 bp, Artificial Sequence
  Description of Artificial Sequence Synthetic primer
ggggcactagagtgggttgccattt

SEQ ID NO: 8 - DNA, 487 bp, Artificial Sequence
  Description of Artificial Sequence Synthetic polynucleotide
tttggagcagtgacaggatcagactgagttttcagaaagcatcagtgtggcggctgggagcaacatgggccgcagccacccagaacagaaaccagaggttcagacagaaggctctcgtggtgcttcgggcgcagctgacactgaatcagaggtatgggcgctagaattcagtgggctggtgggagggcagggtccccggctgatggactgggacacgggagtcaccgaaaggcactcaagggtgactgcgggttttgcaggaacatctggggggcgggaaatggagcctgggtttgcatggccatattaaatcttgagatggtctgggaaagagtcgggaaaggtcactggggtaggttgagaaaccgagaaaacgtcgagtcatggaaactatgaaggagaatgtttccagaaggagggaggaggcgcatgcgtggttggtgagaaggctgctgagaagcagtgtgagataaggacgcaggctctc

SEQ ID NO: 9 - DNA, 15 bp, Artificial Sequence
  Description of Artificial Sequence Synthetic oligonucleotide
gagaaaatgtcgagt

SEQ ID NO: 10 - DNA, 15 bp, Artificial Sequence
  Description of Artificial Sequence Synthetic oligonucleotide
gagaaaacgtcgagt

SEQ ID NO: 11 - DNA, 397 bp, Artificial Sequence
  Description of Artificial Sequence Synthetic polynucleotide
aaactgtccttcaaggtagtcaacaaatatttaggaattcaccctcatgtatactttcttcaattttgcatagaagtttataggaatgacttggtatcagagaaccttgaattcagttcctaattctggctcttctgaagtttgagatcctaaggaagaaatgtaaccactctgcacattagttttcacttctgtaacatgaaatagcagagtttgctgttagaataaaatgacatattattggtaaagtgcctcaaagtaattggcacagggttagagttcagtcacttccctggtggctcagacggtaaagcgtctgcctgcagtgcaggaaacccgggttcgatccctgggttgggaagatcccctggagaaggaaatggcaacccactctagt

SEQ ID NO: 12 - DNA, 19 bp, Artificial Sequence
  Description of Artificial Sequence Synthetic oligonucleotide
aaacccgagttcgatccct

SEQ ID NO: 13 - DNA, 19 bp, Artificial Sequence
  Description of Artificial Sequence Synthetic oligonucleotide
aaacccgggttcaatccct

SEQ ID NO: 14 - DNA, 141032 bp, Bos taurus
atcaaggttgaggcttggtcaagatgagggctcagaggagcctggctaaggacttccctggcagtccagtggttaggactctccactttcactgctgagggtcccagttcagtgcctggtcggggaactaagatctcacaagccgtgctgtctggctgaaaaaaaacaaacccagagacttctccagcttaagactccctgctgtccaatgcagggcacctgggtttgatccctagtcagggagctgaattcaacatgaccctgagcaaactccaggagatagggaaggctggtgtgctgtagtccaaggtcaccaagagttagacgtgacttagccactgaacaacagcagtaaagggaactagatcccgtaggatgcaactaaagcttccacatgccagaattaagacctggagcagccaaacaaacaaatattttaaaaatacaaaaagcagaggggcctgactagactaggtcaagaagagcatctttgtccatgggcaagttaaaagtttgcataatttttgtcaaagtacaaaattcacaccagaggacttaactcatactggagattctgagaaagcagagctttcatctggctgtagggatgtcagtggtctttgaagggtgacgtgattgaaccacaaagctcctatagtttttctgcccaatttctgcatagcagttgagggagcttttctactgcttagacaccttttcatttacgtacagtggttttgcaatagaacagactctatggttttacgcctgagaggtctttgatttcaccaaagcagacttcttcattgaaagtgttggagctattttgttctgaattaatttgatagaaaccagggatttgttctgaccttgaaataattgtcctctctcaggtaatttcagatgatatcgtcagaaacactcttaagaataattccctaaatgaaaattaatggagtgggccaagcgatcactaaaaaatagttttaattcaaactgatatttgtataccagagccggttggagaaggcaatggcaccctactccagtgctcttgcctggaaaatcccatggacggaggagcctggtaggctgcagtccatggggtcgctaagagtcagacacgactgagcaacttcactttcactttcatgcattggagaaggaaatggcaacccactccagtgttcttgcctggagaatcccagggacgggggagcctggtgggcgccatctgtggagtcgcacagagtcggacacgactgaagcgacttagcagcagcagcagccaaaaagtagaaataacccgtcagcagatgaatggataaacacagtgaggtctatacacatgcagtggattatgactcagccttgaaaaggaaggaaattctgacacacgctatatcatggatgaaccttaacagcattatgctaaggggaataagtcagtccaaaaggacagtattgcatgattccacttatatggggtacccaggagtggtccagtccacagagacagaaaagagaaagatagttactagaagctgggggaggagggactgggcttggtgtgtaatggggactgagtttcactctgtgaagatgaaatagttctgatgaatggtgatcatagttgcccaacagtagcagtgaatgtcctgaatgcccttgtcctgtacccttaaaaatgttttatgtggtaaattttgtcaaatacattttaccaaaattttataacaaataaatcttgctcttagatattaaagtcatagcacaaagggtggtgaacaaaagctaaaccttgaaatcagagaagcgttcacgcattttccccacagagtgagaaaactgaccaggaatacctttagcagtgtttacagctccgcctgccttgcaagagacccctccactcagggcgcctcctctgaaagggcccgtggctcagggtcgccctctgcagaaatgtcctcgctgggaggagggcctccaggggcctctaaccagtcagttactctcggccctggtcgccttccgtcattccacgtgttcattgaacatcaagtatgtgcgtccacagtggggaatcctaaagagtgacgccgcccccgcccacagccagcctgcattctagttgggggaaggagatgaggcagcctgcagcttaaccccgtgaggccagatcatggaaccggcttaaggaatcagatgtaagatggtcacgggaggctgcatggcatctgccctcagatctcttcatggcctaggactcttgttgctgtggggcttcccgggtggtgctagtggtaaagagcctgcctgccaatgcaggaagcaggagatccggaaagattcccctggaggagggcacggcaacccactccagtattcttgcctggaaaatccctgtgacagaggagccaggcgggctacagtccacggggtcgcaaagagtcagacacgaccaaagtgacttagcacgcacagcaactggttgccttttacccttttttaaaaagtatttatttctttatttcctgtgggcatcgagtcttagttgcagcacgcgggatctttcgctgcagcgcacaggcaccttaggtacagcactgaggcgttgtcaccctgcagcacttgggatcttggttccctgagcagggatggaacccccgtcccctgcattagaaggcaggttcctaaccactgcatggctggcaggaaggtcctgccttttactcttgaacgtgaatgttcacataacagaccttggattggcatttataactccaagcaggttgaggtggctggagactggccttccccgaggctggtcagtccctccgaatgatatcccggctacagccatgggggagctggccctgcgacagacagcttcatgcagagtgcaggtgctttcccgctttgttccttcagtacttgatctacaagcgtctgcaaatcctgggcttaaaaataaaaaaccaaaagacatcatctggccaactggcttttgacttttcgaataacaaagttgcctttgtggtttaattggcttaacttctataactgacaaatagaagggggaaaaatatgtgttttgaaaacctatttccaagtaattccagccatttgggacgtagggaagttgtctgagctgtctgggggaaattcccggagctaagaattcgcatttggcgttgtctgtagctcgacacgggctcctgaggccaccagcttcgtgtctgaggagattcagtcgttatgccaactattgcttaaatacttgctgaaagtctggggtgagaagtaggtaaatgtgggaaaggactgggagtctgataaacagggacctaaggatgtgggccatcacagggtacgtttgcttttatttcactgagatcctttatattattatattcagaagactcccagagctggaaacctttgatttgttagcagatagaacctgtttggtttctgtatttgaattcttgtcttagccttgtttcctcccactttggagtaattagctgtgctttaattttcagaaatcgcccctggtggatgtccccgtgttgcagttttgctatgcgttcatccaaaggtaacgcagctccctcaaaggcatttctcaattatagatgctgttgcagtgattttaagtaactactctggattgtcctcctttttttcttttaactgaagtgctgttgatttacagtgtcgtgttagtttcaggtgtacggcacagtgactcagtttgatataggtgtgtgtgtgtgtgtgtgccttttcaaatgctttttcattacaagttataagatgttgcgtatagttccctgtgctttacagtgggtccttattgtttacctgttttgtatacagtagtgtgtctgtgttaatcccaacttattctttatccctccctgcctccacccgtgtgtttcttgattactggagagccttagttcagaaagtatctgtgtgttactaacccagccgtgtccaactctttgtgaccccatggactgtagcctgccaagctcctctgtctgtgggattctccaggcaagaatactggagtggcgttgccattctcctcctccaggggatcttgccgacctggggatctgagaggtgtcctaattccctctcaggccatcggacaccccgccagtctcactttttccttctgtagagagtgaatctgacatcttcaagtctacgtgctgtatcttgaacactttgtcagcacagctttgtgtgagacctggtaaacttcagaataaacagtttgcattaccttgataaccaaaaaaaagaggaattttaagaacctcttttaggaaaagaagtgactgttttggatgctctcctcagtgctttgctgtgttttctgctgggcggcttcatgtcgatggactagagtttgagcaagctctgggggttggtgatggacagggaagcctggcgtgctgcagtccatggggttgcaaagagacacgactgagcgactgaactgaactgaggtgtttctgcgtgggcttactgagctgaacacccatctgcaggggtctcagctgcagtggttccccaggtggctctctacagggagagaggacgctttgctggcctcctgcggctcttacggcaggtcctggatacagctcccagatgcgtccactgtgcggggaggttagagctgcagtaacgggagtgaagtgggtgtagccctatctcacggatgcctcgatagcctcacgtgtctcaggggattcacctttagtaaatttgaccaaactccctggctaacaagttcacattccatggttattctgacccatgacaccctagggttatgggaaggtggatggagggtcctagtcccaggagtcccctggcccaggatcgagttcaggccagccattcaccctccagcacgagcatcactgctcttttttgctccaggaggggaagatgagctggtcctttgctgttgttcagtcactcagtcgtgtccaactctttgagaccccatggactgcagcacaccaggcctccctgtccttcactatcacccagagtttactcaaactcatgtccattgagtcggtgatgccatccagccatctcatcctctgtcgtccccttctctcctgccttcaatctttcccagcatcaggatcttttcaaatgagtcagttcttcacatcaggtgaccaaagtattggagcttcagctttagcatcagtccttccaatgaatattcaggattgatctcctttaggattgactggtgtgatgtccaagggactctcaagggtcttctccaacaccacagttcaaaagcatcaatccttcggcgctcagccttccttatggtccaactctcacatccatacatgaccacaggaaaaaccatagccttgactagacggacctttgttgttcaggtcatgtctcagctttttaatacactgtctaggttgatcatagctttttttgcaagaagcaggcatcttttaatttcatggctgcagtcaccatcctcagtgattttggagcccaagaaaataaagtctgtcactgcttccacttttccccatgtccttcctagtgctcactcatacgtgtcataatcccagcaagtgcctgctgcaatggcctgagtgacatcgtccagcggcctcagggaggctgccttctttcagggaggaggccccacccaggaccctcaggatgccgagagtcagggaccagcactcggccctgggacccgctggctgcggcccagctgcctccctgtgtggtgcagcgtcaccccacggcaacaaccccgagtcctcattccccatgaggcctgacctgagagctactaaagatcagcctcctgctgggtgacccctgacctcaccttctgtgacctcccgatggctcactctgcatttccagtgatgttagggaggccaaatgctaaatggagcccgacaggcccagagatccgtcatccagttcaacgctttcactttatagacgaggaaaccaggagtgtctgacctgtttagacagatggctggtatccaaacctccaagttcttgccaaaataccacaatttaacgcatatgtattttaatttaagaagaacaaaacaggtcaggcagataaggcatcgtcctgtgccttctaaccgcctgcgcctaaaatgtgcttataataaaacattttcattagacctgttcatggctggatgtttttttccctactattttttttgttgttgtctctttgtttttattaataggctcccagcaacagccttgcaagagaacttcccctcgctgttgggcgtgttgaaggagtccgtgcagttgaatctcgctccccccggttactttctgcttctcaggtaccgtgttcccagctactcaacgttaccgcgttttttttcttctccaggaacatgtatgttttcctttctgctagcattctcattctgaagccaattgtctctcaacagcatgctgaatgactttgtgacaagaactccaaacctggagagcaagaaggaccaaaaagatctgcaggtctgtatgtgcgtgtccagtaggatggttcctctcccaggagagcattgcccgccccccaccttcagacaatctcactggagatgataaacattaaacatggggcagaaccaggtcttaggtttgcatgtgttcccacatctgactccctgcatcactgtcttcagaggactcgatagagtcttcagatgatggttctcatgtcagccttgagccatccaccttgcaattttcaaggcgcctgctagcttcctcctggaccccgatgtgtgcaggctcactcttcagcatggctggagcagtaaatctgtctttatacacagattcccttccttagaaccttcccccccttctttttaaagcaattcagacttatgggaaacttcaaaaacactgaaaagcaaaagtaaagctcagccactggagaaggaaaatctcctttgagtagaaacccagccaataagtaggatggagtgagaaaaaccagtatgttataacctttataatcatcgagtgaggagcttttcgagtcaaggccttaacttctttttcagtgtcttttattcatgtatttttgttgttggcatgtggttgcctcaacatgttgagttagtttctgctgtagagcatggtgaattcagctgcacatttacatatgtcccttgcatttccttcctttgagtcaaggtcattaaactgtgctgtgtatctcatggccttggcgatactgccgctccctgttgtgacccctggcatttgtgagaccctgaagctctggagctctgcgtaaagcccagtgactcctttggctcccctggtggaggggaaagctccatcccagcgcacgtgctgcagcccagcccgtggtcagcagttcctatgccaagctgaaagccctccatctcgtgcggccagtggcctgccacggccccgtcccctccagtcctccaggccgagctttggccgtggccagtaaggcggctggaggagctcgatgggcccttgcgtccaccccagagggtcccagccctcctgacactgaccccttcctactgcttccaggcaccccaacttccccatctgctcactgccctcgaaatggcagcttctcttaagccctcaaccccttgtcttgatgtttttttcatatttattttactgaagcgtaagcagtgcgtgttaacgtctgctgtacagcacagtggctcagtttgcacgtatgtgcattctttctcctcttagtttccattatggttcatcgcaggatattgaatacagctcccctctgctctacagcaggacctctgtctgtccatcctatatatgtaatagtttgcatctgctgatcccagcctcctgctctgtccctcctgcgccccttcccccttagcagccacaagtctgtcctgtgtgtctgtgagtctgctttctgtttcctaggtaggttccgttgtgttgtatcatagattccacatgtcagtgttatcatctgacatgtgtctttctctgcctggcttcgcttagcatgatcatccccagatccatccgtccttccacaatctcccttgctgccagcgtccacgcctgcccctctgcccgggtgcaccgtggacggttccctcgggtgcgctgcccccgtccctcccaccagggagggacctactcttgcttcatgcccgaacacacctgctgcactcacagcagaagccaggactgcttacgtgtcagggacgtgtagcccctcttagctgatgataccccatgatacctgctccgcgtctccacttttgtcccggcttcttgaccggattctgtgctgagagcactcctatgctcttcaccctcaagtcagtttgaacttactcagttttgtttctggccgtgttcgtcgacggtgcatttctagtacccgtggtcctaaagaaaccagaaatccaacctgccaaatgcgttgtgccgctgtgacttcctgtgtatccgttttacctactgaccatagagcagccacagctcaacgtatggctgctgagcccttgtaatgtggcttgcctgaactgaacgtgccgtaagcataaaacacacatcaggtttaaaagacttagaccaaagaagcgtgccgaagctcactaataattttttatcggttatatgttgagactactttggatgtatcaagatgcacactgtgaacttgtttcttcttttaaggtagctgctcgaagacttcattacctgtatgcttcatagccgtgacttgcattatacttctgtcggatggtgctaccttaggcacttaagactctcttaacttcctttcctcttttctctcggttcctctcaggaagtcactcaaaaaattctggaagctgttgggaacattgctgggtcttccttagaacaaaccagctggctgagcaggaacttggaagtgaaggcccagcctcagatctctctagaagaatccgatgctgaggaagacttgcacggtgggtgtatatggctcagaaagtaatgcctgctgccaggttgcaggtctctctctgcattaaaaatcatctggctttgcttcttacaaaataagttgtagtatcataaacataagttgtagtaaaatgaacagtagaattccactggaaggatcccaagtgacccatgctactgtgttcagccttccatgagcctagatgagggtcctgggagataacttcccaagataatttgcatggactcagtggtagaaatctttgtgaggtaaagaatagggacaatgcatgtgtttttttttgttgttgtttgtttgttttatggagccgtggaaagaaaacttagtaaaccagctcaccgttgtcaagcagttgatgtatttcatttctggcctgccttccagaaatccttgtatttggtcattgcccaggtcattaagattttatcacggcagtcacacacagtaggcacaccccagccttattattagttataacttcctaggcaaatggtcgggagactctcaaaggagactcatctgtttatttggaggaccactgaccagccagaaacccccgaggggtctgaattaaatgccacagtgtggactgtcagttgctggtgccctaaagaccaccgggtgaatctgtcagtcaggcacatctcaggttattggatccgctgcagcaagggagatgcttggctgagtcagaggggtgtctcagcaggagacacttggggaagatggttgggggctgtggggcataggttgagttgaatgtagcagatgtttcaaggtgggtactgggtggagttagtgatcagatacctctgcactggtgggcacagtgctaggcgggggcccacaagtgaatcttcatcaacatgctgcttgttttggtaagtgagccgctttagttggtttagtctttctttcaggattaggcatatcttggagcaagtattaataattactgttgcaagttctcagtattatttaatacacagacactatgcccagtcccgacattgctcagcactagacaagaaaattgggctcccctgagagctcagctggcaaagaatccgcctgcaatgcaggagaccccagttcgattcctgggtcgggaagatccactggaggagggataggctacccactctagtattcttgggcttccctggtggctcagctggtaaagaatccccctgcgatgtgggagacctgggttcgatccctgggttgggaggatcccctggagaagggcaaggctgcccactccagtgttccggcctggagaagtccatgggctgaacagtcatggggtcgcaaagagctgggcacagcagcgctggacacgactgagcgccttgcacttcactttcacctttaccttccctcccattctgatgcgacctcgatcattatcattagcagttgttgtagcagcagaaacaacctgaatccatgggttttaacgtacaaaggacggcgtgtcaagaaatctgggctcttttctcttgcaagtagcagaaacccactccaggaggacagaagcgcagcagcacagggagttctggctcacacgcatgaaatgcccacgcgtgctctccgtgtctggttttgagggcagagtgggaaccaggtgttcagacgcagttcatcaaatcttgtgcgggcgttctggttggttcttcctggtctgcggctggcccagtagcgggcaggaagggctcgtgtcctggagccccgagaagcggtctcccacccaagaggggcatctcttcccaggaggagaagggatggcgttggacagacgtttaaatcatggtgccccgttccatcacttccaccgtatttatttgacagctagctctgcggtttatgtaagaaaaataattgcactttactctgtattgaagatcctgcagctagtgtgctatcgtttgctaacgtaaaccaacaattatgtcttctcaggctatggctctgattataggtgtggttccagggaaggagagtctgtgcctctccttgactcctttgctttcagttactctgccgtagtttgcggtgtaagggttaaactgcctctttaatcctggcgtgctgcagtccgtggggccgcagagagtcagacatgactgagtgactgaacagccaccaacagttcagctgaactgtgtcaaatgttactgggcctgacttttttgccatctttagatttaacctcaagacagtaatgtagtaaaataattatctgatttttttatttaagaaaagaaatcgtaggacttcccgttggttcagtggttaagactcggcgctcccaatgcaggacgcatgggtctggtgcctggttgggcaaccaagaccctacagtgcgacataaaaagaaaaaaaaaatcatagaatttcaaagcagaaatgacttggaggcttcttaatgagcaatttgggtaaactccgggagttggtaatggacagggagggctgacgtgctgcagtccatggggtcgcaatgagtcggacacgacagagcgactgaactgaactaattataattagcaaactgtagagtatcaaaagagtctctggatcataactattttttatttcagtcttgaacaaactaaggatgctggccatatcaagcatgaaacagaatgaggcttcaaatgatgtgcttttttcggcttggagtctttatgcatgtagcagacatgcagggtcttccgtgtatatttgagtctggtgtgtgctacgcaccgtgttctagacactggtgctccagcagcaaacagaatgaacagggtctttgctgttgtgggggtgacatggaaggtgatggtccagagtaaattaacaaaggtatctttaagttcttctggaaatgcaaagtcattccaaatttatatatatatatatatatgtattttttttttaatgggagtggggcagggttcactttagatgttacagaacatgtcccccacggaactgacagttgtgcagagaaaggaaagataaagtggggtgggcggggacttcctcggtggtccagtggttgcgtgtccatctgccagtgcagggggcagggattccatccctggtcagggagcttaagagcccacctgcctcaggacaactaagcctgtgcaccacaactgccgaagcctgcacactgcaatgaagcaagaagtgttagtgtctcagtcacgtctgaccctttgtgaccccatgggctgtagcccgccaggctcctctgtccatggatttctccaggcaagaacactggagtgggttgccatgccctcctccaggggatcttcttgacccagggatcgaacccaggtctcctccctggcaggcagttggatcatgtgagccaccagggaagcccaccacaacagagaggaacccccagttgctgcagctagggaaaatccgtgcacagcagcaaagacccagcacagccaaaaataaaagataagaaggcgagcatgcagagacctaagaaggcgcttcccagggtgggggggcagggatggcaaacccttgggctgggagtgggctaagaccgagaaaccttgagaccagccctctgggagcctaccgagcagtcaggcgaggtccgagagagcgcgggggaggagggccatggggctcgaggctgagctcgcctcggggcttggccgtctgcccgcttgctgcgggaagccattcaagcgtttggagcagtgacaggatcagactgagttttcagaaagcatcagtgtggcggctgggagcaacatgggccgcagccacccagaacagaaaccagaggttcagacagaaggctctcgtggtgcttcgggcgcagctgacactgaatcagaggtatgggcgctagaattcagtgggctggtgggagggcagggtccccggctgatggactgggacacgggagtcaccgaaaggcactcaagggtgactgcgggttttgcaggaacatctggggggcgggaaatggagcctgggtttgcatggccatattaaatcttgagatggtctgggaaagagtcgggaaaggtcactggggtaggttgagaaaccgagaaaacgtcgagtcatggaaactatgaaggagaatgtttccagaaggagggaggaggcgcatgcgtggttggtgagaaggctgctgagaagcagtgtgagataaggacgcaggctctctggacccggctgggggtcatggcatgctgggatcaaggggcgtaaaggactgagatgagaaggacctggcagtgggtgtcgataacccactgaggcttaatcatcagcgggacaggaatgaagtactgaatgcatgctcagtggtgtctggctctttgagaccctatggactgcagcccatatgctaaccctactacagagcggtggtggtgtagtcgctaagtcatgtctgaccctttgtgaccccgtggactatagtccaccaggctcctctgtccaggggattctccaggcaaggatactggagtgggttgccatttccttctccaggggatcttcccaacccaggaatcgaacccgggtctcctgcatcgcaggcagattctttaccaactgagctacagggaagccccatagagaaccgcccccacccccgcaacgtccctcggtgctatgctgccttccccacttgccaacgtaattctccaagaggctcctctgtccacaggagtctccaggcaagaatactggagtgggttgccatttcctcctccagggatcatcctgacccaggggtcagacctgcgttccctgcactgcaggcagattccgtaccacttgagccatcagggaagccctaatgaagtagtactaaggaccaaagaatatcgtttatttggctcatttttcagttagagttcatcgaagcatatttgtatggtgatggaacagtgcagcagagcggtaggaatcaagaccagaaggtgaaaggatagttagaaagcgacgtttttcacggccatccgtccagttgctcagtcatgtccgactctttgataccccatgaaccccagcacaccaggcctcccggtccatcaccaactctcggagtttactcaaactcatgtccatcgagtcagtgatgccatccaaccatctcatcctctgtcgtccccttctcctcccgccttcagtctttcccagcatcagctctttacagccaaagggccagggttaacatggtaaatggaattactgttctttgacaagagccaggaacatgtctatcaaacagacaaggcagagaacacagatatcagtgttctagattaataacgcatggggccaaaccgagggagttcgcatttgtttacattcattttctgaatgagatttgaagtgaggctgccagctgagaaggtacacatggtggtggctctggagggaggaggtggattaggttggagcgtgaggaagccagcatagtgctgagattcacggggaaggtgtatagtagcatcaggtgttttttgaggttcatggtcatgaaatagagtgagatctagttcatagtggtttgatttctccagagcattttgctgtgtggaactaggcaatcgctgggtttagccaagctttgcatgcctctgagtgagtgcttcaggagggagacaggagaaaaagaatgaagaatgctctcaaggtggctgatggaccacaggtatgggtgggaaggaagagacgcgaagacttgggggattgatggctagttaaggtggggtattgcaggggctggaggaagctgtcatagcatcattccaggaataagcgagcaggtggaataagacatggtcacgacaggaggccggagatggagatttgggaagctggcgcagccacgtgtgtgacggtgacagtggctggctgagctgcagcaaaataaaaagatcgctgaaaagacttgcgcagctcagaggccagggtgacaggcgccacccaaggggatgttgaagttgcaagaatgatgatgggaagagaagcggggaggaagatggtgagctggtgctaagcaaaggttttcagttgctgaaactgaacacgtgagaagagaagaatatgtgacacatacttacacgcgtaatcagcgtgaaaggtggagactcgtgactcacgttcacgtgatgctttgggggcataagatggatggatggtgaccaatccatcttgagtttgtttttttttttttggctgtttggcctgcgagatcttagctccctgactagggagtgacccccagccccagcagtgagatcgcagcgtcctaaccactgggccagtgggcacatcccccatccgtcacggttaaaacacaaatgcctctctcagcagatgttgccgccaccgcctcagcgatggtgtccgcctctgcaccctcagtgtacagcgtgcaggccttgtccctcctggcagaggtgagtacgcatctgcctcgtcatccccgccgggggaatgaggcttcacgggggcgcgtccattcttagaggctgggggctcgggctgcaccgccctggctgagccggtaagccaggctgggcctctggctgccccatggggttgccctgctaggctgtctgtggcccacattaccacttcctttaggagagtcctgtgttccgactcctgttctcctatgtctgtggccacactcccacttcctttaggagagtcctgagttccgactcctgttctcctaggtcctggcgtcgctcctggacatggtttaccgaagtgacgagaaggaaaaagcggtgcccttgatctcacgtttgcttcactatgttttcccttacctacgcaatcacaggtatggctgtcaccacctctctgtaaccccccaaacccgtccactcctgcccaggtgtgggaagatggctaatgggaaacgcatccaacagggctcctctcacggcctctgaccttgaccctttgcccttcccacccccttgcagcgcctacaacgcccccagcttccgagcgggtgcccagctgctgagttctctgagcggttatgcctatactaaacgggcctggaagaaagaagccctggagttattcttggatccagccttcttccagatggacacggcttgtgtgcagtaagaaacaccatcctgattaaaaaaaacgggccgggaaccatcaagtagcaaagtgtgtgggtttctacaattagttcccattcctgaaatggtgagcagagctagctattatcatcccaaatcttaggacatccctcgcaggttaacggtgtatttccggggaaggaattgaagtcttctgaaaatgacacaggtgcccttttttttttttaataatcaggagaagaattgttattattcctaaagagcataacaaggggttccagctgtacttggcctagtgctgctctgtcttgtaatcacgagatgatggaccagcactgagacgtcagggctctgtggtcaccctcctgggtccttccccccacttctgggcttacactccacgagtcgtctctgagaccgtgcagccacctgcctctgcgtgggcagcagagatgcgtagaatcaggatgaactgactgcactttctacttggaggtcagcagaaccactcataactctgcccgtggcttggctgaacatgtctttattaacagcaggggtcgagggtagacactggtcagcttctttgaccacagctttcaccctgggtcggtcagatccattggaaaacggacaggctacccactccagtgttcctgggcttccctggtagctcagttggtaaagaatccgcctgcagcgggggagaccacagtttgagtcctgggttgggaagatccgctggagactggataggctacccactctagtatccttgggcttccctgtggctcagctggtaaagaatctgcttgcaatgcaggagacctgggttcaatccctgggttgggaagatcccctggaaaagggaaaggctacccactctaggattctggcctggagaattctgtggactgtatagttcatggggtcgcaaagagtcggacacgactgagcgactttgactttcaccctgtaatccttttgcaccgtgggagagtctgaggaaaactagtgatatttgtcttcagggccccaactgcccttcctcaacacaagtgtaaggacatcatctgtgcaaacacatttaagggcaacccttgcaaaagccctttgattcagatggtaaaggattccgcctgcaatgcaggagacccgggttcaatccctgggttggaaagatccccagagaagggaatggcaacccactccagttttcttgcctggagaatcccatggacagaggagcctgatgggctccatggggttgcagagagtcagatacaacggagtgactaacacttcttttttttttttttttgcaaaagccaaaggactttgacacaagtgctttcttttatggtggtctgggaattattcattactccctgttatttattccctatattaaggttataaatgaagagcttctgcttagtttctccttattcatccctttcactaacttttcagaaagcagtctttaatcttaaagggacatgttatttttccttgatatttttgactgggctgtcaccctttgcatatttaactttgctttttttttttttttttaatatccagttggaagtccattattgaccatcttctgactcatgagaaaactatgtttaaagatctaatgagtaagtttttctgtgttaatgtgcacataagtcatactttaaaggaccgatgccttttggtcataatgagcttttaccacatttcagaacgtggctcaaactttttatggtgcagtttcagccacaaactatactgtgatgcataggatttgccgggactccatgaatgaacctttctggttccagtcggttgacaagaatgcctctcaccaccatttttgcatttttataatggagaaaatacttttgaaagcaataataaagtgaccgtaatttgttaggagtacttttctatactgcatatcgtcttcaaataccctacagttaagcatcactcaagaaataaatacttccataaattttaaatatttggataatagctacatgcccttaatcaagaccatattccaccagggaagataggaatgaacacttgtgtgaatttctcatcacttacctctcattgtaggtttctcactaagtgaaacgtatttgctgtatttattccaggcatgcagagcagttctttaaaactgttctccagttttgagcagaaagccatgctgctaaagcgccaggcgtttgctgtcttcagtggagaacttgatcaataccacctttaccttcctctaatccaaggtaagaaagccccccctcccctccaagccccctccccgcacagacaggtccagagtggccaaaacggctggagaccaggggggcccacagcacatgctggcagaatgggccagtgttagacgagcgttccacaggggcagtcctagaagaactttggtaaatcaggaactttgttttaaaaatcagttactataatgctgttaatctatcaggtcatttttaaagtctggccatacttcttcagcactgtctcctgctagatccctcctgcagtgcagacacttggagggtgcagtgctaacggaggctagtgaaggagcaagactgtggaggagcgcctttattccttagcagttttttcctctccttcaccctatcagtatcctttatctgcctcaaaaatgaatgactggggcttcccttgtggtccagtggtgagcaggggttcggggttcagtctctggttagggaactacgatcccacatctctctgggccaaaaaaccaaaacataaagcagaagcaatactgtaacaaattcaatgctgtgctgtgctcagttgcttcagtcgtgtccgactctttgtgaccctgtggacagcagcccaccaggcacctctgtccacggggaatctccaggcaagaatactggagtgggttgccatgccctcctcctggggatcttcctgacccaggaattaatcccacatctccggcttctcctgcattgcaggcaggttctttacctactgagccacctgggaagcccaagagattcaataaagactttaaaaacagtcaacatcaaaaatcctttttaaaaaaatgaatgtggatattacacatgactttgaagggtttttttcccctccttatgaaatgttaagatgaacattctaattcaccctcctccaagatatgaaagaattctcaaatagaaatgtgttttactcatagcctaaacaagatataatgaagtaaaaaaaaaaaaaaaaacctttcaagatcatgttaaatatcatgagtactagtgtaaaaacagttttataaatactagaattatgatgatgtcagtcattttgggggagggtcaggtttattaaggtataatttacatacaataaagatgacttcccctagtggctcagacggagaaggcaatggcaccccactccagtactcttgcctggaaaatcccatggacggaggagcctggtaggctccagttcatggggtcactaagagtcggacacgactgagcaacttcactttcacttttcactttcatgcattggagaaggaaatggcaacccactccagtgttcttgcctggagaatcccagggacgggggagcctggtgggctgccgtctgcggggtcgcacagagtcggacacgactgaagcgacttagcagcagcagcagcagtggctcagatggtaaagaatccacctgcagtgcaggaggcccaggttcagtccctcagtccagaagatcacccggagaatggaatggctacccactccagtattcttgcctggagaattccatcaacagaggagcctggtgggctacagtctatggggtcgctgagtcagaaacagctaagtgactgacaccttacgtacagtagaagtcacccttctggctgtacagttctgtgaattttgataaacacacacagtcatgtaaccaccactgcagtcaagatacagaacatttccatcacccctgaaagtttgctcttgcccctcagaagtcatctcctcccctgaccccatcaaccactaaatctgctctctgctcgcgtagtttcattttttccagaatgtgaatgtgattagacagtgtagagcccttgagcctggcttcatgctattgtacttacccggagttactcctttttgtttatctacagtgttccattctgtgagttgtttgcccggtcaccagtggaagggcattcaggttgttccacatagaattatgcaatagatctcttaatgctgccctcctaaaaattttcatgtagatatctgtcagttcacgaagtatatgcttttcctaaatattagtactcctagttctcagtccacgtccctacttgattaaccatttgaaaggtcttaatgattttaggccaaaacattaacagctttcccaggtattaattatgatagtgttttacattggcaataataaaaaataccatcttatacttcagctgacataatttctgcaacgtcaagcgcatcatggagatgttcatgccataacatcctgaatacctacaatgtgtgaaagtgaatgttcagataggacaggtccttacttgaagtactaacaggctgatgagatcagagggtgaccaggaatgcccatgaagacggtggcagccccccaaagccttcagctcagagcagagtcagagctgaattttgtggcatccttacaggatttgggagcgaggaagagcgacaggaaccacgtttcctgtggttcacgggcagcatctgggatggtgctttatgcacctggccccgggacaggggcctgaaagggcagcgtcaagccccctgtcccagaagcagagcgcggtggccagtgcaccgggcagtccgccttctcgcccatcagactctctcctaaagcctggggcgagccccagagggctggagagacactgttcagtgtacagagcaagaagaacacagacagtgaccttccttaaccagcggcgtggagggtctgaccacggcaggatgtaaacattcaatttctgagagatcatgagatggggagggaagtagggggccacagaatagtatatacattatatatggtacttttatatatggcatatatgtatatgggggcttccctggtggctcagaggctaaaacgtctgcctgcaatgcgggagacccgagttcaatccctgggtcgggaagattcccctggagaaggaaatggcaacccactccagtactcttgcctggaaaatcccatggatggagaagcctggtaggctgcagtccatggggttgcaaagtcagacacgactgagcgacttcacttttactttcttatatgtatatgaaggtcaccttcatgctgactttaagtaggaaatgtttggaagcccttggagaaagaggaatttctccaaataaataccctccactttggataactcaagggtggttttttgtttttgtttttgtttttttgtttgtttgtttgttttaatactacctgtttgaaaaaatagagtggaactccctggcggtgctggagtggtgggtttcagagtgttctctgacagtcttgggtcactgagtggcctctggagccatgaggtcaagggagtagacaatccagaagggtctaaataattgtacaccatggtggatagtcaaggcctgatcagaagaggtaggaatagggaaggcctggtatttctcctctggggtcccctctagggtcctttccccctgaccaccatcaagcagccctgggtggagggtgatggttgtgagtggagtgcggagctaaataacgccgtatatcttggctcctcatgacataaattttacattgatctcaaaagaatctgtaaatgagctcaaagcaattattattattttataatgggaaggatgatcaaacgctgcttgtctagaattaaaggaagacagtcatcaccccctacaaaggctcctgacgaagggatcagaaaggcaaccctgaggcaacactccatgccctttaattgatgagggcggttccttccaggtctcctgaagtccctcccatcttttaagtcttcctcgttgtcctgtgttagctccattttttacatcaggataccgatgctcagagggattagcacctctgtaaagttgcattgccactacctggcaaaacctggactgaagtctgagctctctggctcccaacagtgggctgtggtcttcaggcggtaccctttgaggcaggtgagtttcacagataggaagcgggctcggtgagattacctggccagcctaaaacgagattccacaaccttggcagtggtgcatggttctggctcagggctgtctcctctgtctactcacaagggctccttaagcctgtgaggtttatcactcagctaagaaaggcgtattagagattctaaaatagatcatcttgactgaagaataatctcatgagtttagttttgatttgaaatatcaatacgaaatatgccctatgtgtacaaaccactttttttaaatctgttttttcttgaaatagattaagactcacaagaagttgcaccagtagtacctagtgttcctgggtaccatttgttcagctttccctgtggtaacatctcacataagcatagtctctcatcaagaccgaaaattgacatgggaatgttactgcaactcaactagagacttgttcagagctcaccagtttctacaatgcattcttcttgtgtatgtttgtgtgtgtgtccagttatgtgaatgaatctcctctacaaattcttgtggacagcaccacaaccaggacatagaactcttccctcaccactaagcaacgccctcaagctactcatttcagttcagtcgctcagtcgtgtctgactctttgcgaccccacgaactgcagcacgccaggcctccctgtctctcaccaactcctggagtttacccaaactcatgtccactgagtcggtgatgccatctcatcctctgtggtccgtttctcctcctgccttcagtctttcccagcatcaagccactcacttctttatagagtcacaccctctacccaggcctaacccttggaaaccgctcacatgttctccatgactttgatttgtcactttgagaatattgtataaatggaatcatgcagcatgtcaccttttgagaatagctttttcattcagtacaatgcctctgagatccatccagaggcaagtaggttcatgggtgtatcaagagtttgctcttttttaatgagtaataatactccattttatggatgtttcacagttcatttatccatttaactggaggacatttgtgttgttccagtttggggttattagaaataaagcctctatgatgcatgcatgtaccatttttgtgtggttgtaaattgccatttctctacacaagggtttgattacagggtcctgtggtaaatgtgtttaactctctagttcagttcagtcactcagtcgtgtttgactctttgcaaccccatggactatagcatgccaggcttccctgtccctcaccaactcctggagcttactcaaactcatgtccatcaagttggtgatgccatccaaccatctcatcctctgtcgtccccttctcctcccgccttcaatctttcccagcatcagggtcttttccaatgagtcagttctttgaatcaggtggccaaagtattggagtttcagcttctgcgtcagtccttccaatgaatattcaggactgatttcctttaggatagactggtttgatctccttgcagtccaagagtcttctccaaccccacagttcaaaagcatcgattcttcggtgctcagctttctttatggtccaactctcacgtccaaacatgattactggaaaaaccatagctttgactagacagacctttgtcatctaactctctaagaaactctctatgaaattgccaaactgtttttcagagtggatgtaccattttgcatcctcatttaaacagtaattcaagtgtcattatcctttttgatgcctatagagataacctttgtttcattcttgatattggcaatttaccttctcttttttggggttagtgtttctagaagtttattggttttattgttcttttcaaattaccaaactttagttttatttgtaatttcatttatttctgctctttatcacgcccttctttatctttgttttgggtttagtctgttctttttctagtctcgtaaggtggtggtggtggtttagtcgctaagtcatgtccaactcttgcaaccccatggactgtagcctgccaggctcctctatccatgggattgtccaggcaagaatactggagtgggttgccatttccttctccaagtatcataagacagaggcttaatttatagaattgagtgctttcttcttttataatgtaagcacttagtgctatacatttccctctcgttactttaactatatcccacaaaatctgataaccttttaattttcattcattgctttatatcttttatttgctttgatgcttcctctcagcccatatcttatttattaatagaagtttgttagtttccaagtgtttccagttgttcttctgtaattaatttccagtttgagtccattatggtcagagtgggcttccctgatggctcagacgataaagaatccacctgcaatgcgggagacctgggtttgatccctgggttgggaagatcccctggaggagggcatggcaaccctggagaatcctcatagacagaggaatctggtgggctacagtctatggggtcacaaagagtcaaacacgactgagcgactaagcacacacacgtggtcagagaacattctttgtagaatttaaatttgttgaagttagattttgtcatgggtcattaagcagtcttggtgaatgttccacgagcagttaatgagatgaattctgctgtgttgagcggagtggtctgcaagtgttgtttagatcctgcaggctggtggtattgcttggttcttctatatccttacttgactgtttttttcctaaggcttctatccgtcatgcagtagggatgttgaacctcagctacagttgtcagaagttattcttctttgagttctggtagttctttcttcttgtaatttagaccgctgttgttcggtacacaggcatttaggatgttacatcttcctgttgaattgacctttcgctctgcagtgtccccctttgaccctggtgctctgctctgctctgcagtccattccgtgttaatagtgccactccagctttcttaaaatgtttgcatggtgtgtcttttcccatctgtttacctttaacctacttatataattatatttgaagtgagttttgtgtaagatagcatacaagttttgtgtaagatagcatacttcttctttctggagaaggcaatggcaccccactccagtacttctgcctggaaaatcccatggatggaggagcctggtggctgcagtccatggggtcactagagtcggacacgactgagcgacttcactttcacttttcactttcatgcattggagaaggaaatggcaacccactccagtgttcttgcctggagaatcccagggacggggaagcctggtgagctgccgtctgtggggttgcgcagagttggacacgactgaagtggcttggcggcggcagcagcatacaagtacatcatttctttcatccattcaaccagtctctctcttttaactggtggatttagacagtttacctttaatgtaattactggcatgtcaggatatattatgcctttttttaaattttctgtttgttccttgtttttaattcctttgtttgccttttttctattttcctgaggttacttgaacattttttcagcattacattttgatttatttaaagtgggtttggggatgtgtctctttttaaagtttttgcagtggtggctatagggattccagtgaacatagaatgtatatacttgtatcaacattttacatcatttgggatgctcttctgttctcccatgtggtataggtgtctgaaatactaccttttcgtattggaaccataagaggtagagataaaactttttgcttttactgtccaacataggttaactactcaggaagaaagaatattgtatgattttaagagaagtttattactaatgtattccagtgaatcttaaccagcatgaccatagttcagctgtttgccctggcaatttgtcatattcctcagaaaccttaaattatgagaagaatcacattctataagcaattatttcagtggagaaatggggaagacttagagactagagagtttcactcaactttttttttcctgcaggaattgaagtttttgtcgtagttgtcaaatctaaactttactgaatcattcttgtgttcactcaacttcacttgtcactcctcacccccaaaatacagtgccatctgccaaccaacctatgtgatcttatagtttgatgtcttgtacttgatattcttagaagaacatttacaggacttccctggaggtccagcggttaggactccatgcttctgccgggggcacaggtttgaccccgggtcagggaactaagatcccgcaagctagatagcatgaccaaaaattaagaattaaaaaaacccaccatcacttacatgctcattgatgatgcagtggatttccggcttgagccggaagttgatgccttcctccctactcagctgtagcaggggaataagaggcccttggctcacttctttagcaaaggccaggaactgctgtgcctcacaaagaagagggctgtgggcaaacatgaagagcagtaccattctcccaacacccagctcacatacagttgttgtcaaatgtcagagcgattgcagttgtgtttttcctctttgtctagtgagaagctccaaactcctcatttgtgtgacatctcgaaaatcttctttccttagaacgcctgacagacaatctcagagtgggacagacatcctcagttgctgctcagatgtttctttttttcagagttttgctgctaagaatatctccccaacatttgacttcactgtggccaataatggtctctgaattggtaagtgcaagtactttattcttgaggtaaaattggagttttttaaaatgcggtttcagcaataatgtacccttttgtgtatgtataatttgatctgtaaaattttcaatcgagttggtagtgcatggtcagctcttggttaagtagtgatattttatcctctaatcttagttgacctctcaattgtagttatttaaattaatttgatattatttgcttttttttttttttggccaccccatgtgacttttgggatctgacttccccagccaaagattgaagccagcagtgaaagcattgagtcttaaccactggactgccatagagcgccctgtttgctatttttcaaactggccttttcccacttttaatgagacataagtgtcatgcagcagcatataagtttaaagtgcaccacgtaatgatttgactgaaatatatcatgagacccgtatcacattcagttaacatccatcatctcatacagatccaaaagaaaagaatttttttccttgtgatgacaactcaggatttacagtctttacttcacctgtgacttacagcagctttgactgcagttgtcacatcctgcgttatccctagtaataacataccggggtccagccccggctgatccagggtattcgaaggagagacggcttcggcgacctatttatttatttatcaaagatataaagagtaatagaatgaggagagctcagtaggaaaattcagtggagataagaggctgagtagcttggtttacgcggaaaatcaatataacccgtgacaccaggttagctctgaccacggaggccacaggcgccctctcgaatagcggaaggtgccccaccttagacaccttctcgagtgagtcttagaagcccaggcaaataaatggccgcagaggatatccgcactccagatggagactcagctggaaattgaggggaagaatgacatggggagaccaggctttggtgagcaaggtccatagctttattttcaacaggggcttttataccctaagttacacatagaggataataggggatgcaaagtcagcagtctttgatccttatcaaaaaccaaggtttctttcctgcaaatttatcgtatacaaatggtttaggtgatttacatcatcttctggccagaaggcctattaacattttatgactcttgacaaagacttatcaacaaagacttattttctctaagagtaattattttaaggtttggcgccatcttccgaagataaaattgcattcctatagggcggatgtgtaatgggtttacaacaaaggaaagaatttattaccttaagggtctaaagttactaacaccaaggccactacttattttttctacataccaactatattaattaatacacattcaaggatacaattcaggggatgtgaaaacttggcaacaagcattggctcatcaatgaaatcttttactagttttattctgacagtttctaactctctgagaggctctaagctatttgaatatcttaagcttcccgtgcctctcgaggctgggagactgtaaacaatcatatgcatagctgtaggagcccgggtaaacttgtcaggcgagttagagagccatctgaggggtttgtatttaaacactcctaattgcccaggaactttattaattggagctgtaagttaactctttgacagagagagcgagatggtggtgggggacagcccccagtaaagtcagaggtgagagcacaaagcaataaagtaggcagactctggttttttgggggtagatgctcgagaatatccagggggactcctgaggctcgatcccgcctttgcgtatgccgagcctccttcctcactacctttgtcacgagtggaatgcctcaccggctcccggcaataacatatgtcttataaatcaacgtttgtatctttgaccaccttcattcaatcttccctcttcccaccccctgcctctgatctcttctgtgagttcagtttggttttgctttgttttgtttcttaggttccccttataagtgagattatacagtatttgtctttctctcctgacttattttatgtagtgtaatgccctcagagtccttccaaattactacagatgtgccaatggcaagattcctccttttcatgctgaatagcattctactgtgtgtgtgtattctcatatacatacatatgcatatgtgtgtaaacatatatacacatgcatgtatgtgtgtatacgcacacatgcatatacatatgtgtatgtgtgtaaacatatatacatatgcgtgtgtgtacatacacatatatgtgcatgtgtatatattcatatatatatatatatgaaacttaatccatttatttgttgatagacacttagaatgtttccatgtcttggctactatattgggttgaccaaaaagttcattcaggttcttcccacgtcacagaaaaacctgaactttttggccaacctagtaaataatgctgccttgaatatgggggtgcacatatctctttgagaatattttcattccttggagtatagtcccagaagtaggtttcgtggatcatacggtagttctattaattttttgaggagcggttatactgttttccatagtgactataccagtttatcatcccaccagcagtgcacgagggctcccttttctccacatcctcaccagcatttgtaatctcgcccgttggatgacagcagttctaacgggtgtgaggtgatgtctcatcgtggttttgatttgcgtttccttgatgattagtgatgttgagcatcttttcatgtgcctgttggccattcctatagcttctttggaaaaatgtcaattcaagtcctctgcccattttttatttgattatttgctttttcgttattgagttatatgagttctttatgtattttggatttatcccttactgcatttggtttgcaaatatttttttcccatttcataggttgtcttttcatatttttgattccttttgttttatttgctaatttttatggacatacaaattttaaacattaagctagatttaacctctctcaaagagtaacttgaattcacttttttttttttttttaagattcaggcattcatacagcttgaagaagagctgaaaaaggacaatgaatcattgaggtaagtagtgtatgagtctgtgcacatatgtgaaggttaatggacatcactgtattttttttgttttgatttttttttttttagatccagactgtgaacatcaatttatgatgtagggttagctcatcctaaacaccattttcttgaagtagttgtgaagggaccagctattctgaccatttttcaaaaggaaaaaaccttcttgtggacatttcataggcaaaaggactacattggatgggaatgaatttctaacaagagtttctttgccaatttatagaaacagcaacaaaatcaacagaatgaaagttgcagcctctgatggaaatgggccgtcggtgggggagataccccagagcgagctggtcctctgtttatcagcttgcaaattcctagacacagcactctcttttccacctgacaagatgccactgtttcagatgtaagtcacaggggaccacgtgtcctctttgaaggaggtgactttctttcctcgtcagcactttcacttgttctttcctgtacagcatgtgggttaccagtggccgaggagcctgctcagagtaccgagtttcagattactcgcaacatctgtggtttggtgaatttggctcacagtagtatttatggatgggtttctgttcgtgtgtgtgtttcagtgggttttgctattgggtggttataaaacagaggcccgcgctggtgatcctcactgcatttacttattttcttttcttggtctctttgtttttaattaactaaatggaacacgctgtctccagttcactctgccactattggctcacattcctgtgggcttaacatggccgctcgtatgtttcatttcatgcctggccccagatgccacttctctctgtcctgtcgccctaaagagaggaatcgtgtagaaatgtttgtggttagaaacacagaatgaaagaagccagtctgaaatggctacataccatatgttttcaactatatgatgatctgtaaaggacaaaactgtaaagacagtgaacagatgagtgtgtcccaggagtggagagggaggcggggcacaggattttcagggcagtgagatgattgtgtgcgattgtgtgtctactatactgtgaacacatgtcgttacacacttgtccaagctgatggaagatgcagctccaagagtgaaccctaagcttaactctggacatggggtgatggtggtgagccagtgtcagttcatcgattgtaacatgtacccttccggtgggagtgttgacagggaggctcagcctagtaaagaaggggcagggggcaaatgggaaacctccatacctgctgctcaattttggatgaacctaaaactgctctaaaaaagaaagttgttaaaaaaaattaaaaaaataaataaataaaaagaaagttgttttttttctttaagtgagttccaatgtacgtgaatatgcatagcatattcactaatcataataggcaaaaagtagagaaaacctaaatgtccatccattgacaagtagataaataacatatggtgtgtccggacaatgcaatagtctacaacacaaagagatggaagtgtcagcacgtggtaacagcatgggcgagccttgacgatagtaggttaagtgagaaaagccaggtacaaacagccatgtagtacatcagttggaatgtaatcgtgtacaaccatgattctaatttatatgaaatgtctagaataaaaaagacctgtacagacagaaagtagttcagtggtggcctcgactgaggagcggattgaaggatgagactgagggatgactggagtggattgaggggtgagtgctaaagggagcagcaattctatttggggttttaaaataccctaaaattgatcacgatggatggtgatggacgtacaattctgtgaatataccaaaagctatggaatggtgcagtctattttttattgtattttgggctgtcctgggtcttagttgcagcatgcaaagtatttatttgcagcgtgtgaactcttaagttgcggcatgtgggatctagttccccgaccaggcattgaacccacattccctacactgggagcgtggagtcttagccactgaaccaccagagagtcctgaatagtacactttaaacgagtgaactgtacagtacgtgaattacatctcagagaagctatttgaaaacctctgggtgggctgtgagcccatgtgcctgacctcggattctcttcctccagttacaggtgggcatttgttcccgaagtggacacagaaagccctgccttctcagatttagaagaggatcaccaagaatgcaagccccacactgtcaggattcttgaacttctaaaattaaaatatggggtaattgttttcctttttacaaaatttgtatttgtggtggaggggaacgggctcagggggtgagtgaagtacttcatatgccatttgatttcaatgactttgaacggggttcaaattgcctcttaagtgttcagacttgaagagggttatacatttcatctttgtcttagcgtttgtatgtcactttctcatcctggatgctaagaggcgaagcatcctcttagcaaagtgtcctgggtagacacagtcctacagaacagcggtccccaacctcctcggcatcaggaggtggttttgtggaaggcactctttccacagaccagggagctgggatgggttgggggcgtggtttcagggtggtttcattacctttattgtgcactttctttctttcatcgttacattgtgataatgaaataatgatccaactcaccataatgcagcatcactgggagccctgagcttgttttcctgcaacctgatggtcccagctgggagtcatgggagacagggacacccgaagtatgttgctaatgtccagtctactctgtaatctcatcttgttcaactgctcaccttctgctttgcagctcggttcctgaatagaccacaggctggggacccctgctgtagaaggagacgtgcctgcagagtccctctgtgactcagaggaagggcctgcaggaggggtggcaggggccccagcaggatggctcacagcgaaatctggtggaagacggtagtggggacgtgggattcgagttaacgctgctgccgtcagggcagacagggagttttcctagaggactcatgtcttagctttcaccacctgcatgagcctctctaatctttagcatgtcgtcgttggttttttgcaggaaatcagcagctcagatgagatcgccttgaagagcgagttccctctgctgcgccagcattctctttccagcgttaggcaactgatgcccttctttgagactctaaactgtgcttttaaaacccagagtcgactgtccaccgatacccaaggccctcctccactcatggagtttcctgtcgcagataacccaagggtcttgaaacaactggaagaatgtgttgaacatgactttttggaacatctggaatgttaaccttggaccggagaatttctgtaagccatttactgagaccttgagaaaagccaggatatactgaaaatggaaaaggagccaggatagtggttctaaacaagtctatctcaatttcaaaagtaaatttcaaatcttttttttttttttggtggccaacaaaacacacctccctgaaatgccttgtaatatatttaggtctgattttgtgtttcttcctcaatgtatgtaaagaagtgaaattaatatatcatctaatggtggcacaaaatttgtaatataaagtaaaccatgaagagattgaaaagttggggttttttgttgcagatgtgtcagtacatctctctaaacgtttttaactcgttgaatattatttttaacctttcatacatgtgtccctttaaatgtttattttggtaccacattttaaaatgaaaatacattattttctgaaaatagtaatagcccgttacctttgactattcagcagaggtgtaagcgtgaagacaatctcagtgtatttaactcttgccgaattaaaatcacacgttcgcaccttctgctgggtaagaaatacaccgttttcaaccctccgacagctgtcacctcagttatacagtgcaatgtgcctaaccagcactttccacttgacataaatcaatcatcatggtcgcagaggctttaatgcctgaaactactgtcggggaaattcactctggactcttcttattactctgtcctctctgggaactctgaacctctgcagacatcgctgttcgtgaaaataattcctaggttcctcagcacagactccttggggctggcagcatgccgtctcactcttttcaagcctctttgtcttaccccagcttccctatttagagtccacttggcacgcgtcctgcacactctctgcctgtgctccaggcttgcctcccagacctgcctgtctcatgttcccttttcagccaccagggcattccatgacatcactttccaggtcaaatgatgctgactgctctgatctgggctctcagatacatcctttcttacagacaagggtgtctggggacttgacaaacactattggtttcgtttcttgtaacatcctaccatgtataagtctgtgagcagggcagaagcaatttgtgtcactggccccaagagaggatgtggggaacacagaaccacttggttccctgctgctgctgctgctaagtctcttcagtcgtgtccgactctgtgcgaccctatagatggcagcccaccaggctccccggtccctgggattctccaggcaagaacactggagtgggttgccatttccttctccaatgcatgaaagtgaaaagtgaaagtgaagtcgctctgttgtgcccgactcttagcgaacccgtggactgcagccttccaggctcctccgtccatgggattttccaggcaagagtactggagtggggtgctaaaagagtcgaaaagcaccatcacatatatgggtattcatacaaatatgtgttaaaaattacacgcagggacttcgtgttggtccagcagcgaagactcggtgttcccagtgcaagaatcccagtttggattcctggtggaggaacgaagatctcgcatgccacatggtacagcaccccccaaactgacaaccaatctgagagcttatttatatcaccagtagctattatggtatgaaagatagttctcgtgtctgtacttagatttacaaattttgttttttcctttttttttctctccagtgtctaaaacaactcaataccatatgtaatttaatccttttttattaagtagaaatcttaaagggagttaataatacatctctgtttagcataaaacagtaaaaaagaagtcttgtaaatgtgatggctgaagctccaatactttggccacctgatgtgaaaagcaactcattggaaaagaccctgacgttgggaaagatggaaggcagaaggagaaggggacaacagaggatgagatggttggatggcatcactgactcgatggacatgagtttgaggactcagttttacccacatgctagactggccatatactttgtgcctcagtcttctcaactacaaagtggaattatcaatggtaattacccctcagagttgtaggaactaatcctctctgtattccctcagtgatctatatattttttttcacttttttggggatcttttgtgtcttatttttttgactttttattttatattggagcatactattaacaatgttgtgttaatttcaggtgtatagcaaagtgatccagttatacgtgtatctattctttctccaattcttttcccatttaggttgttacataacattgagcagagttccacatgctacacagtagatccttgttgattatccattttaaatataccaaggtgtacatgtcaaccccaaactccctaactatacctccccccaacccccacccttgcccctatctgtaaccataagtttgttctcataagcctatgagtctgtttctgttttataagttcctttgtatcttttttttataccacatataagcaatatcatatgatgtctttatctgacttcactaagtatgacaatctctgaactcatcccatgttgcttcagtggtctgtatagttttaatcatgcatgtgtgtgtgctcagtcatatccgactctctgcagctccatggactgtagccctccaggctcctctgttcatggaattttccaggcaagaatactggagtggatttccatttccgactccagggatcttcctgtcccagggatcgaacccacatctcttgcatctcctgcaccggcaagtggattcttcaccactgtgccatctgggaaacccccaccagcgaaagcgaagcttgtgctcactctcagtatccacaagcctatgtgttgcattaattgacctgagtatccacatagattgtgtattttataaaagagaaaatggagatttttttttttttggccacatcacacgatatgtaggatcttagctccccgaccaaggatcaaacctgtggcccctgcagtggaagaataggaacttaatccctgggcagccagggaagtccccgcatttgttgtttttgtttaagtatgaggaaaaacaaaattaccagttttttttttccttcctcatggaggctcttctccaccccattttgaagatgactgttctgactcagttcctcctagaaggtaaaccaggtgtccagcccaaggaccaaagtgccactgctctgggggcaagcagtgagctggaacccctgttctaagaaagcagggtttggaacacacgcggctgcaggggggtgtggtgagaacagagcccatatgaatgggaatgacatggtcgttaccactggggtgctttccaggggctgggcgtggcgctgtgtcatctgggttgtttcctaagctgcggaggaggtaagcagtgttacccaattctacaaagtcaacagagtctgtgttcgtcttggtctagagcactatgtcctcaagcccagtgccaagaagagttcaacaggaaaaccccgggagggctgctgctgcgtggcgaggtatggaagaccacttctgagggcaccctgcccagtgggcttcctgccaaacctggccaccctggggtcatggcagagcgggtggctcccaaggtgtggtgaggagtatcttaaaggagaacaagccttatttcccagtatgtgtgtatgcccagcaaaattagaaggagctggatgggtccagacagttgatgaagcctctaattaaaagctttgaacaacaacctggaagaccagacagaatctggtctttggaataaaattctcatgtaagacaatgccctctactaggtattatttgcagtttaattgttgttcagtcactaagtcctgtccaactctttgcgacccatgggctgtagcacaccaggctttggtctccttcaccatctcctggagtttgctcaaattcatgtccattgagccagtgataccatccaaccatctcatcctctgtcgtcccctgcccctcctctgccttcagtctttcccagcaccaagttcttttccaatgagtcagctctttgaatcaggtgaccaaagtattggagcttcaacatcagtccttgcaatgaacattcagtgttgatttcctttaggattgactagctaaggatctccttgctgtccaagggactcttccaagaattttctctagcatcacaattcaaaagcatcaattcttcagtgttcagcttttctttatggtccaactctcacatctgtacatgactactggaaaaaccataccttcgactacatgaacccacaaacatggaaaagtagcaccatcaaaggagagactggtcatgtggacctggaccagccaaggagtccattctacaccaagggccaactggatgttgtgacaatcactacagtggtatcttggcgatcgggggctcctctctagttgtgctgtgcaggcttctcactgcgggggcttctcgttgcagagcatgggctctaggcacgtgggcttcagtatttgcagcacacaggttcaggagtcgtggtgcacaggtttagttgctccacagcatgtggaatcttctcagaccagggattgaaccagggacccctgcacgggcagggagactcctatccactgaaccacgggggagtctgtattataagttgtttttgatgccagtgcttcctggaccactcttggagaaacactagtttgcagagcaatgagttcacagagatgatggctcttatggaaacaaagaagaagccgcaaggctgaagcttgtctggttacttttcatcctaaactggtagctctttccaagtggtcctctatctaaatcagtatgcctaccttcaagccaagggggaaatggactttaaaatcgggtctaggaagagagtcccctaaagaatttaagatagaattaccctgtgatccagcaattccagtaaaaactgaaagcagagtcacaaacagatacttgtacacccacatccgtggcagcattatccacaacagctgaggtggaaataacctgcatgtccatcaatggatgaatggataaacgtggtatagacatacagtggaagattattcagccttaaaaaggagtgaaattcggacacgagctacaacgtggatgaaccttgagcccatgatgctaagtgaaataaaccagtcacaaaaggacaaatatgattccacgtacatgagctacctgaactagtcaaattcatagaaaaaggaaacagaatggtggttgtcacaggctggaggagaaaggactgaggagttagtgttcaatgggcacagaatttcagttagggttgagaaagtcctggagatggatgctgttgacagttgcataacaatgtgaatgtaattaatgttgctgaattgtccacccaagggctaaaatgataaatgttatgctttgtatataataccacaataaaagttttaaaatactgtgaatttaaagtgagagcaggggagatagcccaggccccaccttatctggcgaacaagcatttgtaccagcccaagtttccccccacccaaagtcaaccagaagcaagtgggagggggaataataagaacaaacttgcccttcagtgtttcagctttagaaaaagaagttgcttcacgttttaccctaaattgaaattataataatgattgttgggacttctctggtggtctggtagttaagactttgccttctactacagggcgtggaggttcaatccctgttgggaagctgatatcccacataccttgtggccaaaaaaaacaaaaacataaaacagaagcaatattgtaacaaattcaataatgagttgaaaaatggcccacattgaaaaaaaaaattttgttttaaagtaatgactcttgatgcctgatggggtttctagcctcctgaaggccttagctctctctcctgtctcctccaaccactcgggatttctcatctgctgcatttcttacatatgttgtttcatccactcgatactgttttcttcttttcacctggaaaagaccagctcgagtcatttattggtttattcaacggatatagaggaacccgtctttgagatccaaactccccaggcctggttctttctacacctcctcaggctcagcaaggcaccttcccagagggaccctatgaaaacacccaataacgcagagcccataattggtccaaggcaagcgcccttttgtggaaaagacagagcctgtctcttcggttcctttgctgtctggcatctacgggcagacggcagagatctaaatgaagaacagaaaggttgacttatctgcccaaggtgctgctggaaggaagaagaagaacgggaagtaaaactaagtttgaatgaatcagtctgtgggactatagagagtgtgggctccggaaactgggttttctgccccaatcttggcctctccatccctctaagaatccccttgctgggattcctgtgacttccctctgtggatacaggagggtgctctgcttggcggcatttcccaccgcatcctgctggtcccagcactgcccgatgacataagtcagcccaacccagctgcagcatctctgtgaggaccacccactctgacttcaccaggctcatgctgaattcttcctggacccttggacaaccaaacgcttccataccatatggagggggagcacgtgactgtgcactaactgcatgctcggggagctttgtgttgtagtaaaggcaccactgtaactgacagcccttgattgcatattctcccatattttccttccaattattttctgtttgtttctgaagctaagccaggctatcaatatacattccatcaggaaaaagacaagttgtttaaaactgttgtgggggggcggcagtagagcttatggccagatgaacagtatgaatggcctaaacacgataaattcaacagaaagtgaacatggggacttccctggtggtccagtagttaagactccacacttggactatggggagcacgggttcgatccttggtcagagaatgaagatctcacatgctctgtggcacagccagaaaaaaaaagttaatatgtacacatcaatatagttttttgatatcaatgtaatatatatatgtgacctgttatgagatagataaatctatcttccaataacaacaataataaaacagggggaaaaaaaactttttaagagctccttgaagacaaccctacgaaagcagacaagaagacttggagctaaagggaagatttactagatcttggagaggaaaattcaacatcatcaagttataaatgttaacttattgaagacttgagagaactaacctcagatctttatattataaagctaaaataattggtaccagtgcagaaatggacaagagatcaataggataaactagtaattctatcagcggaccaaaacacacagaaaagtttaacatgtaataaaagtagaacttcaaactgtagcgggaaagaaaggtatcttattcaacaaatggttttgggagaaccaggtacccaacagggggaaaataagttgtattcatctctcacatctttcaccaaaataaatcccaaacagatcaaagattggaaaccatgactaaaacatgaagacgctagaagaactgaagaaaaggtatttataaaaaataagccaacaatggagatgatctttcttagatgaacataaattctaggagcctaagagaaagttctataagttttggctatgtaaatttcctttttaaaactgcacaacacaaaatcatcagaagcaatgtaaaagacagcagacaaaaatatttgagacatattacaaaggactgatttcttcagtactgaaagaacaatgaaaagaaatagaatagaaataggggcagaagatgtaaatagagggtatgtatacacacacacacaattggcttttacaaatgtaaaatgatacccatacttacaacacgagaaaaacattgctctgagatattagtcactgaccatggcagccaacatcaattataggtgaacagcatctctcctccattgctatagaatataaattggtacaagttctgtggagaaagtgggggcaatttctaccaaaagtaaaaacggatggagacttccctggcggtccagtggttaagactctgggcttctactgtagggggcaggttcaatccctggtcaggggactactatcacacaagccacatgtatatttttgatttcacaattctactgccaggaatttacgatatacatgcatatgtgcaaaatgacttatgtacaagttattcattgtaaatattgataatagcaggaagtttggcaacaacctcagtgtccaccagtaggagattggctacataaatgacaacacaatataataaaatgttaggaaacagtaaaataatgtaatgaaatttttcatgcacagcactgaaggatctctgagagatagtccatggaagtcaagatgcatagctgtgtaggtttttgaaagcaaccagttttcatattgtgtttgatttaggtcagtgggaaattttactctattctgatattcttactcttgtatggaattttaaaatttctcttttcttattttgggctgctgacaccacagattgggtggcttaaacaataaactaatttctcacaattctgtaattgggaagtccaaggtcaaagcaccttggaaatttcccagtggtccagtggttaggacgtggcactctcatctgcccagggtctgggttcaatccctggtcacagaactaagctcccacaagctgtgtggtgtggccaaaaaaacccagcactttcttggttctacctgctgtgtcttcacataacagagggttggttgaggcatctctatagagtctcttttacaaagactctaattccattcatgggcttctctggtggctcagcggtaaagaatctacctgcaatgcaggagatctaggttcactccttaggtcgggaagatcccctggagaagggaatggcagtcctctccagtattctcacctggagaattccatggacagaggagcctggggggctacagtctatgggattgcaaagagttggactccactggactgagtgactaacactttcacactttcaatcccattaatgagggacccaccctcttgacctaattatcctcccaaaggccccacctccaaatgccatcacatggcggggctaggtaacatggatttttttgcagggatgggggatacaaacattcagcactccccaaagaaggtagtttttaactattaaaatcatatcaacctcagctttgacattttttaaaatgtcagctgagttcatctgacataagcaaaagcatttactttcctgaaaaagccccttttataccaatgtggtttaagtttagtccatacatgtctgcctcacatttctttcatatgagtaagccttgagaatgcagaactctgcagtatgcgcaactgggttaaaaaacttttgggaggggggaggcaacaatttgtatgtacaggtaagtatttgcttctacatgaatagactactgtctattcctgttgctgttctaacaaattactgtaatttcgtgtcttcaaactgcaggaatgtattcagaacctagagttctggaagccagaggtccaaaacgagctgaaaccaaggtgtctgcagagactcattctctctgtaaactctagggactctagaatggggtttccttgcctttctagtttctgaagctacagtccctgcattctttggttcatggcccttctccactttccaatccagccacatagcatcttcaagtctctagagagatgcttcagtggtaagtggccttctgtgaccaagtctttctctgcctctgtcttataaagacacttgtgagcacatctagggcacatccggataatccaagctaatctcacttcaaaatccacgttgctgaacatcacatggttaataaatgaataacttggatttgggcccaggcagacactgtagtatgctattctagtttttcttttttttaaaacaaagtgctagttgagactcaccaaattgatctcacaacaaacgagctgcaacttactgaataaacagccaagagactgtagattccacaggaatggaaacctctgttttagtcatcactgcgttctaggccccctcactttgatagacacaaagtaggttctcaataaacagttgatgaaagagcctctgatgtaataatatgatcttaaacttggaaggaactgagaaattacccagtccagagcattgcaaatggtattgactagcattcttacttaggtggtgtttcacatgccatttatcctgtggacaaagaatgttacctgctgtatcagtaaaggaaggatgttgcggccatcaaatcattagccactgcccaccttgaggggattcaggatggggaaaaataggatactggctctaaatagttaaggtgcacatcaaagggttgatttcaataagcccagactcttgcctcttctcatatcaagaaaagcactaaattcactaacttgagatgattggttttctttcagttcagtcgctcagtcgtgtctgactctttgcgaccccaaggactgcagcatgccaggcttccccgtccatcaccaattcccagagcttgctcaaactcatgtccatcaagtcggcaatgccatccaaccatctcatcctttgtcgattaactgtaatcttttgacactctgactacctaggtgttctgcaaaaagccctatcctagctccaccgttacatcttcagaacagtccctcagagctgtctgagaggctgtcttcttggcctcagtcctcagtatgtccactgaataaaacataattcttaacttttagattgtgcatttttttttcagttgagaacccagtggtcaatagtaagggtaacaacagagcacaggtttttttgttgttatttttgattgtatttatttatttttggccgtgttggatcttggttgctgtgtgcaggttttctctcattgtggtgagcaggggctactctctaattgcagtacttgggcttctcattgcagtggcttctcttatttcagagcatgggcattaggacaccacgtgggctagctgtcttgcggcatttgggatcttccaggaccagggatcgtcccctgcattggcaggcagattcttaaccactggaccaccgggaaagtcctgtttgtttttaacatttcctcctttgttcatttattcaaaactatttgggcacttagaaatctcaaactgaaataattaaataaaacaaaaacatgagctgcagaggagtatgtaaggtctagttccatactgtaaatagtatttagcgtatagtaaatatggagtaatgtacaccaaattcatatgttcccgtaaagggattagggtaagtgttgttttatatttccatattatctgaattttacaaggcaaatgtatttcctttacaattataaagatgcaatgaggacttccctggtggtcctgtggctaggactctgcgatcccaatgcagaggcctgggtttcatccccggtcagggaactagatcccacatgctgtgactaaagatcccgcatgccacaagacagaccctggcatagccaaataagtattttttaaaaaacgcagtgaatattttaagaaaaaccttttaactctttatttaagggcagtatctgacagcccttgacttattaatttctttcatttttctttggctgcttgacaagcaggatcttagtttcccacccaggatggaactcccgccccctacagcggaatctccccctacagaggaatctccccctacagaggaatctcagaggcgtaacccctgggccaccaaggaagtcccaacttattcatttctgctgctaagtcacttcagtcgtgtccgactctgtgcgaccccatagatggaagcccaccaggcgcccccgtccctgggattctccaggcaagaacactggagtgggttgctatttccttctccaatgcatcaaagtgaaaagtgagagtgaagacgctcagtcgtgtccgacatcctactaattagcccaggacccagctcaaggtcaatagagatacatgtacaggagcaaaaggactaaactgcatatgagaaaggattctgtttatcaggatgaagggtgataattttttaggcaaatccccaaagtacaagtcttcatctcattaagtgttagtcactcagtcatatccaactctttgcgaccccatagactgtagcccaccaggcttctccgtccatgggattctccaggggagaatactggagtgtgttgccattctcttctccaggggatcttccctacccagggtttgaacccaggtctcctgtcttgcaggcagattcttcaccatctgagtcacctcatctcattaaagtttcacaacatgatgacactggcgggcatgagacggctttcttcagtgtaattcacgtccagcccagtcaagcacttcaagtgcacctttttgggtcaccaacacccatggcacctgtcttcaaacttggggacctcaagctgcaaaaggaaaatcaaaaaagagtcagttttgtactgctaaaagaactctccagagtggagccagaggccattgaggaagtgacttatgtctctgcccagactgaatctgaccttttgaccacttatcgtctaacaaagatatccaggacatttgcatagggttccagaaattcaagacacttatctggcccataccctaaataactggtgacaaatccatcccttaaggacgaatattttcttttactatcaaagtcaatcataattcctgcaatacaactttaacaaaacttgtggattttgcctttataaccccctgactctttctcttccccaaacattctattttgctagaacctgtgtatccccagttgtaatccccaagaccccagacagagctctttattatttgcagcctccatactgattaatatcaattaatcaggagaaatataacctcagatatgcagaagacaccacccttatggcagaaagcaaggaggaattaaagagcctcttgatgaaggtgaaaagaagaaagtgagaaaactggcttaaaactcaacattcagaaaactaagatcatgcatccaatcccatcacttcgtggcaaatagatggggaaacagtgacagactttattttcttgggctctaaaatcactgtggatggtgactgcagccaagaaattaaaagatgcttgctccttggaagaaaagctatgacaaacctagacaacatattaaaacacagagacgttactgcgcctacaaaggtccgtctagtcaaagctacggtttttccagtagtcaggtacggatgtgagaggctgagcaccaaagaactgatgctttagaactgtgctgctgaagatgtttgagtcccctggactgcaaggaaatcaaaccggtcaatcccaaagaaaatcaatcctgactattcattggaaggactgatgctgaagctgaagctccaatcctttggccacctgatgcaaagagccgacttactggaaaagatcctgatgttggagaagattgaaggcgggagaaggggacgacagaggatgagatggctggatggcatcactgactcaatggacatgagtttgagcaaactccaggggatggtgatgaacagggacgcctggtgtgctgcagtccatggggtcgtgaagagtcggatacgactgagcgagtgaacaacaaaataccgattactgtttggcaaagcctgtaggttcagttactacagtcaccaggttgagccttacgcatgcgccttactctgcagggcccccgcccggagcctgaagcccacccacctctctagccgtcccggcttccttaaacccccgggggaatttacctgccctaagctcccaactacaaggcgccgggaggcggaagtcggggggcggggtcagctacacgcgccccgccccggaaataggcgggcgctccgcagccacctcccagtctgattgcggctgaggccggtcctggctttgtaactcgctcaagatggcgacgcagccacccagcgggatccgcctcagcgcggtgagcagccgcgaggggtggagcgggccatgtcccaggagggcgggcaggcgggcaagcgaggcgcgaggggggagaggccggcggtgggcgggggcggctcgtcggcccggcgcggcggcttggggccgtggcggggccggggcaggcggcggcggccatctcgcacccgacggctgagacgcgcggcggcggcggcggcggggcagggccgggccgcgtctcgtttcgtttcctcgggtttgggtttctgtttcgcctccgcggcttcggggcggggaggcccctcctccttggctccctcccccggtttgccccgcagctgtcgtccgagggcgaggcctcggcggccagcccgcccctcggcccccgggggcccctgacccgttcccggcctctgcgggcccccggagcgtcccggactgcggggggagtggccgtcggcggccagaaatgccccgcggccttgacgtcctggcggctggccccggggtgctgcgagctgggactcgtcttcctcattcatttcaattcctgccgccttccctcctgcttccctgggtggctgagacggtcggagcgtccgctcgcaatgcaggaagcccgggttcgatccctgggtgggaagattcccctggagaaagggaatggcaacccagtccagtactctcgcctgggaaatcccatggacggaggagcctcgtaggctgtacagcccacggggtcgccaagagtcggacacgactgagcgacttcacttttccctccacttcatccatcgggaatccagatcgctcctaggtggtcaccgagctctcccggtttgccccgtctcgtctcctggcggtgtctcgttccggagcttctcggcctcttggaaatccttgagcgtgttttatacctgtgcgtttttgtttttgttttttttccggagggggcagccacacaataaatgaaaatttctctgcctcaagagtgtggcagagaacccctacccgattctcctgaaaaagttggccttgttatgcaaaaatatacctcttctcatcagggtgtttgctcaggccagagattggctgtgccagtctcattattcgaatggtaataccattgctttaaaatggtatttttgccttttgactttcatttggcgagattcctaacagacccctcctatcttctgacttttaacattttttcctgatgagtgtgaaattttgctttacatttgactgagaagctgaaagtttgtcacacttcattcacccttttgattttgggttaaaagaaaccttcgtataaagaaaaaaaaaacagccctgcatattccttgcccatttttctattttctgtttcttttgaggggaacttttaaattggtttatgagtattctttacatatctttaataaaggctccttggttcctaagtggtttattgaatgagcttgtatttagtagatggaatacagagagcaattttattctttcttaccgctggccttagacattgttgctaattcaccggagtgaatctgggcatatcctgttgctttccagttagcctcaggtactgtttaatctgggcttccctggtggctcagacagtaaagagtctgtctgcagtgtgggagacccgggttctatccctgggtctggaggatcccctggagaaggaaatggcaacccactccagtattctgaagtgacttagcagcagccagtattcttgcctggaaaatcgcatggacggaggagcctggcagactacagtccatggggtgacaaagagtccgacacgactgagagactttacctgcactttcactgtttaatcaccgggcttcccccgtggctcagatggtaaagaatccacctgcaatgctggagacctgggttcgatccctgggctgggaagagcccctggaggagggcttggcaacccactccagttattcttgcctggagaatccccatggataaggtagcctgagggctatggtctataggatcgcaaagagccggacacgactgagcgactaagcactttagcattgttaaattacttgagaagctcttcttcagtttgaagcccgctccttccctcagattgggccaaattttggtccagaaaactggtgaggaaaagtgaactgggttgtttttgtttttgttttctttcccttgctgtttctgtactttgattctcttccccactccccagcccctcccaagttgctgtctctgtctcttcaggattgagaagacaaagagagcaaagataagggtcagtaccatcttgagagactggcactgcggttagctggttctctgggtgcagcagctgcccatgtgctacatctttgttttgtggcaaatacggactgttttgaagctcactgttggaaatctatctgcagcttctggactgtattccaagtggtctgttgctttaggacaggagttggcaaactgcagtttatgggccaaatccagcctgccacctgtttctgtatggctacaagatccttaaatggttgggggaaaaaaagatcaaagaataatgtttcgtaacatgaaaattatgtgaaatagatatttccgttcccataaatagtttcatttttgaaggtttgatcatccctttggtatagctctcagatgttgtttatggatttgtcaaaaatcacagaacataaagtgacacccataatgtgtgcagatttaaaaaaatcattaggtctgtaaattctgaaatggaatgcagagtgacagaaacttaaaagttttgctttttgcctccatgattctaagtaaatgctgataccattttgttgttttccagaaatcctgctatagatttaagtgacacatacgcacactcttcagttcactttttcttttggttgaattaccatatgatttattatcactcttagcattattcagaaatgagccagccaaaactgtgattgctttttctttgctgtatagctttgtgttttccttgaagttagtaattgatttgtatttgtttgcttagtttttctctgtactgattactctttcatccaaactttcagacaactggaaaatgttttccagatgtgttcaagcacacccaaattttgtcatttttgctggctcagttgtgagttggggacttccctttatcatttttatggacatctccttccccttttctttgtttctccttcctatcacatattcctcttctggggtttactcttggccgccaccaatagcttagtgactgagagcatgggttctggagcccacgtgcctgggttcaaagtctgacagataactggatgtgtgacctgaagagagaataatcatcttaccacctaaaagggctgctttaaaaactgaggcaatacacataaaagtcttataacagtcctctgattgccataaatactcagtaaatatttgctgtctttgttactgttaacagtgaagcctatccgtttttccaaaaacagatgtagcttcttgagaacagagtacatgggaagaacatttatgtgtcttttcttggaaatacgtttatgctacccttgctctttctttacatataatctggctgggcttgaaattctagattagaagtgatttcttgtcagaattttcaaggcatgctttattgtttaatagagtcctatgcattatactgggcttcccctggtgactgagatggtaaagaatccacctgcaatgagggagacctgggttccatccctgggctggggagatcccctggaaaagggcatggctacccactccagattcttgcctggagaatccccatggacagaggagctggctgggctacagtccatggggtctcaaggagtcagacacaactttgtgactaagcacgcctgcaatacattgttctagtacttaggctataaacctgtaataatatcagggaaattttctcagatttttgacagcaatttcctgtctgtttttctgtttttgttcattttgttttctcactgctttttcttttcttttggggagattttctccatttgatttcctaccttataccatgtatattagaaacattgtcttattctccaaatatctttaaaaacactttctccatttgatctaccttttaccatgaatttaattattagaaacattgtcttgttctccgaatatctttaaaaacattttctccatttgatctcctaccttttaccatgaatttaattattagaaacattgtcttattctccgaatatctttaaaaaactcaacaaaataattattcttgtcactggctgtattattcatagtatttttgaagttttcttttgttcactgcattgcctctgtcttctttatgttctttttactgtgtgttgttttttattctaattttaacattctcaagtgtgcttatgtttccagatgagacactaaaagctggatgttcatacctgaatagagcttgtcgtttggtgggtttcagtattgggtgatcaggtaggaagagccagcttgtcgttggagtactctgtgtcagagtctgttgatcctagataataaaaatgttagaccagaaaataaggatgtttcgaataacttttggagaaggcagtggcaccccactccaatactcttgcctggaaaatctcatggacagaggagcctggtaggctgcagtccatggggtcactaagagtcagacatgactgagcgagttcactttaacttttcacattcatgcattggagaaggaaatggcaacccactccagcatacttgcctggagaatcccagggatggtggagcctggtgggctgccgtctggtctgtggggttgcacagagtcggacacgactgaagcgacttagcagcagcagcagcagcagaataactttggggcttccctggtggctcagtgataatgaatccacctaccaatgcaggagttcaataggacaacgggggtggggaaaaaagcagtcaaaaagcaaggaattatcattccccaaatctgtcttcccatgtggcttaggagattcaggttctatccctgggtcaggaagatcccagcagaaggaaatggcaacccactccagtattcttgcctggaaaattccatggacacaggagcctggtgggctacggtccatggtattgcagagattcggatatgactgagcaattgtgcaccagtgcaggagacctgggtttgatccctgggtcaggaagatcccatggagaaggaaatggcaacctacttcagtagtcttgcgtggaaaattccatggacccaggaccctggcaggctacagtctgcagggttgcaaaaatgtcggaaacgacttagcgactaaacaacaataaaataacttttaaaataatttttttttaagtgaggggggaaaatgctgttcagagtagcatggtacaattgtaaaagagccaaataaaaattttagaaatcattcctttttcccagaggggaattctgcatctggtctccagccatggcatagataatcctggctgctagctcttctggtagctcagcaggggacaggagatgggagtgtcacgttgttcagtttgctcacctggtttttgtttattattgtggtaaactacacataacataaaatttaccgtcctaaccattttaagcctgtagctcagtagtgttttgtacatccacattgttgtacagccagtctccagaacccttcatcttgcaaaactaaaactctgaacccatgaaaaaataactcttcgttccactttgcaccagctcctggcagccaccattttaccttctgtgaatttgacatctatgggtacatcatataagtggaatcatacatcatttgtctctttatggttaacttattccactcggcataacgtcctcgaggttcatgcatgttgtagcgtgtgctaaagtttccttcccttttaaggctgaacaacattccgctgtatattcaagtgtgccacatttgttcatttgttcatcagtcggtatttgttttgctttcactttggctgttaggataatgctgctctgatcacaggtgtgcaaatatctcttcaggaccctcctctcactactttacacaaaataaacaaacagtaaggacctacctactgtacagcagcacagggagctatatttaatatcttataataacgtatactggagaagaatctgaaaaagtgtatatgtgtgtgtgtatatatatgtatacatataaaatcactttgaaatacatataagacagaatcaccttgctgtacacctgagactatcacagcattgtaaatcaactgtatttcagttaaggggaaaaagaccctgctttcagttcttttcagtttatacctagaagtcaaattcctagatcatatggtaaatctacatttagttttttgaggaccttccatattgttttccacactggcttcaccatttttgttgttcagtcgctaagtcgtgtccgactccttttgactccatagactgaagcataccaggtttccctgtccttcactatctcccggagttggttcacactcatgtccactgagttggtaatgccatccaaccatctaaactttctgtcgtccccttctcctgccctcaatctttcctggcatcagggtcttttccagtgagtcggctttgtgcatcaggtggccagagtattggagtttcagcttcagtcgttccagtgaatattcagggttgattttctttaggattgactggtttgatctccttgctctccaagggactctcaagagtcttctcaacaccacatttcaaaaacatcaattctttggcactcagccttctttttggtccaattctcacatctgtacacgactactagaaaaactatagctttgactgtacggacttttgtcagcagagtgatgtctttgctttttaatacgctatctaggtttgtcatagcttttcttccaaggagcaagcatcttttaatttcatggctgcagtcactgcctgcagtgattttggagcccaagaaattaaagtttgtctctgtttccattgtttgcctatctgccatgaagtgatgtgactacatgccatgattttagttttttgaatgttcagttctaagccagcttttttgctctcctctttcaccctcagcaagaggctctttagttgtcctcgctttctgtgttagaatggtatcatctgcatgttggaggttgttgatgtatctcctggcaatcgtgattccagcttgtgataagttaaataaccaaggtgacaattttcagtcttgacatactcttttcccaataaccagtctgttgttctatgtccagttctaactgttgcttcttaacctgcatacagctttctcaggaggcaggtaagatgggtctgtctgatattcccatctctttaagaattttccacagtttgttgtgacccacacaaaggctttcacatagtcaatgaagcagatgtttttctggaagtcctttgtttttctgtgatccagtggatgttggcaatttgatctctggttcttctgccttttctaaatccagcttgagtatctagaagttcacggttcatatactattgaaacctggattggaaaattttgggtattactttgctagcttgtgagatgagtgcaattgtgcagtagtttgaacattctttggcattgcctttctttgggactggaatgaaaactgaccttttctagtcctgtggtcgggtgcacaggaattccagttgccttacagcctctccaatacttgttattttctgtttttgttgttgtggggttttttgttgttgtttttaataatgatcatgcctttgagtgtgaggtggtatctcattgtagttttgattttcatttccttaatgattagagatattgagcatctttggagaaatatcttggtgaggcctttgcccatttttaaatctgttgattgtcttcttgttattgagttgtgagaattctttatatattttgcatactacttgatttgctgggttttttcccctgttctgtaggttgccttttcactctgtagaatgtgtcacttgatgtagaggagttttaaattttagtacaattcaatttattttttcttttgtttcttgtgcttttggtgtcatgtcaaaatcattgccaaacccaatgtcataaaatgtttcctctgtgttttcttctaaagattctagagtttcagccctgaaggtaaggtctttgattcattttgagttgatttttgtttatggtgtaggggaagggtatgactttttgtactgtccaggggattcttgtggcaagaatactggagtggtttgccattccctcctccagtggactgtgttttgtcagaactctcctctgtgatccgtctgttttgggtgaccctccacagcatgactcgtaccttcactgagttatgtaagccccttcgccaccagaggggtatgatccatgaagggggttgaaggcaaaaggagaagagggtggcaaaggatgagatagttggatggcatcatcggtacaatggacatgaacttgagcaaactctgggaaatagtggaggatagggaatcggaatccgggcgtgctacagtccattgggtcacaaagagtcagagacaacttagtgactgagcagcaacaagggtacaacttgattctttttatggatatcaagttttcccagcactatttgttgcaaaactgtcttttcctcattaaatggtctttacacacttgtggaaagtaatttgactatttatttctgaactgttgactggcttgcttttaatccacctgtttttggttttctctgagtccatggccatcagtttctccagagaacagacctcttgtcttttcatggtttgctgagtgtagttgaaggacttgctcagggtaaaaggatctgggaatctttcttctttaaatactttcagcctttcccatttttcatatgacatctagtatctttgacttcaaaggagcaaaagtcttatttcctaaatcttcctgggattgccaaggtttctgacctacttgttggttttcccgtctcctgcaggctttgggtttagctctttccaggatattgaagtagttgtcactgctctgtactctccagtttccaagctgctattttgtctactgttgtcaatcactgctttggttctttttgttctcctgtgtttatgcacccccactccctttccttcgttaggtagcagctccaagagggagccaaggtaaacactgtctgtgtgttcaggcgtccacatttaaagtcttatatttaaaggatttttaaaatttgggaccgtttgttgattctaaaaattctgttcattctttaaaatatatggtcttcattgcatctccctttttgtgtgttttttggaggggagtcttactttttaaaggtctagctatttcaagggattcttattttatacggcttccctggtggctcagtggtaaagaatctgcctgcaatgcaggagacccaggttcgatccctgggttggaaagatcccctggaggagagcatggcaacccactccagtactcctgcctagaaaatcccatggacggaggagcctggcaggctacagcccatggggtctcacagagtcggacacaactgaagcgagcagcagcagcagttttataggctgatatttctttttcccagtgttgaagttgggggtgcactaagactactgtgtcttgtctgcagatattttctcctggtggattgttcccttaaacgttatcttagattgtgtactcaaatattccccaagtaatgacccagagaatcgtgtgcagtctggttgaggctgtgtctccacagtggttctgcaggttaaatggaaacacttaacccaagtgaaggcatgcctctttacagaactttatggtacactggtacactttttttttttaaccttgactgcagaccgaaacagacaagcttctcgttgggcatatttcccctgccctcctttgtattgaggtaggagggggcagctaccttcaagtacatgcttctctgccatatttccaatccacagtcaccattaaaatggaaaccttcatattccagttaagcaccccactctagtccctagggtatccctattttcaaatcttatttttgtttttaatttcttcttgacttgaggcccttgagtattatctttatttatatgtgagctcagttgtgcctttaaaggtatgttcatagttcattcatagtcgctcagtcgtgtccaactctctgcgaccccatggactgtagcctgccaggctcctcagtccatagaattttccaggcaagagtactggaatgggttgccatttcctactccaggagaatcttcctgacccagggattgaacccatgtctcttgcatctcctgctttggcactgggattctttaccactgctccacctgggactttaacggtatgcttaccttaaaaaaaaaaaaaaagaacacacacttataatgcttattatgtcccaggtgttcttctgagtatttacaaatattaagttatttaattctcctaacatcttggtaagctgctgttactatcatcctcgttttcacagacgataaaacctgaggcacagaaggttcagttgcttacctgggatcaggatcacacatctagttaaaagatactccagaggctgtgtgtgtgagatcaggatcatgagtctaattaatgatattccattgggctctgtgtgtgtgtgatcggtgtgtatgatctgtgtgtgtgtgtgtgtgtgtgtgatcagatactccatagtctgtctggggtgggtgggggggtgtgtgtgtgtgtgtgtgttggctgttctagggagtttttctttcccgctaattgttttagagatgagaattaataatagctatacataatgcttgtgtttaaatacaccaaaagccactgtttagcattcatcatttttcactgacgccttcttgctttgttccactgggactcttacccaggaaacaatgtgtaccaacttttggaggtggtgtgtagaatgaagtcattaaatgaaagttgacagtttgcacacagtctgccagatatctaaaatccaagtattacaacaggaacctaaatacccagtttctaactccagacttcaccccagtgtcatttgtgcccagttgtgtccggctatctgaaccctgtggactgtagcatgctgggtttctctgtccatggaattttctaggcaatagtactggaatttttgccgtttcctactccaagggatcctcccaacctagggatcaaacctgcatctgctgcagaatacccaatttctaactccagacttcacacctaacaaattcataaaatagagaacaaagacaaactcttctaagggttctggtgtcgattagtcattctttcctttggtagccgacaccccaagaccataccccatagtaggatttagaatggctgaggagtgttcttttataaatggagaagtgatggttgcacaagaggagggcctgggataggactcgggtggactaattgacaggaggtttcttgtagaagatgaaggtctggaaattgattttctttgtaaaaactctttgttttaggaagttctgaaattccttgtacactgttattacatagataaatgctgtagaaagtcagaattccatgtactccagtttgcaaaccactcttgtacagaataccttttctcacatgtgtacttcaacctgatggtaaacatgcttgaggattgtctaaaatcgagggtggacagaggaaaagcagcgctttttctgacaccttgtggcagcagctgaacactacaaatgtttggttatagtcatatctctaggctcttcacatgaaacacttaaattgcctcttttttcttccttaaatatgagataatctgttcaaaatgaacacagaaaagtcctcatttcatgaaaattctctgcatattgaaatatccaagcctcagcctttttaaaccatttgtgcatttttacacatttgatatctacattagtcaggattaagagagtgccctgtatgattaacatactcttccatactttattacgatgaatcagacaaaatctctggaagtcaaggatgtttgttgcatttgagctgttacttgacattgtttgaggactttatagatatgtttattaagcttaatgcacaaaaggtgaaaatatccctttgacaaagtcttagttttatctcatggtaaatttgaggtggctctagtggtaaagaacccacctgtcaatgcaggagacattagagatgtaggttcgatccctggatctggaagatcccctggaggaggacatggccatctactccagtattcttgcctggagaatcccatagacagacgagcctggagggcccatggggtctcagagagtgggacacacctgagcaactaatgctctcaattttgaggcaggtccagcagggtggggcactttggaattgggccctttctgatatgactctggattggtgggcccaaggaggcaaggagtttgtcgcccatcttaatgagaaactggctgattttcagcctcattttccaggaatcgaatctgggttttatcttccaggtatctcctggcacaagcagttaagtcatttttgcttgatcttggtgttaatactgtgatggaagattattttggcttctgtttgcaaaagtgagaccttaattaatttacatgaatcattgatttgtttcagctttgcccgaagtttttacatacaaattctacaagtcacacttggccatttagtgcagtcgctgaattaataggtaagtaaattgatgtttcatatataatctcctgctggcaattgaagttctagaaagctttttattgaaaagtaacatgaagatgtgctttttgaaaaaaaccaagtgtgtcagtgataacattaataaaggttacctagcatcacaaccattcagactagtctccgatcggtcgttgcccaggtttccctgtatactattgttttaagtatttccctatactactgtcagagcaaagtattttaatttgcagagttcctgaagcatttatgttgaaaatatttttttgtctcatcataaaagaaataggtctgttgccaaaaaatgataattatgtttaaagttggaaattatttatagtacttttctgctattagtaattaggaggttttatttatacatacatatgtatttggctgtggtgggtctttgttgctgcacgtaggctttctctagacgtggtgagcaggggctactcttccttgcggtgtgtgggcttctctcgttgcagagcacaggctctcggtgcacgggcctcagcaggtgcagctctcaggttccatcggctcggtagttgtggtctactggcttagctgctctgaggcatgtggggtcttcctcgaccagggatcgaaccagtgtgccttgcgttgcaaggcggattcttaaccactggacctccagggaagtcctaaaagtgttctagtgaacatgagaaaagaggcaaggtcctgattggatttgcattatggtttttctttggtaaaattcatacacatgaactgtttgatttaaatgtgtttaagaaacagataataaaggaaaactatatttttgggaattcttagatacactttagattatttgtcaaggaagggttctaaagtggatgcaccaagaataaggaaagaaagaaaatttgagaacccttcttttctttattcctgatagttcagttggtaaagaatccgcctgcaatgcaggagacccaggtttgattcctgggttgggaagatcctctggagaagggataggctacccattccagtattcttgggcttccctggtggctcagctggtaaacaatctgcccgcaatgtgggagacctgggtttgatccctgggttgggaaaatcccctggaggagggaaaggctatccactccagtattctggcctggagaattccatggattataataggccctgggtcacaaagagtcggacatgactgagtggctttcacttccttcttttctttggtgtagttagattttccaaagtccctaacatgttctgttgagatgtaaactctgaagtgtaaatgagataacattgcagctattcatggtcactctttttccttttttatggcaacccagaggaactactcagctaggattgttcatccgtacatagtacttatctagttttgggataggacaactgaaagccgttatgctctttgtggtgtatcacattttaagtgtgtgtttttttttaatgatgctgaagtggttgatgaaaaagatttattttgttttaattgaaggcttacaagagtttgagagccactttttatattatagtaaagtcttcaaaatatacctcaaaagtatttttagttagttagaaatattcagatagattttagatagtttataagttgattttattactgctactgttgaaatttttttcctttttctttgctgtattccttaatacttaataactgttctgagagaaacaagtatgctagacaaggatgttaaatgatgaggtgtacagagatggcttccctggtggctcagatggtaaagaacccgcctggaattctggagacccggctttgagctctgggttgggaaaattccctggagaaaggaatggctaccaactccagtattcttgcctggagaactccatgtacaaaggagcctggtgggctacagttcatggtgttgcagagtagataccactatgcgactgtgactttcacgaagatgataaaaatgtacctgtcgtcagaagcttatgttagattaaaaagggctaaaatttgaagaaatgtgttgtaggtaaacatctgtgtgtacgttagcatactgctttctcattcattcagtaatcctgtgtggaccttccgtgtcaacttgaagtgctgttgacactcagctctcagtttcctttatgatagctaggctagttctttgggcagagaccttcctgtcttgatcatctcttcattgcagtgattcttctggctttacacttctttagtgtagtgcgttcccatctccattcttcatgtaacaaatggagggctttagatagagttaattctttaagagtcagtgagcagtatacagcatggtacaggttagtttcatgtcatcaaatcttaggagtttggaaacatttcagcttttaaattttccttaaaatatattattctgttgctggttataaagggttgttcaaaatggattgctcttctttaaaatccaaaatcaaaatttttcaaacttttgtttcccagataatgcttatgatcctgatgtgaatgctaaacagatatggattgacaaaacagtaataaatgactgtatatgcctgacattcactgataatgggaacggtatgacttcagacaaattacataaaatgctaaggtacgtaaaagcgtcacactccttaaaatcattgttgctgcctagttgaagtgtttcctgcgccatacttactgcctgtagttcctgctttttaaacatgaaatgttgtacccatagtgtgctctcttctctaagtcttccatcattttctactgctgtcacgttattgggcaatcccggccattctcctcatccctcccatgccttaacttacctaaaattctaccgtccctttagagttgtggcacattgtcctctgttgtttgtgctttagtagaaagatagttttagctaccatatccaataaaacctaacactttggggaatgagaactagtaaatttaaagtgattcaaaggatgaggaagtagcatagccgtaaggccctgggctctagggtcaaagaataggatgtcaggatgaagccaggtgtacctgacatggaagctaaaatacatagtacagaattgagatagagggagggtttggagcagaggcccttctgctgtcctccagcactccgtatgccgccagaaaaggcaggttctccccatcaggaggaagaggtgaaatctcaaggtcccagaagcctaaattagttcatccatctagttaggatgtgccaggcaatgaactcctgttttagtgtgtgtgtggcaaccactccagtatccttgcctgggaaatcccatggacagaggagcctggtgggctagtcgatggggtcacaaagagtgggacatgactgagggactaaaaacagcaacaatccttacatccctatagtgtggatttggcttccttcacttaaaagatggggaaacacatcagtacagatcttccagcaagcaaggacagggccaacattcaaacttaggcattttacttgatattaacaatgtttgaattaaaataaatgaaatttatacttttttctttttagctttggcttcagtgagaaagtcaccatgaatgatcatgtcccagttggattgtatggaaatggcttcaagtcaggatctatgcgtttgggtaaagatgcaatggtttttaccaaaaatgaagaaagcatgagcgtgggtttcttgtctcagacgtacttggaagtcataaaagcagaacatgttgttgtcccaattgtgtcattcaacaaacaacatatccttttgattttgaaatggaaactgaggaagtggtctggaccatcccatccttttggcctcctctggttcttgtttttaaatcataccttccattctagctcttgttcctatccactgcttctactctgcatctgggctaaatgtttgtccctctctggtctctgttggaattgtgccatggaaatactcaatataaggtgattgcatttttattaagcttcccagagctcacctttttatttcagtctttttgtggtcagtaagacttatggtctggcttgaaattttcaccaacaaccatggaaaacaagatatctttaggaaaatgtcttctgtataaacattcaaatatttgaaatcattaaatttgttaggagtcataatatacttgttcatactgcttgttcatttgaaatcacgctgtgattttaaaatttagaggataatttagtagtttttcagtttgagttgatttccatattgtaattgaaaaatgttattggtctaagtctttaacttacgatatacgacaggtattagattcaacagaatcaaaagcaagccttgctgcaattctggaacattctctattttctaaggaacagcagttactggcagaacttgatgctatcataggcaagaagggaaccaggatcatcatttggaatctcagaaggtaaatgaaacggtaaccctcagctggttaaccatttgggaagtaaagtgcatggagtaatttcattatgttcatccattgttacaccgagtagaaatgtattggaagaaatgtattcctcattttgtggcatatttaatgatttaccttttcccaagagtgtttcattgaggcttctttggcttcaatttgagataattttatattattataaataaatttcctggccctgccactctatctacatagcactgtttttttttgctcttggttataccatccctctcctcaaaaaaaaaagacatgacaagataaagcccagccaacaggtcccaacttggtaaagttagaaaaatccttttggtttattacataaagctaagcatgattcagcatctgttgagaaaagtaatatagatgagacttctgtgttgctaaatctttctgaggataaaatcctttccaaatgaagctatgaactttagggtgagtgtataatattgagagatctgattgatctgttgaacaataggagttaagccgaaaaagtgataatcttaaaatcctttaattcttatgcttttctgacctcttgcattccctttttcttttctcttcctttcttccctacccctcccatcttcacccccattttttttttttaaacttaattttgaggtacttgcagactcctgtgtagttgtaagaaatctcaggtactccttccccaggttccccccagtggtaacatcttggaacactgtactaagatagtactgttgatactgacattgatacagtaaggacattttcatcaactcaagaacccttttatatccacacccactccttccttaatccctgccaactgctaacatgtttttccatttctatgtctttgtcattttaaagaatattattatataactggaatcatgtagtgtgaaaccttttaggattggcttttttcattcagcttaattttctgaagattcaggttcttgcgtgtgtctgcttgctccctttttctagctgagcagtagtattgcattgtatgcttggaccacagtgtgtttaccaatctagttgtttacgattttcagttactacaaataaagctgtgaaataagtgcagagaggtttttgtgggaatatggtcatcattttattggataaatgcccaggagtgtaattgctggatcatatggcagttgcttgtttagttttttaggatgctatcagaatgtggctctctcatttgacattcccactggtaacatgagagtaaatacagtttctgcaccttcctggactccttgtcttccttcctccctccaactcctcagatcttgactcttaccatgtatttgtttgtgctttgttttgttaactaaaggttacttgcttgcttctcttatggtggagttaatatttagctctttaaaaactttttttacagctacaaaagtgcaacagagtttgattttgataaggataaatatgacatcagaatcccagaagatttagatgagacagcagggaagaaagggtacaagaagcaagaaaggatggaccaaattgcccctgagagtgactattccctgagggtgcgtattctgctctctgtgggagtgagaaaaccatcaaatgatggaaattacattacctacttgtgttataaatgctgccagaaatctctaaatgaaagtgtctcagacatgaaacagtgtattaaatttgtcatgctcattggctgttttattttcttagaaaagtttttaaattgcaaagtcatcaggtctacagtggttgtaaaagttcttttactgtgtgattaattatttgaattgggaatattgaatcaagatgccctggtgaccatattggttaattcaatcaaaataccaaattgtcctttttaaaaattaaaatattctagaatacatgcattaatcggtactctaaagcaattttacatatagcctacagaaaatgtgcattaatataattttgtggtaaatatatgtcagatttgaccttaaagtgaaatatgcttttaagattgaaagaattaatatctcaatataataatgttgatatgataatactactgtttcttagtcactgtgagacatgcaatactgactctccccgcccccctcctttttcccttttggtcaaccacatcatggcgtgtcagatcttcgttcccaagccaggaactacaggccttccgtgccccctgcagtggaagtgcagagtcctaactactagacctccagagacttgccccattccctttttttgtattaaaattatctcacaacccaagctaaggctaacatttaacattgacagctttactatgtaacttgttctgagatttcagcaattacaatggcagatgttcctatttcttgccttaccccagtgtgatagggaagtaccttagtggttaagaaagcctttctcaagaatatccagaaacagccagagcagatggaatagttttagtataccacaaagtaaaatgtcttaaaatatcggtaataaattttataagtaaaatgtcttaaaatatcggtaataaattttataagtgacaaaacactacttttgaaatcaggagtcaggaggaaaaaagtgagagaaatgctcaaattttagtttcgtattgaatttttcatattaatatatccttattgagttgttcacttcatttcctttagagacttctgatagcttgcaggcaggcacaagagatcatattctaacatgttttaagatgctaagtttggttactattctcaagtatctaagctctgatgcattaagaattaaaaacacatgtaccctcacagcacagtggttaggactcggcgctttcaaactgattgttcaggttcggtcccagtcagggtactaaaaaataaaagaattgaaagcacatttggatgactgctgattgttacagttccatatggaaaaagtgttgtctgtccactcccgttaaatactgctgcatttctttttcttttcaaacttacaatagtcccctttgtttatattagatttttgcttatttctgcacaagtaaaatttattgatgctttaaaattacaataacaatatatgttttgacagcagagtcagaaaatataggtccttttttgtatatatagacattttttacaaatttatttatttattttaattggttttcgccatatatcaacatgaatcagccacgggtgcacatgtattcccccccctccacccccctctctggtttgtcccagagcaccgacttggagtgccctgcttcatgcattggacttgcactggtcatctggtaaaatgagattataccatacatgctctttttaaacttttttccacataacttatcagcatctatgtgggtcagttgattgttttctgtaaaccctttttactggctccagtatattgagcttgtttttaatacagtaatccaatactaactacagcacctttatagtctgtagtattttattgaagagttagtattttattgtcaaagcactgtggtagctttctttttaaaaacatattaagtaatgttgaacaaatgcattggtaattattcagaaacgacagtcagttgttgtgtgtctttgcaggcttactgcagtatattatatctaaagccacgaatgcagatcatcttgcgtggacagaaagtaaagacccagctagtttcgaagagtcttgcctacattgaacgtgatatttatcgacccaagtttttagtatcctttactttgttacgatgctgtgtgtatatatatatatatatagagagagagagagagagagatcaagtgtatttcccttccccacttctctttttatcctgacaacagattctaatcctgtccaactttgcccttttctatgcactgttgaactctatgaaatagagaggatgaattaaaggaagtgagatggtgtgttcagaaatccttaacatttaagactaataaaactgtgagaattacttttggattcaattgcagaaataaagatcattacggaataatgatgtatcacagaaatagactcatcaaagcttatgaaaaagttggatgtcagttaagggtaagcttatatctgaaaatgtctgaatcttttgtaaaagttaggataacatgtttcgggtcaaacatgaaataatgtgctttttgcctaatgatttttatttatttgtcttaaaatatgtatttatttggctgctccgtttcttagttgtgggatgtgggatttcttcgttgtggcatataggatctttagttgcggcacgtgggatccaggtccctgaacagggatcgaacccaggccccatgcattgaaagtgcggagtcttagccactggaccaccatggaagccccctgcctttgcctcaacttctaatatcagtgtttttaacagcattggtgtggggcttgaccgtagagcacttgtgtattatttactaatttggtcttgaataatcttacaaggaggacagagcattttctctgttttacagatgaggaaattggggctcatatgtagcttgccttagatctaaagatgagtgtggctctcaggcacagatgcatatcttgtcgctacacttggataaggatcctggattattttgtatgagaataaaattgatcgattttaattcattgttgctgcagtagcctaaaaaatagtatctgataggtgatttacttttctggagttagttcctaaatatgttagaattttcctcctaaaccactttcatttttgtagagttcagtttaggatgatgaacattgcctactgtgtcagtctgtttatcttttacatgtttccacagaaaggtttttttgatgtgtgtgtgctacgaatttagtgttaaattctaaaattcagctgtgttaaaaagattttaagtccaggcatattttgttttccagttccatgattttcattttctgctcattttattttactctgaattctttgcgacagtgtagatggacctgaagagtattatgcttagtgaagacaaaagactgtattatcatttacgtgtggaatcaaaaaaataaacaactcaataaatgtgatgaaacagaaatagactcaaatatggagaagaaactagtgattaccagtgggcacagggtggtggggagagatgaggcaggggattaagaggcagactactatgtataaaataagctataaggatgtattgtacagcagaggaaatgtagccaatatttaatagtaactttaaatggagtataattaaaaatattggatgtgtcatatacctgaaactagcatagtattgttaatcagctatacttcagtttttcaaattgggattcaaaaaaaccaatagggtctgctacaaaatccacattttttttttcttttaatgaatttgacataagtaagatgtgtagctcatttttctagcccagctaaatttgtgttatttttggttaagtttgacctcataaagaaataagcttttacagccaacttttaatgtttttctgtaccagaagatgaactggtccagtatttggattcagtaaacaatttattaagtatttggtatgatttgccatgctagatgtcagcagaaaaagaggaaaagatacacatgcccccagagctctttttgctgaggggaatatggtatctccttggagcatcaggcttccctgatagctcagttggtaaagaatccgcctgcagtgcaggagaccccggtttgattcctgggtctggaagatccattggagaagggataggctatccactccagtattctggcctggagaattccagggactgtatagtccatggggtcacaaagagttggacatgactgaccaactttcactttcacttggagcatcagaatgggcttcttggagaaagtggtgattgagaggagactgagttaatttactgttttggaaagtctaacaagagttttctaggtccagaaaagaaggtctgcaaaggaaaagaaagaggcttctgagagaatggcaaccttaaagggattatttggaccagatgggcttccctggtggttcagctgttaaagaatctacccagcaatgttaaagaatcccctgcagtgtgagagacctgggttcgatccctgggttagaaagatcccctggagaagggaacggctacccactccagtattctggcctggagacttccatagactgtataatccatggggtctcgaagagtcagacacgactgactttcacttcactttggaccagatggtaattgggaatgcagtaatggggggagaggaaggtaaggccagaaacaacatgaaaagaactataagtggaaaaaaaaaaaaaaagaactataagtgaagggtgctgtgatttacaacttttatgaaatgaggaaccacagatggcttttagtagagtcacattgtgattaggtttttatggttttgactatagctgtatctatgaaaattttctttttatgtgtcagtattttactttttatcattgtacttagactcagctacacttacgaataaaatacacatgttgatcacatatagataatcagaatatgattcagatatgttgagttcaatattttagctagcaggtttaaaaaataaattatgtcagttgaatttaaagtttttatttcatcacaattagcaactgaatatgtaactttggataaatgactttaatttctatctagcaaacgtatggaatattaatgtgccagagcctttgctaggccctgatataggaaggagttagttgtggtttctgcctgctaagatattgcatcccagtagagaggggttttttccccttgtataaaactgggaatgataatacttgtcttaagtgaatgcttgacagtggaagtggttttggaggaaagctattacatttttaggaattatctcccccagaagtaagttacactccagatactgttattcaatctaaatgtcataagaatcatgagattatataggaaaatgtttcataaagttcttaaattttggtgaaggaataattgttttgagcatcatgaagcgcttcccttgtggctcaggtggtaaagaatccacctgcagtgcaggagtatcatatctaatgtttaaaatgccaatttcattttctttttcaggcaaacaacatgggtgttggagtagtggggattatagactgctatttcctaaaaccaactcataataaacaagatttcgattatactaatgaatacaggtatgttacctattaaaatgattgatgactgacttttacaatttattttggaaatacttccttaactttttctaagctaaatttgtttttcttttgtgggaggcttacctagaaaacacatgtaatcattgttttccaccctaggaattatattctactccatcacaaatagaaatatattctcctctgtctttactaatgaaatatttctgagatttgtactaaatagaagaagttcatgctttcttggtaacatttttttgtgtagtataaaatctagaaatttccatcttggatgggaacatcaagaaagtttagtcctctgtactttagacagaaatactcctatcttctaagaaggattagaagccataaagtaaatatgatcatcaattttacttgtaaatttaggaagcaccaacagctgtcctaatgagcatatattacagaatagaatgcaaacttcttataaataaagaacaaataaagtggaaggaaaggaataatagcagaatcagcaaattaggaaacacatgatagagaaaatcaataactatattattcagagtatagtcctgagactacagctacctctgttgtttaaacaaagaaaatataaggagctgttaaccacagtagtagaaggtgattgctcaaagggttaaaaaggacagtagcttgtccagaactgtcaggagcaaaaatacaactgttacctctgggttgaaaaagagtagacagtaaaggaactaggagagggttcccagtcccagcagtcagaggttcagatcccggtagaggctgtgactgacacaggaacacagtacctgccgtggcagggagcttgccagaaggcccgatcagtctctgtctgtagacatggcctgctgttggcaggagatgtggggctgagttgtccttagaggaaacctgtctctgcccaggggtgtgtgtaggctggaacagtctctgctgctggagcgaagagggtggcctgagagcacagctggagctcctccggaagggccaccgggttgagattcagaaagtagttggtgggttggttggcagcatactttctcagcagaggacctgaagtactagtattttctgtcttattcctggtgaccctggctccgttctatgaacttttaaagatacttagaagattgccatgactctgagtgttacttaactgaacattttggagaaccttccactctgccctccccactccctagtgttcacatgatcaataaatactaggttgcttatgctttctttgtgtttgacagagatttaatctttatcattttttttttcttgaattgattttataatttacttctttttgaggttaacaataacagcactaggagataagctgaatgattactggaatgaaatgaaagtgaaaaaacatgcagaatatcctctaaacttgccagttgaagatattcagtaagtacatttaaaaaattattgactttaatatcaggatatggtattggagggctggcggttgtacagtgttcataaactttggaatctaattcatggtaagaaataagtcccacagctcaaatcagcatactgtaagatgaacatatgcatttatatacaaaagattcttgaaacagtatttacctgtattattagtgttgataacacttattttatttaaaaatgctcatcttgacccactaaagtgacttcatgatccactaatgggttgtatctcatagtttgaaaatctctggtcttgtgtaataggatcgagagcctaaagccagatttcagattacattgtagttcttgcttaagtagctgtgtgacattgaacatgttatttgacctcttacaagcttccattttatcatttgtaaaatgggaacagtaatagtctttacctcgtagagttgtaagaatgaaaagataatagtgctccacagtgaaacacataagccctctgaatgaataataactctgtgtactgatggaaggatgtccaagatagataacggaaaacaaaaaggatgcaactcagtgtcttaatttttccattgtgtaggttttttattgtccacattcatatgcatacttttataagcatagaaaatttctaccaggattaaagaagtaagcaataataacctcctgaaacaaggggtttatatttactctataaaattttggattgtttgaagtttctataaaaccatttaaatgtattttttaaaactagattttaaaagttactagcgttaaggtgaattatttgtatgttaaatgagtctacgtaaattctttgcaggatcatagtaatttttgaatgataaaaatgtgaaagccttattttatgaactaaagaaatacgcaaatttaagttgttcagagatatgctatagtagagggtaaaatgtaatacagttatgaaaattgcttaaactgcaataacacaacttgcctattttctgtacttgagttgttattgtgctaagaagtatattggatttgacactacagatgctaacttaatatgaatttcagagtaagtcatttgtagtattttcttaggtttttgagttcttactatagaaaaaaattaataatgtgctattaaacaagacatttaaatcactttgtttaaattagtctgtcacttcaatagtagccagaattgtttttaaaaactaattgtccaccaaagtagtatgttacggaattttcagggacagaattaatagaattttgcgggagaaagtccagtggaaagtccagtgaagtacttaggtcggtgaaagtgaagtcgttcagtcatgtcctactctttgtgaccccatggacgataacctaccaggctcctggtccatgggattttccaggcaagaatactgaagtgggctgccatttccttctccagtggatcttcccaacccaggggtcaaacccaggtctcctgcactgcagacagacactttaccatctgagccactaaggaggccctaatacttaggtcggtatttccatgttaacattgagctgttattttgttacaggaaacgtcctgatcagacatgggttcaatgtgattcctgtctaaagtggcgaaaattaccagatgggatagatcaacttccagaaaaatggtattgctccaataaccctgacccacagttcaggtaccatatagttggtagtacccttttttggaaaggcagaaactactgttcaagatgtatgaataggcactggtgacgtgttattcccatgtgatttttctgactagaaattgtgacgttccagaagaacctgaagatgaagatgtggtgcatcccacttacgagaaaacctacaaaaagaagtgagtgttatattaatgtatgtgggaggataaaacccagatcatccgcagtgctttgtctgcagcatcagtcagtgcgtctctgagagacagcagtatagccacaaaagatgactccaagatgtggagtgtataccatcaagtgctttttaagtttatgctgtggatacctgctggttattgctgtcctgtctacaagatactttgcatatgtggtatagttttgtctttttgggctaaacaccaatgaatcagaagtgaatacagcccttttaacagagaaggtgaaacgtgtgttgttaccagtgtgaagttgacgactcctgtaaaaggcctgactctgtcctgtgaagaagcccataaggataaattcctttggctggggacatggatcaggaaaattcctgatgggtggaatttgttgggtggagagcaatgggatttgttgggtggagagcaatggtttgttcagcttaaccagagtaaataatgtgaatggacctaatggagaatagaggtgtagagagaagctgggaacagcagtgagccacggggaacattaggcttatgtactgctcgtggaaggtcgagttcatgaagatttatagtgcactattcttgaagataaagtttgaagcaagattggaagacattagggtgttactgagataagaggtagaaagcacctacctgcctagggctcagccctgattataaggacactgaagcagagttcagtgtacctacctgtcttagtgacttagtagaagtggcaagtatgtcattggaaaacaattggaaaatgaagtaaatttaaatttgttcatttttctttttcttttttctctctctctttttttttttaagagacagggaaaagttcaggatcagacaactggaaacagcccctcgggtaatgaggccttccatgttacagctgtgtcctggattgggtggtgattaacttagtactcaactttaaacttaggagttatatagcagattgaagcaatcttgttcattgagttcacattctctctcttcttgtacccacttcttaccctctttcccaaccctgacttcctaacccaacaacttcactcagatggttcaatttattcttaagttgggcatctggggaaagacagatgttcttttaagaatcttctcattaggtagacagtcaaggaaaaattaataggaacacccagggggctgcattctagtactgacttagaccaaagctacatcattgaccctccctcctgttctctctctttttttaattgaaagtatagtttattttgtaatattagtttctggtgtgcagcatgtgattcagaatttttacagataatactccaataaaagtgactacaaaacaatggctatagtttcctgtgctgttcaataatatatccttattgcttgtctcttttatatatagtaatttgcctctcttaatcccatagccctatcttgcttaatggtggtgagcttttttttggtgtgttaagtaggtagttgtacttcttatataggaagttgtgggctgctgcattggtatatgtatgcttatcaatacattgaaatgttttcttatataaaggctgagggtttgagagtgagaacagaactgggagaaaaagaatagtgggcttaggttttttaaaggcagtgtctttgtgactttatcttcctttatatttatgtttctttctcttggaaaaatatttttattacgtatctgtgtaagctctctatataatccaaggtcaacagatctttctgtaaaagtccaggtggtattttaggctctgtggaccccacatacagtctccgtggcgtattcattctttgttttgttcatgcgtttaaaaatgcgaaaccagtcttgactcacttgcaggctggacttggctctttgtcgctccctggtgcagtgtacacagcacaagctcacgtggttaggatagttctcagtgcctctggtttagggaacggggaaaaagtactttgtgtttgggacaggacacattttaaaatctcttcttaagatcagtttttttttccttcaacagattcatgccgaactcctgtttccaaaaacccctgtgtcaagtcaaacctttactcccgttaaggaaggtgttccacgaggacatctttcagaagtagcaaatacttactcaacaaggcttataaatcatcatcggggttcaccccagtctgaacctgagaataacaggtcagtggctaaaatgtgtttttcgacttgggaaacaagtatgagacagcctttacttgacacttttatggcaataatgcactgctaagaaagtgtagtttatacgcatattaaacaattcttacggatgagttggtttttgttcagttgctaagtcatgtccgactcttttgcaaccccatggactgtagcgctccaggctccttctgtccatgggatttctcaggcaaggatactggagtgggttgccatttcctaaaccaggggatcttcccgatccaggaatcaaacccatgtctcctgggtgggcaggcggattctttactgctgcgccacaagggaagccataaggatgaggtattttgcttttattgtctaaacttacctctaccctacttatgttactgtttaaatcaattaaaagaatattttttttcattgtggaaattgatcgtattgtcacttctttgcagttctgtatagtgaggaaattcctgcttttaagattatcggtatctgagtaaagaagaacacgaagcaatggcagctcacagtgcaacagtataatgtcagctgtgacccatacgttctcagcatagcccgtgacatgttatgtacttggatgagtggaatgaagaaggagagaaagatgtatgataacctgaagtgaacagttagcgctcttgactggaatggtcagggaatgctttatgatgaggcggtgctaggttggttcctgaaattctaaaagcagtactctgcacaggttgcctttacaaagtgaatagaaggaacacaggtgaagaatgaagactgtgatgccagttaactaatacatcagactctgtatggatttgttgtttgaagagaaacactgaaacgagattctctgaaatagtgataccagacgttctgagaggtttttctttaatttgttaatttctttttgtctgtgctgggtcttcgccaccgcgagggctttttctctagttgcggtgcacgagcctctcgtcgtgcagtggcccctctacttgtggagcaggctccagggctggcgggcttcggtagtcatagcatgtggacccagcagttgtggtgtgtgggccccgctgccccaaggcatgtgggatcctcctgggttaggaattgaacccacgtctcctgtactcgcaggtggattctttaccactgagccgccagggaaggccttcaaaagaatttttgaaagttgagaaaagattcaaaataactgcctttatgcatatatacatcatggagggcatgttttgaaatagtttgctgtttatatatagcctgcattttgagatcttgttttgtggctggattgctgcttaactgagaacatgtgtgaatttttttaaactagtctccatgatattcttggcttttcttcataaagctggttccagacaaagtaagaaaaaagtaaattttttgtaatgtttacagacaaggtaatagaatttatctttaaaagtgactaccatcactagaggagggtatgacaacccactccagtattcttgcctggagaatccggtggacagaggagcctggcaggctgcagtccatggggtcgcaaagagtcagacaggactgaagcgacttgagtgcacacacatagccaccaaaatggtagaagagaacagagcgagatttttcatttgtaaacctctgttcctaaattcatagagccaaagtctatggggagatggtccctaaactaagtcttgacaaacaaataggaattagcctggtaagaaaaagaacagggcaacttctaattaaatgctcaactgcttggatcactagactgtctcagaacttggaagcactggacatttctgaaggtgggaaactgggggtaagaccaggaggctactatgagagttcaggggagggtagttccctgggccttttcacagcctgcaaaggccagaagtgctgctgctcttcctctgtcagaaggtagggagcttactctctggagcagctttcactgccactgtttcggaagcagaaggagtcggggagtgtttgaatactgaatggtgaggagcctgtctcccctcctcccctccctggctcccaagaggaaggccctaggtgatgatgatggcttccttgggctcccacacccagccctcactgcttccagtccgaaatagagatatagccgagggtccccagaggcacatagctcaaacaaagggagcagaagagaccatgcggaactaacttagagctgggcagatgtttaacaggtaactctcccagaaggaggagataaatggaagacaggaaacagaagacagggtactggagggaccaacctctgaataagggtattccaaaatgggacatcagagaagggaggaaataatggaagaaaggcttaatttccacagtgtaatgtctggggaaagataagaaatatcatcatgaagttttaaagcacttggggaaaagaattttttgtttaagagagaatgcaggctatattcaaaagatcaggaatcagagtggcaggaacagtgactggaagataatgatgggatgatgcttttagagtttcgagaaagtaattcccaacctggaagtctgtataaaaaccaaaccattagtgaacttgagaggtgaaaagaatattttcactgtgcaatattttacattttttatgtcatgtgtaccctttctcaagttcttattgaagattatgccccacccccatacgagagagtaagcctaaggaaaggggagatctttctcgagaaatgaggtcttaacacctaagacccaagtggcaaatcagtacaaatatccttcttaggtgggacaaaaagtgatcaattggtgataaattcacagaaaactaatcagatgaagaaaacaaggcagtttttaatgcaggggaaattgttttccatgaaagggtagggcatgctggtttaccttgtgacccacctttgatccctgaatgttgatggaattacgatggacagatgggaatggcagtgccattcagtgacctctaggttacagacagcactcctggggtgagtggtggtattctgtttcaagatgttaaattgaaatgccagaagacatcaaatgagataaattggcagtataaggatgtggtactttgaagaggtcttaatagtcttgtttcctcatctgtaaaaggagacaataatctctgctttattaaatcattgatatttttcttttgatattttgatattcaaaatattctgcaaatatattgtttaaaaaaatgaacgtgggcatttcttagaattcaagatagattacttcaaggtcatcttatttttttccatatctacattttataatcctatatttcatatatgaaattttttccagcctgaaacgaagactccctactcgctcctcaattttgaatgcaaagaatcggagattgagtaatcaagtatttgaaaattctcatcaagatgatgatgatgatgaagatgtcatcatcttagaagaaaacagcactcccaagcctgcaggagatgatgatactgaagtgaaattgcagcagagccccatggagcagagtggtgtggaagtggagcctgtgggtgacagtgaaccttgtggccagactggctccacaggtacttcgacatcccagtgtgatcagggaactaccacagccacccagactgaagtgccaagtttagtcgttaaaaaggaagagactgttgaagatgcaatagatgtaagaaatgacacagccgcactgccagcctgtgtggaaactgaaggaaagacacatgaaacccagggaacctttgataaatctgctagtgacgctagttgcaagttaaatgaactaagaaatgagctgcttctcgttacccaagaaaaagaaaattataaaagacagtgccacatgttcactgaacaaatcaacgtgttacagcagaggatactggaaatgaataacaaatacgtgaagaaggaaacttgccatcagtctactgaaactgatgctgtatttttactggaaagtattaacggtaaacctgaaagtccagaccatgtggtgtctcagtatcgtcaagccttggaagaaatcgaaaggctgaaaaagcagtgtagtgctttgcaacatgtaaaggctgaatgcagtcagtgttccagtagtgagagtaaaagtgaggtggacgaaatggttgtgcagcttgatgacgtatttagacagctggacaaatgcagtgttgagagggaccagtataaaagcgaggtgagttctatccctcagcgtaaggagaggtgtaggtgtccaggggcatcccagccttaactccccagttgctagattgcaataaatcgcttcataatatttccatttctcgactacctgctaatccactggttctcgactgggagtggtcctgatgtgggcatctggaaatgcataaggtcattgtggttctttcagcatcttgaaggcatgggcagtgatggaattgaaagggtggggaccacgtctggtaaatgtcctgtatatacaggaccctgctgtaccacaagggatcatcctgctgattggccctgagacagtcagttactaatatgctgggagttccatgttatttgtaagcttgaaaataaccatatgaggtagatgacgatgtttgttattgttgttcagccactaagtcgtgtccgactcttggtgaccccatggactgcagcacaccaggcttccctgtccgtcaccaactcctgaccttgctgaaactcatgtccatcaagttgatgatgttatccaaccatctcatcctctatcatccccttctcctgtcctcaatctttctcagcatcagggtgttttcaaatgagtcggttcttcacatgaggtggccaaaggattggagtttcagcttcagcatcagtccttccaatgaatattcaggactgatttcctttagggtggactgattggatctccttgcagtccaagggactctctagagatttctccagcaccacaatttaaaagcatcagttctttagcactcagccttcattatgggccaactctcacatccgtacgtgacgactagaaaaaccatagctctgactctttggacctttgttggcaaagtgatgtctctgctttttaataggctgtctaggtttgtcataactttcttccaagaagaaagtgtgttttaatttcatggctgcagtcaccatccctggtgattttggagcccaggaaaataaaatctgtcactgtttccactttttccccatctatttgccatgaagtaacaggaccagatgctataatcttctgttttttgaatgatgagttttaaaccagctttttctctctcctttttcaccgtcatcaagaggctgtttagctttccgccattagggtggtttcatctgcatatctgaggttagtgatatttctccccgcagtcttgagtttcatccagcctggcattttgcattactctgcatgtaagttaaataagcagggtgacaatatatacccttgacgtacttcttttccagttttgaaccagtccattgtttcatgtctgtttttaactctttgcttcttgacctgcatacaggtttctcaggaggtaggtaaggtagtctgtctggtattcccacctctttaagaattttccacagtttgttgtgatccacacagtcaaaggctttagcatagtcagtgaagcagaagtaaatatttttctggaattctcttgctttttctctggtccattggatgttggcaatttgatgtctggttcctctcccttttctaaatccagcttgtacatctgaaagttcttggctcacaaacttttgaagcctagcttgaaggatttcgagcattactttgctagcatgtgaaattagtgcaattgtacagtagtttgaacattctttggcattgcccttctttgggattggaatgaaaattaaccttttccagtcctgtggccactgctgagttttccaaatttgctggcatattgagtacagcactttgacagcataatcttttaggatttgaaatgcctcaattgaaattctgtcaccactactagctttgttcgtagtgatacttcctgaggcccacttgacttcacattccaggatgtctggctctaggtcagtgatcacacattgtgattatctgggtcgtgaggatctttttttgtacagttctgttttcttgccacctcttcatgatatcttctgcttctgttaggtccatactgtttctgtcttttattgtgcccatctttgcttgaaatattcccttgatatctcttattttcttgaaaagatctctagtctttcccattctattgttttcctctgtttctttgcattgttcaccgaggaaggctttcttatctctccttgctattctttggaactctgcattcatatgggtatatctttccttttctcctttgccgttagcttctcttttcccaactatttgtaaggcctcctcagacagccattttgctttcttgcatttctttttcttggggatggttttcatcaccacctcctgtagaatgtcacaaacctccatccatagttcttcaggcactctgtctatccgatctaatcccttgaatctatttgtcacttccattgtatatgcataagggatttgatttaggtcgtacctgaatggtctagtgggtttcccctactttcttcaattcaggtctgaattttgcaataaggagttcgtgatttgagccacaattagctttcggtcttgtttttgttgaatgtgtagagcttctccatcttcggctgcaaagaatataatcaatctgatttcagtgttgaccatctggtgacgtccatgtgtagagtcgtctcttgtgttgttggaagagggtgtttgctatgaccagtgcattctcttggcaaaattctattagccttttccctgcttcattttatactccaaggccaaatttgcctgttactccaggctcttgacttcctacttttgcattccagtgccctatgatgaaaaggacatcttttatggtgttggttctaaaaggtcttgtaggtcttcctttaagtgaaactaaacaagtgaaactttatagcatgctgttttttgtctacgttttcttggttttctttttgagtgtaaagtgaaagtgttaatcgctcagtcatatccgactctttgcgatcccgtagactgtagcttgccaggctcttgtgtccatagaattctccgggcaagaatactggagtgggttgccattcctgtctccaggggttctttccagaccaggtctcctggattgcaggcagattctttactgtctgagccactagggtagtgtcttaattttttgatggagattttcatcaagcagggttttgtacatttcttagcttaaatttgcactttgatagtagtattttaaaattttacagttataaaattaattttttgtcagattgaattattggaagtggaaaaatcacaaatccgttcgcagtgtgaagaactgaaagctgaaattgaacaattaaaatctgcaagtggacaagtaggagctgatgtatcaacttcaagtaacatcgaggagtctgtaaattacactgatggggaaaggtaatatgagatgaggcagtttggctggggctgcagttctggttagcatgatacttgctgctgctaagtcgcttcagtcgtgtccgactgcacaaccccacaggcggcagcccaccaggctcccccgtccctgggattctccaggcaagaacactggagtgggttgccatttccttctccaattcatgaaagtgaaagtgaagtcactcggtcatgtctgactcttcgcgaccccagggactgcagcctaccaggctcctccatccatgggattttccaggcaagagtactggagtggggtgccattgtcttctccctagtactcctcaaagcttgttctatcaaaagaagttgctcactgctgttatcccagcattacgatagatacgaaaagaagtacatgtcatcatcctttccttcagggaactaacagtttagtgaacctaagtaaaataggaattgaaaggaataaagctttgattggtcagagtggttaggagtattttggggtgagaaactgaaaattgtcccagatgttaaagaattaggcagtgactagaggataagggtccatttcatgaaacttagttttttaccttgatttctttctgtatttacttggcaatacttatttatggtttgctgtctcatttaactacatgccttctgcaattttaacttaggaaatccataattactgaaaattgccattacaggtgataataaggaagaagttctgtgaaaataagaaaacaataaatgaatgattactctcagtaaggtattaaatggacttggggttctgacagtattggtggttattattcaaaaatattcattaagaaaaaagaaaagtcagtaagggataaagttgtataacccttcctactaatgtaataagacttggtacctaaaacatactgttgttgggaaatagtttaccatccttacctgacatttaagtttttgaaggatttcactaaacctgtttatggaaatagaatgaatagaatagtacatataactatggggccaaaagttcggaaaataaactcccaactggtgtttcggaaaacgttaaccacaaacatctgtagtacttgtaaaatacagatccttgggccaaagtatgggttattgaattggaaacttttaaacagttttccagatgatccacttgcacactgaactttgagtcattgatgatcacttagatttgtgctttccaagacagtatagccacatttggccactggcacttgacatgtagctagtctgagttgagatgtgctttaagtatgaaatacatgctggattttgatgccctcatattcaaaaaaaggagaagaaaacgcctcaatattttgattatatgatgaaatgatatttcatatgtaacttaaatttattgcttcattttagtttttaatgtggctacattctttaaaattgcatgtggagctcacagtgctttctctcagtgccgttacatacactgaaatagggtcttgtgatgtgtcctgtagagaaggggagttagccaggagagtccaaagacttaaaggataggaaggtcagagatggccgtagggtcatcctgggactttgtctgttttttatatagaaattaggcatctgattgaagatgagtgggtaagcttggacagaaagcaactgttacgggaatggtataaaattttatccaagatccaatggaatcctaagataagctcacaaaccaaatgtaaaatctcatttttgtgtgttgtcaacctaaagatgagcatcttgttccttcagagatttctaccctagaaagatcagtgtctaaactaaagatgagatcagatcagtcgctcagtcgtgtctgactctttgcgaccccatgaatcgcagcacgccaggcctccctgtctatcaccaactcccggagttcactgagactcacgtccatcgagtcagtgatgccatccagccatctcatcctctgtcgtccccttctcctcctgcccccaatccctcccagcatcagagtctttgcttcttatactttggaggagacagaatagaaacaactgtaacattctcttaatccatgtgtttcactcaatcttcaaggaaagaccacttgctgaatagttgtaggtatgtatctaaaatccagaatgttgatattcagatagctacatgtatggaaatcaggaaggaatgataatgatcatttaatctagagtttcccagtgtttataatgaattcatagtacaattaatagtgaactttagtatgtgataggttcagagaaatcctgcatttaaaaaagatcttgtttaacattatttaacccaaagtttattaagcttagttaagcacaaatccatttatacaactatggatatctcataaccagatatctcataccgtaaccagaacatggtttagggaacagttgagtttactttcctcagaaggaagctgaaactcaaagagctacgtcttattcttaggccagtgttctcactagtatatcaatactcttttccaggtaataacaagattttcagttgtaaatagcatcttgttatttatgtatttaccagtccacattttttctagtctcgaattaaaattatattagttggaaccaaacagagccactagttttatgaaacattctatttaccaacagataaatatttagttgttaaataataaattattaagtgaattattaaatccagttctcttgaggaagttttgcttttcatttagtttgtgtgccattctagagattccattttaatatttctcttaaattttaagccctatttaagctgtattattataatgttcatctaataggtgtaatatttgtattttcagcctcaaacttcgatctcttcgagttaacgtaggacagctcctggccatgattgtacctgatctcgatcttcagcaagtgaattacgatgttgatgtagttgatgagatcttaggacaagttgtggaacaaatgagtgaaatcagtagtacttaaagtgtatttgagataatgaaaaatatttgctcagtcttcgggttgtacagctttcaaaatgtaaacaattttgttataagtatgataggtaacagactgaagaaccatattctttgctgtattctatgcattcaagtgtggacacaaatactgtgtacacattatcacaccttttgaaatgcctgtgaatcattgatattgagcagctacacagctaactcatgattctgctttgaaatgtaaatacttataattaagtctgcacatatttttttaatcaccctaatggactcaagtgtttttcaccaagaggttttcaggttgcccctaacctttgtgcaatcttttctggttccctaaagtgtatttttctctgacttgggggctgtgccataatacaaatggaaatgttacctttgattttcttacagaaaagtttaaaacagggttttttaaaatggaataacattcctgataagtttgaagtgaattgagcattaatgtttctttttttataaattcttatttcctgaagtattttattcatgaaaagaaagtaaagcagaaactccatttttgctgggatgttttgtgtgttgagattgccaatcatgatcaaacgcaaagtgttttttacagaaaaaagcaacgatttttagtataaaagtatcaaaaatattaaacgtcccaccacatttaccttgaagcccatttttggctgcttagttcgcatggagtgggcacaataattgtttaatatttctttttgtgaaatttcctgtacagccttttgtagaattactacaggttaatatgagttgaggaagacaatctttctccagcagtactgcatactttttattatattgcaaacctaagtgttttgtatcctggagttaagcagcaaacaacctaggattatagtattacatgccctaaaaattatgctttattggtcccatgttttgtgcaattataaagagatggctttctattaagtataactgtgtatataattaagaatcgtattttccacaactaaaatgtgcattatttttttcaaagttttatcattgctatttatttttacttttgtttctgaacattgaatacatgttccttttatatgcttaattacatgtctgtcaggtacaatgttaaatttttattgtacattaacttctagaaaaagcaaataagtgaaattttatttgcttgataaggtaattgaaagtgtatttttggtatgaagctggttttctgtcacaattgtatctcctcagaatttaaaaatcttgtaataaaataaaaatatatatgatggctaacatttcagatattacttttaaggaattctatattcaagttatattttgaaaactaagattgtgtctgttaagaccctcaagttgagctcattatattcattagaagttacatttagaagactaatactgtttgtttccattaagatgctcagactgggctcaccttgagagtgtgtgtgtgcattttacactgaaaaagaaactttctttgtataaatcattttaaaaaaccttgcctaaatgagagcagctgtgtatagcatgtctaagtttgtgaatatttatgtagcacttatttctggaatttgcaattttccctttccttttggtcagctatattcttgcagaaatccatattatatggctgttaactggcttaatgggaaaattaatcatagaattagtaagagaaacttgcttgctaagttgattctttgactatatttaacctaatattcactctttaagaaaatattgccaagataagactgaaaatgtggctagcagatttaagagaacaacatggacccacatcgcatatattgaggtacctctacttcttttttgttgttgttaaattttttaaaaggctagtgtgggaggagtgtgccgcttaaaccacttgtttaaagctttaaaacaaaaacagtatgaggacgtaaactagaattgcccacagtccctacagacaagacctcaacagagactacagactcagcgaacgctaaatgttctcacagcacggccagtgtggcacatcgctgttgcctttattggcctcaattcctactgtgagcaggaggcgctggtgactgcccacagtctccgacacttgacttgtgctattgcacgacccattaggtcattatttgacatggaggaataatcaaaggtgatacgtactggtggttctccactggctgcattttagaatcacttgtggggttcttaggaatcccagtgtgaggatgctccccgtactataagagttttgagggttgaaccaattgtcaagactttttttaaggcctcccagtgcactatcagagccaagagccattgagctacaccctctagtgatgagtgtgagtgttgaagtcaggtggttcctgaaaccaacccagaatctgcatggccactttctcactgcggtacaaggtcagggaaagggaagcagagctggtgcccaagttagaaatataaataacaacagaatggtatactgtgaaaagacttccccactgcctccctgcaggtgaccagtatttttttgtatgtccttccagaaaatattagctgcatacatatacacattcatatatatgcattcagtttctattaaattttatatctttatatttgattatatatttgtatcaaatctacacaggtattataccatgtaattggtaacatttccatattactctttgaacagctttagtagcattttaaaatgacatgaatacgctcccagtgttccttttctttctttctggaaaaaacactactgtgaaaataaatacccaccacacatactttttcaggcgtatccagaatgtaaattccttgtcatgaatactgattggtcaaagagtatatggtatgttaattttaataaatgttgccatccagaaatcgttgtacttaaactcctatgaaaaataaatgtacgagtgtgtttcctcctatctccatcaaacttactaatagtatgttggaaaatggcagcttgcatttctttgtcccaggttgaacatgttttcatgttttattggccactttaattcctttttctgtgttctccatcataactctctgccacctcttttggattcttcggtttatttttttaaagaagctgagtgtctgttaatgtggccacaaatggctccaagctttcagaaaacctactgttggttaaatcttactttgcacggtgttacacagattcattgggatggaatgagagactcactggagtcgtgtttttctggcccttgaatagcatgtggccttatgggtgctgacccccatttagtaaaaaaatccacatacttttgaccccccaaagttactaataacttgatcagaagacttactgatgaatagtcaagtagcgtatgttgtgtggatcatttactgtattcttaaagtaagctagagaaagagtcataaggaaaatgtttaaagtactgtactaccagaacttgtcatttgtttacaaaaagatggaccatctcagtgtgtacgtcagtattgtcttgtgtgatagatactgaaaatgggaaggtaatgtgaagaattcatatttatttacaggtataatgattcatgcaccgataatgaagaagcaataacatgattgctttatggcagcctagtgtaactgatataattgcttcctggtagcctattctgtacactaataattatcaaatttttatggcatacagtattatagtcatattcttaatacagcattggaaatgttgcaattttttttttttttgaaacttacctgtgatgatacgcagttctaccatactgtatggtaatataattctttgagagcaaagttataaaataggaaacaacacattaattttatattaagtttcactcaccctatagtatctatgtatcttttatacttttatgacatacctaaattttccttattaaataaatcatgcactgttatatatgaattctgaagtataaatgatttacaacatgttaatttcaggagtacagcacagtgattcagttatatatgtatatttttagattcttttctcttataggttattacaaaatactgagtatagttccctgtgctatacagtaggtctcttttggttagtaataagtatatattgatggcagccaactaattcagccctctcctccaactttttctcaaatttttagatatttctgggtcgtgcagctcaactgtatttttaaattgtaatctcgcccccaaattttcagtatattgaaaaaaatccgcatataagtgaacccaggcagttcaagcctgtgttcaaaggtcaacagtatttgtctttcggacattggtcacagctcacaaagaatgccgttaatatccttccatttggcatagaggaaaatatatactgttttccaggattaattttgtgatcacaaagttaatttacatatactggcctctagagagaaattcaagaccagagaagacagtcctggtaaattcaagccattcaaatgcaggtgaagttttcaggtgagtggagtcatagagaatgaattagctattataatgtgtgaatcacaagttaaatttcctgcacaagcattctactaagaatgggctgagtaaatttaattagctatttcaaacagataaaacaagttgaaagttcagtgttaatttcttgcaaagctgtcaggactcagtattacggtaacaatacatagactcaaaagaactttgtaccacaacctggctaatccttaggttgagtgtcttcaataggtaggtgttatccagaactcgtatcctcataacagtgtaatggtgctttatattcacaaagtattgagtacatgagtcaacagtcttacctatgggtttcttgttaaatagtgttgaaaattttggcctgtgttttcaactatgtggccggttttcaaagtgtttgctgtgatctgaaccagggctgactggaaggaagtttgagctttttgttttctttttttgaatagacagttgtcccatgttttttcgagtcccttggtggagtgtctctctgtgcagacagctgagacactagagggggctcttcccaacttgctgtgtaacttgtgagttgtctgagtagctcacagtctgaccttttcctgggcatagaattagaggggtctagggtccaactcagcagtggccacaggactggaaaaggtcagttttcattccaatcccaaagaaaggcaatgccaaagaatgctcaaactaccgtacaattgcactcatctcacacgctagtaaagtaatgctcaaaattctccaagccaggcttcagcaatacgtgaaccatgaacttgcagatgtccaagctggttttagaaaaggcagaggaaccagagatcaaattgccagtatccactggatcatcgaaaaagcaagagagttccagaaaaagatctatttctgctttattgactatgccaaagcctttgtgtggatcacaataaactgtggaaaattctgaaagagatgagaataccagaccacctgacctgcctcttgagaaacctatatgcaggtcaggaggcaacagttagaaccggacatggaacaacagactggttccaaataggaaaaggagtacgtcaaggctgcatattgtcaccctgcttatttaacttctatgcagagtacatcacgagaaacgctgggctggaagaagcacaagctggaatcaagattgccaggagaaatatcaataacctcagatatgcaaatgacaccacccttatggcagaaagtgaagaggaactgaaaagcctcttgatgaaagtgaaagagaatgaaaagttcggcttaaagctcgacattcagaaaacgaagatcatggcatctggtcccagtggaaacagtatcagactttatttttttacgctccaaaatcactgcagatggtgactgcagccatgaaattaaaagatgcttactccttggaaggaaagttatgaccaacctagatagcatattgaaaagcagagacattactttgccaacaaaggtctgtctagtcaaggctatggtttttccagtggtcatgtatggatgtgagagttggactgtgaagaaagctgagtgccgaagaattgatgcttttgaactgtggtgttggagaagactcttgagagtcccttggactgcaaggagatccaaccagtccattctaaaggagatcaatcctgggtgttcattgtaaggactgatgttaaagctgaaactccaatgctttggccacctcctgaggagttgactcattggaaaagactccgatgctgggagtgattgggggcaggaggagaaggggatgatggaggatgagatggctggatggcatcactgactcgatggacgtgagtttgagtgaactccgggagttggtgatggacagggaggcctggcgtgctgcgattcatggggtcgcaaagagtcggacatgactgagctactgaactgaagtgactgaactgagggtccatggagtcgctaagagacacgactgagcgacttccctttcacttttctctttcatgcattggagaaggaaatggcagcccactccagtattcttgcctggagaatcccagggacaggggagcctggtgggctgccgtctatggggtcacacagagtcagacacaactgaagcgacttagtagcagcagggcaataataaatacttactatatatcattacatattatctataattacattatgtaaactatatgataatattcactgtaatcacttaaccggtagtttctcattttctactagtttactgtgttcttaaattttttttttcagtatcagatattttaagctctataatggaaattttgggggttttttttgggaggtgcatactgggtggcttatgggatcttagtttcctgaccagcaatggaaccagggcccccagcagtggaagcacagggtcctaaccactggaccaccagggaattccctctataatgggaattttcaaatgtatacaaaagtacagaaactgttcactgcagccctgtttacaatagcaaggacatggaaacaacctaaatgtccatcaacagaagaatggataaagaagatgtggtacataaatccaatggaatattactgagctataaaaaaagaatgaaataatgccatgtagagcaatgtggatgggacctagagagagtcattgtgaactaagagaaaagacaaatatatgatattggttatatgtggaacctaaaaaagatacaaatgaacttatctgcagaacaaatagatttatagatgtagaaaacaaatttatggttaacggggtaaaaggaagagggagggataaattgggagattgggattgatatatacacaccactatatataaaatagataaccaaggacttactgtatagcatggggaactcttcagtactctgtaatggcctatggggaaaagaatctaaaaaagtgggtgtatgtgtaacattcactttgctgtacacctgaaactaaaaacattgtaaatcaactatactccaataaaaaggaaagtagaaaaactgttacaatgaactcatgtgcacccatcacttcaacagtttccaactcatggtcaatcttgttttacctgtattcccattaacatccctctggtaataatttgtgtgtgtgactgtaaatattgactaattttttattgtggaagtatataccatttttaagtgtatgattcagaggcatgcagtacattcatatcattgggcaactacctacaccatccatttccagaactttttcatcatttcgaataaactgtgtatcctgtaaacaataactccccattcccacctgtcccctggtggcttctgctttctagctctacaaatttgcttattctagatatctaagtggaatcataacgtatttgttcttttgtgtctggcttatttcactttgcataacatttttaaggttcatctatattgttgcaaatatcagtttgattcctttttaaggctgaatgatactccattgtatggatgtaccatattttatccattcatgcatccatggacatttaagttggttccactttttgctattaagaatgctgctatggacactattgtataaatatctattgttgtctttgcttttaattcttttgggtacatacctaggagtggaatcttcctttttttccctcctcttatctgtgcttttcaaacaactagattatctacagcacatattagtcattcccactcccaagtcctctaaattctagccataactcaagagaacatctaaaaactacattgtcctagcttcctgtagacttaaggaccatccagtcgtattcatcaacatgagacatcgtttgtagacaaattgtataccttttgtaaaactggctaaaagcgtttgactagcatttatataagaatccatgacaactcttgactaaaaatgccagaaaagaaaaatttttttaaaagtcatcacaagttggctgctcagataaacttgtatttcttttcatcaaggctttgcatcacagggtatttgtttccttgccttccaggaaactcacaggtaaaatgtcacattcggaaaattaacccatttcaaaaggccttgctacatccatttctgccttctgattaacaacctcactttaaaatgttaaattctatttttaggaagtgttcataagcacatctgttaattgaggtatactgtaagcaatccaggtactgaacaatcacatagttttgaattctccaaaattatctagaaaaaagcaaaatcccaatttttgtttgccaaaggagcaagagacctgattgaattcagggccaagacatccagagagagaagaagggaaaggtggggagtgggagagtcacggggggaggctgacaagccgtcttctgcaaggttgcacaggataggacttgctttacaaaaccccttggatttgaaggcccagaaacctgtcagtcagcacaaggagattgtttatctagatcaatgtcatttcaaccaagctcccagactaatgtatggaatcaggattccaattaatttagaaggagaaatgaaaccccttgaagagggcatggcaacccactccagtattcttgcctggagaatcccatggacagaagagcctggtgggctacagtccatagggtcacaaagagtcagacacaactgaagcaactgagcacacatgctcgcacatgaaacacacatgtccacacgacctgcttgtttatgtccagagacttctgggtcattgatccacccgtcctttcccacggccaggtcccactgcttctttgtatctctattgttgacagtgagtttctttaccttcaattctcatttcccagagctgctcattccaaaactcttatcacttgaaacccactttaagtgtctccttttcaaactttctttgatccttcaaatctaaagtaagctcacattcaggactgtaaaagcacattcttcctttcatgttccttttcactgtattttgttgctgcttttgttcagttgctaagtcatgtctgactctgcaaccccatggactgctgcctgccaggcttccctgtccttcactatctcccagtttgctcagactcatgtccattgagttgttgatgccatccaaccatttcatcctctgtcacccccttcttttgccatcaatctattccagcatcagggtcttttccaataagttggctctgtgcatcaagttgtcaaagtattgaaacttcagtttcagcatcagttcttccaatgactattcaggactgatttcctttaggatggactgattggatctccttgcagtctaagggactctcaagagtcatctccaaccacaattcaaaaaatcagttcttcggcgctcagccttctttatggtccaactctcacatccatacacgactactggaaaaagcatatctttgactatatggacatttgttggcaaagtgatgtctctgcttttaaatatgctgtctaggttggtcacagcttttcttccaaggagcaagcatcttttaatcatgcctgcagtcactgtccacagtgatcttggagaccaagaaaataaaatctgtcactgattccactttttccccatctatttgccatgaagttatgggaccagatgccataatctttgttttttgaacgttgaattttaagccagcttttccactctcctctttcacgctcatcaagagactcgttagttcctcttctctttctgccatttgagtggtatcatctgcacatctgaggttgctgatatttctcccagcaatcttgattccagcttgtgattcctccagcccagtatttcgcatgatgtactctgcatataagttaaataagcagagtggcaatacacaaccttgacatactccttttcactttattttttaattataattatttggatacatgacaggaggcctgaagactcatttctgtggtttatttattttgaaacactagtgaccaaaacaggttatagctgtcagatagtcactgcactagtgaaagtcgtggtagtcttgggtgagcctcagagttacagtgtcgccacctgtcctagaggagggccctcctttttgttttaccagactgcaaattctcctgagggtaagtctgcttttgaactgtggtgttggagaagactcttgagagtcccttggactgcaaggagttccaaccagtccatcctaaaggaaatcagtcctgaatattcattggaaggactgatgctgaagctgaaactccagtactttggctacctgatgcgaagaactgactcattggaaaagaccctgatgctgggaaagattgaaggccggaggagaggggacgacagagcatgagatgtttggatggtatcaccgactcaatggacataagtttgagtaagctccgggagttggtgatggacagcgaggcctggaggtgcggccgtccaaagggtcgcaaagagtcggacacgactgagctactgaactgaactgaagtctgtgtcagcttcgctctccactgtgtatccaggtccagcatagggtccagcatatagcagatgctcagtaaatgtttgctaagaatgtttgctaaattaacaagggaagaagtgattggactccctcattcttccaccgatgcgtcggtccgcagggaaataaaccggcgggagccagcccagtgtctgagggcgctggtcacgcctccgcggcggggcagggcctctgtggagccccgcccacgaggaggtgaacactcagtctttagagccccgcccacgcggaggcggggagtcccgaggccccgcccacaagaaggccgggcctgcggcgcgcgggaagcggtgcgcgcgcggcggcgggagctggtggcccagcggcctgagcgtttctcttcaggtactggttctcccgcgcggactctcgcgtccccacgctttccaggccgggcctgggtgaggggtgagggtggcgagcggccctcgccgagggccagccgcaaggacccgggagagcttcggagaagcgcgcgtgtgcgcggctggcccccggcccggcctcagtgcccccggcggcctcctctcagccaggtctcaggtccggttccggctgccccctcgtccccggcctcggtcggctcgccgagcagcccttcccccgcggccccggccctccctcacccccggtacccgtcgccccggggcgcccaccccctgatccctgggttcgcccccctctcagcttggcctggcccggcccgggccttgctttcccctccctgggtgtccgtccctcgttgcttttccggacgttcccggagaataaactgaaaacaaccgaggactccgcgaaccacttaaggaggagctgcgagggttgcgtcagagggccgcactgacatcctctcccagggcagtgtctttaatggtaggcagggctcctggctcaggcttgagcgttcagtgcctagtcctcggctcgaagattcatctcatcactaaccatttgcagctctctctcctccgtctttggagaaccaacgggggcactccccaccaccccctgaggaactgtgtttcgctccttgaagaccccggaggatgaaagtcatcacttgcgaaatcgcctggcacaacaaggagccggtgtacagcctggatttccagtatggagccgccgggaggatccaccggctggcctccgctggggtggacacggctgtcagggtaagtggggcacggggaatgggaagggcccaggaagcaacgttgaagtggcgttctccactcttgtgtttttataatgactgttaaacgcagggcttcccaggtggccggctcggcggtaaagaatctgccaatgcaggagacgcaggttcgatctctgagtctggaagatccctctggaggagggaatgcagcccactgcagtatttttttcccctgggaaatcccatggacacaggagtctggggttgcaaagagtcggacaggactgagcatgcacactgcaggcaatcatcacaaattattgtactaagttacctagcccaggggcttcccaggtggcactagcggtaaagaattcgcttgctaaaggcaggaggtgccagttggatccctgggtggggaagatctcctggaagaggaaatggcaaatccactccagtattttggcctggagaatcctatggacagaggaacctggtggactacagtccatgggggtctcaaagagtcggacacaactgagcaacaaagtgtatgtatatgtgtgtccagcccagatggataatttgtcaggaccttgagtcaacagtgaggcaatagagagcaactttcttctagggtcctgttacatagactgatgtcactggatgtggtgatgttattatttcagaaccctgaggccagtttcctctaggggaagagtttgcacaaggaggaggtgggaggcagtggcacaggaggtttatgtttcagataggagttacttcctcttacgtttcgaaacagaaactaaattttctttctgaaaatcagtacagggttgttttagtaggtgcagtatgaagcctgctcactacatgtttctctgaatgagtgtttttgtatcaacaggtctggaaggtagaaaaaggaccagatggaaaggccatcgttgaatttttgtccaatcttgctcgccataccaaagcggtcaatgtcgtacgtttttctccaaatggggaaattttagcatcaggaggggatggtgagtattttactttgaatctttagaaaccagatacacacatgtctcatagtcaatactgagatttgagctagatatactttctcatttcagaggttgcttgaccctctgaatctgtcatttagataatttaggggagttacggggatatgttatggggagtatttcattctttttttatagctcagtaatattccattggatttatgtaccacatcttctttatccattcttctgttgatggacatttaggttgcttccatgtcctcgctattgtaaacagggctgcagtgaacagtttctgtacttttgtatacgtttgaaaattcccattatagagggaattccctggtggtccagtggttaggaccctgtgcttccactgctgggggccctggttccattgctggtcaggaaactaagatcccataagccacccagtatgcacccccccaaaaaaaccccaaaatttccattatagagcttaaaatatctgatactgaaaaaaacaatttaagaacacggtaaattagtagaaagttttgttcagaagggaaacttgctgttcctgcccgaattcttagaggtaaatcctcatggccatagtgtctgtagatattgtagatattgtttaatgtcctaaaaatccagaagtgaagaggccacacaggaaagcatgacttgcgtttaatcgactcactctgcagatgctgtcattctgctctggaaggtgaatgataacaaggagccggaacaggttgcttttcaggacgaggatgaggctcagctgaacaaggagaactggactgtcgtaaaaaccctgaggtaaccttgggagggacctgcggaggggttagctttgggacctgaatgcattcagctgataggcatttagactttcatatctcacttaaaacttttacttgtgcaagacagtaatgcaggcatagtttaaaacagcagccccccaatatttttggcaccagggacaggttttgtggaagacagtttttccacagactaggggtgaggggtgatttcaggctgattcaagtgcattacattttttgtgcactttatttctgttattattatgacattataatatataatgaaataattatacagttgtcacttgctgctcagtgatagagtttgcatatgagtctgcaggcagttgattcttcatggtctcctgcagtcctcctccccaagccactccccagagctcacatcatggcctgagctccacctcggatcatcaggcattaaattctcacaaggagcctaccacctgggtacctctcaagcaccatcagggctcgaggccctgtgagaatccagagccacctctgacctgagaggaggcggggctcaggcagtaatacgatcgccagggagtggctggaaatacgcaggaagcttcactcggctcctcctgcctcctcaggcctcctcctagcctcctgccggccagcttagttcctaacaggccacgggctggtaccagtccaggggttggggacccctggcttaaaaagataaaaatatggattaaaatggattttccattaaaagccattaaaataggttttcctagtttctcttataaaattttctttagtaaacttgaatcaattttaaagatacaagtatccttgaaggtagtagtttatagctacagctaatatagctatgtagctctagtgggaattctacaatcccattgcatgggccttgagtttgcctttaaatttttgacatttgttttttttcctctcctcaagacaagaattgctttttcttttctttttcctctttggattctaggggccacttagaagacgtgtatgatatttgctgggcaactgacgggaatttaatggcttctgcctctgtggacaacacggccatcatgtgggacgttagtaaaggtagctgatgtgtttttgctattgttgggggaaaagtgcaagtgttagtcgttcagtcatgtccgattctctgcaaccccgtgtactacagcctgccagactcctctacccatggaatttcccaggcaagagtactgaagtgggttgccatttcctcctccaggggatcttcctgacctagggttggaaccaggtcttctgcattgcaggcagggtctttgctgtctgagccaccaggaaagcccattattaggggagcaatattccaagtggctgaaaagccgaaccatgaaacgcttcagaagagtttctttagcgaactgaaacagaagtttagctggacagcttagggattatccccgtatgccagggatgcagtcggaatgtatgaggtttagcagtggtgttatgagtcatgagttgctgatgccaaagcaaggtagacaccgaacgcctgggggtgcagacatgacgagggagcaggtggggagggacagggctgatcccggccttcacggacctgaggatcttggaaggttatcaggtcatcatcatcagtggggcattgcctgctcgatgtagtggtagctgtgatcaggtttcccttgtagctcagctggtaaagaattcacctgcaatgtgggagacctgggtttgatccctgggttgggaagaccccctggagaagggaaaggctacccaccccagtattcaggcctggaaaataccatggactgtatagtctttggggtcacaaagactcggacacgactgagtgactttcacttcacttcatgggcaggtttacatttcaggacatcaaataaagcaaaggatacctcgctgctttgaaagaacctgttttggaggggtgtgggaggaggtgggaggtaaacatgtgagaagttaactacaatttgaaagtagcagtggtggccagtgcctaaccacttttccccgccgcagtcctgcagtcaagcttggtgttttctcccaaacctcttcctctcctctcctttatctggtcattctccaccagcccagatacccaggtcaggggtcaggctgtggctgttggcatcctctgcagtctcagtgctgcatccgcccagtttccgtccacgctgtaaaagcctctcttgtgttaatagttccgtgctgtccactgaagcagcttctcttgcctcctctgtgtcacacacactctttctaatcagtccgcgacatttctgccgaggggaggaccagcgtgaccagcatcctcatcttctttttcccaaaccttgatattttggtgtctgcggacactccatccagcacctctgaaaccagctgtaatgaccttgaacactgccagcactctgggcactcttgcaggtgctggaaaatacccaaatatgatggtcaagttctagcaggttgtgaataagtacgcaaacgtccatccaatgtcaggaagtgatagcgccctgagggggtcagagagtggtcgggtgatcaagaaggcttctctgaacacgtggagtttgaaaagaaactggaatgaagttggggagtgagctgtgtgcattgttcggggaacggtgttcccgcgtgtgggaacagcaagtgcggaggccctgtggctggaacggcttgttgtgtccaggcaacaggaaattgagggtgtgcaggagcaggagggggaaggaggacctgaggctggagggtggtggtgggcccggccacccgggtcaggtctcaggttatcctaggtgtgatcgtgagctgccaaagggctttgagcaatagagcaacctgattttacttattagcacgtggcatgctgatcctgggacaacagagaagagggcagggggaggaacagtttttaaggagtgtggaaatagagactttattgtaagcctttgttttaaacgtgtaacatgggcaaggttattcaaagtcagtaagtttttgtatttgtggatttgcacgtgctgtgattctatctactgcccatcaccttcagttcagttcggtcgctcagtcctgtcctactctttgtgaccccacagactgcagcaggccgggcctccctgtccatcaccaactcccagtttactcaaactcatgtccattgagccagtgaggccatccaaccatctcatcctctgtcatccccttctcccgccttcaacccttaccagcatcagggtcttttcaaatgagtcagttctttgcatcaggtgccaaagtattggcgtttcagcttctgcatcagtccttccaatgaatattcaggactgatctcctttaggatggactggttggatctccttacagtccaagggactctcaagagtcttctccaacaccacagctcaaaagcatcaattctttggctcttagctttatttacagtccaactctcacatccatacatgaccactggaaaaaccatagctttgactagatggacctttgttggcaaagtaatgtctctgctttttaatatgctgtctaggtttgtcataacttttcttccaaggagcaagtgtcttttaatttcatggctgcagtcaccatctgcagtgattttgactccagagctgaaaaatgcccacttggtccgcacttgtcttgtctgctgtccgctgtcccagagctgggcatctgtctctgaaggcctgtcctctgacagctctgatagggacacctcaggccaggcggcctgccccccacatcagcacatctgcggctggtccctcagcacctccacttcttgctcgtctccttccaggagccccgcagtccactgagccctctgcttcctggtctgtcggcccctccggcctcttgtattctccctgtccagcttgtttcccaaggggatcatgtacgaacacagactgtcttcgtccacctgccccaccaagacaaaccccagcccaggatcggctgtggtcctggtcccgggctgagcacagctggtcacagacccacacgctgcaccccgagcccctgcaaatgcgtccccagcagcctccgggggcgggtgggtggtcccaaggtccctgccccttttgcttgtgaggtccagtcccacccctgtttcctcaatagcctcccccacaaacctccgaagatctcctgaaactacttcttcacccccagccaccttaatctcagcagactcccagagaagagcctcatgaaggggagggggacggacagttactctgacatctgtggtgtgcacacgaaggtccctggcgaccttgaggcctgcggttttagggggcagtggtgggtgtggggagcgggtgcccctggcagaagattgagtaagtgagaggcgaggaaatcgccagagtgagtggccaggtgtagagtgctccccccaaaagagggttttgtttctcaaagactggccaaggcttcctaaccccctcctcggttcttccaggttgtctttgtgtgcgactggttttatcctccacgtttctcccccaaagccctttcccatcctggcccgccttgctttggggcagacccttggctctcacccagtctcctgtccccttcctactagtgacacttcctgtgtgtgctctcttcaaccctcagcttcatccttgacatgcaggtcacttcccagctcagcgttgcgccgagccgcactgtgagcccccagaacctttctctcgcacatgagggtgttgcacacaccagcggtctcagctgcctgtcatttccccgaatgccaagctggctttcaccccgtgggtttgctcagaatgctgccttctctccccgctggccccgcatgtcctgccttacctggagacctgaactctgagctcacgccttcactcttgccccctgctttcttgcggctgcccctcgtcctccccatggtgtctgtgtgtgccctgtggtggtctgttcccccctcccactgaccctgccactcgtattctttctctccagcttgctctccaaggggattgtgtgtgcatagctgccaccgggagctctgagctccctgggagcgggcaccacgtgaagttatctttgaccccagagcctgacgtgggatttggtacagaggctcaatcgcccggtgctgaatgatggaacagcaaaggaataatacttaaattgtggtttataccatttgtgcattagcgggttttagtaatgtgttgtatgctttatccataggccagaagatctcaatttttaatgaacataaaagttacgtacaaggagtaacctgggatcctttgggtcaatatgttgctaccctgagctgtgacaggtaagttagtatttgatgagttgtcctcatctctgttagctttaaattttcttcagaggtatttttattacagcttaaccaatataaaccacaatctagctttgattcttttgttcctgatttcatagaaagcgttttattttactgttattcttaaaacagagtaagaaattggaaactgattaggacaaaagccttacacaaagttagtacttaagtctttattgaatgggtgagtgaatgcagaaatggctctgagttaaatctgaacttttttaaagaaaaagtcttttataaatatgacttaggaaagaagagatacctagtggtaaggttggatatttatttcatttttttgattagtgtttactgctacagtgattttaacaactccgtttaaattaatttgaaaagaagatattttgtcttgttttcctccttgtttcaaagaggtcctgttgctgtatttactggtgtaattaaatacagtaatttaattgtgttcttggaacaaagaatttttaaaaagccaggaggatttccctggtggtccagtagttaagactctgtgctcccactgcaggggacctgggttcaattcctggtcagggaactagatcctacagctgcaacaaggcctggtgtggccaaattaaaaacaacaacaacaacagaggcaccagtcattgctcaccagtcataacagtcctttttcacgtctcttcagctttgcttctcagatttataatggtcctgctctagccatttgcagatttgttttagattgaaaattatcaccagaaaaaaagtacataatgacatttttaatatagagcatatagagccaaattatactaaagtcttttgttaggtttattactgccataattgagaagtgcctcccctggtgactccaatggttaagaatctgcctgcagtacaggaaacccaggttcaatctctgggtcgggaagatcccctggggtaggaaatggcaacccactccaatgttcttgcctgcagaattccatggacaaaggagcctggtgggctacagtccatggggtctcagaatcaagacacaactgagtgattaagcacatacagctaattttaagtataacaaactggatttcagataaccctggaatgaaaacactaaggaagaattagtgtttatcttttgaagaatctcctgtaacatttcagatgtcatttattttctggtatatgtgggggaattctttggctttgccccagtgacatgtgggatcttagttccccaaccggggattgaacccatgccctctgcagtggaagggctgagttataaccactggactgccagggtaatcccctggtgtatgtgttttattcaacactcaggagtgaaacagtctgattggcttggggaataatatattaccaacaaccagtttacctgctagtgttttccttactcttgcaaatccctgaggccaagacccttccacacaaaatcaacttcacaacagtcgtgcagatggccattgggccacacgtccaagacctgaagttcatctcccttgacaagttgaaacattccataaaaaagtgaaattacagcgtatatccccccttcattagcagtagcctttcaattgttaatatttgcccgagtcattcagttaagtgtgaaagtctttggtcagtggttttgcaggccgcaagttgttgaagcagtctttagttctggtactagattgtttctatgggaaacgtaattcacttactagaagagataggtcagacttgtcttctttgtttaggatcctgagggtgtacagcacgcagaagaaacgtgtggcttttaacgtctcaaagatgctgtctggagtgggggccgaaggagaggtacaggattgacattcatcccacctgtgcttaagttgggacccaggaagccgtgagcacctctgtatcctgcggctaagcccctggcggccaggcccccacacctgaactgcagtgtgacttcctctcagactgccgagtctcagtgtgggggtgctgggctgacctgagtgggagtggggacctctgggggccaggggaagagtaggtggtgaagctcagtggtttccagtcctgcatgagctatggagtcacctgggcagcatgtaaaagtacaggtgcctgggcctcacccccagagatgcgtttcatgtggctggaggcagccagggccccaggtctgaggtgcagtgagggccaggaaccactgaccaggagcatcccagccctaggcaacaggaaggcaggtgggtccagcaccttccaggcaggtagaggggcaacgcgggccactgggctggtacccaggaatgggaagtgttctgatctttgggaagaagcggcaaataaaggtagcaccctggtccttgagatgtcagatgacggagtactaaggcagacaaggagctgaaagggagcgagctggttactggagcagaggagggataactgcaaagcagtcctgggcagcaggttaataaggcagagtgagctgcgggctgggctgggtctggagggttaaaagcttagtgtttgtaaagtgttaggtgcatggcagcaaaaactgttccacataggtgttcaagcagtgttgtctgggaagatgatggggttcctgttttctttttaaaaagggttcattcagtgggagattctaaatatatacacacagataacaaacaaaagggctcctagttaccttcctgccttttcccagtctcatttcccaggcaaagcactgttcacagcatggtgtgttcttacagaggtatatcatttaataattgttaaaccacacaaacacgtagacccacttcggtattcttgcctggggaatcccagggacagaagagcccggcaggtcgcagagtcagacacaactgaacaaatttacactttcactttcaaacacgtagatacacacacacatgcacacggctttccttaccaacagtgggagatgagttcaagactgatggcctttgcagtcctgtgtgattatttgagttgtaatgtgtttcaatttcatacttattattatgtcagtaatagatgacaatatgtagtgaagagataggaattggtaagcctttattgtgaatgtgaagattcgggataacatgtaaacttcatgtgctgcaggcgagaagttaccggatgttccacgacgacagcatgaagtcgttcttccgaagactgagtttcactcctgatggatctttgctcctcacgccaggtgtgtttcacgaagctttggatttgagggcaggagggcagcagtgctggggtctgtgctcctcattctgggctgctttttgcagctcagggaattctgatgaaagcactgatcccaacttgtccacacactgaactctgttaagtcgcttcagttgtatctgactctttgcaaccccatgtagcccgccagacccctctgtccacaggattctccagaaaagaatactggagtgggttgccatgccctccaaggggtcttcccgacccagggattgaacccacgtctcttaggtctcctccattggcagatgggttctttaccactagcgccacctgggaagcccttccaaaacctccagggagcttccaaaagtcctggtacttgggtcccaccccgctagagattgtattttaactggtttggagggtgtgatctgggttttggaatttcaaaaagatccccagtggtattaacacacagacaatttgggagccactagactagagtgataaatgcattctcttagataaattcaattaaaaaaatttttttaacaaattccttttccctagacatgtataccgaggtcatagttctaaaactaatgagaaagtgcccttatcgtgtttattctgagtaatgaagcatcctattgtgacagggtctagacacagacatgtcaggtcggaataacttgtggcagtgtagtggtcaagtctgacaggtcagcagaatcagtgaggacatgtcaggctcgttgcctgcaggagggcaagagggtgagcctagcagaatcagccaggacagtgctatggccagtgtggtctcagagaccaacccctgcgctccactgctcaggtaccatcctgtgtgtgattcagtgatgtgaaccccgggctcagccaaacgggaaggcgtgggtcaggtttgccattacgtgttccagccaggctgggtgggggccccaggagtgtgtgaatcacacagacgtgtggatcagactgtaaaaggacaacagagaacatgtgggaatgacattggcttgggggtggtcccaggaccaagagtaaatagttgcttaagtatattaaaagttacttactaaactcagtatcaaatcctttttctaaagggcagctctaaagacattttcagttcaaatcggataccagatgggtttctactgtcactgttaacgattctcagtggtgttaataaatgcaggaaatgcaatgacagagattggaaaagaagagagaaaatgttgtgataggattatgtttctagaaaatccaagagattaaaaaaatactattgggataaaaaaagaatttgataaagtggctaaatattgtaaaaatacataaattctgtatctgctacactagaataagtacttagaaatgtgaaaaggacagttgttccattcataatagtgcaaagtccagaagacatgggactacataggaaaaaatttaataaggcacagagccttgatgaagaagactagaaagtcttattgatggttataaaacaagttctgaacaaatagatatttcgtgttctaaaaattaacatactcattgttttccatttaggatcccatttaaggtttttttggttgtttcttttggttttctgttttaattggatgacatctcagatttgtttagttgagtaaatgcaaagaataatgcaaccagcttcccatgtggctcagtggtaaagaatccacctgcaatgcaggagatgcaggttcaatcctgggagtcctcatccagggagtccggaagatcccctggagaaggaaatggtaacccactccagtattcttgcctggaaaattccatgcacagaggagcctggcaggttacagtctatggggtcacaaagagtcaaacatgactgagcgactgagaatgtagcacatatgaaagaagaataacacttgagcagcatctccttgtctgaatgtcagagattttatagtgcaaaaaaagatgtgcttttcatcataggggactagaatgcagaagtaggaagtcaagagacacctggggacaggcaagtttgatcttgggataccaaatgaagcaggacaaaggctaacggacttttgccaagagaacacactggtcatagcaaacactgtctttccagcaacacaagaaaagactataaacgtgcatcaccagatggttaatactgaaatcagattgattatattctttgcagctgaagatggagatgctctatacagtcagcaaaaacaagaccaggagctgactgtcgctcagatcacgaactccttattgccaaagtcagacttaaaatgaaaaaagtagggaaaaccactgggccattcaggtatgacctaaatcccagcccttacaattatacagtggaagtaagaaatagattcaagggattagatatgatagagtgcctgaagaactatggatggaggttcgtaacattgtacaagaggtggtgatcaaaaccatcctcaagaaaaagaaatgcaaaaaggcaaaatggttgtctgaggaggacttacaagtagttgagaaaagaagagaagcgaaaggcaaaggagaaaaggaaagatatatctatttgaatgctgaaagatatacgcatctgaagagttccaaagaatagcaaggagagataagaaagccttcctcagtgaacaatgcaaagaattagagaaaacaatagaatggggaaaactagagatcttttcaagaaaattagagataccaaggaagcatttcacacaaacaagggtacaatagacagaaatggtatggacgtaacagaagcagaagatattaagaagaagtggcaagaatacacagaagaaccatacaaaaaagatcttcacgacccagataaccacaatgatgtgatcactcacctagagccagacatcctggaatgtgaagtcaagtgggccttaggaagcatccctacaaacaaagctagtggaggtgatggaattccagttgagctgtttcaaatcctaaaagatgatgctgtgaaagtgctgcattcaatatgccagcaaatttggaaaactcagcagtggccacaggactggaaaaggtcagttttcattccaatcccaaaggaagataatgccaaagaatattcagactgccatacagttgcactcatctcacacgctagtaaagtaatgctcaaaattatccaagctaggcttcaacaatacgtgaaacaagaaattccagatgttcaagttggatttagaaaaggcagaggaaccagagatcaaattgccaacatccattggatcatagaaaaggcaaaaggattccagaaaaacatctgcttcactgactacgccaaagcctttgactgtatggatcacaacaaactggaatattcttaaagagatgggaataccagaccaccttacctgcctcctacgaaacctgttatgcaggtcaagaagcaacagttagaaccagacagagaacaatggactggtttccaaattgggaaaggagtacgtcaaggctgtatattgtcaccttgcttatttcacttatatgcagagtacatcatgtgaaatgccgggctggatgaagttcaagctggaatcaagattgcgaggagaaatatcaataacctcagatacgcagatcacaccacccttatggcagaaagccaagagaaactaaagagcctcttgatgaaggtgaaagacaagaatgagaaagctggcttaaaactcaacatttaaaaaatgaagatcatggcatccgattccatcacttcatggcaaatagttggggaaataatggaaacagtgacagacttcatttttctgggctccaaaatcactgtgaatgatcactgcagctaggaaattaaaagacacttactccttggatgaaaagctatgaccaacctagacagcacgttaaaaagcagagagattattttgctggcaaagatctgtctagtcaaagctatggttttttccagtagtcacatggatgtgagagttggaccataaagaaagctgaatgcttaagaattgatgcttttgaactgtggtgttggagaagactcttgagagtcccttggactgcaaggagatcaaaccagtcaatcctaaaggaaatcaatcctggatattcattggaaggactgatgctgaagttccaatactttggccacctgatgcagagtcgactcattggaagagaccctgatcttgggaaagattgaaggcaggaggagaaggggatgatagagaatgagatggttgggtggcatcaccaactcaatggacatgagtttgagcaaactcagggagatattgaagaacaacagcgaattctggtgtgctgctgtccgtggggtcacaaagagttggacagaactgagtgactgaacaacaataactgttatcaaatcagtatggtattggtatgggtgctcagtcacttcagtcatgtccgactctgccaccctatggactagctcaccaggctcctctgtacccgtattggtataggaataagcgtatagattagtggaccagagcagaatccagaaatagatcttgggacaaaaggaaatttagtaggtaacctgagggtatttcagtgggaagaatgggttagtgagtgaagaatgctggaatgtctagatgtatggaagaaaatgaaattggattcctagcttatgtataaaaataaatctcaggtgcactccgtacttaaatataaaaagtgaaaatgtaaaactcttagaagaaaatctagaaggcctttctaggactgggaaggaccaagcaaaagactggagacccagaagctcttaaatgacagacctacttggctgtagaatgtagatcttgcctataggcaagagatgcagtagacaatatcagtagtcatgatggagctggaaaataatgttcatggaacaggaaagaaaatgctaaatgataccgtatgtgagcgctcttccaattggcaagaagaacagaaaaacaaagggaaaaaatctggaagagagaaatgagagccagatacaaaaggcagtttgtagaaaagcagatcttcatgtgatatcagcacacacaggagaatgttcagaaacccagcaacagtcaaggcagtgccggttaccttgatataagttaggtatggctctattcatgtcacactgaaaaacagtgaaaatacctatggctgaagggtgtgggtgtggctattgctggagggagtgtggattatcagagcctttctggaaagcagtctaataaccgttcagtgcatctgctttgctgagtcgtctgagattcacttcgttaagaaatgaaaacagttcatagggtatatgtccaagtgtgtttattgcaaacatggtttgtagttaaaaaaaaaaacaaaaagcagtactcacttagagaggatgaattacggtattttcataccagtgcacagcaggcagctactaacgaaaggatccattggtgccatgcaggtaatgagcacaagtgtgaataacatctcattttgtaaaacagattttaaaaagccactgtataatatacctgcagttgatttataactggttgtgatgattatgtgagcatggaggaaattagcagtgacccaatggtttgttaagatggattatttagggatggaaggatgggtgagcgggagagggtagggagacactgggtctgtctgctgccctcccctgggggtatggaatggtcatgtggaagactgtgtggttgtataggtgtgtggaaatgttagtataaaacagggagatgttcccaagagtagctgtgtgtgactggcattctccaacaaggtctgacctggttttgagtatagcttgaggtgagctctcagatgtggactgctccccagggcaaatgcagcttctgcgggggctggagatgtcagcagagggctggtctcctggcagcctcagggcgccatcaggcagattgaagaaacccaagtctcagtgccggggaccagccccggctgatccagggtattcaaaggagagatggcataggcgaagatcaggaaacaattgcttaattaaacgttaattaaggatataaagagtaataaaatgaggatagctcagtgaggaaattcagtggagaaaagaggctgaaataaggatagctcagtgaggaaattcagtggagaaaagaggctgaataattcagccagaaggtaagagaaagaacgacatggtgagaccaagtttcggtgaacaaggcccgcactttattttccaaagtagtttttataccttaagttatgcatagaggataatgggggaaggggtagagtcatgcagcaagccaggctttcttcctgcagatttatcatatgcaaaagcttaggtgatttgcatcatcttctggcccggaggcctgttaacattttaagaccctttcttcagaaaacttatttttctctaaaggtgattggtcaggagccaccctccaaaagcattagataaagttgcattcctacagagcagaggtgtggtgggctataacatgaaaaagaattaactcaagggtcctaggttacaaacattaaagctactacttacaccaattatattaatcactacactgccagggacacagcaggtaagggatatggaaacttagcagcaaacattggcccaacaagtgaaaatcccttcaccaatacaatttctaatcaatcttttaactactcaaaggaatctgtatttagacagtttagaacatctcatgcctctcacagttgggaggctctgaacaatcacatgtggccggaaaaacctattcaggcaggctagaggatttccaaaggagtttgtaggttaaacactgtcacacccaggaattattaactggagctgtaagctaagtctttttttcagagaggtagtggggcacagcccctcgtaaagtcagaggtgtaggtgaaggcacaaagcagaaagtaggcagattctggttttgaggatatatgctcgagaatttccagggggactcctgaagctcgatcccgcctttgcgtatgccgagcctccttcctcatgacctttgtcatgggcggagttcctcacactggctcccggcaatgatagaattccagttgagctattccagatcctgaaagatgatgctgtggaaagtgctgcactcaatatgcaatatgcaatatgcactcaatatgcgatatgccggctcccggcatctcaggctttgcgtggcagctggggtcagaatcgagggggacagcaagccgagcgtctgatcacagcgcttcaccacagggattagctggattcccaggcagaggtccgctgaggccagggtccctgctttctgttcttcctgcccaggcctggtaggggctcccaggtggctgagccccttgggaggtgacactcaggggggtgacaggtggctagaaccgcagtaagggctgagcccagacagggacgagatgtttgcatttatcctcagacatcaaacattgtacaggaaataatctttctggggaatcttatataaaccagaactgatgctcttgcgtctctgctggtgaacagtctgataataacacaggcctcaccttcctctcctgcttcttcagctgggtgcgtggaatccggtgaaaatgtgacaaacacgacttacgttttctccaggaaaaatcttaaaaggtaggcagttaaagaaatatatgaaacgtttacccattctttttttttttgcttttgttaagtgacagtgcctttttgcaaaaccgcaattcatcttccaggcccatcgcccaccttccgtgtcctgcgaaggcgacgctcgctgtccgctgctgccccgtctactttgagctcaggccggtggcagaagcaggtgtgttaagtgaccagggcagggagggaagaggtgagtatgggggttagttccatttgaccaatcttaggagcaaagatggagaaggcagtggcaccccactccagtactcttgcctggaaaatcccatggacagaggagcctcataggctgcagtccgtgaggtcgctaagagtcggacacaagtgagcgacttcactttgacttttcactttcatgcattggagaaggaaatggcaacccactccagtgttcttgcctggagaatcccagggacaggggggcctggtgggctgccatctatggggtcgcacagagtcggacacaactgaagcgacttagcagcagcagcagcagcagcagcaggagcaaagattgacgttatcaaaatacgtgtcagcctacgcattgtctagaatacactctttttcttttgctactagcatctgtaaaggaatgaaagtaggagctttgtctctctgctgcttggctgactgtcattcatcatcagttccctgtgcaggaaaactgatacatgaactggatttctgtgtaactgttttccctccactggctgcttttaagatttttctgtttgttggtggctgtaagtagtcctatcccagtgtgcctgccgcagttttctttgtgtttcctgtgcttggggttcattgactggcttagatctacaagtttatagtttccatcaaattagaggagcttctaaaaagaatatatttatttatttggttgtacctgatcagcggcttccctggtgactcagatggtaaagcatctgcctgcagtgcaggagacctgggtttgatccctgggtcgggaagatcccctggggaaggaaatggcaacccactccagtgttcttgcctggagaatcccatggacggaggagcctggtaggctactgtccatggggttgcaaagagtcagacacgactgagcaacttcacccagtcttagctgtgggcacgggatcttcagttgcggcacgagggaactagttccctgaccagggattgaatcccaggcccccggtgttgggaacacagaatcttaggcactagacctccaggaaagtcccggatgtggggaacttcgccagttacttcttcagagactttctgctatcatcctctctcccctcttcttcagggactccagccacacatgtatcccgctgcttgaagccacccacggctcactgatgctctgttactggactcccccacctccgccccctgcctgccagcgttttattttggatagtttctacagccgtgttttgagttcacgcaccttttcttctctgtcacctgcccttggtgccatccagtgtattcctgtgacagtgtggttttcatctccagagttcaagttggttcttccttatgcatatctgctttccttttgcttctagaacacgtggactacagtgttttttgttttttttttggactacagtttttaaaacaactcttttaatggctttgtgtacaaagggttggtttcgattgactttttttctcctcattatggatatttttctgcttcttggcgtgcctgataattttttattagacattgtggggattttaccttagtggatacaggtgtattttcattcttttttttttctttctaaattttggctgtgctccctggcttcagcatttgttgtctgtagccttttaacagttgccgctctgaccagtgtgaggtggtagtttagatttgcatttctctattaattagcgacgttgagcagcttttcatctgggccatctggatgtcttctttgcagaaatgcctattcaggtctcctggccgtttctgatagggttgttttgttgttgttgttgagttgcatgagctgtttgtgtatttgggaaatcaagcccttgtccgtagcatcagttgcagatacttcctgattgtctgtctgattgctggcctccttagggatggctctttctttcccatagttttctcacgtgtgtttactgccagcatcggctgaagactctggaggcccccgcagttctctctctgccccgccaccgcatgctccaccaggcctcttggctcccagctcggggcgtctgctgggcgccgcctgggtctcctccacctttgcggggcctggaacctacaggcagaaagcgggggcagtggcggggctcgggtctcttgcttcccatctctgtgagattgctgtccttcatgggctcgtggacagtgtcccccggaccctggtttcagtgttctgccaggtattttaattctttcaggggaagggtcagttggtccctgttactccaaggctagatgcagaagctattttcaactttctaaacctttacatgaatgactttcctgctagctcagctggtaaaagaatccgcttgcaatgcaggatatctcggtttgattcctgggtcggaaagatcttctggagaagggataggccacccactccagtactcctgggcttccctggtggctcagctggtaaagaatccgcctgcaatgcgggagacctgagttcaatccctgggttgggaagatcccctggaataggaaatgggaactcactccagtattcttgcctggagaatccccacggacagagcagtctggtgggctacagtccatggggtcgcaaaaagtcgcccatgactgagcaacttagcacagcgcagacagcatgaaaacatcccttgataaggcgtttctatgtgaccctgatttttcctcgtcaggtgctgaccacagggtgacttgcctcatccacacaccttccttcccttcttttcaggtctcaggtgcctgtgagtctcagatcagaacatggtgtcctcattggttcatgcatggttctccttgattcttacatgtgtgttagtcgctcagtcacacccaactctttgtgaccccatggactgtagcccaccgggctccactgtccatgggattctccaggcaagagtgctggagtggggtgccattgtcctctccagatgttgttatcttagtctgctgctgctgctaagtcgcttcagtcgtgtccgactctgtgaccccatagatagcagcccaccaggctcccctgtccctgggattctccaggcaagaatactggagtaggttgccatttccttctccaggggatcttcccaacccagagatcaaagcctggtctcccacactgcaggcagattctttactatctgagccacctgggaagccctggattctgatattcatatataaaataagacaaaagtgaattctgtaaggcatacagcaaactagtcttttatgaaattatgtcacttatgtccccaggggcagggctttgtgggggaggggtggagatgtgtttgatctgagagcttggcttgtttgaccttctctgcccctgtgttcgcccgtcaccagaaccaggcatggagctggtcagcctgccgtaccgcctggtgttcgccgtcgcttccgaggactcggtgcttctgtacgacacccagcagctgttcccgttcggttacgtgtccaacatccattaccacaccctgagtgatatttcctggtgagtggacagtgaggacacgtaccctcccctggggtagccctgcagttggcttggttggctcttatttaccagcttttttttttttgaaaaaaagtattaacaaggaaagctgtgtctcacttgctacaatactgcttctggtggtcatctcagtgggtgttgatgccacacggatcgccctgtggattcttctcattctcataaaccgaagggaagacgcctgggctccctggtcgtccagggacttgccaggcccccgggaggaaggaaccccagacacagagagggccccagaagtgggcctgtcctggggggtcgccgccctggccttcctgcttggggttctcagcaccccgggcgccttcctccgtctccccgcccgctggggcccagcctgggcctggccgccccacacccggttcttgctctttgtaactcttgatcttcttttagtcctcttctgatcttggtgggcgagaggaggccgtctcacctagattccctggtgaagttcatgacgaggcagagatgctggggccctgagaagctcgaggcgggtggagcggggggtggggaaggggtcacactagccctttctcagccgggccccctccccacaacgactcttgtcttcaaacaaagctggggacggtgctcgcacttccattttcctgcctggtgatgagcgcgcccagagtggccaacagcgttcggtaaacttcctcccttctgcttctttttgggaacgaaggtccagcgatggggccttcctggccatctcttccacagacggttattgctccttcgtgacgttcgagaaagatgagcttggaatccccctgaaagagaagccggttctgagcctgcgaactcccgatacagcgaagaaaaccaagggtcagaccccctctggatcttcgccaggacccaggcttggggaagggacccccaccggcagagtccaggaccccagcagcccttccaccacccctccacaggcgaaacagtctcaggcccccccggcggccaaggacacccccatggccgctcttggtgccagaggctccccagcagggcctcccaaggagaagcccctgcagcccagcggtcagaacgccaaggcccagccgtcccggagggtcaccctgaacaccctgcaggcctggagcaagcccacatcccggtaagcacgcagacgcagaaagaacgcgtccttgcggttggaaaaacagcaagagctgaaagccccccgccttgtactgtgtgagctcccacacggcggccagagaaacgtttcctgatgtccgaggggccctgtccttgctcagacctccgtccacctcacttctggtggtggaggggagtgggacggtgtttgaagtctttttgatattaaagtccccccttaatttcctgtgagcttcttgcagttagaattttaaaaaagtaactgttgagaattgagactgtggatttttttaaaaagaaccatccgtgtcttagagctttcatactggaagccacatccgataggctttatatgaagcacctagcagggtggttagtgtgtcattggaagttaacaaagcatggattttgaagcttttagatgactggttgactattatttatgtgacgatttaactgagtacatgatccttatttactgagtaaggaggccttggtttccttgtttcctcggaaacttggtaccatatacggtcaacctctgtgatgctcacgaccttttacttctctttttaatccaggagaataaacttaatacccttaaagacagattctccactgaactccatatcaacccctttaacttccagcccttccacagaggaaattcaatcaggtaagtgacactgtgggcttccctggtggctcagctggtaaagaatctgcctgcaatgcgggagacccaggtttgatccctgggtcgggaacatcccttggagaagggaatggccacctactccagtattgttgcctggaaaatcccgtggacagaggagcctggtgggctgcagtccatggggtcacaaagagtcagacacgactgagtgacttaacactttcactttcaagtgacactgttactggtctaatataattaacgagaatattgtctcttttggaaatgaacactgcgtgactttaagtcacttctgagaaagaaggatgcatcttgggacttcctggtggtccagtggctaagactccactctcccagtgcatggggtctgggttcagtccctggttggggaactagatcctgtgtgtctccactgagaccccacacagccatataaataaaaataaataaaacctggctttaaaaaaggatgcatcttaccaggcttgtcgtttaaacctgaggtggggagatggtgtagagctggttctcagttcagccctgttggcgctgtgggtgtcagtcctcgctgtggggctgtcctgtggatgcaggtggcgtagcagcacccctgggcactctcatcccttccccctgctggtgacattctgcagtgtcaccagactctgcccactgtccctcgaggggcacagccacccctggctgcaaaccgctgggtcagatctccccctcagatccacgaaggaccaggccaggcagtccagttcctttgtctggacacgaaatttatctcgtaaacttcagctcttaacttagagacgtcttcactcacctgccaaccttgtgtgtgtttaagcctcagagacgcctgcagaccctcaggtcagccccctggagctgaagcggccccgactcggtgagcatggagaaggaccccgagtcatggaccctcaatgatacctgtcctgtgcctgaaggcggctagctctggggcacccgggtgccagggaggggcgggcctgaaggcagctgagagcagcaccgtccgtcggaaatggatttctgtaacggaagatacacgtggactggtcttctccagagatatgtcacctcttgtatttaggatacatttttccctcggagatgtgaacatcttaatagattatattctctttttcttgagtttctgagatgggagccctgtatcatctgtggggtcgcaaagacttggatacgactgagcgcctttcactttcagtgttacctgatgtgaaaatgagtgactcctgccttggaatatttgtatgaaaacgcagacagttgacggtggactctcctccagccggaattagaggagacagtactttgaggtttattccattgggttttagtgtgttggattatttgatcaacttagcttctaaatagttgattaacttttggaagtttgttatgtttgctttcagattttaaaggtaataatgctctttttagaaagatagtaaactagagtgaattaggggaaaaaaacggggttggaggtagaaatcatccatctcaccacctgaaggaaacccttgctaccgtttcagcatatttcctttttaaaatcttttttttttctgttatttttgggaagaggaagggttgcaattttactgtatatgcagtttggttaacacataggttaccttcttgatttttcaaagcaaaagaagtacctatccttggtggcctgtgcttctgctcactcagcacctgggctgctggtgtggtccttttcctcctgtgggtaaatgggccttgcgggctgagatgtgtgatgtgccgtccctgcagcgggcgtcacacatttggaggctcctcttagcacaagagacgctggctacgtgactgagaggtggcgacgggcacagccccaggcgccggtgcggctgcctcacgctgaacggagctaacagctgcttccacgacgcctgaggccggcaccccgcatgaagcccccgggtggggctgcaccagcgccccctgccctgtctgagccagaccagagggcaagttcaggacccgcacatcccacctctggcggggagcccctgagggtgggagaacgaccctcctcagctcaggccagcagcaggcaagagagttggaaagaatttggttctttgttgcctcatccacagcctgtgcacgtgtgtgcgctcagttgtgtccgactctctgcgaccccatggactgtagcccccgacagcctccttaggccatgggatgctccaggcaagaatactggagtgggttgtgtgtcctgctccaggggatcttcccgaacccgcctctcctgcatcgtcaggcagattcttcaccgctgagtcacctgggaacctccgtccacaacccagttatcaggaaaaagagcccgcctccctctccacgggaaagaggcaaagggggtgatttacaacgtggagagcttgcaggtgaatcgacgaagagttatagtaacacgagtgtgggtcacatccaatacaaagatgttttgagaggtcgaagagcagctgttttgaccagggttccaccttgaggtcccccaaagacctgtcagacgtcgatggccgggtcctgcggcccaagtctggccttcttggtctggggtgccagccgaacctcaggattcttgaaggcccctttggcgatctgaggcgcagccaggattgagaagtcaagagagggaagcttcccctggaggcagtctctgctggttaaatgcatctgcatggaaagctttctcttgtttctctggtccatcactgagggacagcagcacttctatccaaaccgaaaggataacaatgtgtgggagatgccatcagctcttcagaggaaaaatgaccttaaatttcctgcagctagggacttccttggcggtctgatggctaagactccggcgtgcttccaatgcaggggcagggcacgggttcaacctctattcggggaactaagatcacatgtgataggcagctcagccaaaaaataaaaatatcctgcagctggagcgacgtatcttttgtagggattggtcccatgcaaacaggagtcactgtgggtcctgcgtgcctcggtgagcaggtctgcttctctgaccacccgcagcagaggagcgacagcaggaatggggaggaggcagggtgcttgtttataggcacagttattacccgtttgccaccctgactataaattacacactcggcaataagagccccagcagcagcacccccacgtcctgatccaaagcctcctgcttccacccaggtggtggtgggggagctgctgctgtatccctgctgagtgcagcgacctgctcgccgttctgatctgtaggcaggtggggtttgtgtctgaatcccggctgcctgctgggtggagagaggcctgggagaacttgataacaagtttcctggagtctggggaggagtggcctgtaggccttcggggaagccagcagcagaggtgatggcttcatcacaccaggcctgggtgaccaggcctgtcagggcgcccaccagggccagggcccgccgtggggaagaggcaggaagggagcaggagggtgagtcaatgtctagtcctctgactcatttctgccccctgccagccactgtcgtccttgctgaaagcacccctgtctgcatgatcatgatgctatgggcctttcttaccaaaatgatttgtcagtgtccatttaagtaacgaaggtcttgagtcctgggggtgagtgaggggtgcaggatgaacgggtctggccccccgtcccacctagactgtcccagcactcctgcatctccccttttgggttatttttgctgtattgcagtctcagctccctctcccctgatgtgtgtgtgtctgtcagtctgtgtgggagaggcaggacctcggacaagccgcttcatctgagggagtttcttcacagttctaaagactatgcgtcctttggtaggggagaagggtcagagaaaaggcaggagtgcagaaaggagaaacagaattaaagggctgagggtaaaccagatccagtctcctccttttttatgggggtcataggcacaagcgcagaaggcaatggcaccccactccagtactcttgcctggaaaatcccatggacagaggggcctggtaggctgcagtccatggggtcgctaagagtcagacaggaatgagcgacttcaatttcacttttcactttgatgcattggagaaggaaatggcaacctactccagtgttcttgcctggagaatcccagggacgggggagcctggtgggctgccgtctgtggggtcgcacagaatcggacacgactgaagtgacttagcagcagcagtagcataggcacaagggcttccctggtggctcagctggtaaagaatccgcctgcaatgtgggagacctgggttcagtccctggattgggaagatcccctggagaagggaaaggctgcccactccagtattctggcctggagaatcccatggacagtcgatggggttgcaaagagtctgcatgactgagtgactttcaataatagacacctggtgtcacctatgtatgaactctcatgcgcactagagggtgctctctgccctgacccagcctggtactagagtctgggctggtactggagtctggtactgcctggctcggagtctctgctgcctacttagttccatctctcccggaggggtccacgttcctgtcaggcttgacatgagtgtccttggatgccaacttcagcccctgctggccagacagagcaacagatggagaagggagaggattatggaagtaatccatgtgtcctcatctgctcacttggttcatctattcattggatacctagataccctgatagctcagctggtgaagaatccgcctgccatgtgggagatgtgggttcgagccccgggttgggaagatcccctggagaagggcatggcaacccactccagtattcttgcctggagaatccctgtggacagaggagcctggtaggctacagtctatggggtcacaaagagttggacacgactgagcaactaagcacagcacagcatgcagagaccagtggatacacgagcacaaaacagacaaagctctgccctccctcccgcagcttacgttctgcctgggggagaccaaccataaagctagtgcatacaaaaagatgacttagggaaaaaagagaaaggttaggggtgttatgggagctgagtggagggtgggttgcagctttcagtggggaaggcctctcgggaaggcgggaaaaacaataaagaagggggcgaggaaggaaggaggcagatcaggctttctaggcattctcaagagcaagacacccttctcagctcatctctgatgcatccagtgggcatcccctgggctcaggctgcttctgagtgatgggggtgaggacagatgcagtcccctgccagtgaggagcccaccaccaactcggcctcccttggcgcctcaagctcacttggcattgagaatgggatcagttcggttcagttcagtcgctcagtcgtgtctgactctttgcgaccccatggactgcagcacgccaggcctctctgtccatcaccaactcccggagtttactcaaactcatgtccattgagtcagtgatgccatccagccatctcatcctctgttgtccccttctcccaccttcagtctttcccagcatcagggtcttcaaatgactcagcccttcgcatcaggtggccaaagtattggagtttcagcttcaacatcagtcctttcaatgaacactcaggactgatctcctttaggatggactggttggatatccctgtggtccaagggaatgggtaggcgttgtgaaatccggatgatatgccaatgattggcattaaaattctttggactcactgatttcataacctccatattcaactactcaagaagaagccgaatcctgaagaaaactttgctcttcccccgcgccccagtgtcactctgcttctaacccagaagttacaggaatgagttaatgctgtgctgttggagccgcccccaaagcgagaaccaactctcggttacgggtgggacgagcgctccaacaagccactagggggcgatattgccaggtcttagctccactgtcaaatttattaactcatttcaaatattcactgaaagggtttctaatcccgttgttatggaagtagtatattatctattagttttcacgtgtattactttctagataattaaatgctattttgtcttctcaaagagctctataaatccaagtcacatgctgactcagagcacaccaggagaacttcacatacatgtaagctaggttgaaaaggcacttatagtcagcgtaccaaaagtataatgagcttgaagacactaacttcagaagggttttaaaatttttaaaccaaaaaatacagccttaataatgacatttctctacattagcagcaaaggtaatatccactacacctcccaactgtacaaagacatgcacaaagattcgtcacaatgtttggaaatggaaacaaagtgtctgccaaaaggagattaaggcacagtgtagctatatgtgatgcatatatgtgatgcagctgttagctggaccttgttgctgctgtggaaaaatgttaatactgaccatagtaatgtgctttttgttacccaaacgttactgaccagagatgcttcggtattcacctacataataaatcattggccacagtctttagtttctgcaaaaatcccctgttcaataatatgacaagtgcagaaacaaagatgagattctatttatcacattccctgcaagctgacgggactgacccgaaaggctcaggggtaagcaggcaccgtgctgggatcggaggagcgtttggctctgaaccttttcctcgggagccgagtgagagggtagcttctgcttaggacactcgagacccttccagttttattttttaaatttttccaggctcctctgtccacgggattctccaggcaagaatactggagtgggttaccattttctactccaggagatcttcccaatccagggatcaaacccgggtctcctgcattgcaggcacgttctttaccttctgagtcaccagggaagcccctttttaatttaaatttatttatttttaattgggggataattgctttacattaccgtgttggtttctgccgtacatcaacatgaacccttccggctttagacttcaagccagcacctaacaattttagccctcgttccacttgctggcaagggcgtgatcctagatggtggcgggggagcttaggccagcacgccaagtgactgtggggttctcaccacaaatggggctgccaagtgtatgaggcggtgtgaagagggtccgggggagcataaccaggcatggactttgggtctagaaagggtagggttttcgttcaggctcaactgctaaactgctgtatgacttggcgtgttttacgtcatctccctaggcctcagttttcccatctgtaaaacaggaatccagcctttgcctcatatggtggttgaaaggattgagtgggacatgtactttcctcttaaactgagacttgcattttctcaggggagaaattttgtgatccctggggacatatttaatcactatactagggttctccggacaagtacacgataggatatgtgtgtatacataaatatgcacatatctctatatatgtacatataaatatacacacgcttcccgggtggcactggtgctaaagaatccacctaccaatgcaggagactcagtagatgcaggttcgattcccgggtcgggaagatctcctggagaaggaaatggcaacccactctagtattcttgcctgaaaaatgctatggacagaggatcctggtgcgatacagtccatggggtcgcaaagagttggaaatgacttagttgactgagcatgcattcacgtaaatatgcacacatatatatgtacacaagtatacacacatgtatgtatataactatacacacgtatgtgtatataaatatacaaatatgtgaatttatacatacgtataaatacacaagtgagagggagagagattcactataagaattggctcacatgattatggaggctggcaagtaccgaatctgcagtgtgggccagccgcctagagacccaggaaagctgatgctgaagttccagtccaaaggctggcaagtggaaacccgggagagctccctcttgcttagtgaggctggactttttgttctactcagctcttcaactgattggacgagccccacccccaagttagggagaacaatccacttactcagagttcacaaatttaaatgttactctcaccccaaaacactctctaaacgcacacttaaaattagccatcacgtaaatgacaagggcctactgtatagctcagggaactatatttgatatctatatattagggatcaaacctgggtctcctgcactgcaggcagattcttatctgagccacaggggaagctccatataatgaaaagaatctgaaaaaaaaatgacaactgaatcaactctctgtagatctgaagccaacacaacattacaagtcaactccacttccctaaaaaattcgcataaaaaatattcaccaccacaaccaccagttttggctaaccttggaaattgagaatgctcatggtctggcgagcagaccagcagactggactctggagaggaggagatctgggtccctaattcatggttagttcaatatatcttagatatttaggtagaatgccttctctccacccaaataactaatctatgagactacacgatattctgacagctcctcgaagagcaattcaggagctattaaaatagcatggagctggtttactatgagaggtacaagcggaagcacctgtgagttaacctgtggcagctggacccagcagaacccaggttctcagcaatcgcagggtttgaaggtcatgggctctggggccagccagcctgtctgggcttgaaccctaactctgccccttgctgtgtggccaagggtttgttgctcagccttgctgagccttgcctgtcttgctcacctgtaacatgagatgacagagtgccccttcctcacggacttgcaatgccgattcagtgagaagcatggagtaggtgccccccacccgccgagcctcaaacacatcagccattcctgctccagtaacacctccatcgcctccaccactgagtgtgacgcctcctcatgggtgcatgctacagggctttaggatggggtagggcaaggacttggagaaggcaatggcaccccactccaatactcttgcctggaaaatctcatggacggaggagcctggtgggctgcagtccatggagtcactaagagtcggacacgactgagcgacttcactttcacttttcactttcatgcattggagaaggcaatggcaacccactccagtgttcttgcctggagaatcccagggacgggggagcctggtgggctgccgtctatggggtcacacagagtcggacatgactgaagcgacttagcagacttagcagcagggcaaggactgacttgaagatatttaagccaaggcttgaaatatgaggaggtatgggtagaagaaacagccagaaagatcccaaggtgggaaggcctgacctttcaggaatagacagatagactgatattcatgtggatggagctttgtgaaccagccagtaaatggtgtgtgatgagttcagagatgggggcagttaagcatggagaggagcctgaatatattcaaagtacaaaggggaacccctcataggcacccactccccgctgtgttttagaaagagccctctgcatgcacactgagaatggtgcagagaacccctcaggaggcgactgcaatggtccgagcagtggtgatgggcctgggccagaatgttggcctggtgtgggaatggggggtgcccacaattgaggagcattctggaaatcaaggtgacagtccctgtagctactctgactctgatgactgtgattattcactcgtgacatgaaattccaagactgcacaccaggagggccttagagattggagttacagagctggaagccatcagcgtggtgggattcgcgcatgtttggccaattgcccttcttacaggaagaggagggtccgaggcccaggaggggtagatgcccacgctgggctgtatgaggggcagaggccgaactgtcttccaggaaaacagagccccagggccaggcccttgaccaccaccagcagtcctgctcaggtaattccccctgctcccaagtgtggttgctgtgtgttgaggcttcatctcacccagcctcaacctggaccctcccagtgatggggagctcaccaccctgtgctccatccctgctcagcctaggtggacgcatctcccacctatggaatcaaacccaggtctgcttatgtccacacagagcagcgcacacccttgttccaatgtgtcagggaattccaccgcctcattcagggtgctctcttttccaggctaaacatgctccaggaagagcagggtctccgaacatacagctgccaaactgctggcccgccccggtggtggaaaccacgccggcggcagcagcagcagaaatgacagcgtcgaggggcagcctggcctgaaaacaaacccgggtctgtccggtccccgctcggctgtgagtcactggccatttttacttctaaggccagaccttcagcttcatcaggggtctcctgtctcctggctgacgcagaagacggaggagacaccatctggccatccagctgctactgctgctaagtcgcttcagtcgtgtccaactgtgtgtgaccccatatatggcagtccaccaggctcccctgtccctgggattctcccggcaagaacactggagtgggttgccatttccttctccaatgcatgaaagtgaaaagtgaaagtgaagtcgctcagttgtgtctgactcttagcgaccccatggacttcatcctaccaggctcattcgtccatgggattttccaggcaagagtgctggagtggggtgccattgccttctccagaggcctccgcaaaaggtgtgagacccgcagggtgtttggggggtcgcggccggcgaggtgggcggacaccaggtctgttaccctgacggcctggccttcacctgcagctttgggttggcttcttgcgcttatctgctcagcagcgcagcgaattgtgcaaccagagctctcttctgtgtgacatgggggtgccatgctgtgcttcactaaacgagagagttgaaagaagggccttgggacttcgtggggtgatccagtggcaaagactctgcactcccaatgcggtggagcggggggtccaggttcgattcctggtcagggaactagatggcacgtgccacaaacacaatgaagatcctgcatgcggcagctacgacccagggcagccaagtaaacaaatactaatatttaaaaagaaagcaggaaagagggcctcggtggggacgcacacctaccagccaccttggggagggaagttggtttctctgggcacgagccattcaaggctcagctgagatgctggaggaatcagggctggtccgttgtcctgcggtctttttttctgttcctagatgtgcccgcttgagccctgagtgctggcagaatatgcagtggagggcctggggctgccaacacactcagggggaccccaaaggggagactgagcttgtggccgcctgcctggcctggcccgggaccccttcctggcagctaggatggatctgctgggctctccttggcagaaagggggcagccctgggcccagagaactggcctggaggcccgtccccaccgattaatgcttgtggccgtgggcaagtcacgtggcctcgctgcacctcgggttccctggggaaggccagggtgccgaccgctctcatctccttggttctctgagaaggaaatgagatggtgaagggccttctgcatagtagacgctcactgagcatcaggaatggtgaaataaaattcatatttttggcaagacaagaaagtttagacgtaacgttttcttggctcctttgggcctcctccctccccttccgtgtgtcttatgctgcattaaccagacttccttaacttcctgcttaatttcataaagggtcacaactccttaggagataaccttcctttttaattgttaagggaccacagtgagccataacccactacttgctttgtacatgtagctctagattgtatgaactgtcaatgtgtataatgatgtataatgctttgtcttaaaaatgtatataactgtgctgtaacaggcagaacgatcctcagagctttctggaagattgtctcccaggttgtaaccctcaggttggtgcaaataaaattttccatgcctatcttgctgctgctaagttgcttcagtcatgtccgactctgtgcgaacccatagacggcagccagccaggctcccccgtccctgggattctccaggcaagaacactggagtgggtgcctatcttgctaagttgcttcaattctgtctgactctttgcgaccccatcgactgtagcccaccagggtcctctgtccatgggattctggtaagaatactggagtggattgccatgccctcctccaggggatcttcctaacccagagatcgaattagagtcttttaagtctcctgtgtcgacaggcaggttctttaccactagggccagtagtgtctgactacttagtagctcagtcatgtctgactctttgtgacccccatggatggtagccagccaggctcttctatccatgggatttcccaggcaaggatattggagtgggttgccattcccttctccagggggtcttcctgacccaggggttgaacccagctctcctgcattacaggcagattctttatcttctgagctaccagggaagcccatttctttcttaaattgattgatgtttttcctttaacagtgttttgcttttagatttgaaaaccattgacaagaagtcaccttgatctctcttggacaagcccttgcatggcctggggttcccacccatcaccacctgaacctccagagtgcctgggagccactccttccggcccttgtccagggtgatgctgcccccaagtccgctcccaagggggatttctggagcagaaccagctctctctccagctccatctctatgccactcccaccccaccacccatctgagagcccatagaaagcagggactggatcttctcttttaaagttatgaaagaaagtgaaagtgaaagttgctcagttgtgtctgactctgcaaccccatggcctatgtagtccatggaattctccaggctggaatactggagtgggtagcctttcccttctccaggggatcttcccaacccaggagtcgaagcggggtttcctgcatttcaggcagattctttaccaactgagctatcagggaagcctgaagttatcgtaacatatataaaacataagatttgtcattttagccattttaatttattattgtcaaagatgtatcaatgttttttgttaccagaactttattcaccagagatgcttcaatagtcacttacgtaacgaatcaccacagacattaatttctgcaaaaatccccctgatcaacaatgtgttaaagtatacaactcagtggcattaattacattcattatattgttaccaccaccaccatctccagatcttttccatcaccccaaacagaagctctgtactcattcaacagcaactccccattcctctcaccccacagcctctggcaacctctgttctactttgtctgtatgaatttgcctattctgtatgttttgtataactggaatcatcactatctgtccttctgtgactggcttacctcacttagcataatatttttgaggttcatccatgttgcagtatgtactagaacttaacttcttcttatgaccaaataatattccagtgtataggtataacacattgtgtttattcattcatttgcttatgaacacttggattgtctccactttggggctagtatgaatgatgttgctatgaaccctagtatacaagtatccatctgagtctgtcatcagttctcttttcttgggctccaaaatcactgtggatggtgactgcagacacaaaattaaaagatggttgctccatgaaagaaaagctacgagaaacctagacagcatattaaaaagcagagacatcactttgaggacaaatgtccttatagtcaactatggtttttccagtagttatgtacagaggtgagagttggaccataagaaggctgagcgctgaagaattgatgcttttgaattgtgctagaaaagactcttgagagtcacttgggcagcaaggatatcaaatcagtcaatcctaaaggaaatcaaccttgaatattcactggaaggactgacgctgaaactctagtactttggctgcctgatgtgaagagtcgactcattggaaaagaccctgatactggaaaagattgaaggccagaggagaatgggacgacagaggatgagatggttggatggcatcactgactcaacggacatgaatttgagcaaactccgggagacagtgaaggacaaggaagcctggcgtgctgcagtccatggggttgcaaacagctgaacacgactgagcgactgaacaacaagaatagcacaacaggttgaattgtgtcccccaaaaagatacattggaatcctaagcccagcagctcagaatgtaattttatttagagataggatctttatttagaaataggatctttacagagacaatcgagttaaaacgaggtcagtgtgggcctttatccaacacgcctgtgttcttatgagaaagggaaatctggacacaggggtggagatgcacaaagggcagacacatgaagacccagggagaagaggaccacggagaagcccgcgagaaaggctc

(19) United States
(12) Patent Application Publication
(10) Pub. No.: US 2007/0224623 A1
(43) Pub. Date: Sep. 27, 2007

(54) Simplified QTL mapping approach for screening and mapping novel markers associated with beef marbling

(76) Inventors: Zhihua Jiang, Pullman, WA (US); Jennifer J. Michal, Albion, WA (US); Qianjun Xiao, Madison, WI (US); Tito A. Wibowo, Pullman, WA (US)

Correspondence Address: FROMMER LAWRENCE & HAUG, 745 FIFTH AVENUE- 10TH FL., NEW YORK, NY, 10151, US

(21) Appl. No.: 11/709,307

(22) Filed: Feb. 21, 2007

Related U.S. Application Data
(60) Provisional application No. 60/775,625, filed on Feb. 22, 2006.

(51) Int. Cl. C12Q 1/68; G06F 19/00; G06Q 10/00

(52) U.S. Cl. 435/6; 702/20; 705/1

(57) ABSTRACT

Particular aspects provide an inexpensive QTL mapping approach for genome-wide scans of QTL linked markers and for narrowed locations of QTL regions, which may comprise integration of the amplified fragment length polymorphism (AFLP) with DNA pooling and selective genotyping and comparative bioinformatics tools. AFLP simultaneously screens high numbers of loci for polymorphisms and detects many more polymorphic DNA markers than any other PCR based detection systems, and provides for identification of “responsible mutations” for a QTL. Aspects of the present invention also provide novel compositions and methods based on novel DOPEY2 and/or KIAA1462 single nucleotide polymorphisms such as AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene, which may provide novel markers for beef marbling and subcutaneous fat. Additional aspects provide for novel methods which may comprise marker-assisted selection to improve beef marbling and subcutaneous fat in cattle.

### INCORPORATION BY REFERENCE

This application claims benefit of U.S. provisional patent application Ser. No. 60/775,625 filed Feb. 22, 2006.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relate generally to genome-wide scans of quantitative traits loci (QTL) linked markers, and more particularly to novel and inexpensive QTL mapping approaches for genome-wide scans of QTL linked markers and for narrowed locations of QTL regions.

The present invention also relates to the identification of genetic markers (single nucleotide polymorphisms (SNPs)) within the bovine dopey family member 2 (DOPEY2) gene and the bovine KIAA1462 gene and their associations with economically relevant traits in beef cattle production. The invention further relates to methods and systems, including network-based processes, to manage the SNP data and other data relating to specific animals and herds of animals, veterinarian care, diagnostic and quality control data and management of livestock which, based on genotyping, have predictable beef marbling and/or subcutaneous fat, husbandry conditions, animal welfare, food safety information, audit of existing processes and data from field locations.

### BACKGROUND OF THE INVENTION

Marbling is a term commonly used to describe the appearance of white flecks or streaks of fatty tissue between the muscle fibers in meat. As an indicator of intramuscular fat, this trait has attracted a great deal of publicity and interest for many years, since deposition of fat in the muscle of a beef carcass contributes very positively to the taste, texture and flavor of the meat (Elias Calles et al., 2000, J Anim Sci. 78, 1710-1715). Obviously, beef marbling is of high economic importance, but progress is currently limited because selection of beef marbling requires tremendous effort, expense and time. A trait such as beef marbling is, therefore, ideally suited to capitalize on molecular genetic technologies (Parnell, 2004, Aust. J. Exp. Agr. 44, 697-703). Identifying, mapping, and understanding the function and control of genes for beef marbling will permit the development of new genetic technologies and open the way to realize the full genetic potential for improvement of beef production for maximum profits.

As usual, multiple genes and environmental factors determine complex genetic traits such as beef marbling. The individual loci that make up the genetic component of a quantitative trait are called “quantitative traits loci (QTL).” QTL mapping is defined as a process to localize chromosome regions harboring genetic variants that affect a continuously distributed, polygenic phenotype (DiPetrillo et al., 2005, Trends Genet. 21, 683-692). Genome-wide linkage studies of complex traits conducted by utilizing highly informative microsatellite markers have proven to be a feasible means of detecting QTLs in different species. However, due to costliness and high labor requirements, these genome scans were usually performed with relatively few markers spanning the whole genome, and thus provided a low resolution of mapped QTL locations, perhaps 20 cM or more. These distances make it difficult to move from the mapped QTL to identification of actual genes. For example, although QTL analysis started in the early 1990s, investigators have identified only ˜30 causal genes underlying QTL in mice so far (Flint et al., 2005, Nat Rev Genet. 6, 271-286). Thus, the major hurdle to identifying QTL genes is not identification and localization of a QTL in genome, but rather to the expensive and time-consuming process of narrowing QTL to a few candidate genes for a detailed characterization and functional analysis.

It remains advantageous to provide further SNPs that may more accurately predict the beef marbling and/or subcutaneous fat phenotypes of an animal and also a business method that provides for increased production efficiencies in livestock cattle, as well as providing access to various records of the animals and allows comparisons with expected or desired goals with regard to the quality and quantity of animals produced.

Citation or identification of any document in this application is not an admission that such document is available as prior art to the present invention.

### SUMMARY OF THE INVENTION

The present invention relates to a simplified, inexpensive QTL mapping approach for genome-wide scans of QTL linked markers and for narrowed locations of QTL regions. This simplified approach involves integration of the amplified fragment length polymorphism (AFLP) with DNA pooling and selective genotyping and comparative bioinformatics tools. AFLP simultaneously screens high numbers of loci for polymorphisms and detects many more polymorphic DNA markers than any other PCR based detection systems. Thus, the technique provides a capable power to reveal “responsible mutations” for a QTL. Meanwhile, DNA pooling and selective genotyping are applied to reduce the numbers of samples. The main mechanism involved in the DNA pooling and selective genotyping is that the distributions of the genotypes are quite similar close to the means, but very different in the tails of the distributions. Therefore, the individuals with the extreme phenotypic values represent the most information for the QTL. Comparative bioinformatics tools take advantage of genome sequencing and conservation in human, cattle and other mammalian species. In particular, retrieval of the same gene sequences in the target species or orthologous sequences in other species can immediately place QTL-linked markers to narrowed chromosome regions. Using such a simplified approach, QTL-linked AFLP markers for marbling were identified in Wagyu x Limousin F₂ crosses, which have relevant evidence for obesity observed in humans.

The present invention relates to the identification of genetic markers (single nucleotide polymorphisms (SNPs)) within the bovine genes encoding a bovine dopey family member 2 (DOPEY2) gene and/or a bovine KIAA1462 gene and their associations with economically relevant traits in beef cattle production.

The invention encompasses a method for sub-grouping animals according to genotype wherein the animals of each sub-group have a similar polymorphism in a DOPEY2 gene and/or a KIAA1462 gene which may comprise determining the genotype of each animal to be sub-grouped by determining the presence of a SNP in a DOPEY2 gene and/or a KIAA1462 gene, and segregating individual animals into sub-groups wherein each animal in a sub-group has a similar polymorphism in a DOPEY2 gene and/or a KIAA1462 gene.

The invention also encompasses a method for sub-grouping animals according to genotype wherein the animals of each sub-group have a similar genotype in DOPEY2 gene and/or a KIAA1462 gene which may comprise determining the genotype of each animal to be sub-grouped by determining the presence of a single nucleotide polymorphism(s) of interest in DOPEY2 gene and/or a KIAA1462 gene, and segregating individual animals into sub-groups depending on whether the animals have, or do not have, the single nucleotide polymorphism(s) of interest in DOPEY2 gene and/or a KIAA1462 gene.

The genetic polymorphism(s) of interest may be selected from the group consisting of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene. The invention further relates to a method for sub-grouping animals according to genotype wherein the animals of each sub-group have a similar genotype in a DOPEY2 gene and/or a KIAA1462 gene which may comprise determining the genotype of each animal to be sub-grouped by determining the presence of any one of the above SNPs, and segregating individual animals into sub-groups depending on whether the animals have, or do not have, any one of the above SNPs in a DOPEY2 gene and/or a KIAA1462.

The invention also relates to method for identifying an animal having a desirable phenotype as compared to the general population of animals of that species, which may comprise determining the presence of a single nucleotide polymorphism in a DOPEY2 gene and/or a KIAA1462 gene of the animal, wherein the presence of the SNP is indicative of a desirable phenotype.

In an advantageous embodiment, the animal may be a bovine. In another advantageous embodiment, a DOPEY2 gene and/or a KIAA1462 gene may be a bovine DOPEY2 gene and/or a bovine KIAA1462 gene.

The invention also encompasses computer-assisted methods and systems for improving the production efficiency for livestock having desirable beef marbling and/or subcutaneous fat and in particular the genotype of the animals as it relates to DOPEY2 and/or KIAA1462 SNPs. Methods of the invention encompass obtaining a genetic sample from each animal in a herd of livestock, determining the genotype of each animal with respect to specific quality traits as defined by a panel of at least two single polynucleotide polymorphisms (SNPs), grouping animals with like genotypes, and optionally, further sub-grouping animals based on like phenotypes. Methods of the invention may also encompass obtaining and maintaining data relating to the animals or to herds, their husbandry conditions, health and veterinary care and condition, genetic history or parentage, and providing this data to others through systems that are web-based, contained in a database, or attached to the animal itself such as by an implanted microchip. An advantageous aspect of the present invention, therefore, is directed to a computer system and computer-assisted methods for tracking quality traits for livestock possessing specific genetic predispositions.

The present invention advantageously encompasses computer-assisted methods and systems for acquiring genetic data, particularly genetic data as defined by the absence or presence of a SNP within a DOPEY2 gene and/or a KIAA1462 gene related to beef marbling and/or subcutaneous fat traits of the breed of animal and associating those data with other data about the animal or its herd, and maintaining those data in ways that are accessible. Another aspect of the invention encompasses a computer-assisted method for predicting which livestock animals possess a biological difference in beef marbling and/or subcutaneous fat, and which may include the steps of using a computer system, e.g., a programmed computer comprising a processor, a data storage system, an input device and an output device, the steps of: (a) inputting into the programmed computer through the input device data that includes a genotype of an animal as it relates to any one of the a DOPEY2 and/or KIAA1462 SNPs described herein, (b) correlating beef marbling and/or subcutaneous fat predicted by the DOPEY2 and/or KIAA1462 genotype using the processor and the data storage system and (c) outputting to the output device the beef marbling and/or subcutaneous fat correlated to the DOPEY2 and/or KIAA1462 genotype, thereby predicting which livestock animals possess a particular beef marbling and/or subcutaneous fat.

Yet another aspect of the invention relates to a method of doing business for managing livestock comprising providing to a user computer system for managing livestock comprising physical characteristics and genotypes corresponding to one or more animals or a computer readable media for managing livestock comprising physical characteristics and genotypes corresponding to one or more animals or physical characteristics and genotypes corresponding to one or more animals, wherein a physical characteristic intake, growth or carcass merit in beef cattle and the genotype is a DOPEY2 and/or KIAA1462 genotype.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIGS. 1A and 1B illustrate identification and selection of visually significant AFLP markers for beef marbling. FIG. 1A is an example that shows presence/absence patterns of a particular AFLP band between high (two DNA pools, HM1 and HM2) and low (two DNA pools, LM1 and LM2) animals. FIG. 1B is an example that shows high/low frequency patterns of a particular AFLP band between high (two DNA pools, HM1 and HM2) and low (two DNA pools, LM1 and LM2) animals.

FIGS. 2A and 2B illustrate characterization of an AFLP marker derived from primer combination E+AGT/T+CAT on BTA1. FIG. 2A: AFLP marker and its flanking sequences (SEQ ID NO: 8). A mutant site is underlined. FIG. 2B: A C/T mutation is detected in the AFLP fragment, but located in the selective primer extension region (SEQ ID NOS 9-10, respectively, in order or appearance).

FIGS. 3A and 3B illustrate characterization of an AFLP marker derived from primer combination E+AGT/T+ACT on BTA13. FIG. 3A: AFLP marker and its flanking sequences (SEQ ID NO: 11). Mutant sites are underlined. FIG. 3B: Two G/A mutations were detected in the AFLP fragment, one occurred within the TaqI cut site and one occurred in the selective primer extension region (SEQ ID NOS 12-13, respectively, in order or appearance).

FIGS. 4A-4AF illustrate a partial genomic DNA sequence of the bovine DOPEY2 gene (SEQ ID NO: 14). Primer sequences used for the PCR amplification are underlined. The SNPs are highlighted and they are AAFC03071397.1:g.12881G>C, g.12925G>A, g.12951T>C, g.13013A>G, g.13125G>A and g.13173C>T.

FIG. 5 illustrates a flowchart of the input of data and the output of results from the analysis and correlation of the data pertaining to the breeding, veterinarian histories and performance requirements of a group of animals such as from a herd of cows and the interactive flow of data from the computer-assisted device to a body of students learning the use of the method of the invention.

FIG. 6 illustrates potential relationships between the data elements to be entered into the system. Unidirectional arrows indicate, for example, that a barn is typically owned by only one farm, whereas a farm may own several barns. Similarly, a prescription may include veterinarian products.

FIG. 7A illustrates the flow of events in the use of the portable computer-based system for data entry on the breeding and rearing of a herd of cows.

FIG. 7B illustrates the flow of events through the sub-routines related to data entry concerning farm management.

FIG. 7C illustrates the flow of events through the sub-routines related to data entry concerning data specific to a company.

FIG. 8 illustrates a flow chart of the input of data and the output of results from the analysis and the correlation of the data pertaining to the breeding, veterinarian histories, and performance requirements of a group of animals.

### DETAILED DESCRIPTION

The present invention relates to a simplified, inexpensive QTL mapping approach for genome-wide scans of QTL linked markers and for narrowed locations of QTL regions. This simplified approach involves integration of the amplified fragment length polymorphism (AFLP) with DNA pooling and selective genotyping and comparative bioinformatics tools. AFLP simultaneously screens high numbers of loci for polymorphisms and detects many more polymorphic DNA markers than any other PCR based detection systems (Vos et al., 1995, Nucleic Acids Res. 23, 4407-4414). Thus, the technique provides a capable power to reveal “responsible mutations” for a QTL. Meanwhile, DNA pooling and selective genotyping are applied to reduce the numbers of samples (Darvasi & Weller, 1992, Heredity 68, 43-46). The main mechanism involved in the DNA pooling and selective genotyping is that the distributions of the genotypes are quite similar close to the means, but very different in the tails of the distributions. Therefore, the individuals with the extreme phenotypic values represent the most information for the QTL (Plotsky et al., 1993, Anim. Genet. 24, 105-110; Lipkin et al., 1998, Genetics 149, 1557-1567). Comparative bioinformatics tools take advantage of genome sequencing and conservation in human, cattle and other mammalian species. In particular, retrieval of the same gene sequences in the target species or orthologous sequences in other species can immediately place QTL-linked markers to narrowed chromosome regions. Using such a simplified approach, QTL-linked AFLP markers for marbling were identified in Wagyu x Limousin F₂ crosses, which have relevant evidence for obesity observed in humans.

Herein disclosed is a simplified QTL mapping approach in the study by integration of AFLP, selective DNA pooling and bioinformatics tools. The first step is to apply the AFLP technique in screening of QTL linked markers for a complex trait on DNA pools of animals with extreme phenotypes.

In the second step, the potential QTL-linked markers are validated individually on high and low performance of animals and truly significant QTL linked markers are further characterized by DNA sequencing.

The in-silico tools are then employed in the third step to identify same gene sequences of AFLP markers in the targeted species or orthologous sequences in other species and place the AFLP markers in the targeted genome.

Finally, the flanking sequence of an AFLP marker is used to design primers for revealing molecular causes responsible for the amplified fragment length polymorphisms and thus determining the genotype assay for marker-trait association analysis. Clearly, this simplified QTL mapping approach has several advantages.

The simplified QTL mapping approach is neither expensive nor time-consuming. In the present study, a genome-wide scan was performed using 64 primer combinations on four DNA pools. Theoretically, this process just requires a total of 256 PCR reactions. It was estimated that these 64 primer combinations would generate a total of 3840 (64×60) fragments. If it is assumed that 10% of these fragments (Ajmone-Marsan et al., 1997, Anim. Genet. 28, 418-426; Felip et al., 2005, Aquaculture 247, 35-43) are polymorphic, the screening was done with a total of 384 markers. If a conventional genome-wide screening approach was pursued with 384 markers on 250 F₂ progeny used in the present study, at least 96,000 PCR reactions would have been conducted. An additional 480 reactions were used for individual validation of ten potential QTL linked markers. The in silico retrieval of flanking sequences of AFLP markers and the in silico mapping of them to the target genome was free. Therefore, this simplified QTL mapping approach saves a lot of time and laboratory expenses. The approach is particularly powerful when one or two traits are handled at a time.

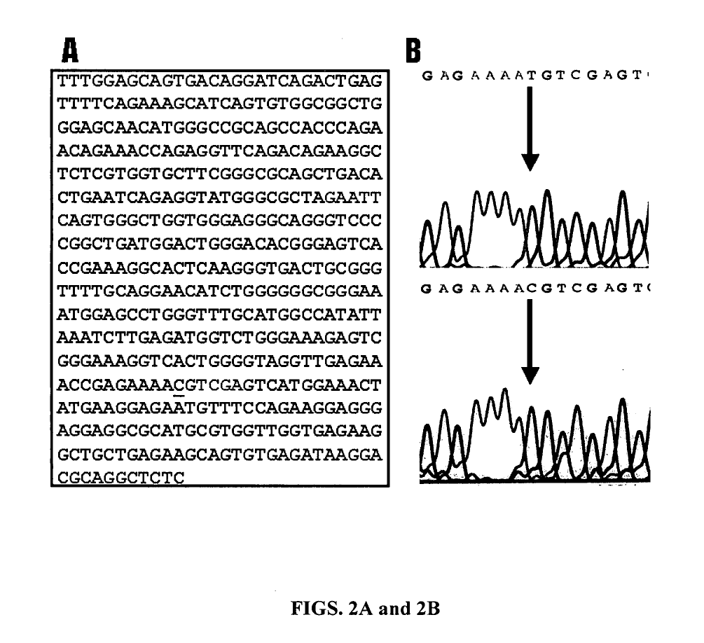

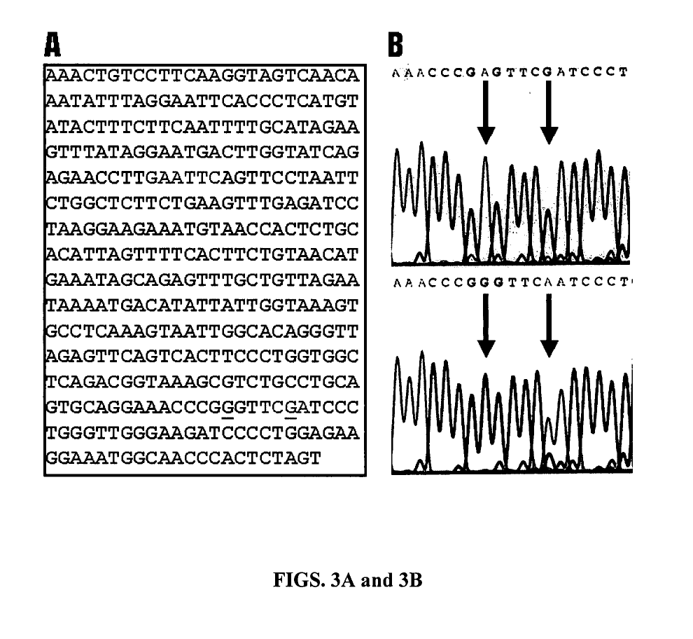

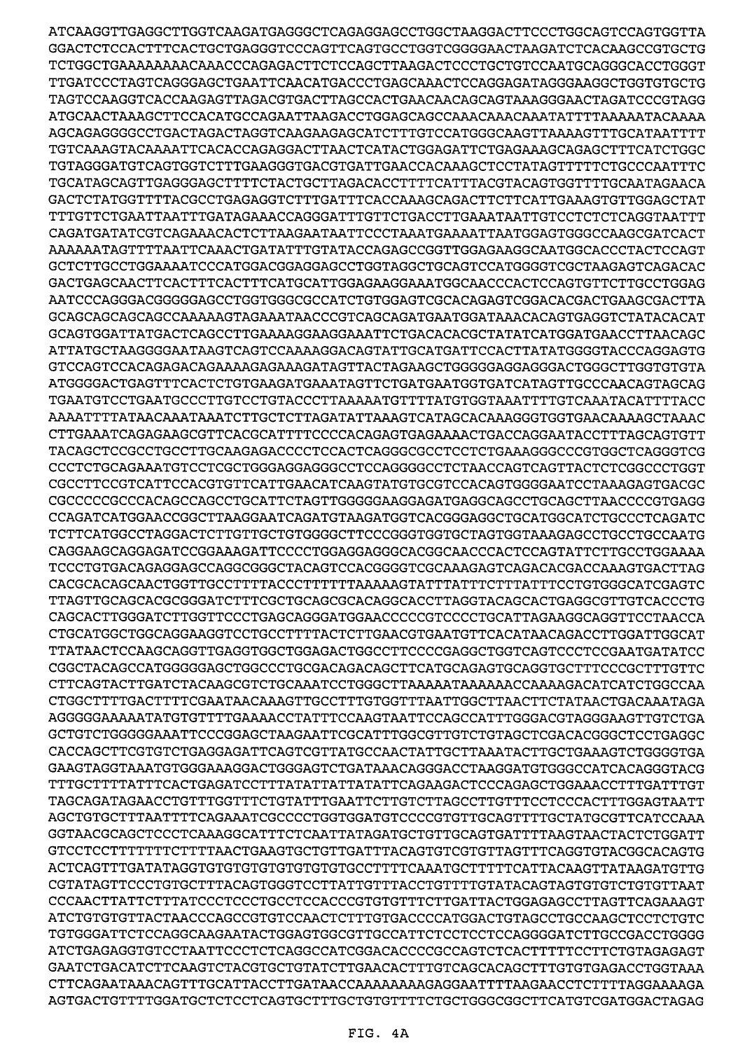

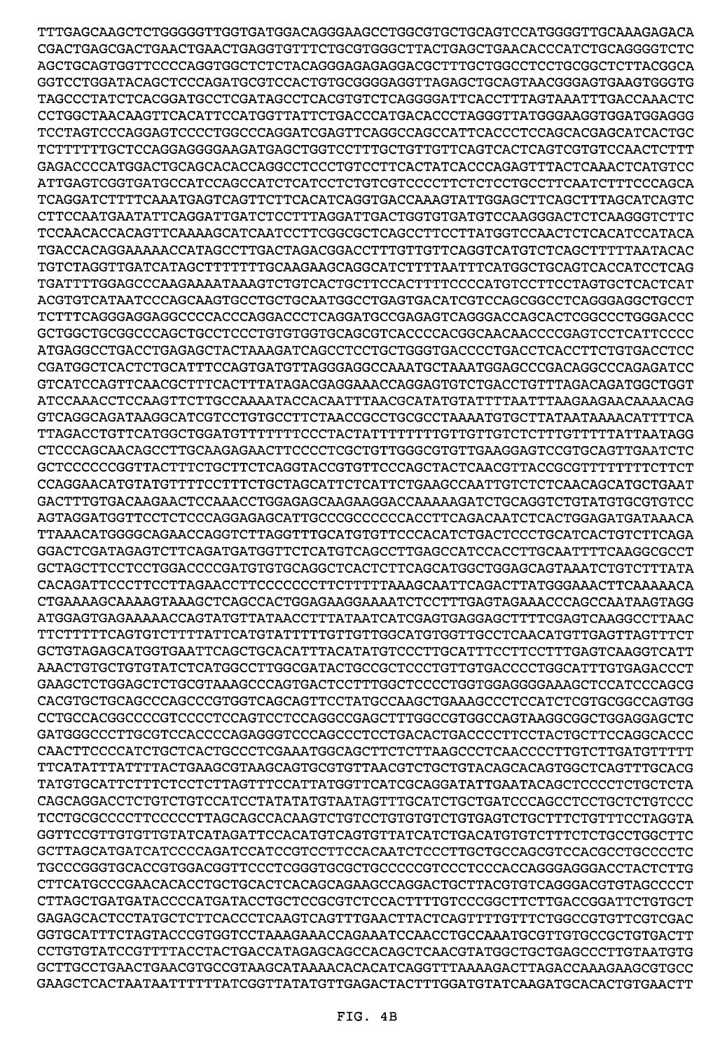

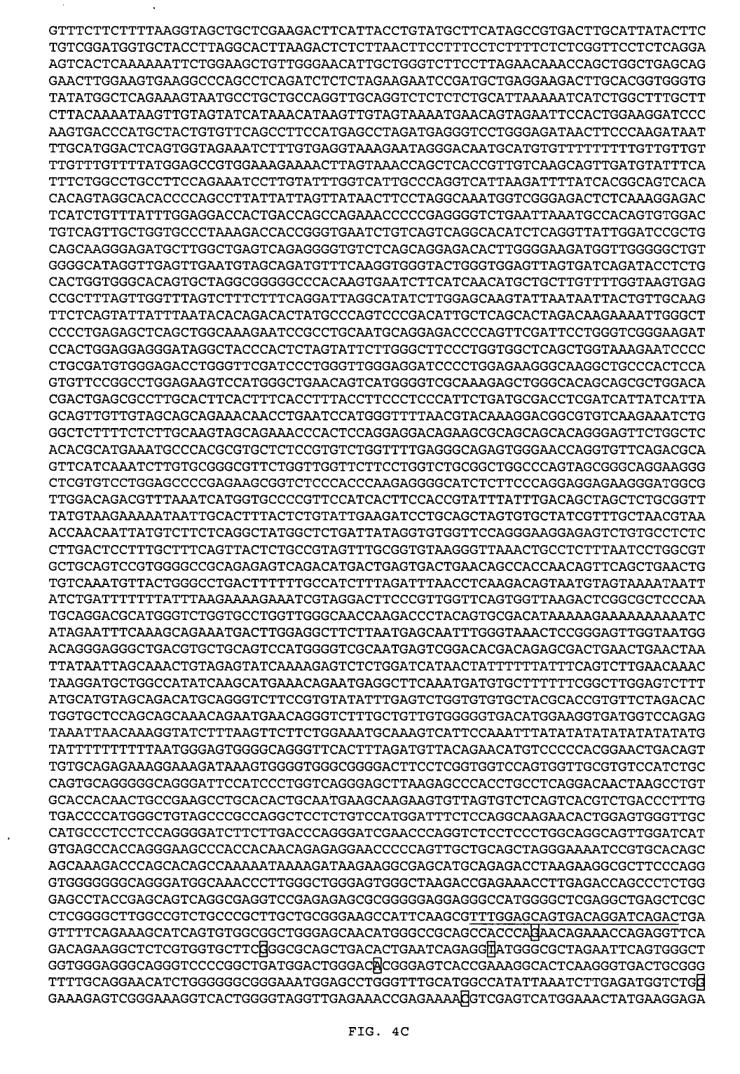

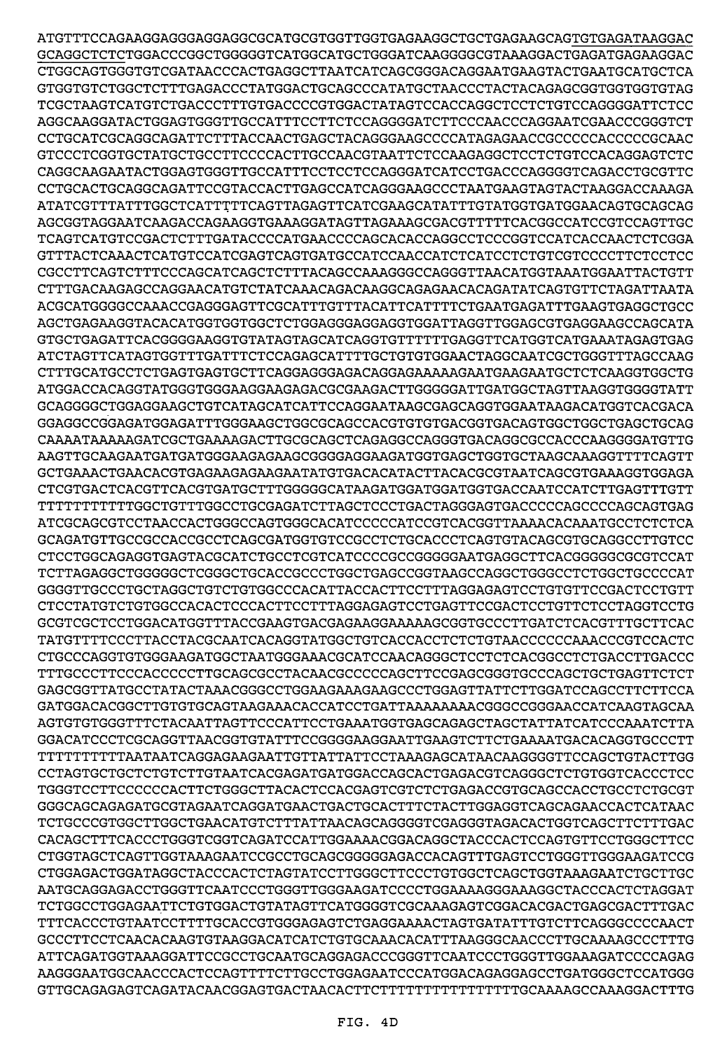

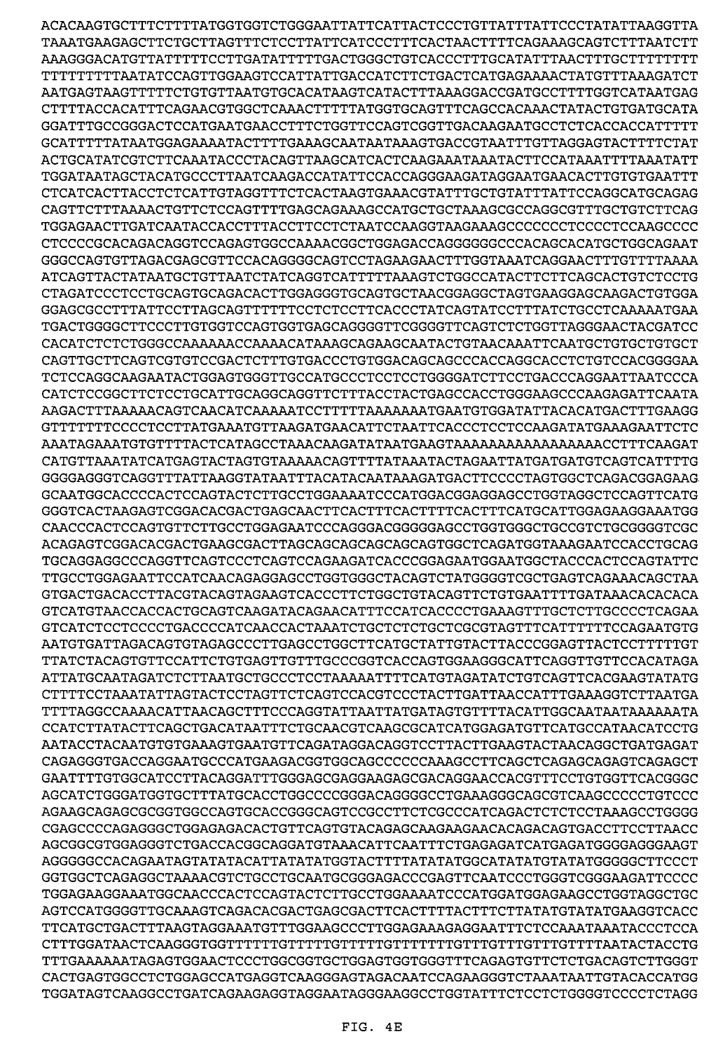

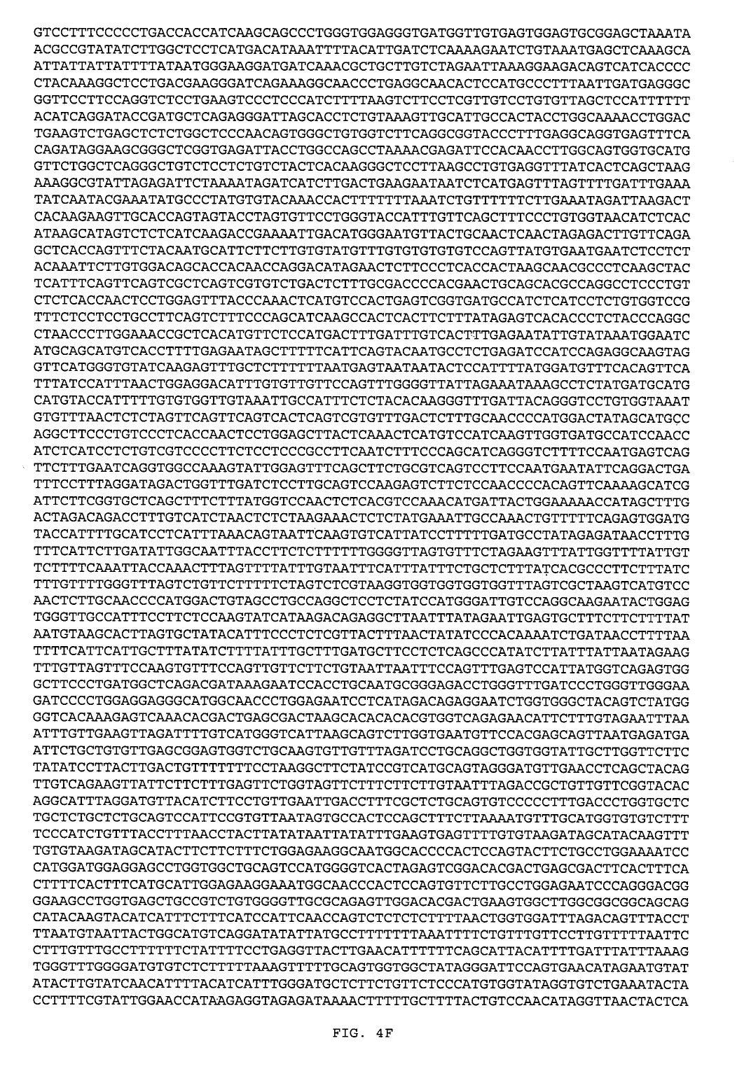

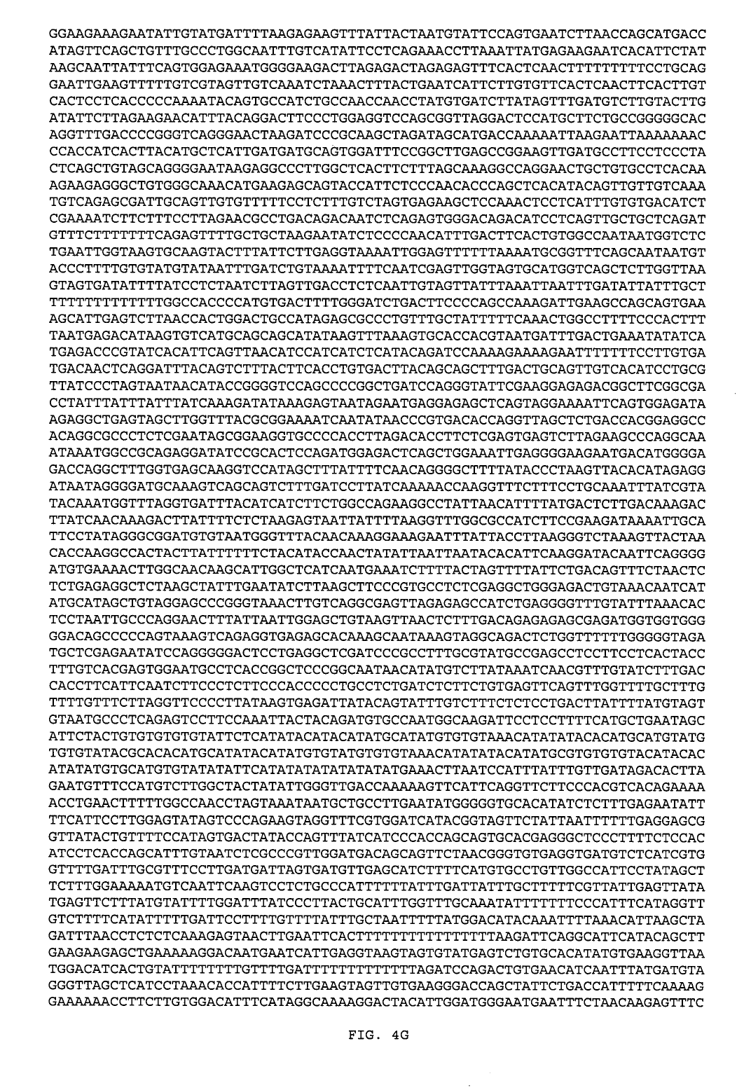

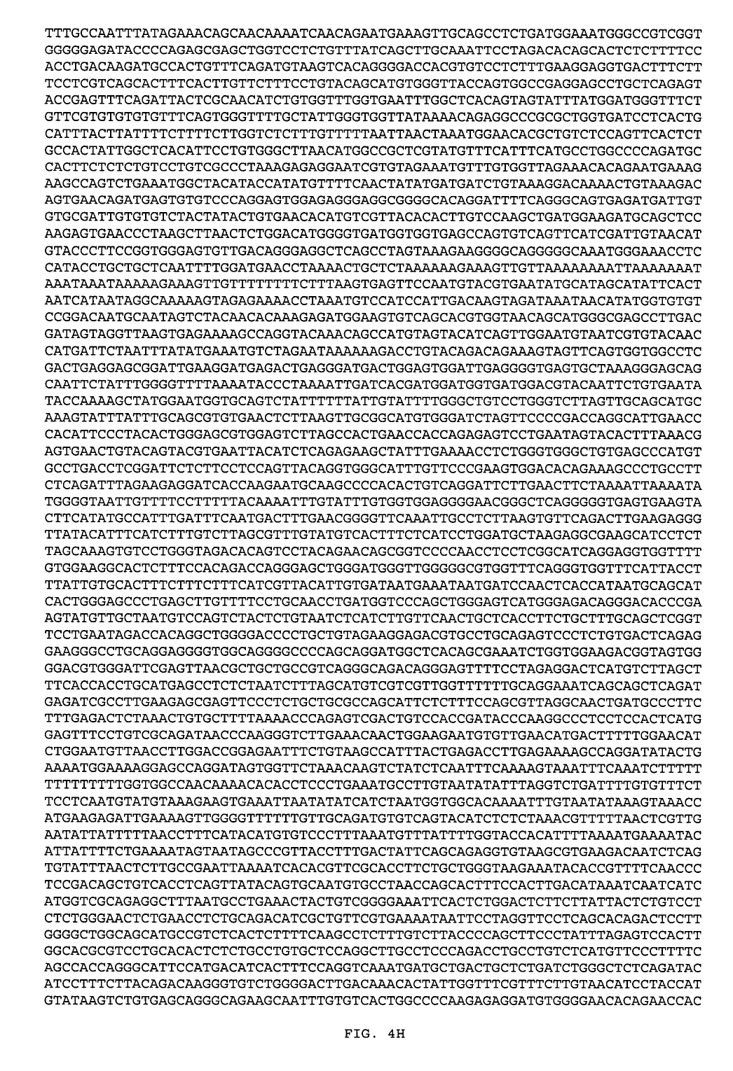

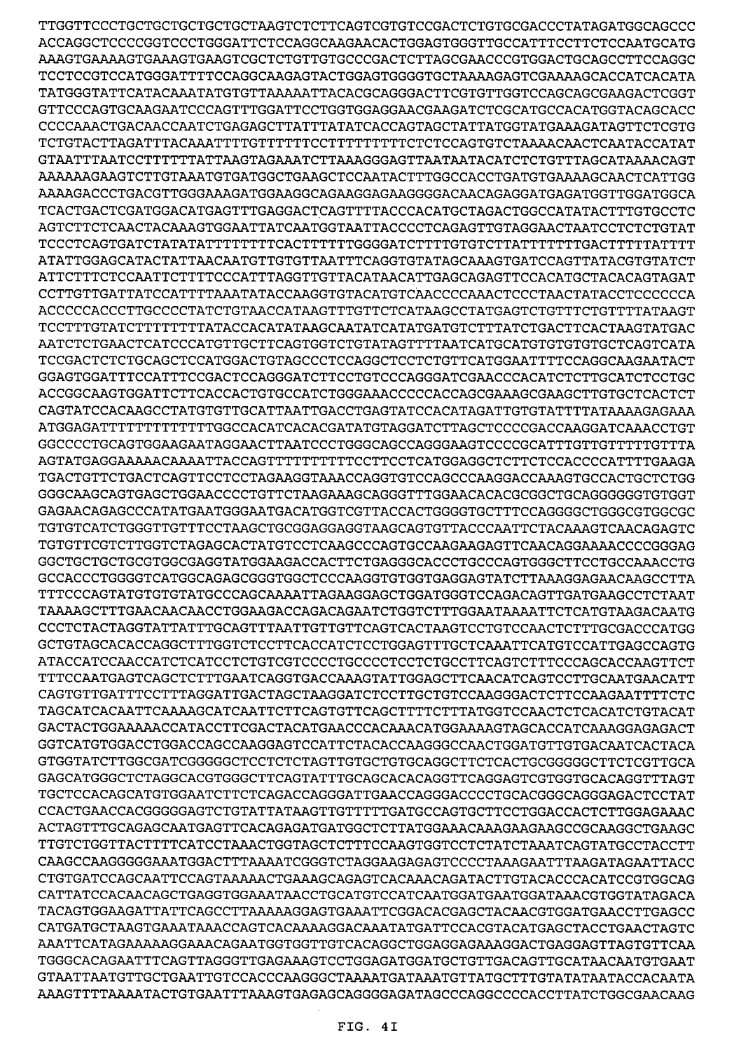

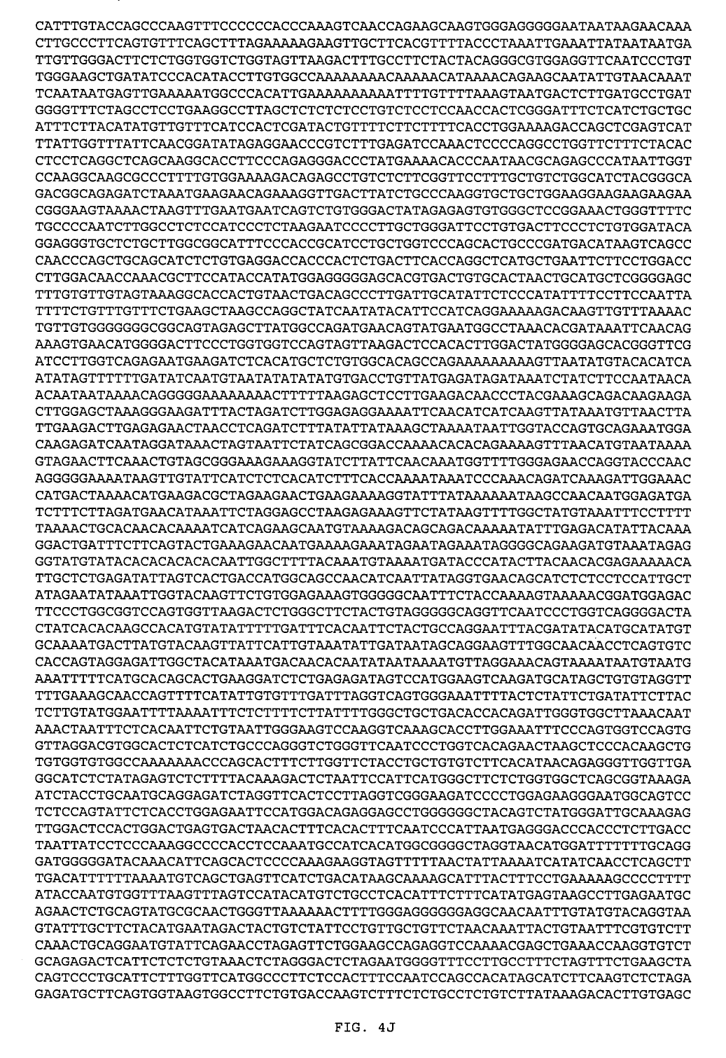

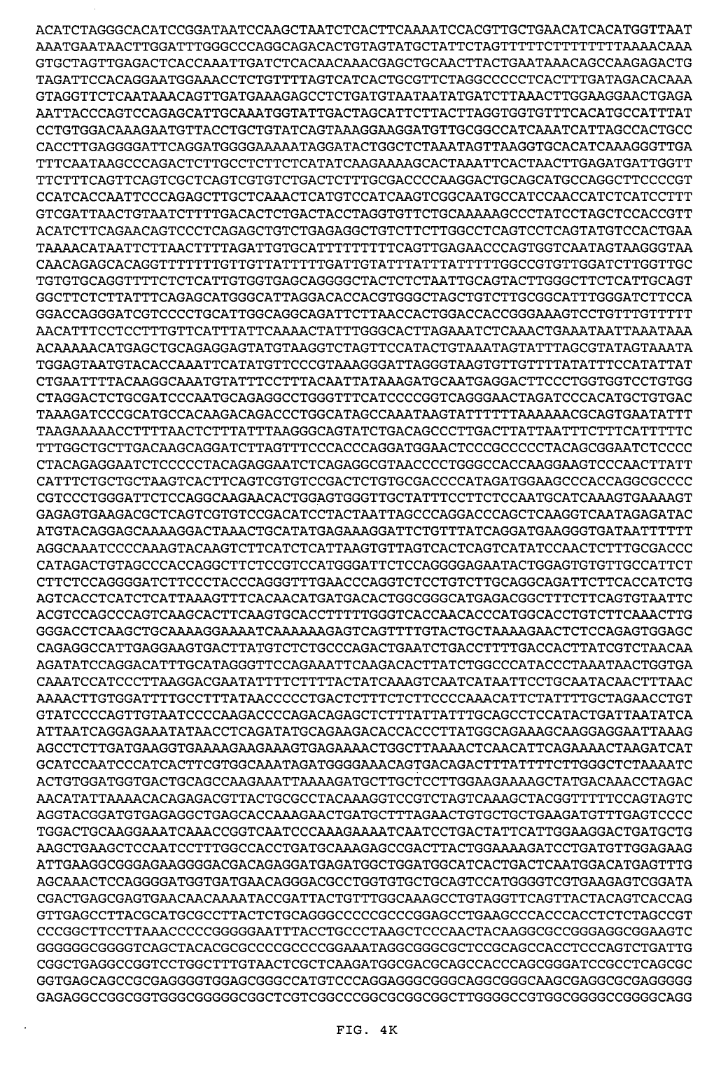

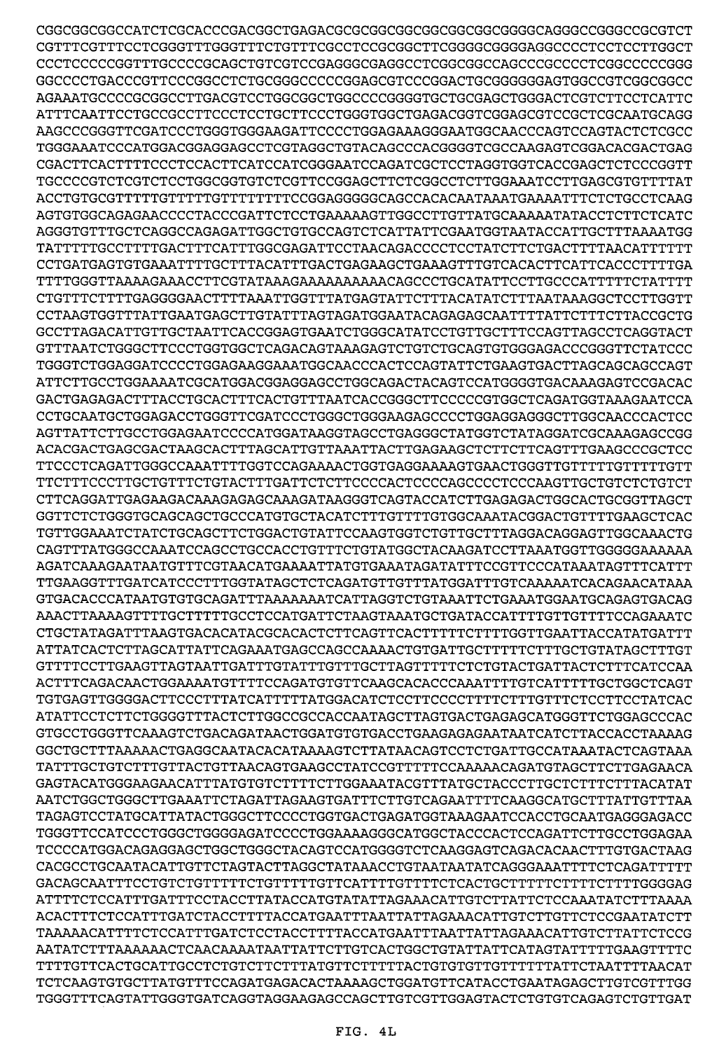

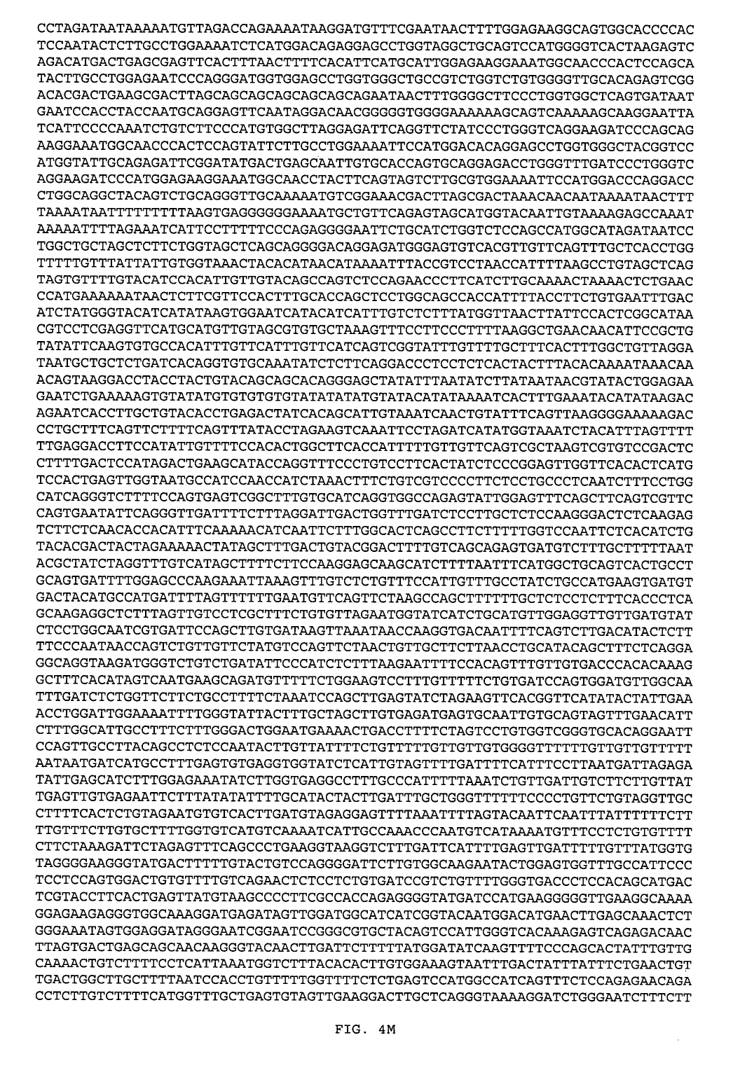

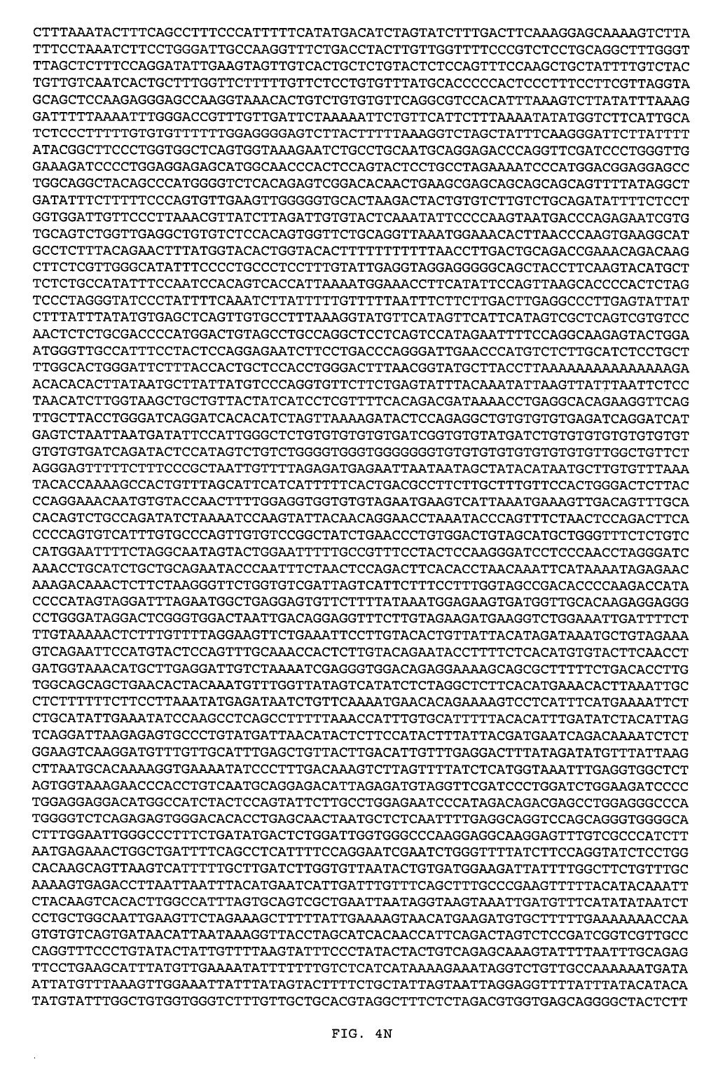

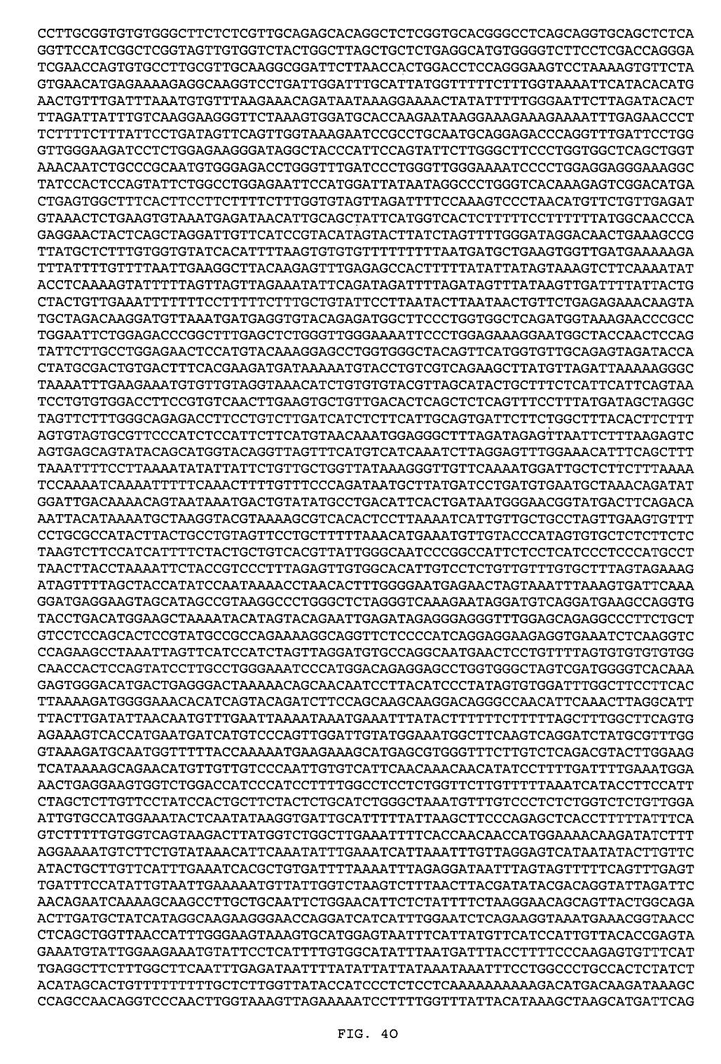

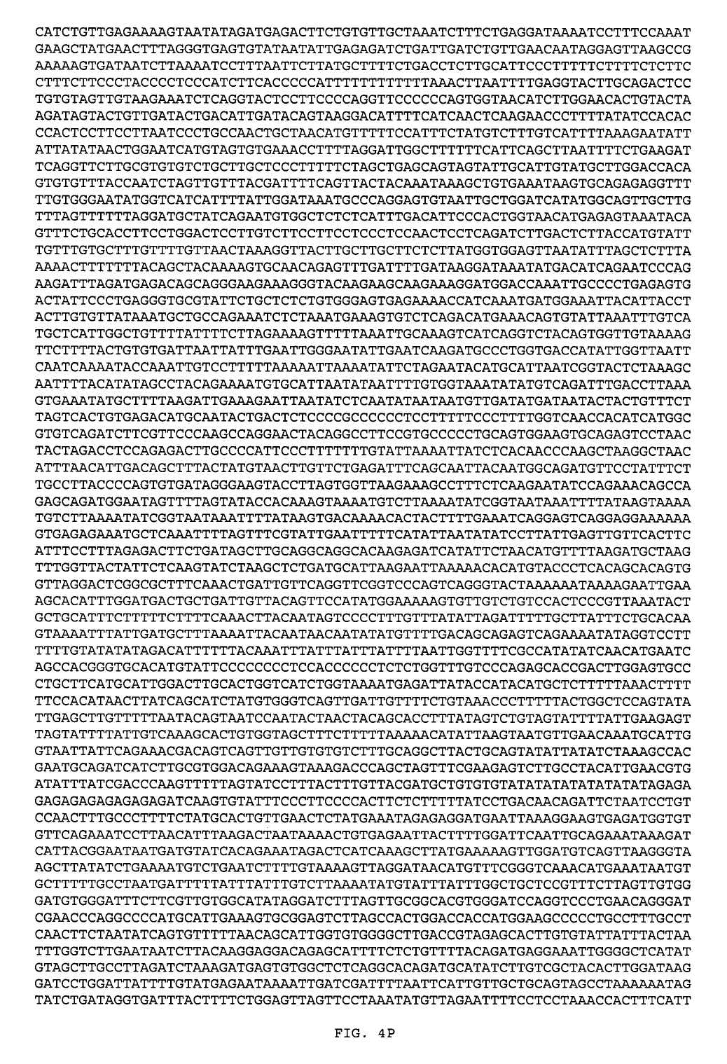

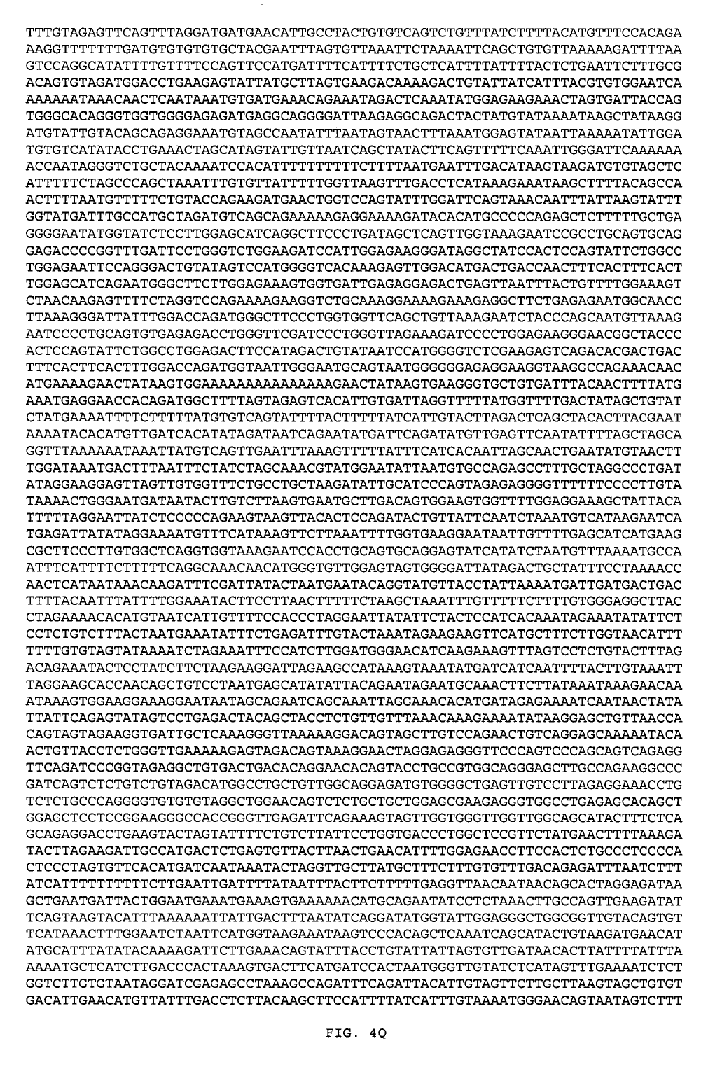

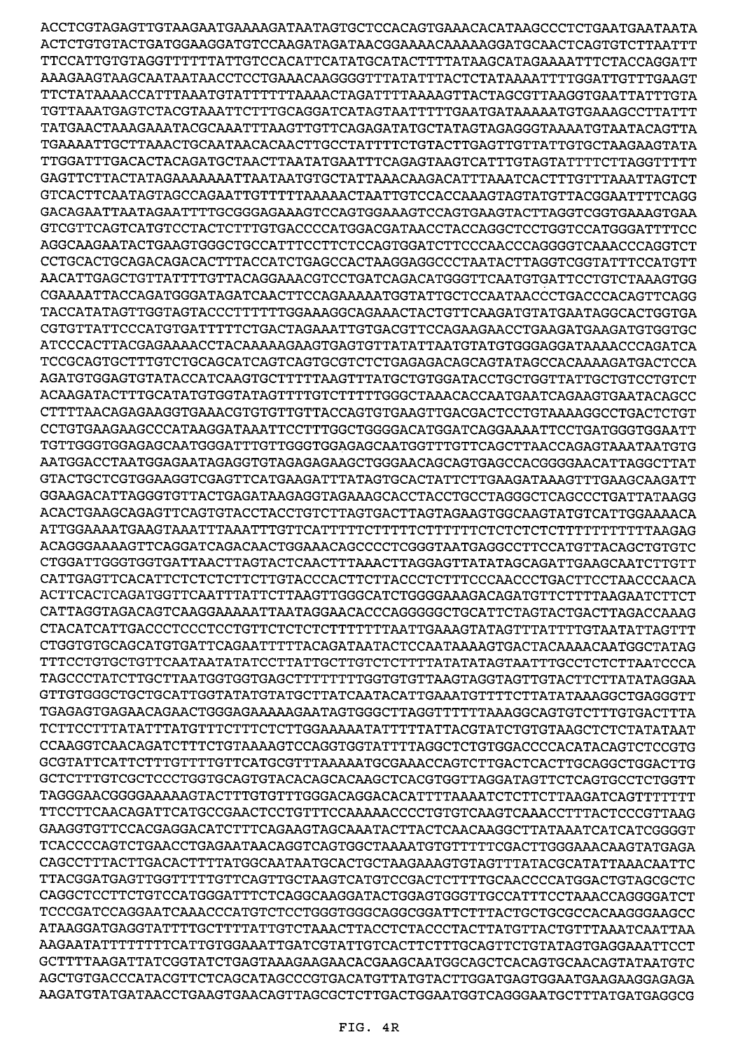

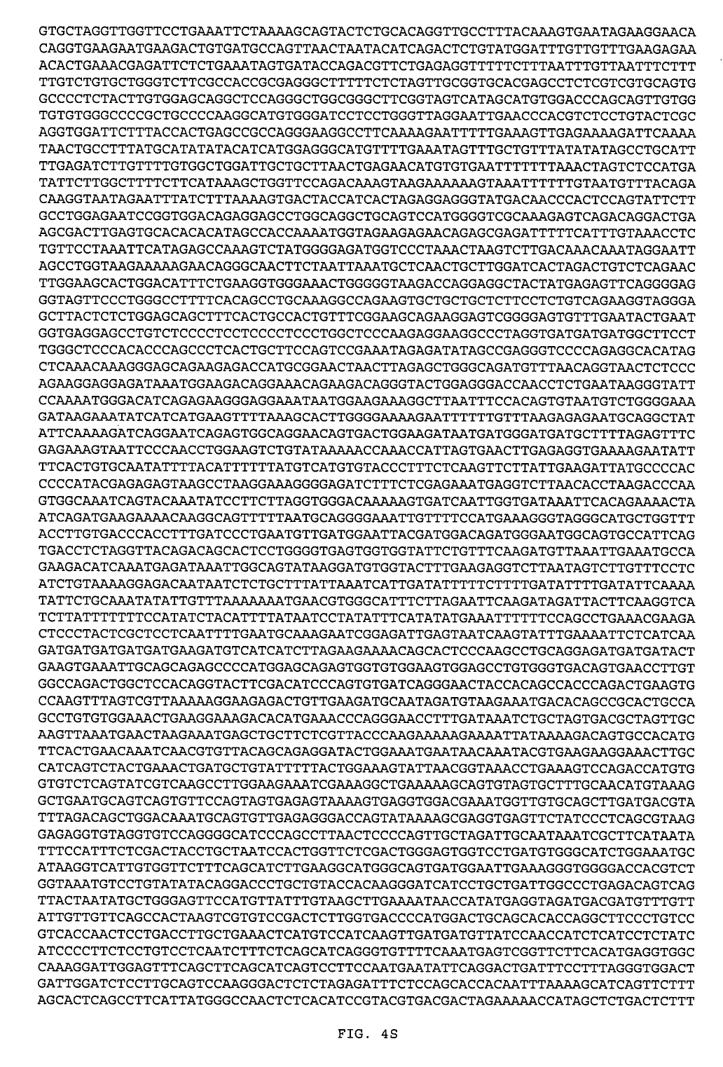

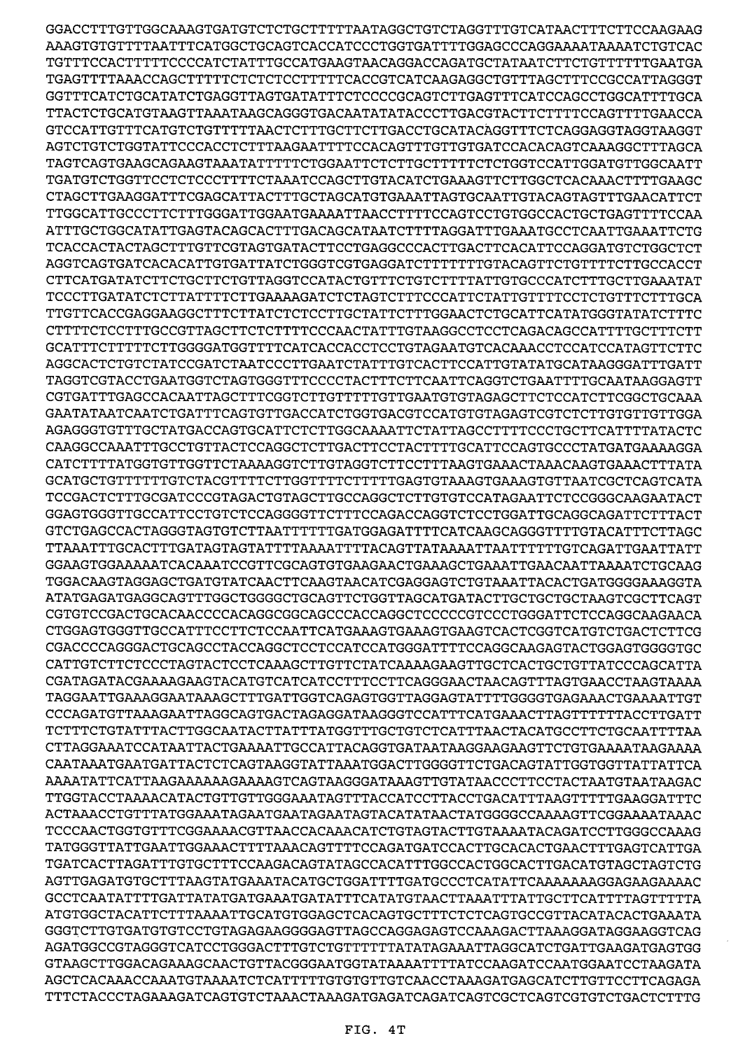

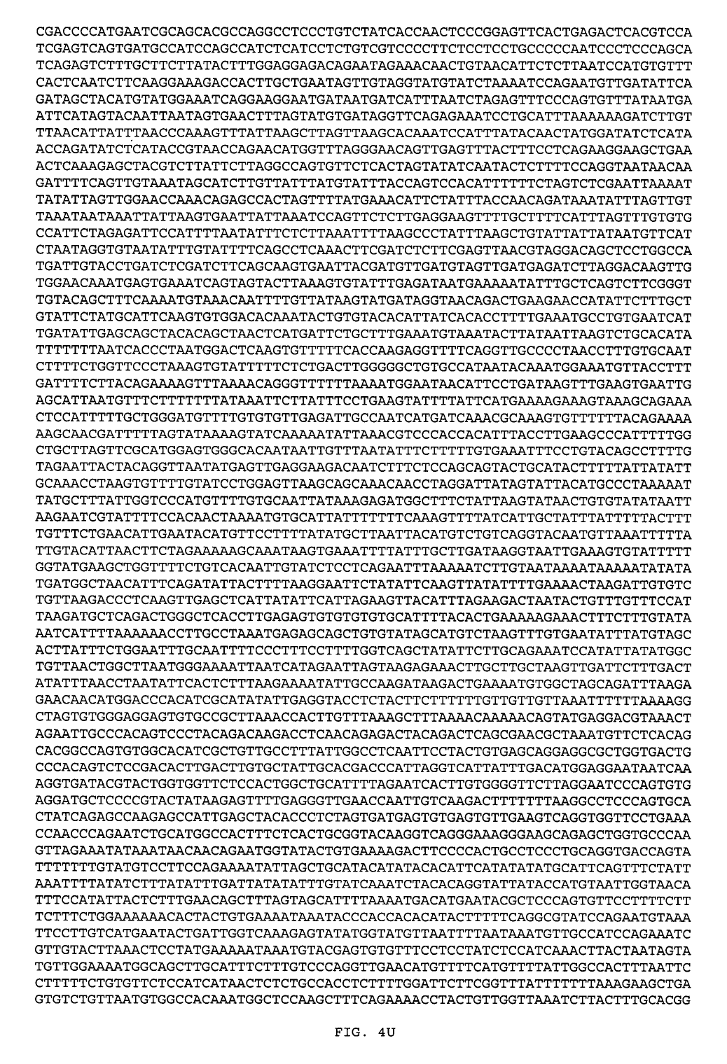

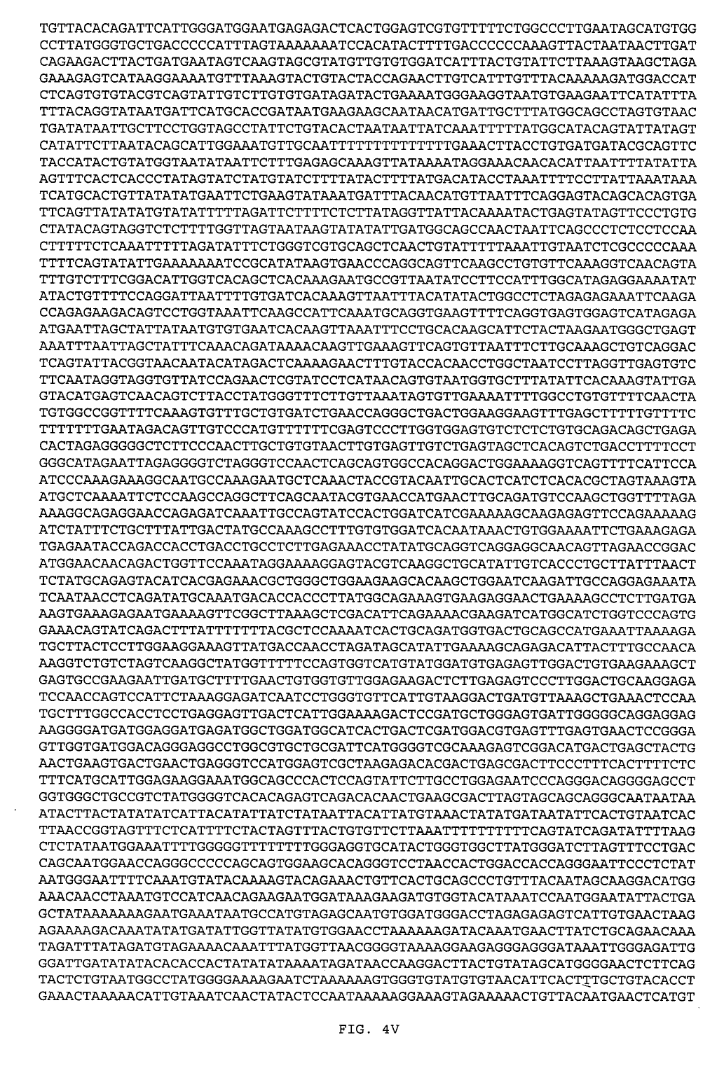

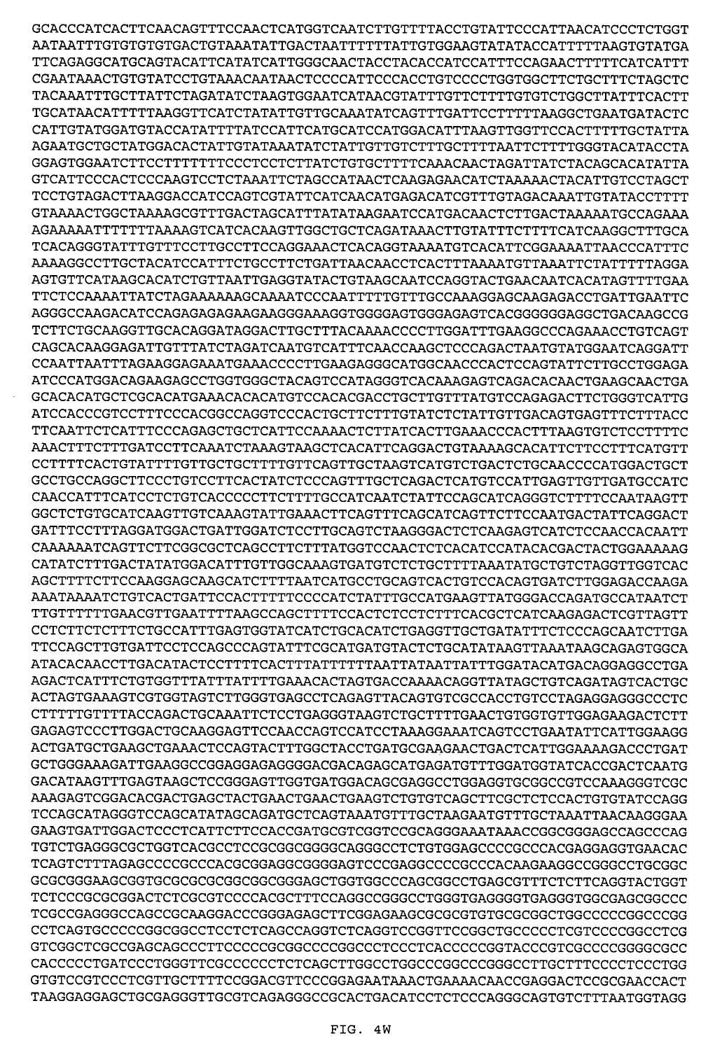

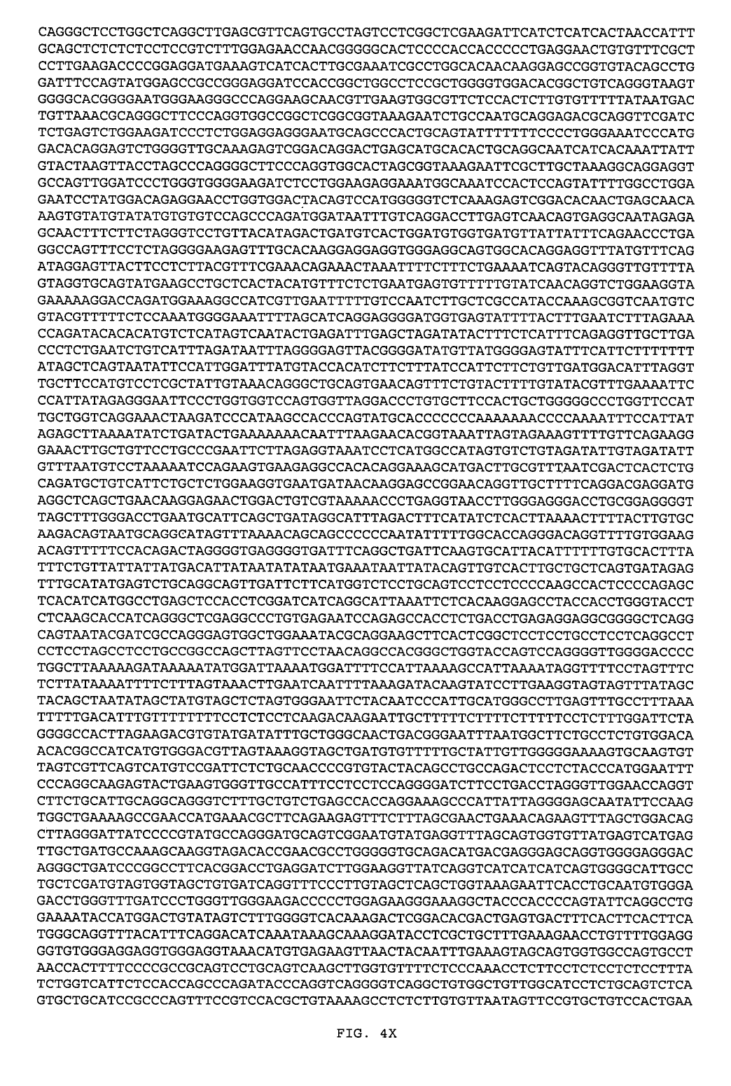

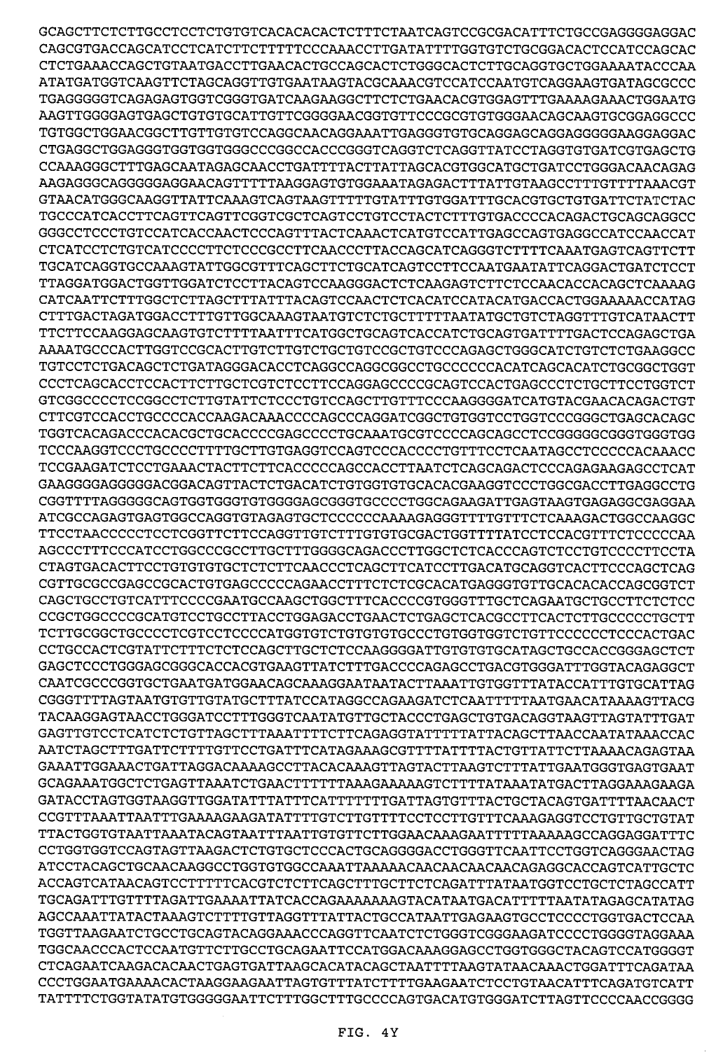

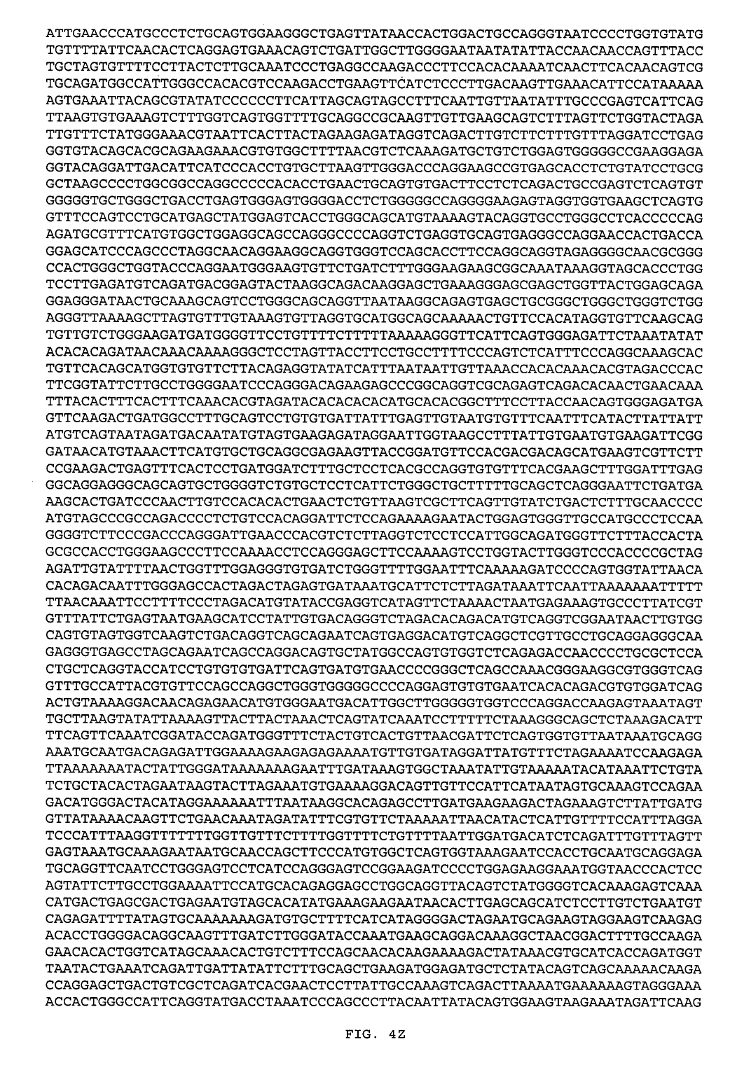

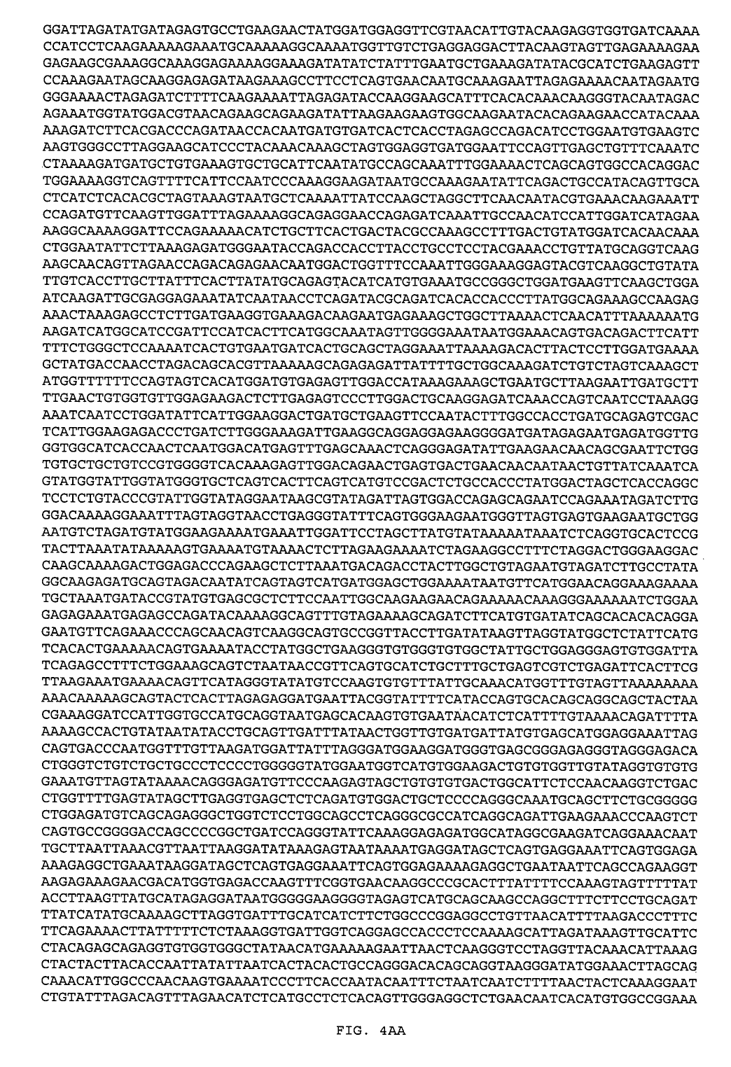

AFLP assay detects variety of genetic polymorphisms in the genome. For the AFLP derived from primer combination E+AGT/T+CAT on BTA1, a C/T substitution was detected within the extended region. Clearly, the C/T mutation does not affect enzyme digestion, but it does affect the selective primer binding, which caused the amplified fragment length polymorphisms. For the AFLP derived from primer combination E+AGT/T+ACT on BTA13, two G/A mutations were confirmed by sequencing six F1 Wagyu x Limousin bulls. One G/A occurred within the TaqI enzyme recognition site, and another G/A just five bases apart from the first mutation, affected selective primer binding. Therefore, these results provided clear evidence that the AFLP assay can detect polymorphisms within the restriction enzyme recognition sites as well as the surrounding areas where the selective primer can reach. Theoretically, any deletion/insertion or short tandem repeats with the AFLP fragment can also be detected by the technique (Savelkoul et al., 1999, J Clin Microbiol. 37, 3083-3091). Interestingly, of the three mutations identified in these two AFLP markers, none were located in the EcoRI cut site or flanking regions. Rather, they were all located in the TaqI cut site and the surrounding regions. The reason could be due to a dinucleotide CpG that presents at the center of TaqI recognition site. When it is present, the cytosine within the dinucleotide is usually methylated. The methylated cytosine has a propensity to undergo spontaneous deamination to form thymidine (Caiafa and Zampieri, 2005, J. Cell. Biochem. 94, 257-264). That is why C/T (G/A) transition is so frequent in mammalian genomes. In fact, among three mutations derived from these two AFLP markers, two happened at the CpG sites (FIGS. 2B and 3B).

AFLP driven markers for beef marbling make sense. The human orthologous regions for both AFLP markers associated with beef marbling were determined in this study: one AFLP marker derived from the primer combination E+AGT/T+CAT on BTA1, was orthologous to a novel gene DOPEY2 on HSA21q22.2, while another AFLP marker amplified with primer combination E+AGT/T+ACT on BTA13 was orthologous to a novel gene KIAA1462 on HSA10p11.23. Both regions in the human genome harbor QTLs for obesity-related phenotypes. On HSA21q21-23, quantitative trait loci were discovered to have effects on diabetes, obesity, or total cholesterol and triglycerides (Lindgren et al., 2002, Diabetes 51, 1609-1617; Li et al., 2004, Diabetes 53, 812-820; North et al., 2004, Atherosclerosis 179, 119-125). Also, in mice, a significant linkage was found to adiposity chromosome 16 (Reed et al., 2003, Mamm Genome 14, 302-313) as this mouse chromosome is orthologous to HSA21. On HSA10p 12-11, a major quantitative trait locus was revealed significant linkage to obesity and was confirmed in different populations (Hager et al., 1998,; Hinney et al., 2000, J Clin Endocrinol Metab. 85, 2962-2965; Price et al., 2001, Diabetologia 44, 363-366).

In silico mapping of AFLP markers points to candidate genes for beef marbling. Using the AFLP technique to screen QTL-linked markers for longevity in Drosophila melanogaster, Luckinbill and Golenber (2002, Genetica 114, 147-156) found that the furthest distance between an AFLP marker and a known QTL was less than 3.0 cM. Most were within 1.0 or 1.5 cM and even two were within the peak of mapped QTL limits. Evidence has shown that a reduced fatty acid oxidation by muscle mitochondria is a likely mechanism involved in intramuscular fat (marbling) accumulation (Goodpaster and Wolf, 2004, Pediatr Diabetes 5, 219-226). Therefore, two candidate genes—holocarboxylase synthetase ligase (HLCS) on HSA21q22.13 and poly(A) polymerase associated domain containing 1 (PAPD1) on HSA10p11.23 have drawn our attention because they are involved in mitochondrial biogenesis. As well, they are located in the vicinity of the AFLP marker orthologs (0.60 Mb apart between DOPEY2 and HLCS, and 0.30 Mb apart between KIAA1462 and PAPD1, respectively). HLCS catalyzes the covalent binding of biotin to apocarboxylases, while biotin is the cofactor of the human mitochondrial enzymes propionyl-CoA carboxylase, 3-methylcrotonyl-CoA carboxylase, pyruvate carboxylase, and the cytosolic enzyme acetyl-CoA carboxylase (Santer et al., 2003, Mol Genet Metab. 79, 160-166). PAPD1 has a role in mitochondrial RNA processing (Tomecki et al., 2004, Nucleic Acids Res. 32, 6001-6014). Indeed, significant associations were confirmed between PAPD1 gene and marbling (P<0.05) in this population (Xiao et al., 2006, International Journal of Biological Sciences 2, 171-178). Recently, two other nucleus encoded mitochondrial genes—mitochondrial transcription factor A (TFAM) and fatty acid binding protein 4 (FABP4) were found to be associated with marbling using the same population of cattle as described above (Jiang et al., 2005, Biochem. Biophys. Res. Commun. 334, 516-523 and Michal et al., 2006, Animal Genetics 37, 400-402.). In mice, a profound decrease of approximately 50% in the levels of transcripts for nuclear-encoded mitochondrial genes was found to accompany the onset of obesity (Wilson-Fritch et al., 2004, J Clin Invest. 114, 1281-1289). These studies demonstrated the essential role and function of some nucleus encoded mitochondrial genes in muscle lipid metabolism.

No doubt, this simplified QTL mapping approach; in particular the AFLP assay itself has several drawbacks. First, of ten potential QTL linked AFLP markers identified for beef marbling, only four were confirmed to show significant differences between high and low groups of animals by individual AFLP genotyping. This means that AFLP screening on DNA pools generated a relatively high percentage of false positive markers. Second, as over 600 genes, markers and chromosomal regions have been identified as associated or linked with human obesity phenotypes (Perusse et al., 2005, Obes Res. 13, 381-490), the two enzyme (EcoRI and TaqI) combination will not detect all markers linked to beef marbling in the population. Third, among three readable sequences of AFLP markers generated in the study, one (E+AAG/T+CAT) completely hit the SINE element and another (E+AGT/T+ACT) has almost of half of its sequence relevant to SINE element. This implies that EcoRI-TaqI combination favors amplification of repetitive regions. Therefore, the enzyme combination best suitable to mammalian genome needs to be further considered.

All together, two novel positional candidate gene regions, one on BTA1 and one on BTA13 were identified to have significant effects on beef marbling score in Wagyu x Limousin F₂ crosses through a simplified QTL mapping approach. Further studies are needed to confirm and characterize these genes on how they are functionally involved in the genetic control of beef marbling variation. In addition, this study may also provide important information to unravel genetic complexity of obesity on these two chromosomal regions in humans.

The practice of the present invention will employ, unless otherwise indicated, conventional techniques of molecular biology, microbiology, recombinant DNA technology, and immunology, which are within the skill of the art. Such techniques are explained fully in the literature. See, e.g., Sambrook et al. (2001) Molecular Cloning: A Laboratory Manual, 3rd ed., Cold Spring Harbor Press; DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

Before describing the present invention in detail, it is to be understood that this invention is not limited to particular DNA, polypeptide sequences or process parameters as such may, of course, vary. It is also to be understood that the terminology used herein is for the purpose of describing particular embodiments of the invention only, and is not intended to be limiting.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed and are intended to be defined as indicated below.

The term “cow” or “cattle” is used generally to refer to an animal of bovine origin of any age. Interchangeable terms include “bovine”, “calf”, “steer”, “bull”, “heifer” and the like. it also includes an individual animal in all stages of development, including embryonic and fetal stages. The animals as referred to herein may also include individuals or groups of individuals that are raised for other than food production such as, but not limited to, transgenic animals for the production of biopharmaceuticals including antibodies and other proteins or protein products.

By the term “complementarity” or “complementary” is meant, for the purposes of the specification or claims, a sufficient number in the oligonucleotide of complementary base pairs in its sequence to interact specifically (hybridize) with a target nucleic acid sequence of the gene polymorphism to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

By the term “detectable moiety” is meant, for the purposes of the specification or claims, a label molecule (isotopic or non-isotopic) which is incorporated indirectly or directly into an oligonucleotide, wherein the label molecule facilitates the detection of the oligonucleotide in which it is incorporated, for example when the oligonucleotide is hybridized to amplified gene polymorphic sequences. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent, fluorescent or luminescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

“DNA amplification” as used herein refers to any process that increases the number of copies of a specific DNA sequence by enzymatically amplifying the nucleic acid sequence. A variety of processes are known. One of the most commonly used is the polymerase chain reaction (PCR) process of Mullis as described in U.S. Pat. Nos. 4,683,195 and 4,683,202. Methods, devices and reagents as described in U.S. Pat. Nos. 6,951,726; 6,927,024; 6,924,127; 6,893,863; 6,887,664; 6,881,559; 6,855,522; 6,855,521; 6,849,430; 6,849,404; 6,846,631; 6,844,158; 6,844,155; 6,818,437; 6,818,402; 6,794,177; 6,794,133; 6,790,952; 6,783,940; 6,773,901; 6,770,440; 6,767,724; 6,750,022; 6,744,789; 6,733,999; 6,733,972; 6,703,236; 6,699,713; 6,696,277; 6,664,080; 6,664,064; 6,664,044; RE38,352; 6,650,719; 6,645,758; 6,645,720; 6,642,000; 6,638,716; 6,632,653; 6,617,107; 6,613,560; 6,610,487; 6,596,492; 6,586,250; 6,586,233; 6,569,678; 6,569,627; 6,566,103; 6,566,067; 6,566,052; 6,558,929; 6,558,909; 6,551,783; 6,544,782; 6,537,752; 6,524,830; 6,518,020; 6,514,750; 6,514,706; 6,503,750; 6,503,705; 6,493,640; 6,492,114; 6,485,907; 6,485,903; 6,482,588; 6,475,729; 6,468,743; 6,465,638; 6,465,637; 6,465,171; 6,448,014; 6,432,646; 6,428,987; 6,426,215; 6,423,499; 6,410,223; 6,403,341; 6,399,320; 6,395,518; 6,391,559; 6,383,755; 6,379,932; 6,372,484; 6,368,834; 6,365,375; 6,358,680; 6,355,422; 6,348,336; 6,346,384; 6,319,673; 6,316,195; 6,316,192; 6,312,930; 6,309,840; 6,309,837; 6,303,343; 6,300,073; 6,300,072; 6,287,781; 6,284,455; 6,277,605; 6,270,977; 6,270,966; 6,268,153; 6,268,143; D445,907; 6,261,431; 6,258,570; 6,258,567; 6,258,537; 6,258,529; 6,251,607; 6,248,567; 6,235,468; 6,232,079; 6,225,093; 6,221,595; D441,091; 6,218,153; 6,207,425; 6,183,999; 6,183,963; 6,180,372; 6,180,349; 6,174,670; 6,153,412; 6,146,834; 6,143,496; 6,140,613; 6,140,110; 6,103,468; 6,087,097; 6,072,369; 6,068,974; 6,063,563; 6,048,688; 6,046,039; 6,037,129; 6,033,854; 6,031,960; 6,017,699; 6,015,664; 6,015,534; 6,004,747; 6,001,612; 6,001,572; 5,985,619; 5,976,842; 5,972,602; 5,968,730; 5,958,686; 5,955,274; 5,952,200; 5,936,968; 5,909,468; 5,905,732; 5,888,740; 5,883,924; 5,876,978; 5,876,977; 5,874,221; 5,869,318; 5,863,772; 5,863,731; 5,861,251; 5,861,245; 5,858,725; 5,858,718; 5,856,086; 5,853,991; 5,849,497; 5,837,468; 5,830,663; 5,827,695; 5,827,661; 5,827,657; 5,824,516; 5,824,479; 5,817,797; 5,814,489; 5,814,453; 5,811,296; 5,804,383; 5,800,997; 5,780,271; 5,780,222; 5,776,686; 5,774,497; 5,766,889; 5,759,822; 5,750,347; 5,747,251; 5,741,656; 5,716,784; 5,712,125; 5,712,090; 5,710,381; 5,705,627; 5,702,884; 5,693,467; 5,691,146; 5,681,741; 5,674,717; 5,665,572; 5,665,539; 5,656,493; 5,656,461; 5,654,144; 5,652,102; 5,650,268; 5,643,765; 5,639,871; 5,639,611; 5,639,606; 5,631,128; 5,629,178; 5,627,054; 5,618,703; 5,618,702; 5,614,388; 5,610,017; 5,602,756; 5,599,674; 5,589,333; 5,585,238; 5,576,197; 5,565,340; 5,565,339; 5,556,774; 5,556,773; 5,538,871; 5,527,898; 5,527,510; 5,514,568; 5,512,463; 5,512,462; 5,501,947; 5,494,795; 5,491,225; 5,487,993; 5,487,985; 5,484,699; 5,476,774; 5,475,610; 5,447,839; 5,437,975; 5,436,144; 5,426,026; 5,420,009; 5,411,876; 5,393,657; 5,389,512; 5,364,790; 5,364,758; 5,340,728; 5,283,171; 5,279,952; 5,254,469; 5,241,363; 5,232,829; 5,231,015; 5,229,297; 5,224,778; 5,219,727; 5,213,961; 5,198,337; 5,187,060; 5,142,033; 5,091,310; 5,082,780; 5,066,584; 5,023,171 and 5,008,182 may also be employed in the practice of the present invention. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves a cyclic enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); Qβ replicase amplification (QβRA); self-sustained replication (3SR); and NASBA (nucleic acid sequence-based amplification), which can be performed on RNA or DNA as the nucleic acid sequence to be amplified.

A “fragment” of a molecule such as a protein or nucleic acid is meant to refer to any portion of the amino acid or nucleotide genetic sequence.

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). In general, an animal's genetic characteristics, as defined by the nucleotide sequence of its genome, are known as its “genotype,” while the animal's physical traits are described as its “phenotype.”

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” or “homozygous polymorphism” is meant that the two alleles of a diploid cell or organism at a given locus are identical, that is, that they have the same nucleotide for nucleotide exchange at the same place in their sequences.

By “hybridization” or “hybridizing,” as used herein, is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

A “hybridization complex”, such as in a sandwich assay, means a complex of nucleic acid molecules including at least the target nucleic acid and a sensor probe. It may also include an anchor probe.

As used herein, the term “locus” or “loci” refers to the site of a gene on a chromosome. Pairs of genes, known as “alleles” control the hereditary trait produced by a gene locus. Each animal's particular combination of alleles is referred to as its “genotype”.

Where both alleles are identical the individual is said to be homozygous for the trait controlled by that gene pair; where the alleles are different, the individual is said to be heterozygous for the trait.

A “melting temperature” is meant the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this invention be from about 1° C. to about 10° C. so as to be readily detectable.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. “DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). An “isolated” nucleic acid molecule is one that is separated from other nucleic acid molecules that are present in the natural source of the nucleic acid.

A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

A “polymerase” is an enzyme that catalyzes the sequential addition of monomeric units to a polymeric chain, or links two or more monomeric units to initiate a polymeric chain. The “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers. A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides linked by a phosphodiester backbone. A “modified polynucleotide” refers to a polynucleotide in which one or more natural nucleotides have been partially, substantially, or completely replaced with modified nucleotides.

A “primer” is an oligonucleotide, the sequence of at least of portion of which is complementary to a segment of a template DNA which is to be amplified or replicated. Typically primers are used in performing the polymerase chain reaction (PCR). A primer hybridizes with (or “anneals” to) the template DNA and is used by the polymerase enzyme uses as the starting point for the replication/amplification process. The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotides nucleic acid sequences of variable length, used in the detection of identical, similar, or complementary nucleic acid sequences by hybridization. An oligonucleotide sequence used as a detection probe may be labeled with a detectable moiety.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support.

An “isolated” polynucleotide or polypeptide is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

An “isolated” nucleic acid molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

The term “polynucleotide encoding a protein” as used herein refers to a DNA fragment or isolated DNA molecule encoding a protein, or the complementary strand thereto; but, RNA is not excluded, as it is understood in the art that thymidine (T) in a DNA sequence is considered equal to uracil (U) in an RNA sequence. Thus, RNA sequences for use in the invention, e.g., for use in RNA vectors, can be derived from DNA sequences, by thymidine (T) in the DNA sequence being considered equal to uracil (U) in RNA sequences.

A DNA “coding sequence” or a “nucleotide sequence encoding” a particular protein, is a DNA sequence which is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

“Homology” refers to the percent identity between two polynucleotide or two polypeptide moieties. Two DNA, or two polypeptide sequences are “substantially homologous” to each other when the sequences exhibit at least about 80%, 81%, 82%, 83%, 84%, 85%, 86%, 87%, 88%, 89%, preferably at least about 90%, 91%, 92%, 93%, 94% and most preferably at least about 95%, 96%, 97%, 98%, 99%, 99.5%, 99.9% sequence identity over a defined length of the molecules. As used herein, substantially homologous also refers to sequences showing complete identity (100% sequence identity) to the specified DNA or polypeptide sequence.

Homology can be determined by hybridization of polynucleotides under conditions that form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al. supra; DNA Cloning, supra; Nucleic Acid Hybridization, supra.

Two nucleic acid fragments are considered to be “selectively hybridizable” to a polynucleotide if they are capable of specifically hybridizing to a nucleic acid or a variant thereof or specifically priming a polymerase chain reaction: (i) under typical hybridization and wash conditions, as described, for example, in Sambrook et al. supra and Nucleic Acid Hybridization, supra, (ii) using reduced stringency wash conditions that allow at most about 25-30% basepair mismatches, for example: 2×SSC, 0.1% SDS, room temperature twice, 30 minutes each; then 2×SSC, 0.1% SDS, 37° C. once, 30 minutes; then 2×SSC room temperature twice, 10 minutes each, or (iii) selecting primers for use in typical polymerase chain reactions (PCR) under standard conditions (described for example, in Saiki, et al. (1988) Science 239:487-491).

The term “capable of hybridizing under stringent conditions” as used herein refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of a nucleic acid or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g. low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° Celsius in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al. (1984) Anal. Biochem. 138: 267-284; the content of which is herein incorporated by reference in its entirety. Protocols for hybridization techniques are well known to those of skill in the art and standard molecular biology manuals may be consulted to select a suitable hybridization protocol without undue experimentation. See, for example, Sambrook et al. (2001) Molecular Cloning: A Laboratory Manual, 3rd ed., Cold Spring Harbor Press, the contents of which are herein incorporated by reference in their entirety.

Typically, stringent conditions will be those in which the salt concentration is less than about 1.5 M sodium ion, typically about 0.01 to 1.0 M Na ion concentration (or other salts) from about pH 7.0 to about pH 8.3 and the temperature is at least about 30° Celsius for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° Celsius, and a wash in 1-2×SSC at 50 to 55° Celsius. Exemplary moderate stringency conditions include hybridization in 40 to 45% formamide, 1 M NaCl, 1% SDS at 37° Celsius, and a wash in 0.5-1×SSC at 55 to 60° Celsius. Exemplary high stringency conditions include hybridization in 50% formamide, 1 M NaCl, 1% SDS at 370 Celsius, and a wash in 0.1×SSC at 60 to 65° Celsius.

Methods and materials of the invention may be used more generally to evaluate a DNA sample from an animal, genetically type an individual animal, and detect genetic differences in animals. In particular, a sample of genomic DNA from an animal may be evaluated by reference to one or more controls to determine if a SNP, or group of SNPs, in a gene is present. Any method for determining genotype can be used for determining the genotype in the present invention. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, microsatellite analysis, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties. Genotypic data useful in the methods of the invention and methods for the identification and selection of animal traits are based on the presence of SNPs.

A “restriction fragment” refers to a fragment of a polynucleotide generated by a restriction endonuclease (an enzyme that cleaves phosphodiester bonds within a polynucleotide chain) that cleaves DNA in response to a recognition site on the DNA. The recognition site (restriction site) consists of a specific sequence of nucleotides typically about 4-8 nucleotides long.

A “single nucleotide polymorphism” or “SNP” refers to a variation in the nucleotide sequence of a polynucleotide that differs from another polynucleotide by a single nucleotide difference. For example, without limitation, exchanging one A for one C, G or T in the entire sequence of polynucleotide constitutes a SNP. It is possible to have more than one SNP in a particular polynucleotide. For example, at one position in a polynucleotide, a C may be exchanged for a T, at another position a G may be exchanged for an A and so on. When referring to SNPs, the polynucleotide is most often DNA.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such a DNA strand may be single-stranded or it may be part of a double-stranded DNA template. In applications of the present invention requiring repeated cycles of polymerization, e.g., the polymerase chain reaction (PCR), the template strand itself may become modified by incorporation of modified nucleotides, yet still serve as a template for a polymerase to synthesize additional polynucleotides.

A “thermocyclic reaction” is a multi-step reaction wherein at least two steps are accomplished by changing the temperature of the reaction.

A “variance” is a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same polynucleotide. A “point mutation” refers to a single substitution of one nucleotide for another.

As used herein, the terms “traits”, “quality traits” or “physical characteristics” or “phenotypes” refer to advantageous properties of the animal resulting from genetics. Quality traits include, but are not limited to, the animal's genetic ability to efficiently metabolize energy, produce meat or milk, put on intramuscular fat. Physical characteristics include, but are not limited to, marbled, tender or lean meats. The terms may be used interchangeably.

A “computer system” refers to the hardware means, software means and data storage means used to compile the data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT, XP or IBM OS/2 operating systems.

“Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, the data compiled on a particular animal can be routinely accessed by a user, e.g., a feedlot operator.

The term “data analysis module” is defined herein to include any person or machine, individually or working together, which analyzes the sample and determines the genetic information contained therein. The term may include a person or machine within a laboratory setting.

As used herein, the term “data collection module” refers to any person, object or system obtaining a tissue sample from an animal or embryo. By example and without limitation, the term may define, individually or collectively, the person or machine in physical contact with the animal as the sample is taken, the containers holding the tissue samples, the packaging used for transporting the samples, and the like. Advantageously, the data collector is a person. More advantageously, the data collector is a livestock farmer, a breeder or a veterinarian

The term “network interface” is defined herein to include any person or computer system capable of accessing data, depositing data, combining data, analyzing data, searching data, transmitting data or storing data. The term is broadly defined to be a person analyzing the data, the electronic hardware and software systems used in the analysis, the databases storing the data analysis, and any storage media capable of storing the data. Non-limiting examples of network interfaces include people, automated laboratory equipment, computers and computer networks, data storage devices such as, but not limited to, disks, hard drives or memory chips.

The term “breeding history” as used herein refers to a record of the life of an animal or group of animals including, but not limited to, the location, breed, period of housing, as well as a genetic history of the animals, including parentage and descent therefrom, genotype, phenotype, transgenic history if relevant and the like.

The term “husbandry conditions” as used herein refers to parameters relating to the maintenance of animals including, but not limited to, shed or housing temperature, weekly mortality of a herd, water consumption, feed consumption, ventilation rate and quality, litter condition and the like.

The term “veterinary history” as used herein refers to vaccination data of an animal or group of animals, including, but not limited to, vaccine type(s), vaccine batch serial number(s), administered dose, target antigen, method of administering of the vaccine to the recipient animal(s), number of vaccinated animals, age of the animals and the vaccinator. Data relating to a serological or immunological response induced by the vaccine may also be included. “Veterinary history” as used herein is also intended to include the medication histories of the target animal(s) including, but not limited to drug and/or antibiotics administered to the animals including type of administered medication, quantity and dose rates, by whom and when administered, by what route, e.g., oral, subcutaneously and the like, and the response to the medication including desired and undesirable effects thereof.

The term “diagnostic data” as used herein refers to data relating to the health of the animal(s) other than data detailing the vaccination or medication history of the animal(s). For example, the diagnostic data may be a record of the infections experienced by the animal(s) and the response thereof to medications provided to treat such medications. Serological data including antibody or protein composition of the serum or other biofluids may also be diagnostic data useful to input in the methods of the invention. Surgical data pertaining to the animal(s) may be included, such as the type of surgical manipulation, outcome of the surgery and complications arising from the surgical procedure. “Diagnostic data” may also include measurements of such parameters as weight, morbidity, and other characteristics noted by a veterinary service such as the condition of the skin, feet, etc.

The term “welfare data” as used herein refers to the collective accumulation of data pertaining to an animal or group of animals including, but not limited to, a breeding history, a veterinary history, a welfare profile, diagnostic data, quality control data, or any combination thereof.

The term “welfare profile” as used herein refers to parameters such as weight, meat density, crowding levels in breeding or rearing enclosures, psychological behavior of the animal, growth rate and quality and the like.

The term “quality control” as used herein refers to the desired characteristics of the animal(s). For non-poultry animals such as cattle and sheep for example, such parameters include muscle quantity and density, fat content, meat tenderness, milk yield and quality, breeding ability, and the like.

The term “performance parameters” as used herein refers to such factors as beef marbling, subcutaneous fat, meat yield, breeding yield, dairy form, meat quality and yield, daughter pregnancy rate (i.e., fertility), productive life (i.e., longevity) and the like that may be the desired goals from the breeding and rearing of the animal(s). Performance parameters may be either generated from the animals themselves, or those parameters desired by a customer or the market.

The term “nutritional data” as used herein refers to the composition, quantity and frequency of delivery of feed, including water, provided to the animal(s).

The term “food safety” as used herein refers to the quality of the meat from a livestock animal, including, but not limited to, preparation time, place and manner, storage of the food product, transportation route, inspection records, texture, color, taste, odor, bacterial content, parasitic content and the like.

It will be apparent to those of skill in the art that the data relating to the health and maintenance of the animals may be variously grouped depending upon the source or intention of the data collector and any one grouping herein is not therefore intended to be limiting.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

In an embodiment wherein the gene of interest is bovine DOPEY2, the bovine DOPEY2 nucleotide sequence can be selected from, but is not limited to, the sequence corresponding to GenBank Accession No. AAFC03071397.1, bovine chromosome 1 (BTA1) or a fragment thereof or a region of the bovine genome that comprises this sequence.

In an embodiment wherein the gene of interest is bovine KIAA1462, the bovine KIAA1462 nucleotide sequence can be selected from, but is not limited to, the sequence corresponding to GenBank Accession No. AAFC02113318.1, bovine chromosome 13 (BTA13) or a fragment thereof or a region of the bovine genome that comprises this sequence.

The present invention, therefore, provides isolated nucleic acids that may specifically hybridize to the nucleotide sequence can be selected from, but is not limited to, the sequence corresponding to GenBank Accession Nos AAFC03071397.1, AAFC02113318.1, BTA1, BTA13 or the complement thereof, and which comprises the polymorphic site corresponding to the DOPEY2 and/or KIAA1462 SNPs.

The single nucleotide polymorphism(s) of interest may be selected from the group consisting of AAFC03071397.1:g.12881G>C, g.12925G>A, g.12951T>C, g.13013A>G, g.13125G>A and g.13173C>T for the DOPEY2 gene and AAFC02113318.1:g.1367G>A and g.1372G>A for the KIAA1462 gene.

The SNP advantageous in the present invention is associated with certain economically valuable and heritable traits relating to beef marbling and/or subcutaneous fat in bovines. Therefore, it is an object of the present invention to determine the genotype of a given animal of interest as defined by the DOPEY2 and/or KIAA1462 locus SNP according to the present invention. It is also contemplated that the genotype of the animal(s) may be defined by additional SNPs within the DOPEY2 and/or KIAA1462 gene or within other genes identified with desirable traits or other characteristics, and in particular by a panel or panels of SNPs.

There are many methods known in the art for determining the sequence of DNA in a sample, and for identifying whether a given DNA sample contains a particular SNP. Any such technique known in the art may be used in performance of the methods of the present invention.

The methods of the present invention allow animals with certain economically valuable heritable traits to be identified based on the presence of SNPs in their genomes and particularly SNPs of the DOPEY2 and/or KIAA1462 genes. The methods further allow, by computer-assisted methods of the invention, to correlate the SNP-associated traits with other data pertinent to the well-being and productive capacity of the animals, or group of animals.

To determine the genotype of a given animal according to the methods of the present invention, it is necessary to obtain a sample of genomic DNA from that animal. Typically, that sample of genomic DNA will be obtained from a sample of tissue or cells taken from that animal. A tissue or cell sample may be taken from an animal at any time in the lifetime of an animal but before the carcass identity is lost. The tissue sample can comprise hair, including roots, hide, bone, buccal swabs, blood, saliva, milk, semen, embryos, muscle or any internal organs. In the methods of the present invention, the source of the tissue sample, and thus also the source of the test nucleic acid sample, is not critical. For example, the test nucleic acid can be obtained from cells within a body fluid of the animal, or from cells constituting a body tissue of the animal. The particular body fluid from which cells are obtained is also not critical to the present invention. For example, the body fluid may be selected from the group consisting of blood, ascites, pleural fluid and spinal fluid. Furthermore, the particular body tissue from which cells are obtained is also not critical to the present invention. For example, the body tissue may be selected from the group consisting of skin, endometrial, uterine and cervical tissue. Both normal and tumor tissues can be used.

Typically, the tissue sample is marked with an identifying number or other indicia that relates the sample to the individual animal from which the sample was taken. The identity of the sample advantageously remains constant throughout the methods and systems of the invention thereby guaranteeing the integrity and continuity of the sample during extraction and analysis. Alternatively, the indicia may be changed in a regular fashion that ensures that the data, and any other associated data, can be related back to the animal from which the data was obtained.

The amount/size of sample required is known to those skilled in the art and for example, can be determined by the subsequent steps used in the method and system of the invention and the specific methods of analysis used. Ideally, the size/volume of the tissue sample retrieved should be as consistent as possible within the type of sample and the species of animal. For example, for cattle, non-limiting examples of sample sizes/methods include non-fatty meat: 0.0002 gm-10.0 gm; hide: 0.0004 gm-10.0 gm; hair roots: at least one and advantageously greater than five; buccal swabs: 15 to 20 seconds of rubbing with modest pressure in the area between outer lip and gum using, for example, a cytology brush; bone: 0.0002 gm-10.0 gm; blood: 30 μl to 50 ml.

Generally, the tissue sample is placed in a container that is labeled using a numbering system bearing a code corresponding to the animal, for example, to the animal's ear tag. Accordingly, the genotype of a particular animal is easily traceable at all times. The sampling device and/or container may be supplied to the farmer, a slaughterhouse or retailer. The sampling device advantageously takes a consistent and reproducible sample from individual animals while simultaneously avoiding any cross-contamination of tissue. Accordingly, the size and volume of sample tissues derived from individual animals would be consistent.

DNA can be isolated from the tissue/cells by techniques known to those skilled in the art (see, e.g., U.S. Pat. Nos. 6,548,256 and 5,989,431; Hirota et al. (1989) Jinrui Idengaku Zasshi. 34: 217-23 and John et al. (1991) Nucleic Acids Res. 19:408, the disclosures of which are incorporated by reference in their entireties). For example, high molecular weight DNA may be purified from cells or tissue using proteinase K extraction and ethanol precipitation. DNA, however, may be extracted from an animal specimen using any other suitable methods known in the art.

In one embodiment, the presence or absence of the SNP of any of the genes of the present invention may be determined by sequencing the region of the genomic DNA sample that spans the polymorphic locus. Many methods of sequencing genomic DNA are known in the art, and any such method can be used, see for example Sambrook et al. (2001) Molecular Cloning: A Laboratory Manual, 3rd ed., Cold Spring Harbor Press. For example, as described below, a DNA fragment spanning the location of the SNP of interest can be amplified using the polymerase chain reaction. The amplified region of DNA form can then be sequenced using any method known in the art, for example using an automatic nucleic acid sequencer. The detection of a given SNP can then be performed using hybridization of probes and or using PCR-based amplification methods. Such methods are described in more detail below.

The methods of the present invention may use oligonucleotides useful as primers to amplify specific nucleic acid sequences of the DOPEY2 and/or KIAA1462 gene, advantageously of the region encompassing a DOPEY2 and/or KIAA1462 SNP. Such fragments should be of sufficient length to enable specific annealing or hybridization to the nucleic acid sample. The sequences typically will be about 8 to about 44 nucleotides in length. Longer sequences, e.g., from about 14 to about 50, may be advantageous for certain embodiments. The design of primers is well known to one of ordinary skill in the art.

Inventive nucleic acid molecules include nucleic acid molecules having at least 70% identity or homology or similarity with a DOPEY2 and/or KIAA1462 gene or probes or primers derived therefrom such as at least 75% identity or homology or similarity, preferably at least 80% identity or homology or similarity, more preferably at least 85% identity or homology or similarity such as at least 90% identity or homology or similarity, more preferably at least 95% identity or homology or similarity such as at least 97% identity or homology or similarity. The nucleotide sequence similarity or homology or identity can be determined using the “Align” program of Myers and Miller, (“Optimal Alignments in Linear Space”, CABIOS 4, 11-17, 1988) and available at NCBI. Alternatively or additionally, the terms “similarity” or “identity” or “homology”, for instance, with respect to a nucleotide sequence, is intended to indicate a quantitative measure of homology between two sequences. The percent sequence similarity can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2). Alternatively or additionally, “similarity” with respect to sequences refers to the number of positions with identical nucleotides divided by the number of nucleotides in the shorter of the two sequences wherein alignment of the two sequences can be determined in accordance with the Wilbur and Lipman algorithm (Wilbur and Lipman, 1983 PNAS USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity with DNA sequences, thymidine (T) in the DNA sequence is considered equal to uracil (U) in the RNA sequence.

A probe or primer can be any stretch of at least 8, preferably at least 10, more preferably at least 12, 13, 14, or 15, such as at least 20, e.g., at least 23 or 25, for instance at least 27 or 30 nucleotides in a DOPEY2 and/or KIAA1462 gene which are unique to a DOPEY2 and/or KIAA1462 gene. As to PCR or hybridization primers or probes and optimal lengths therefor, reference is also made to Kajimura et al., GATA 7(4):71-79 (1990).

RNA sequences within the scope of the invention are derived from the DNA sequences, by thymidine (T) in the DNA sequence being considered equal to uracil (U) in RNA sequences.

The oligonucleotides can be produced by a conventional production process for general oligonucleotides. They can be produced, for example, by a chemical synthesis process or by a microbial process that makes use of a plasmid vector, a phage vector or the like. Further, it is suitable to use a nucleic acid synthesizer.

To label an oligonucleotide with the fluorescent dye, one of conventionally known labeling methods can be used (Tyagi & Kramer (1996) Nature Biotechnology 14: 303-308; Schofield et al. (1997) Appl. and Environ. Microbiol. 63: 1143-1147; Proudnikov & Mirzabekov (1996) Nucl. Acids Res. 24: 4532-4535). Alternatively, the oligonucleotide may be labeled with a radiolabel e.g., ³H, ¹²⁵I, ³⁵S, ¹⁴C, ³²P, etc. Well-known labeling methods are described, for example, in Sambrook et al. (2001) Molecular Cloning: A Laboratory Manual, 3rd ed., Cold Spring Harbor Press. The label is coupled directly or indirectly to a component of the oligonucleotide according to methods well known in the art. Reversed phase chromatography or the like used to provide a nucleic acid probe for use in the present invention can purify the synthesized oligonucleotide labeled with a marker. An advantageous probe form is one labeled with a fluorescent dye at the 3′- or 5′-end and containing G or C as the base at the labeled end. If the 5′-end is labeled and the 3′-end is not labeled, the OH group on the C atom at the 3′-position of the 3′-end ribose or deoxyribose may be modified with a phosphate group or the like although no limitation is imposed in this respect.

During the hybridization of the nucleic acid target with the probes, stringent conditions may be utilized, advantageously along with other stringency affecting conditions, to aid in the hybridization. Detection by differential disruption is particularly advantageous to reduce or eliminate slippage hybridization among probes and target, and to promote more effective hybridization. In yet another aspect, stringency conditions may be varied during the hybridization complex stability determination so as to more accurately or quickly determine whether a SNP is present in the target sequence.

One method for determining the genotype at the polymorphic gene locus encompasses obtaining a nucleic acid sample, hybridizing the nucleic acid sample with a probe, and disrupting the hybridization to determine the level of disruption energy required wherein the probe has a different disruption energy for one allele as compared to another allele. In one example, there can be a lower disruption energy, e.g., melting temperature, for an allele that harbors a cytosine residue at a polymorphic locus, and a higher required energy for an allele with a different residue at that polymorphic locus. This can be achieved where the probe has 100% homology with one allele (a perfectly matched probe), but has a single mismatch with the alternative allele. Since the perfectly matched probe is bound more tightly to the target DNA than the mis-matched probe, it requires more energy to cause the hybridized probe to dissociate.

In a further step of the above method, a second (“anchor”) probe may be used. Generally, the anchor probe is not specific to either allele, but hybridizes regardless of what nucleotide is present at the polymorphic locus. The anchor probe does not affect the disruption energy required to disassociate the hybridization complex but, instead, contains a complementary label for using with the first (“sensor”) probe.

Hybridization stability may be influenced by numerous factors, including thermoregulation, chemical regulation, as well as electronic stringency control, either alone or in combination with the other listed factors. Through the use of stringency conditions, in either or both of the target hybridization step or the sensor oligonucleotide stringency step, rapid completion of the process may be achieved. This is desirable to achieve properly indexed hybridization of the target DNA to attain the maximum number of molecules at a test site with an accurate hybridization complex. By way of example, with the use of stringency, the initial hybridization step may be completed in ten minutes or less, more advantageously five minutes or less, and most advantageously two minutes or less. Overall, the analytical process may be completed in less than half an hour.

In one mode, the hybridization complex is labeled and the step of determining the amount of hybridization includes detecting the amounts of labeled hybridization complex at the test sites. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. The labeled portion of the complex may be the target, the stabilizer, the probe or the hybridization complex in toto. Labeling may be by fluorescent labeling selected from the group of, but not limited to, Cy3, Cy5, Bodipy Texas Red, Bodipy Far Red, Lucifer Yellow, Bodipy 630/650-X, Bodipy R6G-X and 5-CR 6G. Colormetric labeling, bioluminescent labeling and/or chemiluminescent labeling may further accomplish labeling. Labeling further may include energy transfer between molecules in the hybridization complex by perturbation analysis, quenching, electron transport between donor and acceptor molecules, the latter of which may be facilitated by double stranded match hybridization complexes. Optionally, if the hybridization complex is unlabeled, detection may be accomplished by measurement of conductance differential between double stranded and non-double stranded DNA. Further, direct detection may be achieved by porous silicon-based optical interferometry or by mass spectrometry. In using mass spectrometry no fluorescent or other label is necessary. Rather detection is obtained by extremely high levels of mass resolution achieved by direct measurement, for example, by time of flight (TOF) or by electron spray ionization (ESI). Where mass spectrometry is contemplated, probes having a nucleic acid sequence of 50 bases or less are advantageous.

The label may be amplified, and may include, for example, branched or dendritic DNA. If the target DNA is purified, it may be un-amplified or amplified. Further, if the purified target is amplified and the amplification is an exponential method, it may be, for example, PCR amplified DNA or strand displacement amplification (SDA) amplified DNA. Linear methods of DNA amplification such as rolling circle or transcriptional runoff may also be used.

Where it is desired to amplify a fragment of DNA that comprises a SNP according to the present invention, the forward and reverse primers may have contiguous stretches of about 8, 9, 10, 11, 12, 13, 14, 15, 16, 17, 18, 19, 20, 21, 22, 23, 24, 25, 26, 27, 28, 29, 30 or any other length up to and including about 50 nucleotides in length. The sequences to which the forward and reverse primers anneal are advantageously located on either side of the particular nucleotide position that is substituted in the SNP to be amplified.

A detectable label can be incorporated into a nucleic acid during at least one cycle of an amplification reaction. Spectroscopic, photochemical, biochemical, immunochemical, electrical, optical or chemical means can detect such labels. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C, ³²P, etc.), enzymes (e.g. horseradish peroxidase, alkaline phosphatase etc.) calorimetric labels such as colloidal gold or colored glass or plastic (e.g. polystyrene, polypropylene, latex, etc.) beads. The label is coupled directly or indirectly to a component of the assay according to methods well known in the art. As indicated above, a wide variety of labels are used, with the choice of label depending on sensitivity required, ease of conjugation with the compound, stability requirements, available instrumentation, and disposal provisions. Non-radioactive labels are often attached by indirect means. Polymerases can also incorporate fluorescent nucleotides during synthesis of nucleic acids.

Reagents allowing the sequencing of reaction products can be utilized herein. For example, chain-terminating nucleotides will often be incorporated into a reaction product during one or more cycles of a reaction. Commercial kits containing the reagents most typically used for these methods of DNA sequencing are available and widely used. PCR exonuclease digestion methods for DNA sequencing can also be used. Many methods of sequencing genomic DNA are known in the art, and any such method can be used, see for example Sambrook et al. (2001) Molecular Cloning: A Laboratory Manual, 3rd ed., Cold Spring Harbor Press. For example, as described below, a DNA fragment spanning the location of the SNP of interest can amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, (2000) Genome Res. 10: 1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, (2001) Methods Mol Biol. 167: 153-70 and MacBeath et al. (2001) Methods Mol Biol. 167:119-52), capillary electrophoresis (see, e.g., Bosserhoff et al. (2000) Comb Chem High Throughput Screen. 3: 455-66), DNA sequencing chips (see, e.g., Jain, (2000) Pharmacogenomics. 1: 289-307), mass spectrometry (see, e.g., Yates, (2000) Trends Genet. 16: 5-8), pyrosequencing (see, e.g., Ronaghi, (2001) Genome Res. 11: 3-11), and ultrathin-layer gel electrophoresis (see, e.g., Guttman & Ronai, (2000) Electrophoresis. 21: 3952-64), the disclosures of which are hereby incorporated by reference in their entireties. The sequencing can also be done by a commercial company. Examples of such companies include, but are not limited to, the University of Georgia Molecular Genetics Instrumentation Facility (Athens, Ga.) or SeqWright DNA Technologies Services (Houston, Tex.).

A SNP-specific probe can also be used in the detection of the SNP in amplified specific nucleic acid sequences of the target gene, such as the amplified PCR products generated using the primers described above. In certain embodiments, these SNP-specific probes consist of oligonucleotide fragments. Advantageously, the fragments are of sufficient length to provide specific hybridization to the nucleic acid sample. The use of a hybridization probe of between 10 and 50 nucleotides in length allows the formation of a duplex molecule that is both stable and selective. Molecules having complementary sequences over stretches greater than 12 bases in length are generally advantageous, in order to increase stability and selectivity of the hybrid, and thereby improve the quality and degree of particular hybrid molecules obtained. One will generally prefer to design nucleic acid molecules having stretches of 16 to 24 nucleotides, or even longer where desired. A tag nucleotide region may be included, as at the 5′ end of the primer that may provide a site to which an oligonucleotide sequencing primer may hybridize to facilitate the sequencing of multiple PCR samples.

The probe sequence must span the particular nucleotide position that may be substituted in the particular SNP to be detected. Advantageously, two or more different “allele-specific probes” may be used for analysis of a SNP, a first allele-specific probe for detection of one allele, and a second allele-specific probe for the detection of the alternative allele.

It will be understood that this invention is not limited to the particular primers and probes disclosed herein and is intended to encompass at least nucleic acid sequences that are hybridizable to the nucleotide sequence disclosed herein, the complement or a fragment thereof, or are functional sequence analogs of these sequences. It is also contemplated that a particular trait of an animal may be determined by using a panel of SNPs associated with that trait. Several economically relevant traits may be characterized by the presence or absence of one or more SNPs and by a plurality of SNPs in different genes. One or more panels of SNPs may be used in the methods of the invention to define the phenotypic profile of the subject animal.

Homologs (i.e., nucleic acids derived from other species) or other related sequences (e.g., paralogs) can be obtained under conditions of standard or stringent hybridization conditions with all or a portion of the particular sequence as a probe using methods well known in the art for nucleic acid hybridization and cloning.

The genetic markers, probes thereof, methods, and kits of the invention are also useful in a breeding program to select for breeding those animals having desirable phenotypes for various economically important traits, such as beef marbling and/or subcutaneous fat. Continuous selection and breeding of animals, such as livestock, that are at least heterozygous and advantageously homozygous for desirable alleles of the DOPEY2 and/or KIAA1462 gene polymorphic sites associated with economically relevant traits of growth, feed intake, efficiency and/or carcass merit, and reproduction and longevity would lead to a breed, line, or population having higher numbers of offspring with economically relevant traits of growth, feed intake, efficiency and carcass merit, and reproduction and longevity. Thus, the DOPEY2 and/or KIAA1462 SNPs of the present invention can be used as a selection tool.

Desirable phenotypes include, but are not limited to, feed intake, growth rate, body weight, carcass merit and composition, and reproduction and longevity, and milk yield.

Specific carcass traits with desirable phenotypes include, but are not limited to, additional carcass value (additional carc value, $), average daily gain (ADG, lb/d), backfat thickness (BFAT, in), calculated live weight (Calc Lv Wt, lb), calculated yield grade (cYG), days on feed (DOF, d), dressing percentage (DP, %), dry matter intake (DMI, lb), dry matter intake per day on feed (DMI per DOF, lb/d), hot carcass weight (HCW, lb), hot carcass weight value (HCW value, $), intramuscular fat content (IMF %, %), marbling score (MBS, 10 to 99), marbling score divided by days on feed (MBS/DOF), quality grade, less than or equal to select versus greater than or equal to choice (QG, <Se vs, >Ch), ribeye area (REA, in²), ribeye area per hundred weight HCW (REA/cwt HCW, in²/100 lb hot carcass weight (HCW) and subcutaneous fat depth (SFD).

One aspect of the present invention provides for grouping animals and methods for managing livestock production comprising grouping livestock animals such as cattle according the genotype as defined by panels of SNPs, each panel comprising at least one SNP, one or more of which are in the DOPEY2 and/or KIAA1462 gene of the present invention. Other SNPs that may be included in panels of SNPs include, but not limited to, SNPs found in the CAST gene, diacylglycerol O-acyltransferase (DGAT1) gene, GHR gene, FABP4 gene, ghrelin gene, leptin (LEP) gene, NPY gene, ob gene, TFAM gene, CRH gene, UASMS1 gene, UASMS2 gene, UASMS3 gene and/or the UCP3 gene. The genetic selection and grouping methods of the present invention can be used in conjunction with other conventional phenotypic grouping methods such as grouping animals by visible characteristics such as weight, frame size, breed traits, and the like. The methods of the present invention provide for producing cattle having improved heritable traits, and can be used to optimize the performance of livestock herds in areas such as beef marbling and/or subcutaneous fat. The present invention provides methods of screening livestock to determine those more likely to develop a desired body condition by identifying the presence or absence of one or more gene polymorphisms correlated with beef marbling and/or subcutaneous fat.

As described above, and in the Examples, there are various phenotypic traits with which the SNPs of the present invention may be associated. Each of the phenotypic and genetic traits can be tested using the methods described in the Examples, or using any suitable methods known in the art. Using the methods of the invention, a farmer, or feedlot operator, or the like, can group cattle according to each animal's genetic propensity for a desired trait such as growth rate, feed intake or feeding behavior, as determined by SNP genotype. The cattle are tested to determine homozygosity or heterozygosity with respect to the SNP alleles of one or more genes so that they can be grouped such that each pen contains cattle with like genotypes. Each pen of animals is then fed and otherwise maintained in a manner and for a time determined by the feedlot operator, and then slaughtered.

The individual genotypic data derived from a panel or panels of SNPs for each animal or a herd of animals can be recorded and associated with various other data of the animal, e.g. health information, parentage, husbandry conditions, vaccination history, herd records, subsequent food safety data and the like. Such information can be forwarded to a government agency to provide traceability of an animal or meat product, or it may serve as the basis for breeding, feeding and marketing information. Once the data has or has not been associated with other data, the data is stored in an accessible database, such as, but not limited to, a computer database or a microchip implanted in the animal. The methods of the invention may provide an analysis of the input data that may be compared with parameters desired by the operator. These parameters include, but are not limited to, such as breeding goals, egg laying targets, vaccination levels of a herd. If the performance or properties of the animals deviates from the desired goals, the computer-based methods may trigger an alert to allow the operator to adjust vaccination doses, medications, feed etc accordingly.

The results of the analysis provide data that are associated with the individual animal or to the herd, in whole or in part, from which the sample was taken. The data are then kept in an accessible database, and may or may not be associated with other data from that particular individual or from other animals.

Data obtained from individual animals may be stored in a database that can be integrated or associated with and/or cross-matched to other databases. The database along with the associated data allows information about the individual animal to be known through every stage of the animal's life, i.e., from conception to consumption of the animal product.

The accumulated data and the combination of the genetic data with other types of data of the animal provides access to information about parentage, identification of herd, health information including vaccinations, exposure to diseases, feedlot location, diet and ownership changes. Information such as dates and results of diagnostic or routine tests are easily stored and attainable. Such information would be especially valuable to companies, particularly those who seek superior breeding lines.

Each animal may be provided with a unique identifier. The animal can be tagged, as in traditional tracing programs or have implant computer chips providing stored and readable data or provided with any other identification method which associates the animal with its unique identifier.

The database containing the SNP-based genotype results for each animal or the data for each animal can be associated or linked to other databases containing data, for example, which may be helpful in selecting traits for grouping or sub-grouping of an animal. For example, and not for limitation, data pertaining to animals having particular vaccination or medication protocols, can optionally be further linked with data pertaining to animals having food from certain food sources. The ability to refine a group of animals is limited only by the traits sought and the databases containing information related to those traits.

Databases that can usefully be associated with the methods of the invention include, but are not limited to, specific or general scientific data. Specific data includes, but is not limited to, breeding lines, sires, dames, and the like, other animals' genotypes, including whether or not other specific animals possess specific genes, including transgenic genetic elements, location of animals which share similar or identical genetic characteristics, and the like. General data includes, but is not limited to, scientific data such as which genes encode for specific quality characteristics, breed association data, feed data, breeding trends, and the like.

One method of the present invention includes providing the animal owner or customer with sample collection equipment, such as swabs and tags useful for collecting samples from which genetic data may be obtained. Advantageously, the packaging is encoded with a bar code label. The tags are encoded with the same identifying indicia, advantageously with a matching bar code label. Optionally, the packaging contains means for sending the tags to a laboratory for analysis. The optional packaging is also encoded with identifying indicia, advantageously with a bar code label.

The method optionally includes a system wherein a database account is established upon ordering the sampling equipment. The database account identifier corresponds to the identifying indicia of the tags and the packaging. Upon shipment of the sampling equipment in fulfillment of the order, the identifying indicia are recorded in a database. Advantageously, the identifier is a bar code label which is scanned when the tags are sent. When the tags are returned to the testing facility, the identifier is again recorded and matched to the information previously recorded in the database upon shipment of the vial to the customer. Once the genotyping is completed, the information is recorded in the database and coded with the unique identifier. Test results are also provided to the customer or animal owner.

The data stored in the genotype database can be integrated with or compared to other data or databases for the purpose of identifying animals based on genetic propensities. Other data or databases include, but are not limited to, those containing information related to SNP-based DNA testing, vaccination, Sure Health pre-conditioning program, estrus and pregnancy results, hormone levels, food safety/contamination, somatic cell counts, mastitis occurrence, diagnostic test results, milk protein levels, milk fat, vaccine status, health records, mineral levels, trace mineral levels, herd performance, and the like.

The present invention, therefore, encompasses computer-assisted methods for tracking the breeding and veterinary histories of livestock animals encompassing using a computer-based system comprising a programmed computer comprising a processor, a data storage system, an input device and an output device, and comprising the steps of generating a profile of a livestock animal by inputting into the programmed computer through the input device genotype data of the animal, wherein the genotype may be defined by a panel of at least two single nucleotide polymorphisms that predict at least one physical trait of the animal, inputting into the programmed computer through the input device welfare data of the animal, correlating the inputted welfare data with the phenotypic profile of the animal using the processor and the data storage system, and outputting a profile of the animal or group of animals to the output device.

The databases and the analysis thereof will be accessible to those to whom access has been provided. Access can be provided through rights to access or by subscription to specific portions of the data. For example, the database can be accessed by owners of the animal, the test site, the entity providing the sample to the test site, feedlot personnel, and veterinarians. The data can be provided in any form such as by accessing a website, fax, email, mailed correspondence, automated telephone, or other methods for communication. These data can also be encoded on a portable storage device, such as a microchip, that can be implanted in the animal. Advantageously, information can be read and new information added without removing the microchip from the animal.

The present invention comprises systems for performing the methods disclosed herein. Such systems comprise devices, such as computers, internet connections, servers, and storage devices for data. The present invention also provides for a method of transmitting data comprising transmission of information from such methods herein discussed or steps thereof, e.g., via telecommunication, telephone, video conference, mass communication, e.g., presentation such as a computer presentation (e.g., POWERPOINT), internet, email, documentary communication such as computer programs (e.g., WORD) and the like.

Systems of the present invention may comprise a data collection module, which includes a data collector to collect data from an animal or embryo and transmit the data to a data analysis module, a network interface for receiving data from the data analysis module, and optionally further adapted to combine multiple data from one or more individual animals, and to transmit the data via a network to other sites, or to a storage device.

More particularly, systems of the present invention comprise a data collection module, a data analysis module, a network interface for receiving data from the data analysis module, and optionally further adapted to combine multiple data from one or more individual animals, and to transmit the data via a network to other sites, and/or a storage device. For example, the data collected by the data collection module leads to a determination of the absence or presence of a SNP of a gene in the animal or embryo, and for example, such data is transmitted when the feeding regimen of the animal is planned.

In one embodiment where the data is implanted on a microchip on a particular animal, the farmer can optimize the efficiency of managing the herd because the farmer is able to identify the genetic predispositions of an individual animal as well as past, present and future treatments (e.g., vaccinations and veterinarian visits). The invention, therefore also provides for accessing other databases, e.g., herd data relating to genetic tests and data performed by others, by datalinks to other sites. Therefore, data from other databases can be transmitted to the central database of the present invention via a network interface for receiving data from the data analysis module of the other databases.

The invention relates to a computer system and a computer readable media for compiling data on an animal, the system containing inputted data on that animal, such as but not limited to, vaccination and medication histories, DNA testing, thyroglobulin testing, leptin, MMI (Meta Morphix Inc.), bovine spongiform encephalopathy (BSE) diagnosis, brucellosis vaccination, FMD (foot and mouth disease) vaccination, BVD (bovine viral diarrhea) vaccination, Sure Health pre-conditioning program, estrus and pregnancy results, tuberculosis, hormone levels, food safety/contamination, somatic cell counts, mastitis occurrence, diagnostic test results, milk protein levels, milk fat, vaccine status, health records, mineral levels, trace mineral levels, herd performance, and the like. The data of the animal can also include prior treatments as well as suggested tailored treatment depending on the genetic predisposition of that animal toward a particular disease.

The invention also provides for a computer-assisted method for improving animal production comprising using a computer system, e.g., a programmed computer comprising a processor, a data storage system, an input device and an output device, the steps of inputting into the programmed computer through the input device data comprising a breeding, veterinary, medication, diagnostic data and the like of an animal, correlating a physical characteristic predicted by the genotype using the processor and the data storage system, outputting to the output device the physical characteristic correlated to the genotype, and feeding the animal a diet based upon the physical characteristic, thereby improving livestock production.

The invention further provides for a computer-assisted method for optimizing efficiency of feedlots for livestock comprising using a computer system, e.g., a programmed computer comprising a processor, a data storage system, an input device and an output device, and the steps of inputting into the programmed computer through the input device data comprising a breeding, veterinary history of an animal, correlating the breeding, veterinary histories using the processor and the data storage system, outputting to the output device the physical characteristic correlated to the genotype, and feeding the animal a diet based upon the physical characteristic, thereby optimizing efficiency of feedlots for livestock.

The invention further comprehends methods of doing business by providing access to such computer readable media and/or computer systems and/or data collected from animals to users; e.g., the media and/or sequence data can be accessible to a user, for instance on a subscription basis, via the Internet or a global communication/computer network; or, the computer system can be available to a user, on a subscription basis.

In one embodiment, the invention provides for a computer system for managing livestock comprising physical characteristics and databases corresponding to one or more animals. In another embodiment, the invention provides for computer readable media for managing livestock comprising physical characteristics and veterinary histories corresponding to one or more animals. The invention further provides methods of doing business for managing livestock comprising providing to a user the computer system and media described above or physical characteristics and veterinary histories corresponding to one or more animals. The invention further encompasses methods of transmitting information obtained in any method or step thereof described herein or any information described herein, e.g., via telecommunications, telephone, mass communications, mass media, presentations, internet, email, etc.

The invention further encompasses kits useful for screening nucleic acid isolated from one or more bovine individuals for allelic variation of any one of the mitochondrial transcription factor genes, and in particular for any of the SNPs described herein, wherein the kits may comprise at least one oligonucleotide selectively hybridizing to a nucleic acid comprising any one of the one or more of which are DOPEY2 and/or KIAA1462 sequences described herein and instructions for using the oligonucleotide to detect variation in the nucleotide corresponding to the SNP of the isolated nucleic acid.

One embodiment of this aspect of the invention provides an oligonucleotide that specifically hybridizes to the isolated nucleic acid molecule of this aspect of the invention, and wherein the oligonucleotide hybridizes to a portion of the isolated nucleic acid molecule comprising any one of the polymorphic sites in the DOPEY2 and/or KIAA1462 sequences described herein.

Another embodiment of the invention is an oligonucleotide that specifically hybridizes under high stringency conditions to any one of the polymorphic sites of the DOPEY2 and/or KIAA1462 gene, wherein the oligonucleotide is between about 18 nucleotides and about 50 nucleotides.

In another embodiment of the invention, the oligonucleotide comprises a central nucleotide specifically hybridizing with a DOPEY2 and/or KIAA1462 gene polymorphic site of the portion of the nucleic acid molecule.

Another aspect of the invention is a method of identifying a DOPEY2 and/or KIAA1462 polymorphism in a nucleic acid sample comprising isolating a nucleic acid molecule encoding DOPEY2 and/or KIAA1462 or a fragment thereof and determining the nucleotide at the polymorphic site.

Another aspect of the invention is a method of screening cattle to determine those bovines more likely to exhibit a biological difference in beef marbling and/or subcutaneous fat comprising the steps of obtaining a sample of genetic material from a bovine; and assaying for the presence of a genotype in the bovine which is associated with beef marbling and/or subcutaneous fat, the genotype characterized by a polymorphism in the bovine DOPEY2 and/or KIAA1462 gene.

In other embodiments of this aspect of the invention, the step of assaying is selected from the group consisting of: restriction fragment length polymorphism (RFLP) analysis, minisequencing, MALD-TOF, SINE, heteroduplex analysis, single strand conformational polymorphism (SSCP), denaturing gradient gel electrophoresis (DGGE) and temperature gradient gel electrophoresis (TGGE).

In various embodiments of the invention, the method may further comprise the step of amplifying a region of the DOPEY2 and/or KIAA1462 gene or a portion thereof that contains the polymorphism. In other embodiments of the invention, the amplification may include the step of selecting a forward and a reverse sequence primer capable of amplifying a region of the DOPEY2 and/or KIAA1462 gene.

Another aspect of the invention is a computer-assisted method for predicting which livestock animals possess a biological difference in beef marbling and/or subcutaneous fat comprising: using a computer system, e.g., a programmed computer comprising a processor, a data storage system, an input device and an output device, the steps of: (a) inputting into the programmed computer through the input device data comprising a DOPEY2 and/or KIAA1462 genotype of an animal, (b) correlating beef marbling and/or subcutaneous fat predicted by the DOPEY2 and/or KIAA1462 genotype using the processor and the data storage system and (c) outputting to the output device the f beef marbling and/or subcutaneous fat correlated to the DOPEY2 and/or KIAA1462 genotype, thereby predicting which livestock animals possess a particular beef marbling and/or subcutaneous fat.

Yet another aspect of the invention is a method of doing business for managing livestock comprising providing to a user computer system for managing livestock comprising physical characteristics and genotypes corresponding to one or more animals or a computer readable media for managing livestock comprising physical characteristics and genotypes corresponding to one or more animals or physical characteristics and genotypes corresponding to one or more animals.

The invention will now be further described by way of the following non-limiting examples.

### EXAMPLES

### Example 1

Animals, marbling scores and genomic DNA. The animals used in the present study were F₂ progeny derived from Wagyu x Limousin F₁ sires and F₁ dams at the Fort Keogh Livestock and Range Research Laboratory, ARS, USDA. The Wagyu breed of cattle has been selected for high marbling for a long time, whereas the Limousin breed has been selected for heavy muscle, which leads to a low marbling score. The difference in marbling scores between these two breeds makes them very unique for mapping QTL for this economically important trait in beef cattle. Development of the reference population has been described previously by Wu and colleagues (2005, Genetica 125, 103-113). Beef marbling score (BMS) was a subjective measure of the amount of intramuscular fat in the longissimus muscle based on USDA standards (http://www.ams.usda.gov/). Subcutaneous fat depth (SFD) was measured at the 12-13^{th} rib interface perpendicular to the outside surface at a point three-fourths the length of the longissimus muscle from its chine bone end. The phenotypic data has been adjusted for effects of year, gender, and age at harvest (linear) before they were used in the associate analysis. Both DNA samples and performance data on these F2 animals were kindly provided by Dr. MacNeil. Based on the availability of both data and DNA samples, 246 F₂ animals were used in the proposed project. Thirty samples with the highest BMS were used to form two high pools whereas 30 samples with the lowest BMS were used to form two low pools by adding equal amounts (20 ng) of DNA from each of 15 individuals to a pool.

AFLP analysis. The AFLP analysis on these four DNA pools was performed using a procedure including adapter and primer sequences described previously by Ajmone-Marsan and colleagues (1997) with minor modifications. In brief, 200 ng of genomic DNA was digested with two restriction enzymes: EcoRI and TaqI (New England Biolabs, Beverly, Mass., USA) based on the manufacturer's instruction. The digested products were then ligated to 5 pMol EcoRI-adapters and 50 pMol TaqI-adapters in 50 μl of solution containing 25 U T4 ligase and 1× T4 ligase buffer under incubation overnight at room temperature. The ligated DNA templates were diluted 1:10 with 10 mM Tris-HCl, 0.1 mM EDTA (pH 8.0) for pre-amplification.

The pre-amplification PCR condition was: 10 ng of DNA template, 1× Platinum Taq Buffer (20 mM Tris-HCl, PH8.4, 50 mM KCl; Invitrogen), 3.0 mM MgCl₂, 0.3 mM each of the four dNTPs, 1 U Platinum Taq polymerase (Invitrogen), 0.2 pMol of pre-amplification EcoRI primer (E01: 5′-GAC TGC GTA CCA ATT CA-3′ (SEQ ID NO: 1)) and 2 pMol of pre-amplification TaqI primers (T01: 5′-GAT GAG TCC TGA CCG AA-3′ (SEQ ID NO. 2) or T02: 5′-GAT GAG TCC TGA CCG AC-3′ (SEQ ID NO: 3)) in a total volume of 50 μl. The PCR program is as follows: 2 min at 94° C., 2 min at 72° C., 25 cycles of 10 seconds at 94° C., 30 seconds at 56° C. and 2 min at 68° C., followed by 30 min at 60° C., and ended at 4° C. PCR products were analyzed using 1.6% agarose gels, stained with ethidium bromide and photographed. The pre-amplification products were diluted 1:20 with 10 mM Tris-HCl, 0.1 mM EDTA (pH 8.0) and then used as selective amplification DNA templates.

For selective amplification, 8 EcoRI primers and 8 TaqI primers were used, resulting in 64 primer combinations. EcoRI primers were 5′ end fluorescently labeled. The following PCR reaction mix was used: 3.0 μl of diluted pre-amplification products, 1× Platinum Taq Buffer (20 mM Tris-HCl, PH8.4, 50 mM KCl; Invitrogen), 3.0 mM MgCl₂, 0.3 mM each of the four dNTPs, 1 U Platinum Taq polymerase (Invitrogen) and 5 ng of selective-amplification EcoRI primer and 25 ng selective-amplification TaqI primer in a total volume of 20 μl. A touchdown thermal protocol was used in the selective PCR amplification (Ajmone-Marsan et al., 1997). The selective-amplification products were prepared as a mix of the following: 1.0 μl of each fluorescently-labeled PCR product, 12.0 μl of formamide, and 0.5 μl of Gene Scan™ 500 LIZ™ size standard (Applied Biosystems). The mixed products were then separated on an ABI 3730 sequencer in the Laboratory for Biotechnology and Bioanalysis (Washington State University) using a standard protocol. Data were collected automatically and analyzed using software GeneMapper3.7 (Applied Biosystems).

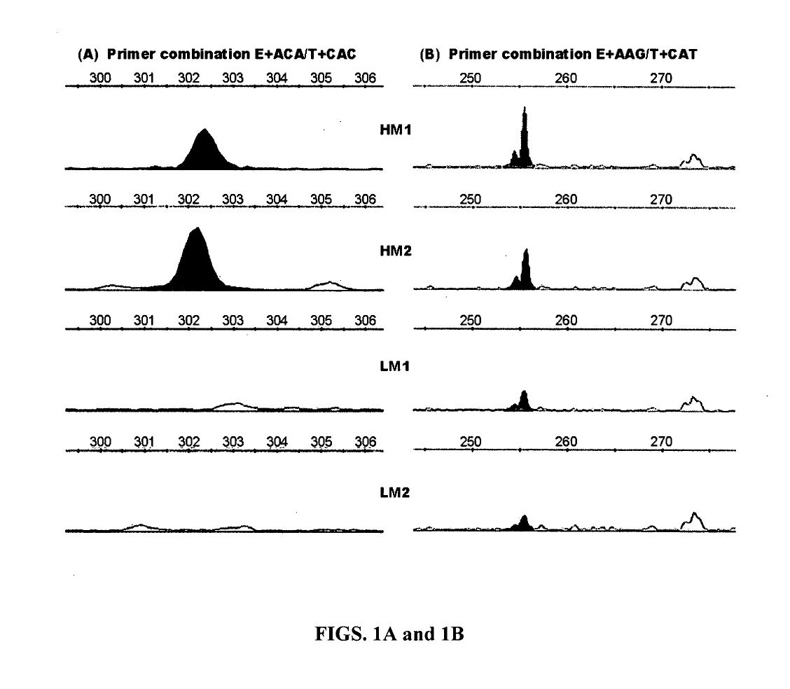

AFLP markers sequencing, in silico flanking walking and PCR-RFLP genotyping. Sixty-four primer combinations were used to generate AFLP patterns on four DNA pools, including two high-marbling pools and two low-marbling pools. Comparison of peak heights yielded ten potential AFLP markers that had the most visual-striking differences between high pools and low pools (see examples, FIG. 1), which were then selected for individual AFLP validation using a same protocol as described above. The validation was performed on 24 high and 24 low BMS samples and the presence or absence of AFLP bands of interest was scored individually. The Fisher's exact test was used to test the difference in fragment frequencies between the extreme individuals. The significant primer combinations, fragment frequencies and significance levels are listed in Table 1.

To isolate the AFLP fragments, selective amplification products that contained the marker fragments of interest were separated on a 5% polyacrylamide gel. The bands representing AFLP fragments of interest were excised using a scalpel. After excision, gel fragments were placed in 15 μl of 1× TE and frozen at −80° C. for ˜30 min, followed by one thawing-refreezing step at −20° C. After thawing, samples were centrifuged for 15 min at 15 000 g and 4.0 μl was taken for PCR re-amplification using pre-amplification AFLP primers. Fragments were sequenced directly using the same pre-amplification primers on ABI 3730 automatic capillary sequencing system following standard Big Dye protocols.

Among these four fragment isolates, only a fragment obtained from primer combination E+AAC/T+ACA did not yield a readable sequence. Three readable sequences of AFLP markers were used as queries to perform BLAST searches against the 6× bovine genome sequence database (http://www.hgsc.bcm.tmc.edu/projects/bovine/) in order to obtain the flanking sequence of the same locus in cattle. Unfortunately, the sequence derived from the primer combination E+AAG/T+CAT hit a SINE (short interspersed nuclear elements) and hence it was discarded for further use. Sequences obtained from primer combinations E+AGT/T+CAT and E+AGT/T+ACT each hit a bovine genomic contig with highly matched sequences. Primers were designed to further characterize these two AFLP fragments. The primer sequences designed for the primer combination E+AGT/T+CAT were: forward, 5′-TTT GGA GCA GTG ACA GGA TCA GAC-3′ (SEQ ID NO: 4); and reverse: 5′-AGA GAG CCT GCG TCC TTA TCT CAC-3′ (SEQ ID NO: 5) (GenBank accession no. AAFC03071397) (FIG. 2A). The primers designed for the primer combination E+AGT/T+ACT were: forward, 5′-AAA CTG TCC TTC AAG GTA GTC AAC A-3′ (SEQ ID NO: 6) and reverse, 5′-GGG GCA CTA GAG TGG GTT GCC ATT T-3′ (SEQ ID NO: 7) (GenBank accession no. AAFC02113318) (FIG. 3A). PCR amplification was performed and sequenced on six F₁ Wagyu x Limousin bulls in order to reveal molecular causes for the AFLP polymorphisms and determine the strategies for genotyping the markers on all F₂ progeny.

Statistical analysis. The marker-trait association analysis was based on the following mixed model as
y=Xβ+Zu+e
where y is a vector of observations, β is a vector for all fixed effects including the overall mean and the candidate gene effect (exactly, marker associated effects), u˜N(0, Aσᵤ²) is a vector containing residual genetic effects, other than the current gene (marker) effect, with A being the additive genetic relationship matrix of all individuals, X and Z are incidence matrices relating the effects in β and u, respectively, to observations in y, and e˜N(0, Iσₑ²) is a vector of residual errors. The association analysis was conducted using the PROC GLM procedure in the SAS system (SAS Institute, Cary, N.C., USA). The additive effect was estimated as one half of two homozygous markers means, and the dominance effects as the deviation of the heterozygous mean from the average of homozygous means, under the assumption of complete linkage between the marker and the candidate gene.

In total, 64 AFLP primer combinations with eight EcoRI primers and eight TaqI primers were employed in a genome-wide screening of QTL linked markers for beef marbling on two-high and two-low BMS DNA pools derived from a population of Wagyu x Limousin F₂ crosses. Each primer combination generated about 30-120 clearly scorable fragments with a size range of 75-500 bp. The primer combination of E+AAC/T+ACT and E+AAG/T+ACT yielded more fragments (˜100) than other primer combinations. Fluorescence may be a reason affecting the fragments numbers, since it was found that PET labeled primers yielded the least fragments among the four types of fluorescenly labeled primers. Analysis using GeneMapper3.7 (Applied Biosystems) demonstrated ten potential AFLP markers with the most striking visual differences in terms of peak height between high and low performance pools. These markers can be classified into two categories. One category of markers is that they are present in both high pools but absent in both low pools or vice versa, such as primer combinations E+ACA/T+CAC (FIG. 1A as an example), E+AGA/T+ACT, E+ACA/T+AAC, E+AAC/T+ACA and E+AAG/T+AAC. Another category of markers showed the differences in peak heights: the peaks in both high pools are remarkably higher than those in both low pools, or vice versa, such as primer combinations E+AAG/T+CAT (FIG. 1B as an example), E+AGT/T+ACT, E+ATC/T+ACT, E+AGT/T+CAT, and E+ATC/T+CAC.

In order to exclude any false positive markers, individual AFLP analyses were performed on the 24 top and 24 bottom-marbling samples. Among the ten markers identified above, only four consistently showed differences in AFLP fragment frequencies between high and low groups of animals (Table 1). Fragments derived with primer combinations E+AAC/T+ACA, E+AGT/T+CAT and E+AGT/T+ACT were significantly different (P<0.01) between high and low marbling groups. In comparison, Fisher's exact test revealed that the difference between high and low animals only approached significance (P<0.1) when the primer combination E+AAG/T+CAT was used. All four AFLP fragments were excised from a 5% polyacrylamide gel, re-amplified and sequenced. All primer combinations, except E+AAC/T+ACA produced products that generated readable sequences. Among the three readable sequences, BLAST search indicated that most of the sequence from the E+AAG/T+CAT marker was SINE related and could not be used as a marker and was subsequently discarded. Products amplified with primer combinations E+AGT/T+CAT and E+AGT/T+ACT each hit a bovine genomic contig, respectively.

TABLE 1
Selection of AFLP markers based on fragment frequency
between high and low performance animals.

| | Fragments Size | High | Low | Fisher's |
|---|---|---|---|---|
| Primer Combination | (base pairs) | group | group | exact test |
| E + AAC/T + ACA | 251 | 0.08 | 0.23 | p < 0.01 |
| E + AAG/T + CAT | 256 | 0.67 | 0.54 | p < 0.1 |
| E + AGT/T + CAT | 239 | 0.5 | 0.19 | p < 0.01 |
| E + AGT/T + ACT | 260 | 0.21 | 0.02 | p < 0.01 |

BLAST search using the sequence derived from the primer combination E+AGT/T+CAT retrieved a contig Ctg87.CH240-46804 (GenBank accession number: AAFC03071397) from the 7.15× bovine genome sequence database. Both EcoRI and TaqI restriction enzymes make cuts for a fragment of 217 bp in length (FIG. 2A), which corresponded exactly to this AFLP marker of 239 bp (217 bp+22 bp) identified in the gel when extra adaptor sequence of 22 bp was included in the product (Table 1). The bovine genomic contig Ctg87.CH240-46804 was then found to be homologous to human genomic contig AP001725, containing dopey family member 2 (DOPET2) on HSA21q22.2. The current draft map of the bovine genome (NCBI builder 3.1), which is incorporated herein by reference, indicated that the bovine ortholog of human DOPEY2 is located on BTA1 at position 137.84 Mb, (http://www.ncbi.nlm.nih.gov/projects/genome/guide/cow/). A pair of primers was designed to amplify this AFLP marker, but sequencing of PCR products on 6 F1 Wagyu x Linmousin bulls failed to show any mutations at either EcoRI or TaqI cut sites. Instead, a C/T transition occurred at the 2^{nd} extended base for the selective TaqI primer, which certainly caused the AFLP polymorphism at the locus (FIG. 2B).

FIG. 3 illustrates both the AFLP (E+AGT/T+ACT) marker sequence and its flanking sequence with primers designed to further characterize the AFLP fragment. The flanking sequence was simply extracted from a bovine genomic contig-Con118216 (GenBank accession number AAFC02113318, which is incorporated herein by reference). Both EcoRI and TaqI restriction enzyme recognition sites were clearly identified in the fragment (FIG. 3A), which span a sequence of 238 bp in length. By adding 22 bp of adaptor sequence to the enzyme cut fragment, the total length exactly matched the AFLP size of 260 bp (22+238 bp) identified on gels (Table 1). BLAST search found the bovine genomic contig-Con118216 (which is incorporated herein by reference) is orthologous to a human genomic sequence with GenBank accession number AL158036, which is incorporated herein by reference, which contains a novel gene KIAA1462 on HSA10p11.23. In-silico mapping could place this AFLP marker or the bovine genomic contig to a region of 45.589 and 46.63 cM on bovine chromosome 13 (BTA13). Sequencing analysis of the amplified products spanning the AFLP (E+AGT/T+ACT) marker on six F1 Wagyu x Limousin bulls revealed a single nucleotide polymorphism (SNP) at the TaqI cut site, but nothing at the EcoRI cut site (FIG. 3B). Interestingly, an additional SNP was also detected within the selective primer-extended region beside the TaqI cut site. Therefore, two G/A SNPs are responsible for the AFLP at this locus (FIG. 3B).

As the AFLP marker derived from primer combination E+AGT/T+CAT on BTA1 is caused by a C/T substitution that is not located in the enzyme recognition site (FIG. 2B), this marker was genotyped on animals using a DNA sequencing approach. As indicated above, two SNPs were responsible for the AFLP marker derived from primer combination E+AGT/T+ACT on BTA13 (FIG. 3B). Obviously, the G/A substitution within the TaqI restriction site could be genotyped with the TaqI enzyme. Fortunately, the G/A substitution within the primer extension region could be revealed by digestion with restriction enzyme MspI. Initially, both SNPs were genotyped on 30 high and 30 low BMS individuals, however, it was found that the SNP with TaqI cut site was not very informative between the high and low BMS individuals. Therefore, restriction enzyme MspI was used to genotype 246 F2 individuals by PCR-RFLP (restriction fragment length polymorphisms).

The association analysis was carried out using the data from all individuals, based on an animal model with the marker as the fixed effect variable and residual individual genetic effect as the random effect variable. The latter was included in the model in order to account for effects of genes other than the one under investigation. Based on the F statistics constructed for the fixed effects, both AFLP markers were significantly associated with BMS (AFLP marker on BTA1, F=3.62, P=0.0284 and AFLP marker on BTA13, F=4.68, P=0.0102), but not with SFD (AFLP marker on BTA1, F=0.79, P=0.4550 and AFLP marker on BTA13, F=0.500, P=0.610), respectively.

Least square means of BMS for the AFLP marker on BTA1 were estimated to be 5.683±0.149, 5.935±0.131, 6.250±0.204 for genotypes CC, CT and TT, respectively. The cattle with the homozygote (TT) genotype had an additional 0.567 score of marbling compared to the CC homozygotes (P<0.05). Candidate gene effects were estimated under the assumption of complete linkage. The additive effect of this BTA1 AFLP marker on BMS was estimated to be 0.2777±0.1075 (P=0.0105). However, estimated dominance effect of this AFLP marker on BMS was not significant (P>0.05) (Table 2).

TABLE 2
Additive and dominance effects of
both AFLP markers on BMS and SFD.

| | | | | | |
|---|---|---|---|---|---|
| | Genetic | | Standard | | |
| Traits | effect | Estimate | Error | t Value | Pr > \|t\| |

AFLP marker on BTA1

| | | | | | |
|---|---|---|---|---|---|
| BMS | Additive | 0.2777 | 0.1075 | 2.58 | 0.0105 |
| | Dominance | 0.0083 | 0.0713 | 0.12 | 0.9077 |
| SFD | Additive | 0.0085 | 0.0145 | 0.58 | 0.5612 |
| | Dominance | −0.0062 | 0.0096 | −0.68 | 0.4966 |

AFLP marker on BTA13

| | | | | | |
|---|---|---|---|---|---|
| BMS | Additive | 0.5437 | 0.2130 | 2.55 | 0.0114 |
| | Dominance | −0.1582 | 0.2445 | −0.65 | 0.5183 |
| SFD | Additive | −0.0330 | 0.0349 | −0.95 | 0.3452 |
| | Dominance | 0.0184 | 0.0400 | 0.46 | 0.6463 |

The same trend was observed in the AFLP marker on BTA13. Estimated marker means of BMS for the AFLP marker were 5.807±0.072, 6.192±0.155, 6.894±0.414 for genotypes GG, GA and AA, respectively. Obviously, the AA genotype was associated with significantly higher marbling score than the GG genotypes (P<0.05). The additive effect on BMS was estimated to be 0.5437±0.2130, which was close to the highly significant threshold level (P=0.0114) (Table 2). However, estimated dominance effect on BMS was not significant (P>0.05) (Table 2). These results would strongly suggest that both AFLP markers on BTA1 and BTA13 affected BMS in an additive genetic mode.

### Example 2

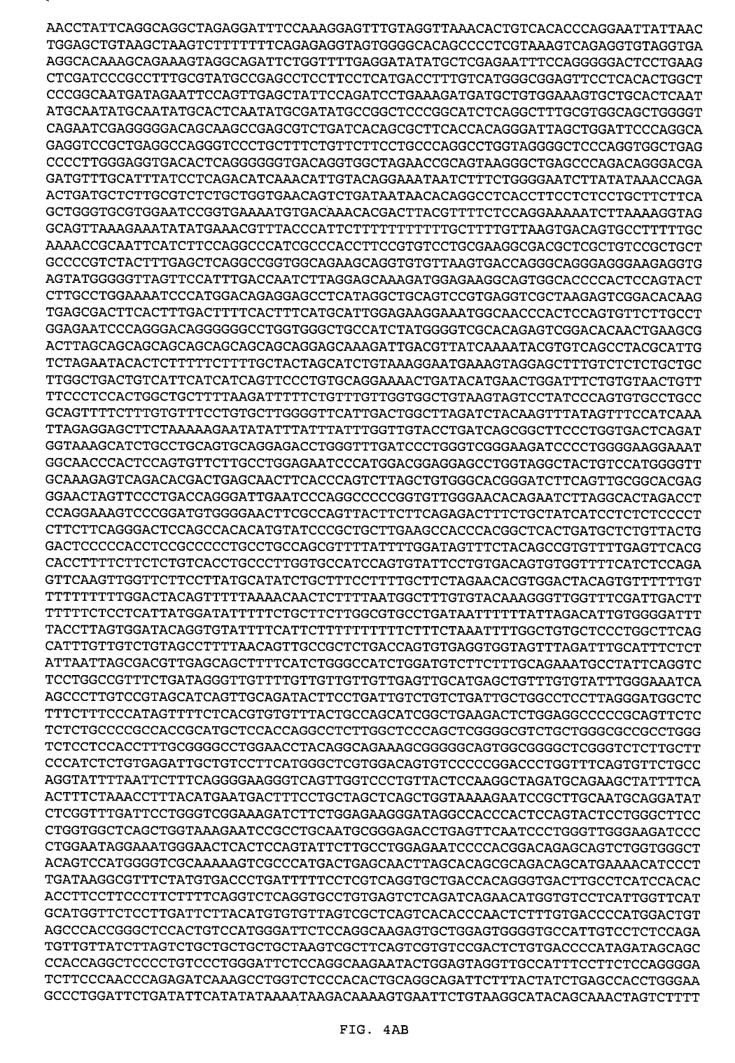

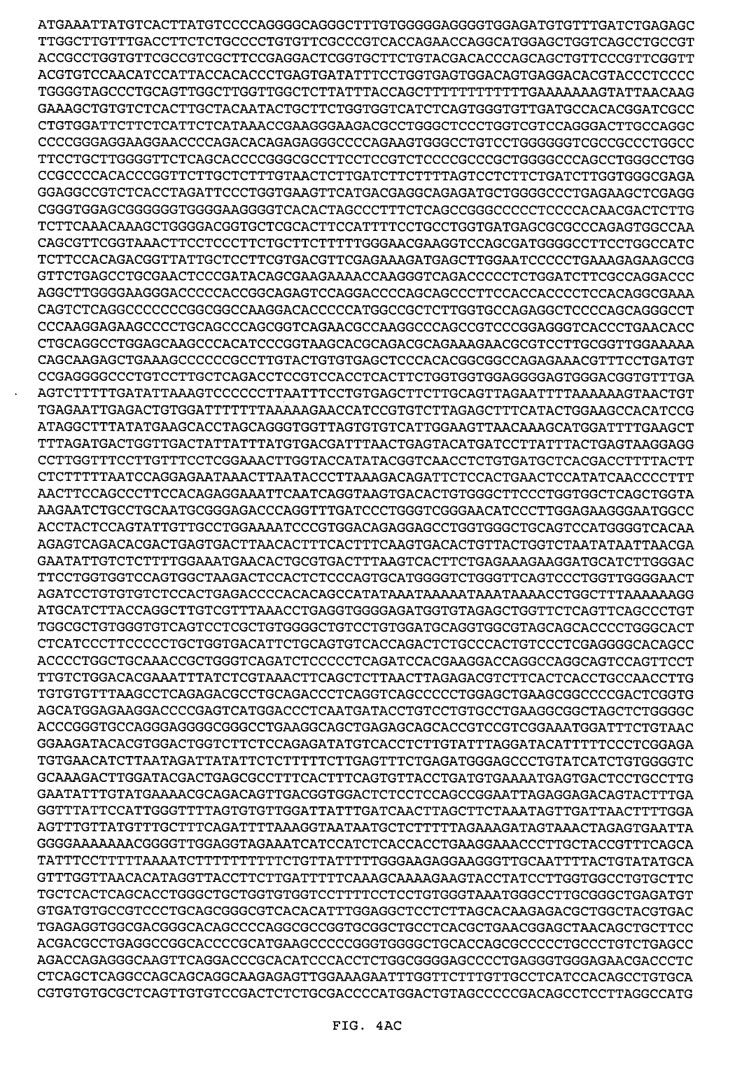

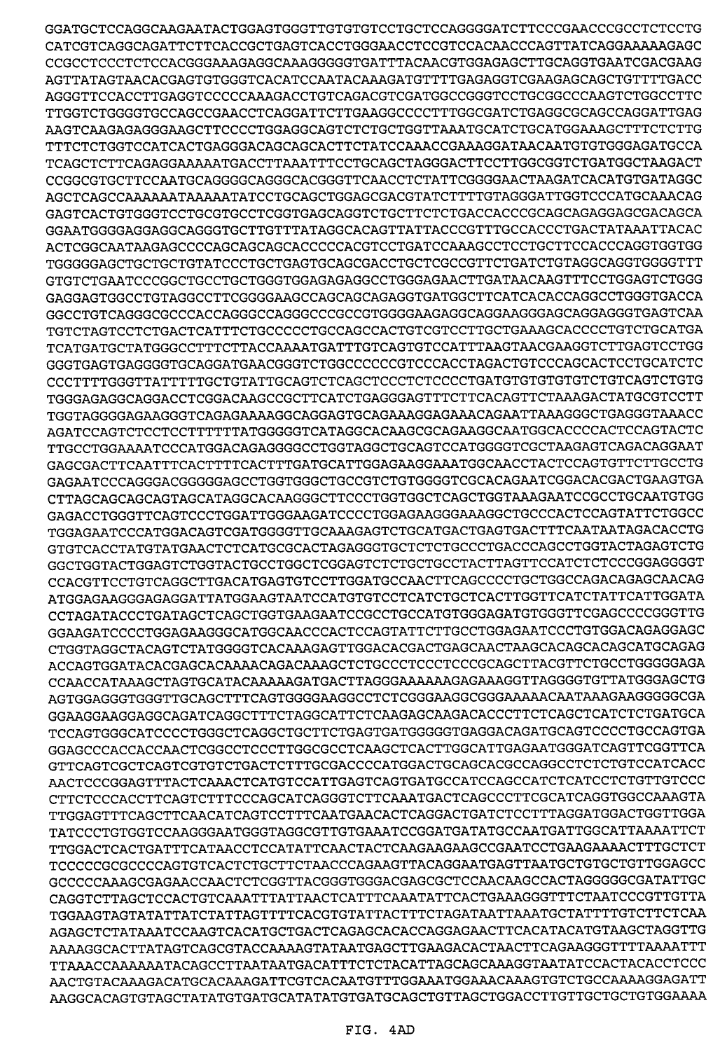

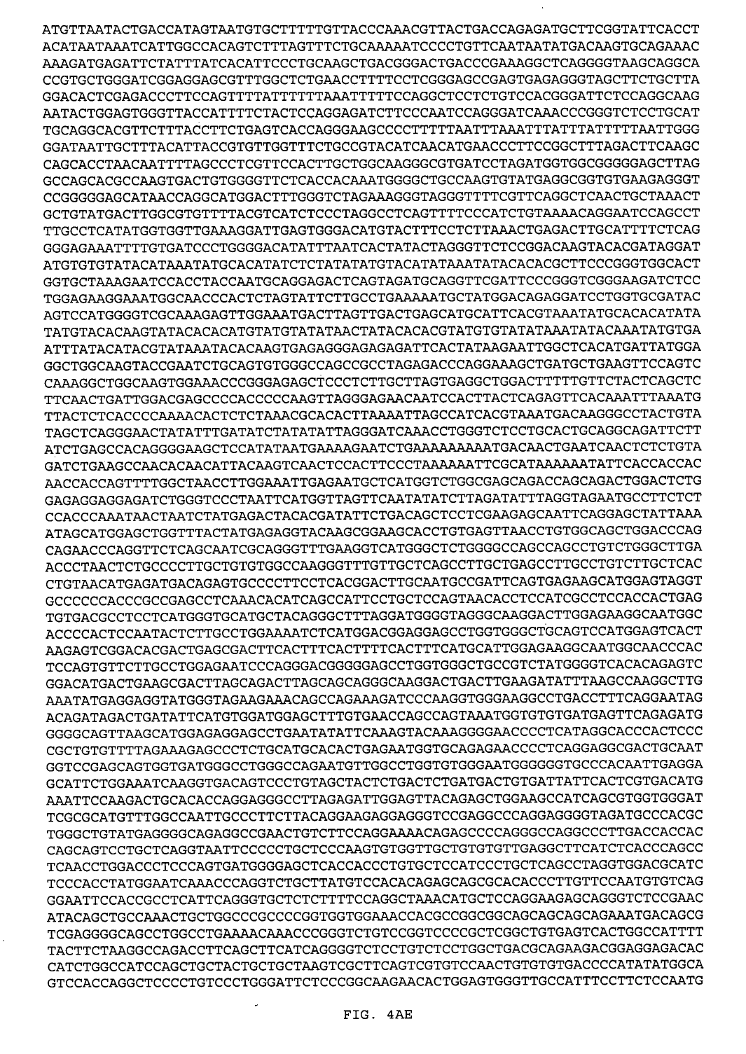

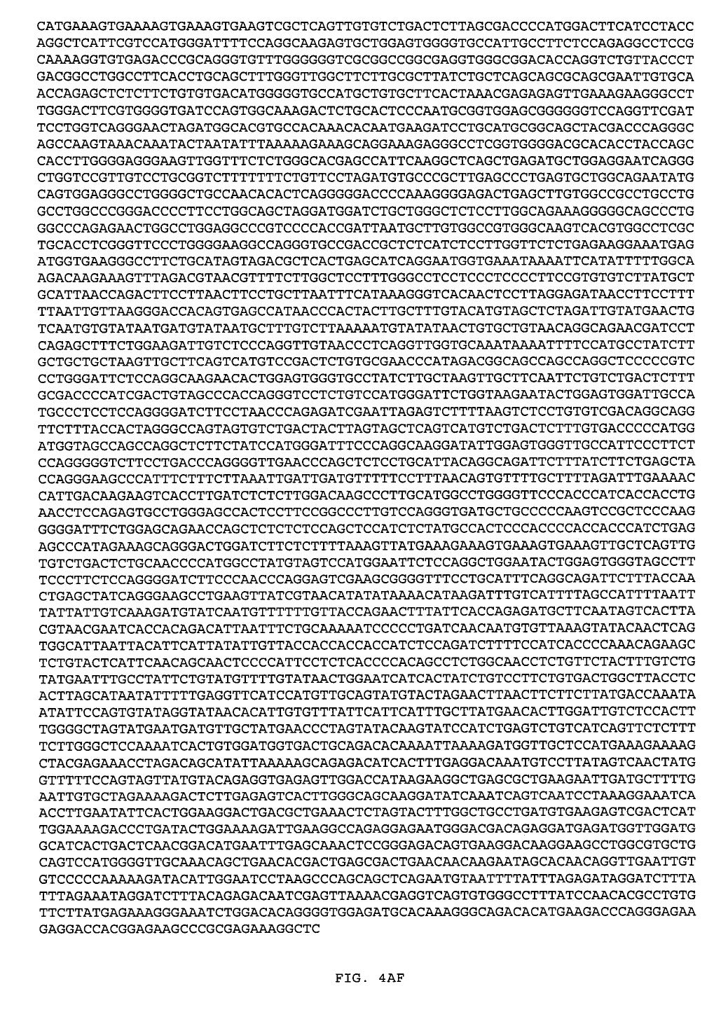

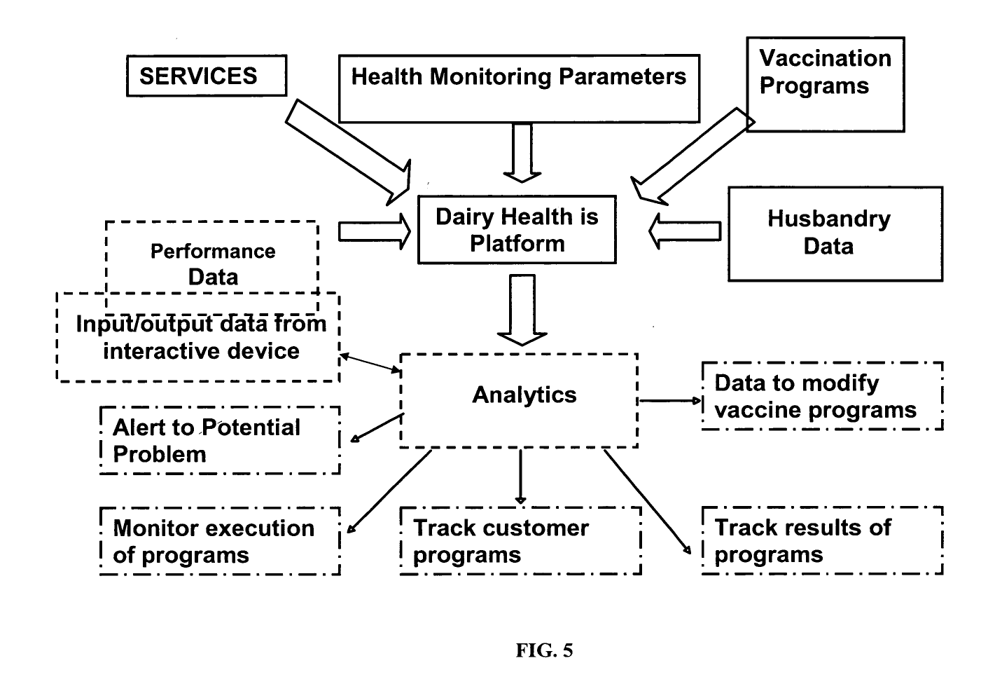

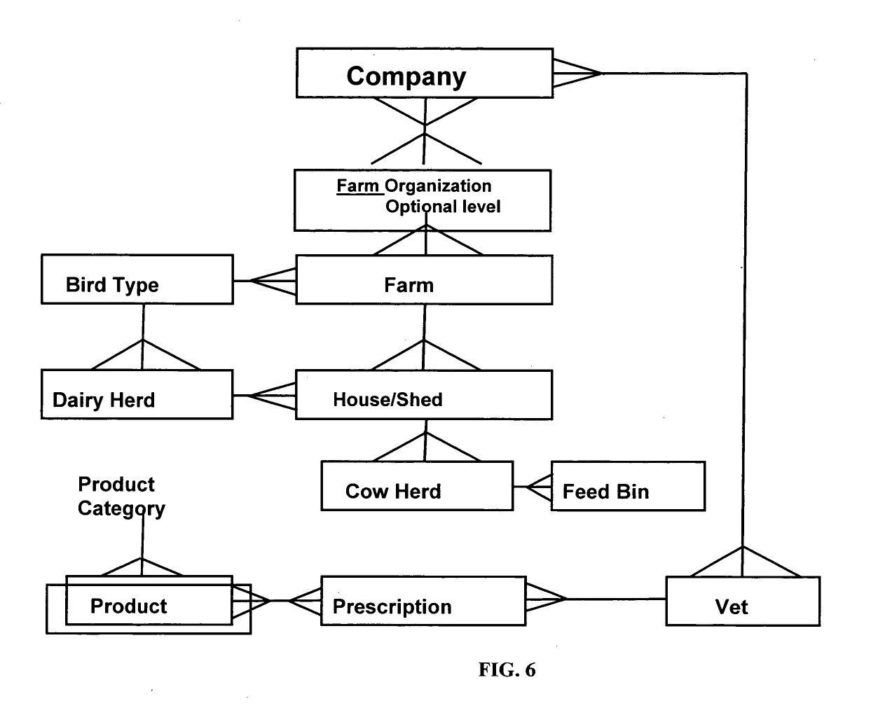

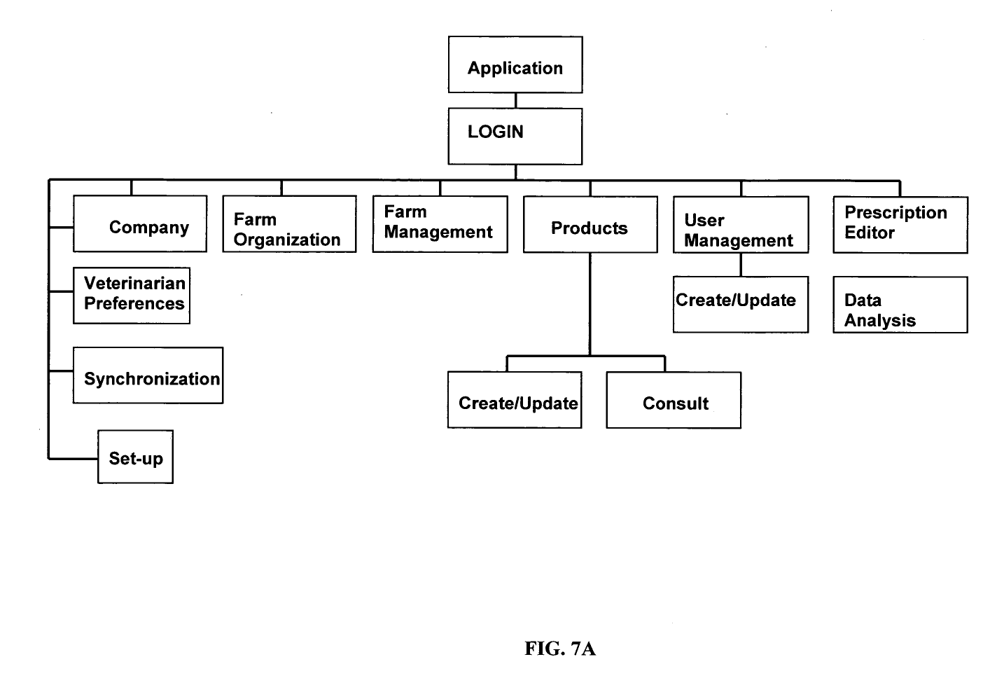

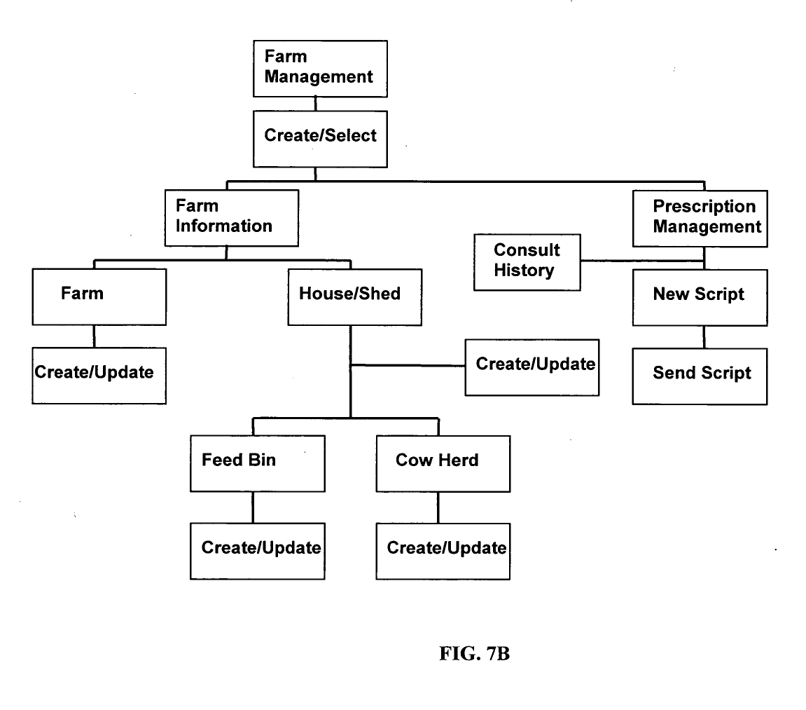

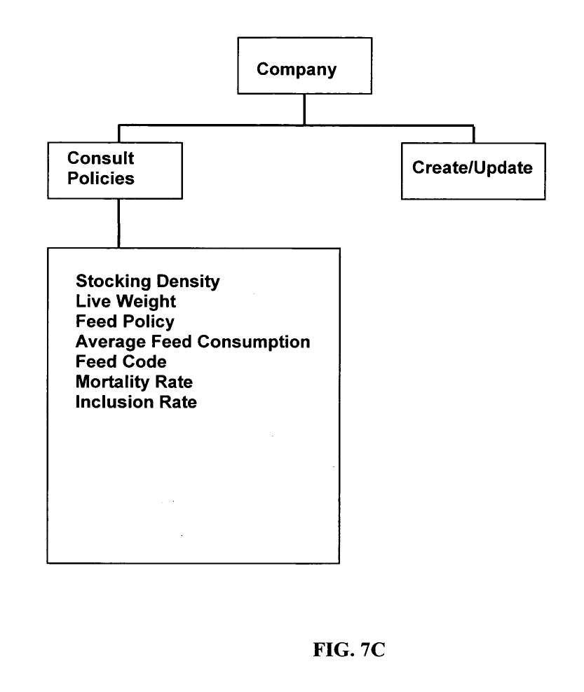

FIG. 5 shows a flowchart of the input of data and the output of results from the analysis and correlation of the data pertaining to the breeding, veterinarian histories and performance requirements of a group of animals such as from bovines. The flowchart illustrated in FIG. 7 further indicates the interactive flow of data from the computer-assisted device to a body of students learning the use of the method of the invention and the correlation of such interactive data to present an output as a pie-chart indicating the progress of the class. The flowchart further indicates modifications of the method of the invention in accordance with the information received from the students to advance the teaching process or optimize the method to satisfy the needs of the students.

FIG. 6 illustrates potential relationships between the data elements to be entered into the system. Unidirectional arrows indicate, for example, that a barn is typically owned by only one farm, whereas a farm may own several barns. Similarly, a prescription may include veterinarian products.

FIG. 7A illustrates the flow of events in the use of the portable computer-based system for data entry on the breeding and rearing of a herd of cows. FIG. 7B illustrates the flow of events through the sub-routines related to data entry concerning farm management. FIG. 7C illustrates the flow of events through the sub-routines related to data entry concerning data specific to a company.

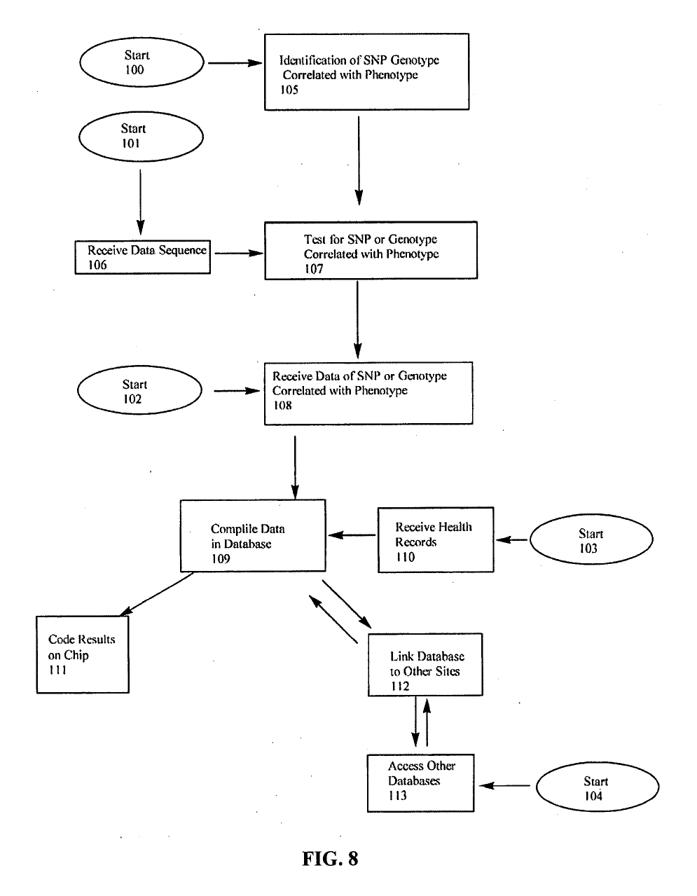

FIG. 8 illustrates a flow chart of the input of data and the output of results from the analysis and the correlation of the data pertaining to the breeding, veterinarian histories, and performance requirements of a group of animals.

The invention is further described by the following numbered paragraphs:

1. A method for sub-grouping animals according to genotype wherein the animals of each sub-group have a similar polymorphism in a DOPEY2 and/or KIAA1462 gene comprising:

(a) determining the genotype of each animal to be sub-grouped by determining the presence of a single nucleotide polymorphism in the DOPEY2 and/or KIAA1462 gene, and

(b) segregating individual animals into sub-groups wherein each animal in a sub-group has a similar polymorphism in the DOPEY2 and/or KIAA1462 gene.

2. A method for sub-grouping animals according to genotype wherein the animals of each sub-group have a similar genotype in the DOPEY2 and/or KIAA1462 gene comprising:

(a) determining the genotype of each animal to be sub-grouped by determining the presence of a single nucleotide polymorphism(s) of interest in the DOPEY2 and/or KIAA1462 gene,

(b) segregating individual animals into sub-groups depending on whether the animals have, or do not have, the single nucleotide polymorphism(s) in the DOPEY2 and/or KIAA1462 gene.

3. The method of paragraphs 1 or 2, wherein the single nucleotide polymorphism(s) of interest is selected from the group consisting of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene.

4. A method for sub-grouping animals according to genotype wherein the animals of each sub-group have a similar genotype in the DOPEY2 and/or KIAA1462 gene comprising:

(a) determining the genotype of each animal to be sub-grouped by determining the presence of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene, and

(b) segregating individual animals into sub-groups depending on whether the animals have, or do not have, AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene.

5. A method for identifying an animal having a desirable phenotype as compared to the general population of animals of that species, comprising determining the presence of a single nucleotide polymorphism in the DOPEY2 and/or KIAA1462 gene of the animal, wherein the polymorphism is selected from the group consisting of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene, wherein the single nucleotide polymorphism is indicative of a desirable phenotype.

6. The method of paragraph 5, wherein the desirable phenotype is beef marbling, subcutaneous fat or any combination thereof.

7. The method of any one of paragraphs 1 to 6 wherein the animal is a bovine.

8. The method of any one of paragraphs 1 to 7 wherein the DOPEY2 and/or KIAA1462 gene is a bovine DOPEY2 and/or KIAA1462 gene.

9. An interactive computer-assisted method for tracking the rearing of livestock bovines comprising, using a computer system comprising a programmed computer comprising a processor, a data storage system, an input device, an output device, and an interactive device, the steps of: (a) inputting into the programmed computer through the input device data comprising a breeding history of a bovine or herd of bovines, (b) inputting into the programmed computer through the input device data comprising a veterinary history of a bovine or herd of bovines, (c) correlating the veterinary data with the breeding history of the bovine or herd of bovines using the processor and the data storage system, and (d) outputting to the output device the breeding history and the veterinary history of the bovine or herd of bovines.

10. The method according to paragraph 9, wherein the computer system is an interactive system whereby modifications to the output of the computer-assisted method may be correlated according to the input from the interactive device.

11. The method according to paragraph 9 or 10, further comprising the steps of inputting into the programmed computer diagnostic data related to the health of the cow or herd of cows; and correlating the diagnostic data to the breeding and veterinary histories of the cow or herd of cows.

12. The method according to any one of paragraphs 9 to 11, wherein the veterinary data comprises a vaccination record for a cow or herd of cows.

13. The method according to any one of paragraphs 9 to 12 wherein the health data is selected from the group consisting of husbandry condition data, herd history, and food safety data.

14. The method according to any one of paragraphs 9 to 13, further comprising at least one further step selected from the group consisting of inputting into the programmed computer data related to the quality control of the bovine or herd of bovines and correlating the quality control data to the breeding and veterinary histories of the cow or herd of cows, inputting into the programmed computer performance parameters of the cow or herd of cows; and correlating the required performance parameters of the bovine or herd of bovines to a specific performance requirement of a customer, correlating the vaccine data to the performance parameters of the bovine or herd of bovines, correlating herd to the performance parameters of the bovine or herd of bovines, correlating the food safety data to the performance parameters of the bovine or herd of bovines, correlating the husbandry condition data to the performance parameters of the bovine or herd of bovines, inputting into the programmed computer data related to the nutritional data of the bovine or herd of bovines; and correlating the nutritional data to the performance parameters of the bovine or herd of bovines, and alerting to undesirable changes in the performance parameters of the bovine or herd of bovines.

15. The method according to any one of paragraphs 9 to 14, further comprising the steps of inputting into the programmed computer through the input device data comprising a genotype of a bovine; correlating a physical characteristic predicted by the genotype using the processor and the data storage system; and outputting to the output device the physical characteristic correlated to the genotype for a bovine or population of bovines, and feeding the animal(s) a diet based upon the physical characteristic, thereby improving bovine production.

16. The computer-assisted method according to any one of paragraphs 9 to 15 for optimizing efficiency of feedlots for livestock comprising outputting to the output device the breeding and veterinary history of the bovine or herd of bovines and feeding the animal(s) a diet based upon their breeding and veterinary histories, thereby optimizing efficiency of feedlots for the bovine or herd of bovines.

17. A method of transmitting data comprising transmission of information from such methods according to any one of paragraphs 9 to 15, selected from the group consisting of telecommunication, telephone, video conference, mass communication, a presentation, a computer presentation, a POWERPOINT™ presentation, internet, email, and documentary communication.

18. An interactive computer system according to any one of paragraphs 9 to 15 for tracking breeding and welfare histories of cows comprising breeding and veterinarian data corresponding to a bovine or herd of bovines, and wherein the computer system is configured to allow the operator thereof to exchange data with the device or a remote database.

19. The interactive computer system according to paragraph 18, wherein the input and output devices are a personal digital assistant or a pocket computer.

20. A method of doing business for tracking breeding and welfare histories of livestock comprising breeding and veterinarian data corresponding to one or more livestock animals comprising providing to a user the computer system of paragraph 18.

21. A method of doing business for tracking breeding and welfare histories of livestock comprising breeding and veterinarian data corresponding to one or more livestock animals comprising providing to a user the computer system of paragraph 19.

22. The method of doing business according to paragraph 20, further comprising providing the animal owner or customer with sample collection equipment, such as swabs and tags useful for collecting samples from which genetic data may be obtained, and wherein the tags are optionally packaged in a container which is encoded with identifying indicia.

23. The method of doing business according any one of paragraphs 9 to 15, wherein the computer system further comprises a plurality of interactive devices and wherein the method further comprises the steps of a receiving data from the interactive devices, compiling the data, outputting the data to indicate the response of a student or class of students to a question relating to the operation of the computer-assisted method, and optionally modifying the operation of the computer-assisted method in accordance with the indication of the response.

24. The method of any one of paragraphs 9 to 24 wherein the data comprises presence or absence of one or more of a single nucleotide polymorphism(s) of interest in the DOPEY2 and/or KIAA1462 gene.

25. The method of paragraph 24 wherein the single nucleotide polymorphism(s) is selected from the group consisting of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene.

26. A method for the diagnosis or monitoring of beef marbling and/or subcutaneous fat in a subject, comprising: obtaining a biological sample from a subject; and determining, using a suitable assay, a presence or absence in the sample of one or more DOPEY2 and/or KIAA1462 SNPs, as described herein.

27. The method of paragraph 26, wherein the subject is bovine.

28. A method for marker-assisted selection to improve beef marbling and/or subcutaneous fat, comprising screening, as part of a selection scheme, based on one or more DOPEY2 and/or KIAA1462 SNPs, as described herein, to enhance selection for beef marbling and/or subcutaneous fat.

30. The method of paragraph 29, wherein selecting is to enhance beef marbling and reduce subcutaneous fat.

31. A method of screening and mapping novel markers associated with beef marbling and/or subcutaneous fat comprising:

(a) applying an amplified fragment length polymorphism (AFLP) technique in screening of quantitative trait loci (QTL) linked markers for a complex trait on DNA pools of animals with extreme phenotypes,

(b) validating potential QTL-linked markers individually on high and low performance of animals, wherein truly significant QTL linked markers are further characterized by DNA sequencing,

(c) identifying same gene sequences of AFLP markers in the targeted species or orthologous sequences in other species and placing the AFLP markers in the targeted genome,

(d) using the flanking sequence of an AFLP marker to design primers for revealing molecular causes responsible for the amplified fragment length polymorphisms and

(e) determining the genotype assay for marker-trait association analysis.

32. The method of paragraph 31 wherein step (c) comprises in silico retrieval of flanking sequences of AFLP markers and in silico mapping of AFLP markers.

33. The method of paragraph 31 or 32 wherein the AFLP marker is derived from the primer combination E+AGT/T+CAT on BTA1.

34. The method of paragraph 31 or 32 wherein the AFLP marker is derived from the primer combination E+AGT/T+ACT on BTA13.

35. The method of paragraph 33 wherein the AFLP marker is orthologous to a novel gene DOPEY2 on HSA21q22.2.

36. The method of paragraph 34 wherein the AFLP marker is orthologous to a novel gene KIAA1462 on HSA10p11.23.

37. The method of any one of paragraphs 33 to 36 wherein in silico mapping of AFLP markers identifies candidate genes for beef marbling.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for identifying an animal having desirable beef marbling and/or subcutaneous fat, as compared to the general population of animals of that species, comprising determining the presence of one or more single nucleotide polymorphisms in a DOPEY2 and/or KIAA1462 gene of the animal, wherein the single nucleotide polymorphism is indicative of beef marbling, subcutaneous fat, or a combination thereof.

2. The method of claim 1 further comprising sub-grouping animals according to genotype, wherein the animals of each sub-group have a similar polymorphism in the DOPEY2 and/or KIAA1462 gene, said method comprising:
(a) determining the genotype of each animal to be sub-grouped by determining the presence of a single nucleotide polymorphism in the DOPEY2 and/or KIAA1462 gene, and
(b) segregating individual animals into sub-groups depending on whether the animals have, or do not have, the single nucleotide polymorphisms in the DOPEY2 and/or KIAA1462 gene.

3. The method of claim 1, wherein the single nucleotide polymorphism(s) is selected from the group consisting of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene.

4. The method of claim 1 wherein the animal is a bovine.

5. The method of claim 1 wherein the DOPEY2 and/or KIAA1462 gene is a bovine DOPEY2 and/or KIAA1462 gene.

6. An interactive computer-assisted method for tracking the rearing of livestock bovines comprising, using a computer system comprising a programmed computer comprising a processor, a data storage system, an input device, an output device, and an interactive device, the steps of: (a) inputting into the programmed computer through the input device data comprising a breeding history of a bovine or herd of bovines and a genotype of a bovine; correlating a physical characteristic predicted by the genotype using the processor and the data storage system, (b) inputting into the programmed computer through the input device data comprising a veterinary history of a bovine or herd of bovines, (c) correlating the veterinary data with the breeding history of the bovine or herd of bovines using the processor and the data storage system, and (d) outputting to the output device the breeding history, the veterinary history of the bovine or herd of bovines and the physical characteristic correlated to the genotype for a bovine or population of bovines,
wherein the physical characteristic is desirable beef marbling, subcutaneous fat, or a combination thereof, as compared to the general population of bovines, and the genotype is a single nucleotide polymorphism in a DOPEY2 and/or KIAA1462 gene.

7. The method according to claim 6, wherein the computer system is an interactive system whereby modifications to the output of the computer-assisted method may be correlated according to the input from the interactive device or
wherein the method further comprises the steps of inputting into the programmed computer diagnostic data related to the health of the cow or herd of cows; and correlating the diagnostic data to the breeding and veterinary histories of the cow or herd of cows or
wherein the veterinary data comprises a vaccination record for a cow or herd of cows or
wherein the health data is selected from the group consisting of husbandry condition data, herd history, and food safety data or
wherein the method further comprises at least one further step selected from the group consisting of inputting into the programmed computer data related to the quality control of the bovine or herd of bovines and correlating the quality control data to the breeding and veterinary histories of the cow or herd of cows, inputting into the programmed computer performance parameters of the cow or herd of cows; and correlating the required performance parameters of the bovine or herd of bovines to a specific performance requirement of a customer, correlating the vaccine data to the performance parameters of the bovine or herd of bovines, correlating herd to the performance parameters of the bovine or herd of bovines, correlating the food safety data to the performance parameters of the bovine or herd of bovines, correlating the husbandry condition data to the performance parameters of the bovine or herd of bovines, inputting into the programmed computer data related to the nutritional data of the bovine or herd of bovines; and correlating the nutritional data to the performance parameters of the bovine or herd of bovines, and alerting to undesirable changes in the performance parameters of the bovine or herd of bovines or
wherein the single nucleotide polymorphism(s) of interest is selected from the group consisting of AAFC03071397.1:g.12881G>C, g.12925G>A, g.12951T>C, g.13013A>G, g.13125G>A and g.13173C>T in the DOPEY2 gene and AAFC02113318.1:g.1367G>A and g.1372G>A in the KIAA1462 gene.

8. A method of transmitting data comprising transmission of information from such methods according to claim 6, selected from the group consisting of telecommunication, telephone, video conference, mass communication, a presentation, a computer presentation, a POWERPOINT™ presentation, internet, email, and documentary communication.

9. An interactive computer system according to claim 6 for tracking breeding and welfare histories of cows comprising breeding and veterinarian data corresponding to a bovine or herd of bovines, and wherein the computer system is configured to allow the operator thereof to exchange data with the device or a remote database.

10. The interactive computer system according to claim 9, wherein the input and output devices are a personal digital assistant or a pocket computer.

11. A method of doing business for tracking breeding and welfare histories of livestock comprising breeding and veterinarian data corresponding to one or more livestock animals comprising providing to a user the computer system of claim 9.

12. The method of doing business according to claim 11, further comprising providing the animal owner or customer with sample collection equipment, such as swabs and tags useful for collecting samples from which genetic data may be obtained, and wherein the tags are optionally packaged in a container which is encoded with identifying indicia or
wherein the computer system further comprises a plurality of interactive devices and wherein the method further comprises the steps of a receiving data from the interactive devices, compiling the data, outputting the data to indicate the response of a student or class of students to a question relating to the operation of the computer-assisted method, and optionally modifying the operation of the computer-assisted method in accordance with the indication of the response.

13. A method of screening and mapping novel markers associated with beef marbling and/or subcutaneous fat comprising:
(a) applying an amplified fragment length polymorphism (AFLP) technique in screening of quantitative trait loci (QTL) linked markers for a complex trait on DNA pools of animals with extreme phenotypes,
(b) validating potential QTL-linked markers individually on high and low performance of animals, wherein truly significant QTL linked markers are further characterized by DNA sequencing,
(c) identifying same gene sequences of AFLP markers in the targeted species or orthologous sequences in other species and placing the AFLP markers in the targeted genome,
(d) using the flanking sequence of an AFLP marker to design primers for revealing molecular causes responsible for the amplified fragment length polymorphisms and
(e) determining the genotype assay for marker-trait association analysis.

14. The method of claim 13 wherein step (c) comprises in silico retrieval of flanking sequences of AFLP markers and in silico mapping of AFLP markers.

15. The method of claim 13 wherein the AFLP marker is derived from the primer combination E+AGT/T+CAT on BTA1.

16. The method of claim 13 wherein the AFLP marker is derived from the primer combination E+AGT/T+ACT on BTA13.

17. The method of claim 15 wherein the AFLP marker is orthologous to a novel gene DOPEY2 on HSA21q22.2.

18. The method of claim 16 wherein the AFLP marker is orthologous to a novel gene KIAA1462 on HSA10p11.23.

19. The method of claim 14 wherein in silico mapping of AFLP markers identifies candidate genes for beef marbling.
